(19) United States
(12) Patent Application Publication
(10) Pub. No.: US 2006/0074103 A1
(43) Pub. Date: Apr. 6, 2006

(54) Cyclic beta-amino acid derivatives as factor Xa inhibitors

(76) Inventors: James R. Corte, Lawrenceville, NJ (US); Yun-Long Li, Wilmington, DE (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/243,242

(22) Filed: Oct. 4, 2005

Related U.S. Application Data
(60) Provisional application No. 60/616,521, filed on Oct. 6, 2004.

(51) Int. Cl. A61K 31/4743; A61K 31/4709; A61K 31/454; A61K 31/42; A61K 31/4196

(52) U.S. Cl. 514/301; 514/310; 514/314; 514/319; 514/379; 514/383; 514/323; 546/114; 546/176; 546/143; 546/205; 546/201; 548/263.2; 548/241

(57) ABSTRACT

The present application describes cyclic β-amino acid derivatives or pharmaceutically acceptable salt forms thereof, wherein the central core is a non-aromatic carbocycle or heterocycle. Compounds of the present invention are useful as inhibitors of trypsin-like serine proteases, specifically factor Xa.

### CROSS REFERENCE TO RELATED APPLICATIONS

This application claims a benefit of priority from U.S. Provisional Application No. 60/616,521 filed Oct. 6, 2004, the entire disclosure of which is herein incorporated by reference.

### FIELD OF THE INVENTION

This invention relates generally to cyclic β-amino acid derivatives that are inhibitors of trypsin-like serine protease enzymes, especially factor Xa, pharmaceutical compositions containing the same, and methods of using the same as anticoagulant agents for treatment of thromboembolic disorders.

### BACKGROUND OF THE INVENTION

Activated factor Xa, whose major practical role is the generation of thrombin by the limited proteolysis of prothrombin, holds a central position that links the intrinsic and extrinsic activation mechanisms in the final common pathway of blood coagulation. The generation of thrombin, the final serine protease in the pathway to generate a fibrin clot, from its precursor is amplified by formation of prothrombinase complex (factor Xa, factor V, Ca²⁺ and phospholipid). Since it is calculated that one molecule of factor Xa can generate 138 molecules of thrombin (Elodi, S., Varadi, K.: Optimization of conditions for the catalytic effect of the factor IXa-factor VIII Complex: Probable role of the complex in the amplification of blood coagulation. Thromb. Res. 1979, 15, 617-629), inhibition of factor Xa may be more efficient than inactivation of thrombin in interrupting the blood coagulation system.

Therefore, efficacious and specific inhibitors of factor Xa are needed as potentially valuable therapeutic agents for the treatment of thromboembolic disorders. It is thus desirable to discover new factor Xa inhibitors. In addition, it is also desirable to find new compounds with improved pharmacological characteristics compared with known factor Xa inhibitors. For example, it is preferred to find new compounds with improved factor Xa inhibitory activity and selectivity for factor Xa versus other serine proteases (i.e., trypsin). It is also desirable and preferable to find compounds with advantageous and improved characteristics in one or more of the following categories, but are not limited to: (a) pharmaceutical properties (e.g., solubility, permeability, and amenability to sustained release formulations); (b) dosage requirements (e.g., lower dosages and/or once-daily dosing); (c) factors which decrease blood concentration peak-to-trough characteristics (e.g., clearance and/or volume of distribution); (d) factors that increase the concentration of active drug at the receptor (e.g., protein binding and volume of distribution); (e) factors that decrease the liability for clinical drug-drug interactions (e.g., cytochrome P450 enzyme inhibition or induction); (f) factors that decrease the potential for adverse side-effects (e.g., pharmacological selectivity beyond serine proteases, potential chemical or metabolic reactivity, and limited CNS penetration); and, (g) factors that improve manufacturing costs or feasibility (e.g., difficulty of synthesis, number of chiral centers, chemical stability, and ease of handling).

### SUMMARY OF THE INVENTION

The present invention provides novel cyclic β-amino acid derivatives that are useful as factor Xa inhibitors or pharmaceutically acceptable salts, solvates, or prodrugs thereof.

The present invention also provides processes and intermediates for making the compounds of the present invention or a stereoisomer or a pharmaceutically acceptable salt, solvate, or prodrug form thereof.

The present invention provides pharmaceutical compositions comprising a pharmaceutically acceptable carrier and a therapeutically effective amount of at least one of the compounds of the present invention or a pharmaceutically acceptable salt, solvate, or prodrug form thereof.

The present invention provides a method for treating thromboembolic disorders comprising administering to a mammal in need of such treatment a therapeutically effective amount of at least one of the compounds of the present invention or a pharmaceutically acceptable salt, solvate, or prodrug form thereof.

The present invention provides a novel method of treating a patient in need of thromboembolic disorder treatment, comprising: administering a compound of the present invention or a pharmaceutically acceptable salt, solvate, or prodrug form thereof in an amount effective to treat a thromboembolic disorder.

The present invention provides a novel method, comprising: administering a compound of the present invention or a pharmaceutically acceptable salt, solvate, or prodrug form thereof in an amount effective to treat a thromboembolic disorder.

The present invention provides novel lactam-containing compounds and derivatives thereof for use in therapy.

The present invention provides the use of novel lactam-containing compounds for the manufacture of a medicament for the treatment of a thromboembolic disorder.

These and other objects, which will become apparent during the following detailed description, have been achieved by the inventors' discovery that cyclic β-amino acid derivatives as defined below, or pharmaceutically acceptable salt or prodrug forms thereof, are effective factor Xa inhibitors.

### DETAILED DESCRIPTION OF PREFERRED EMBODIMENTS

In a first embodiment, the present invention provides a novel compound, or a pharmaceutically acceptable salt, solvate, or prodrug form thereof, wherein the compound is selected from: 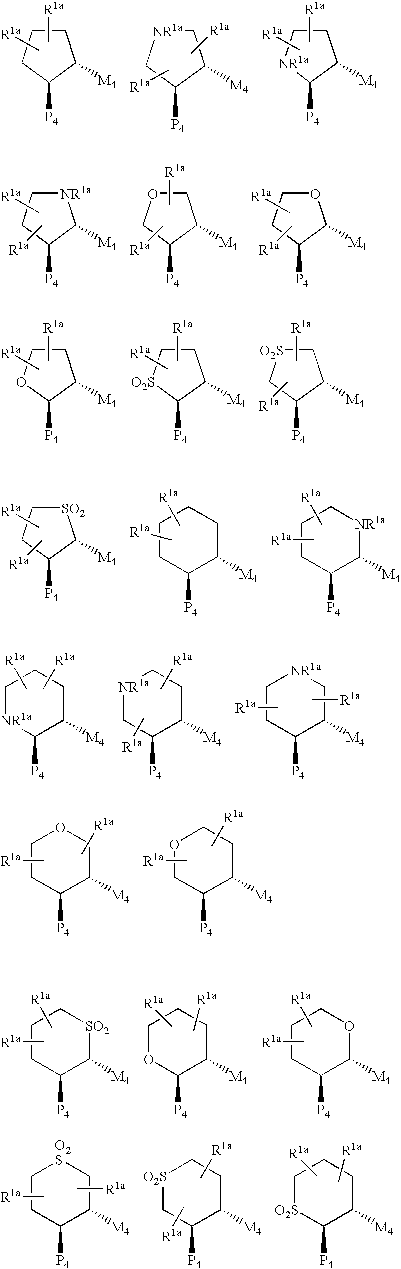 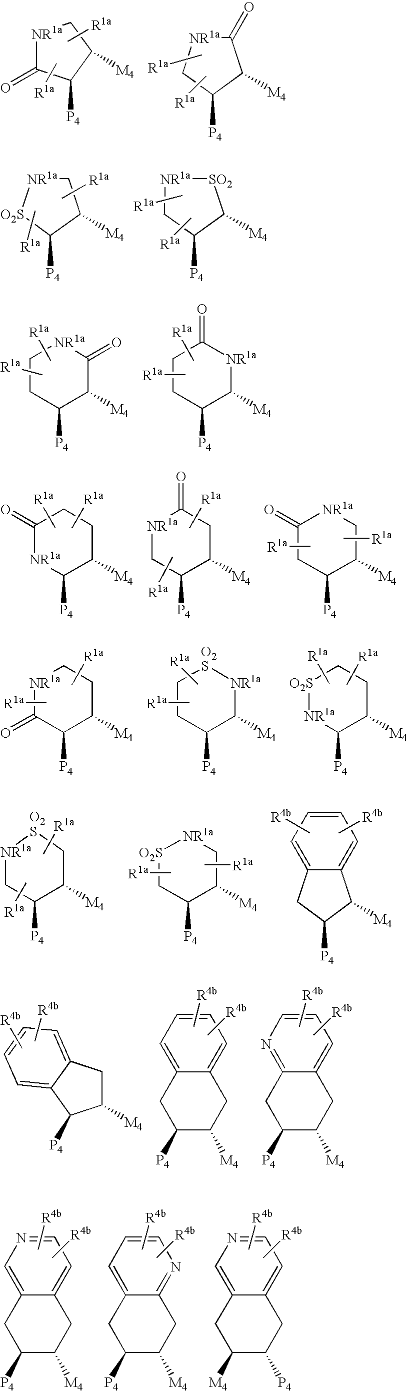 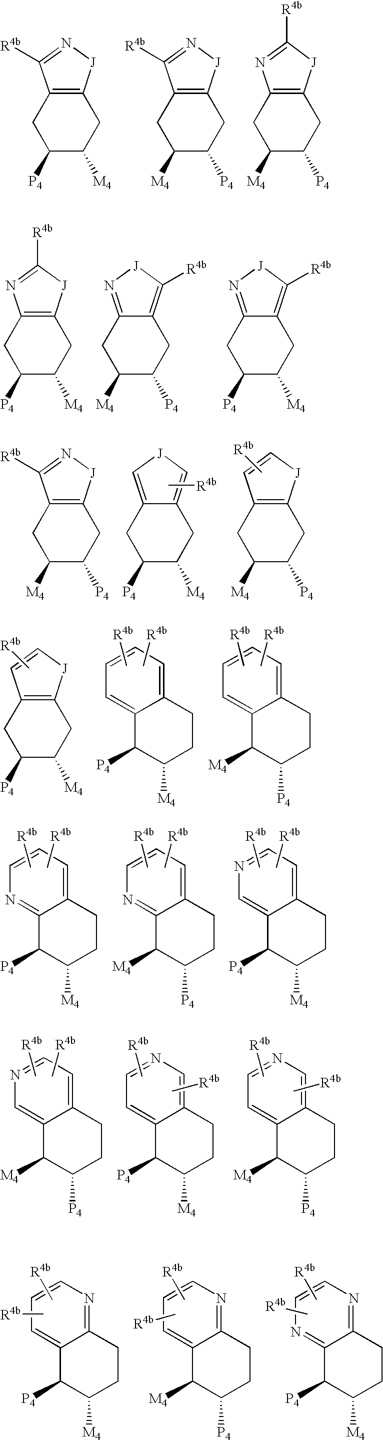 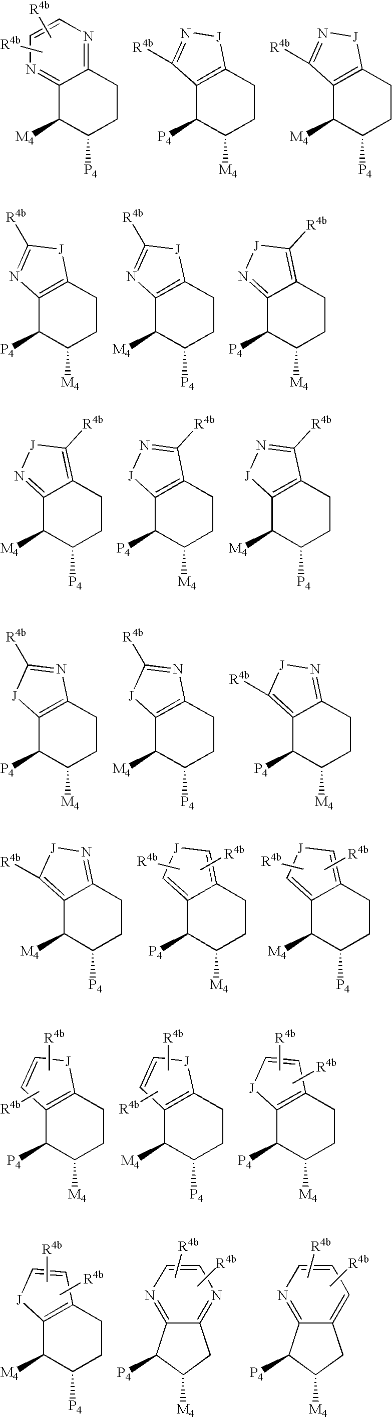 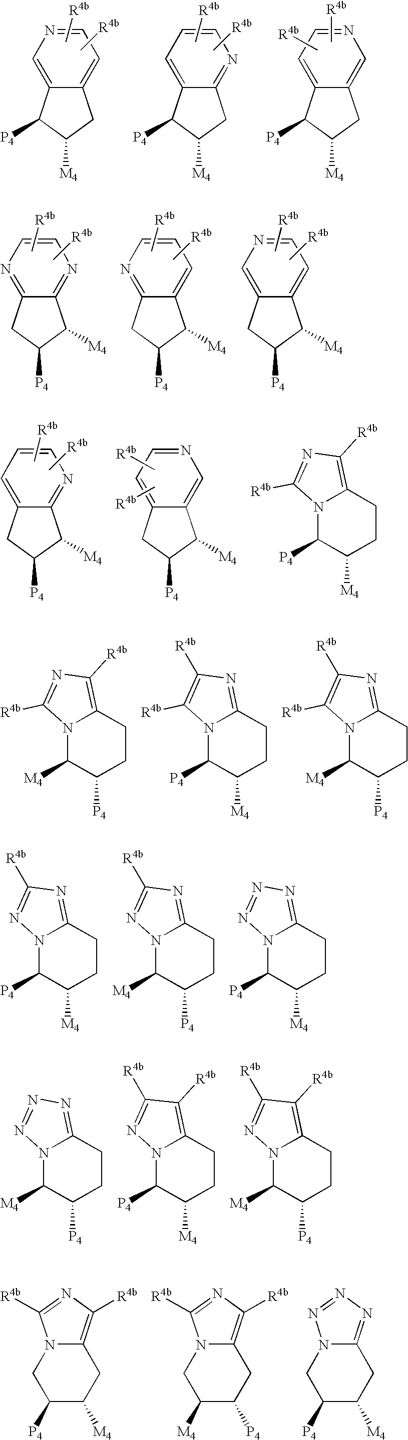 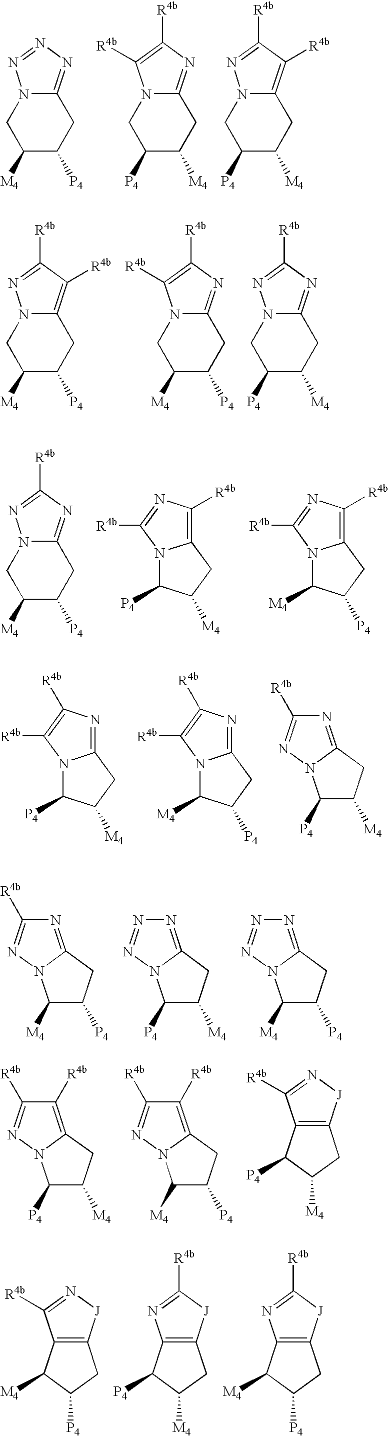 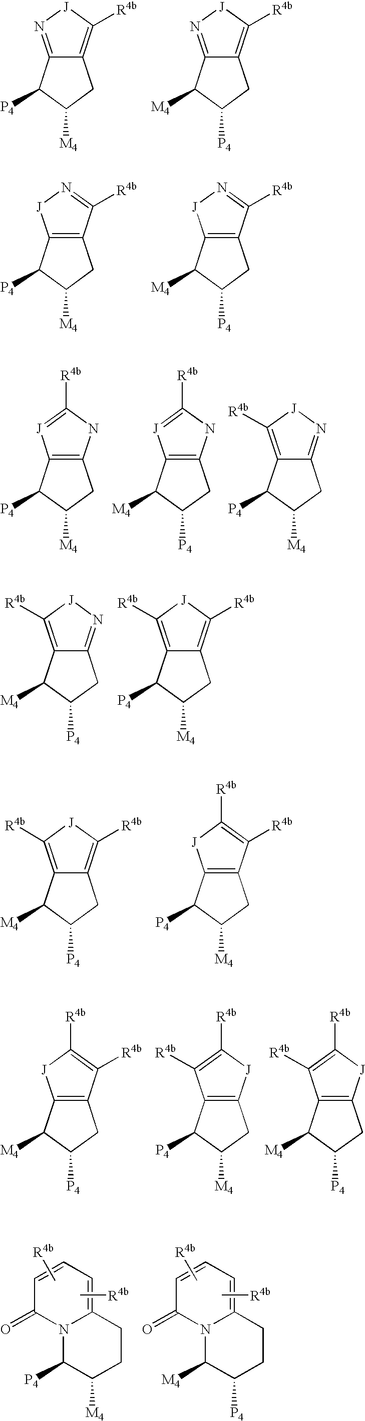 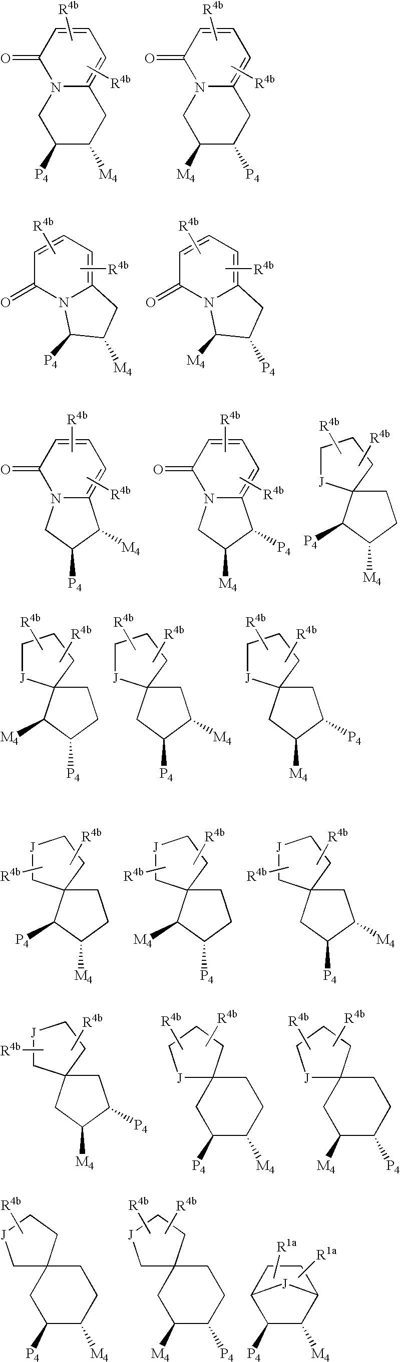 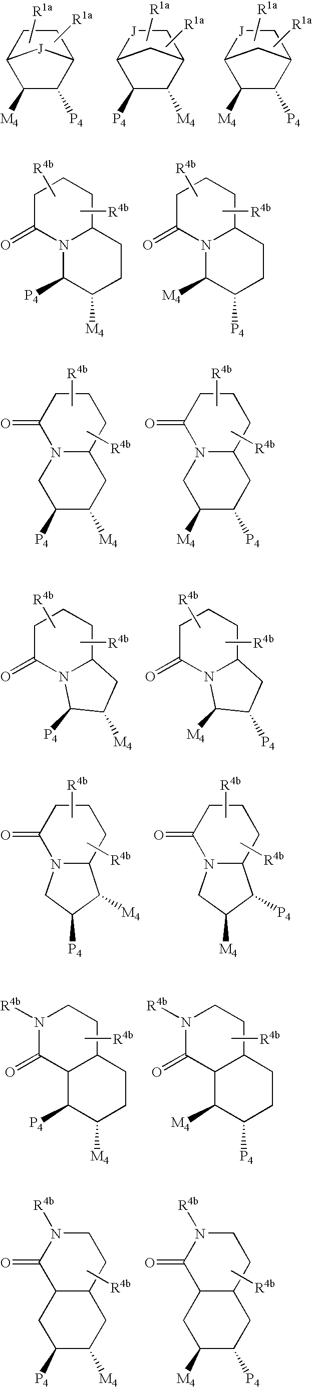 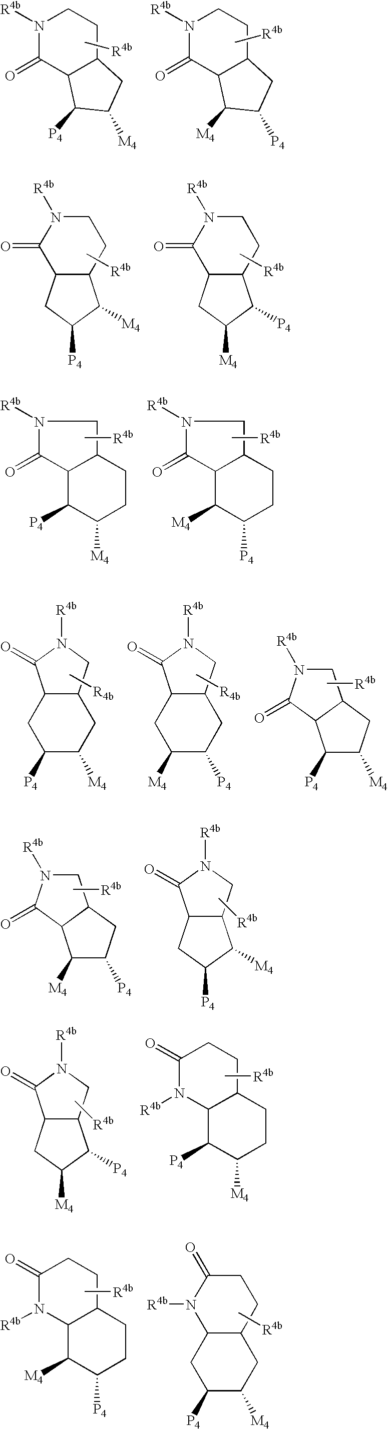 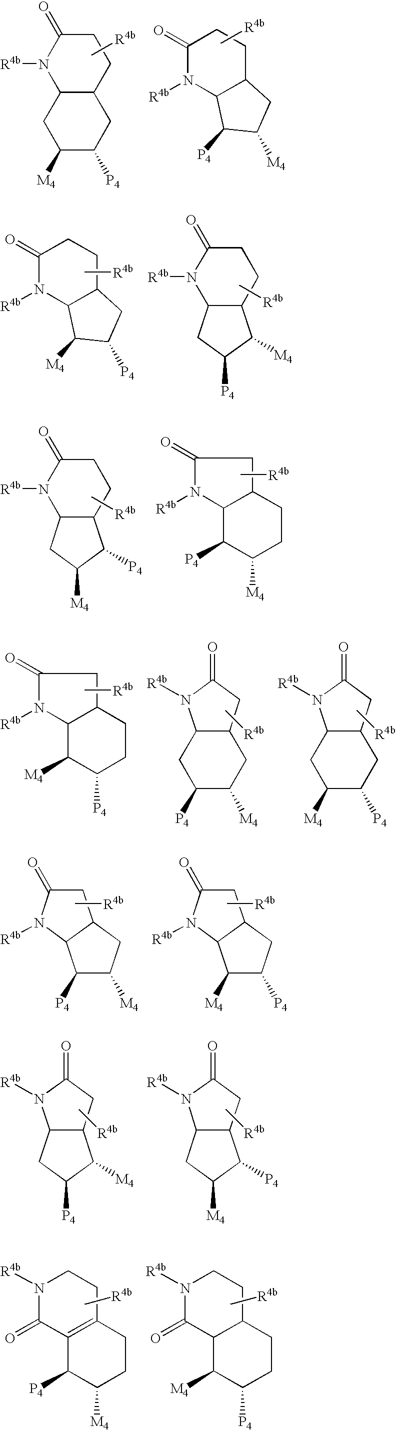 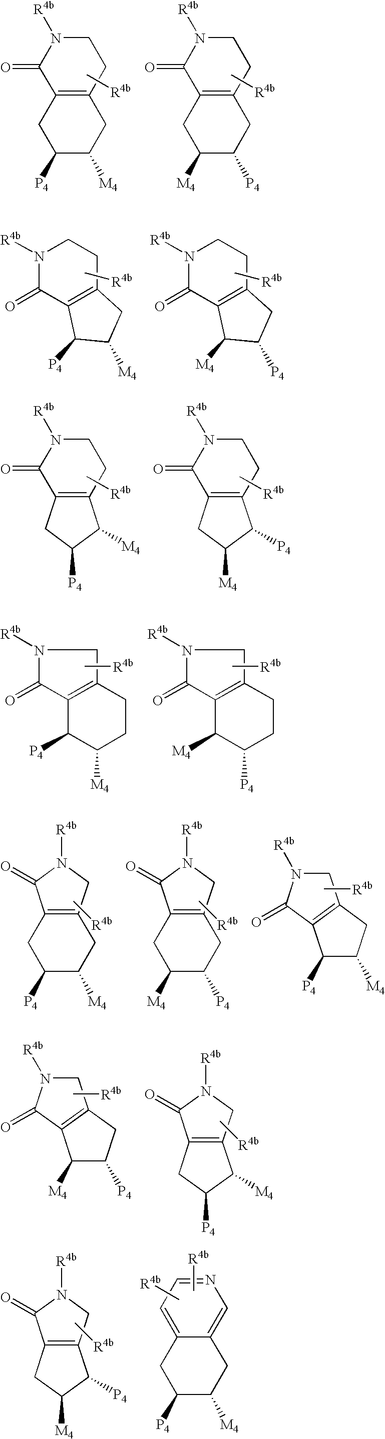 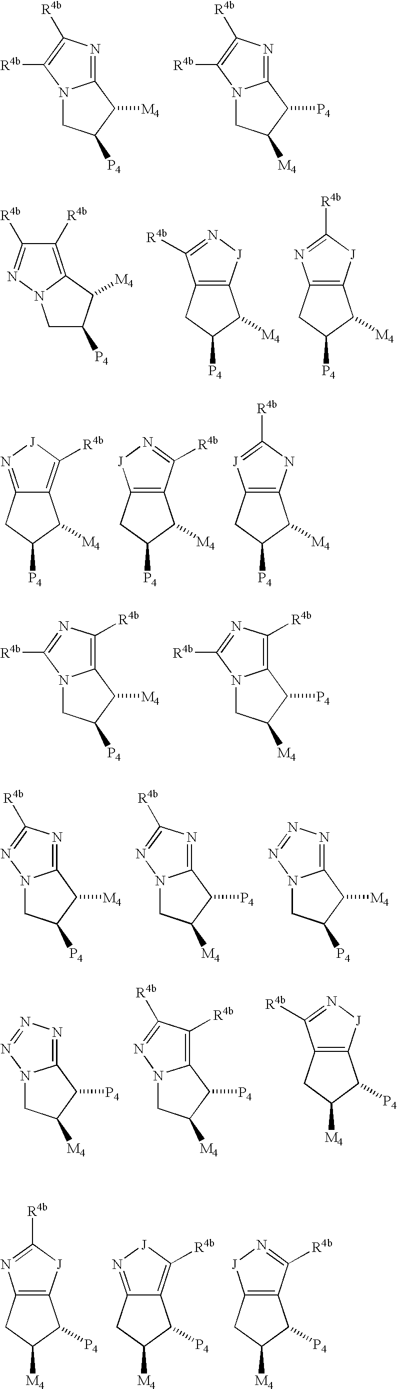 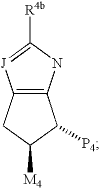
or a pharmaceutically acceptable salt thereof, wherein:

J is selected from O, S, S(O)₂, CR^{1a}, and NR₁ₐ;

one Of P₄ and M₄ is -Z-A-B and the other -G₁-G;

G is a group of formula IIa or IIb:

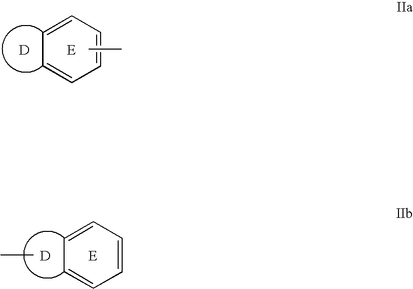

ring D, including the two atoms of Ring E to which it is attached, is a 5-6 membered ring consisting of carbon atoms and 0-2 heteroatoms selected from the group consisting of N, O, and S(O)ₚ;

ring D is substituted with 0-2 R and there are 0-3 ring double bonds;

E is selected from phenyl, pyridyl, pyrimidyl, pyrazinyl, and pyridazinyl, and is substituted with 1-3 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-3 R;

alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-2 carbonyls and 1-3 R and there are 0-3 ring double bonds;

R is selected from H, C₁₋₄ alkyl, F, Cl, Br, I, OH, OCH₃, OCH₂CH₃, OCH(CH₃)₂, OCH₂CH₂CH₃, —CN, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)2, C(═NR⁸)NR⁷R⁹, NHC(═NR⁸)NR⁷R⁹, ONHC(═NR⁸)NR⁷R⁹, NR⁸CH(═NR⁷), (CR⁸R⁹)ₜC(O)H, (CR⁸R⁹)ₜC(O)R^{2c}, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜOR³, (CR⁸R⁹)ₜS(O)ₚNR⁷R⁸, (CR⁸R⁹)ₜNR⁷S(O)ₚR⁷, (CR⁸R⁹)ₜSR³, (CR⁸R⁹)ₜS(O)R³, (CR⁸R⁹)ₜS(O)₂R³, and OCF₃, provided that S(O)ₚR⁷ and S(O)₂R³ form other than S(O)₂H or S(O)H;

alternatively, when 2 R groups are attached to adjacent atoms, they combine to form methylenedioxy or ethylenedioxy;

one of G₁ and Z is selected from CR³R^{3a}CR³R^{3a}, CR³═CR³, C≡C, CR³R^{3a}C(O), C(O)CR³R^{3a}, C(O)O, OC(O), CR³R^{3a}O, OCR³R^{3a}, CR³R^{3a}NR^{3b}, NR^{3b}CR³R^{3a}, CR³R^{3a}NR^{3e}, NR^{3e}CR³R^{3a}, C(O)NR^{3b}, NR^{3b}C(O), CR³R^{3a}S, SCR³R^{3a}, CR³R^{3a}S(O), S(O)CR³R^{3a}, CR³R^{3a}S(O)₂, S(O)₂CR³R^{3a}, S(O)NR^{3b}, NR^{3b}S(O)₂, and S(O)₂NR^{3b}, and the other of G₁ and Z is selected from CR³R^{3a}CR³R^{3a}, CR³═CR³, C≡C, CR³R^{3a}C(O), C(O)CR³R^{3a}, C(O)O, OC(O), CR³R^{3a}O, OCR³R^{3a}, CR³R^{3a}NR^{3b}, CR³R^{3a}NR^{3e}, C(O)NR^{3b}, CR³R^{3a}S, SCR³R^{3a}, CR³R^{3a}S(O), S(O)CR³R^{3a}, CR³R^{3a}S(O)₂, S(O)₂CR³R^{3a}, S(O)NR^{3b}, and S(O)₂NR^{3b}, wherein the right side of Z/G₁ is attached to ring A/ring G, provided that neither Z nor G form an N—S, NCH₂N, NCH₂O, or NCH₂S bond with either group to which it is attached;

A is selected from: C₃₋₁₀ carbocycle substituted with 0-2 R⁴, and 5-12 membered heterocycle consisting of: carbon atoms and 1-4 heteroatoms selected from the group consisting of N, O, and S(O)ₚ and substituted with 0-2 R⁴;

B is selected from Y, X—Y, N(B¹)C(O)C(R³R^{3g})₁₋₄NB²B³,C(B⁵)═NB⁴, and 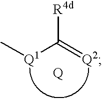
provided that Z and B are attached to different atoms on A and that the R^{4d} shown is other than OH;

B¹ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂)₀₋₂-C₃₋₇ carbocycle substituted with 0-2 R^{4b}, and —(CH₂)₀₋₂-5-6 membered heterocycle consisting of: carbon atoms and 1-4 heteroatoms selected from the group consisting of N, O, and S(O)ₚ and substituted with 0-2 R^{4b};

B² is selected from H, C₁₋₆ alkyl substituted with 0-2 R^{4c}, C(O)R^{2e}, C(O)OR^{2d}, C(O)NR^{2d}R^{2d}, C(O)NH(CH₂)₂NR^{2d}R^{2d}, SO₂NR^{2d}R^{2d}, C(O)NHSO₂-C₁₋₄ alkyl, and S(O)ₚR^{5a};

B³ is selected from H, C₁₋₆ alkyl substituted with 0-2 R^{4c}, —(CH₂)₀₋₂-3-6 membered carbocycle substituted with 0-2 R⁵, and a —(CH₂)₀₋₂-4-6 membered heterocycle consisting of: carbon atoms and 1-4 heteroatoms selected from the group consisting of N, O, and S(O)ₚ and substituted with 0-2 R⁵;

B⁴ is selected from H, SO₂R^{3b}, C(O)R^{3b}, SO₂NR³R^{3b}, C(O)NR³R^{3b}, OR², SR², —CN, and NO₂;

B⁵ is NR²R^{2f} or CR³R²R^{2f};

Q¹ and Q² are each N;

alternatively, Q¹ is CR³ and R^{4d} is NR²R^{2a} or NR^{3a}B⁴, provided that when Q¹ is CR³, then this R³ group optionally forms a ring with the R² group of R^{4d}, this ring is a 5-6 membered ring consisting of, in addition to the C—C—N shown, carbon atoms and from β-1 additional heteroatoms selected from N, O, and S(O)ₚ, and this ring is substituted with 0-1 R⁵;

ring Q is a 5-8 membered ring consisting of, in addition to the Q¹-CR^{4d}=Q² group shown, carbon atoms and 0-2 heteroatoms selected from N, O, and S(O)ₚ, and the ring is substituted with an additional 0-2 R^{4d};

Y is selected from: CY¹Y²R^{4a}, NR³R^{3a}, C(O)NR³R^{3a}, C₃₋₁₀ carbocycle substituted 0-2 R⁴ and 0-1 R^{4a}, and, 3-10 membered heterocycle consisting of: carbon atoms and 1-4 heteroatoms selected from the group consisting of N, O, and S(O)ₚ and substituted with 0-2 R⁴ and 0-1 R^{4a};

Y¹ and Y² are independently C₁₋₄ alkyl substituted with 0-2 R⁴;

X is absent or is selected from —(CR²R^{2a})₁₋₄—, —CR²(CR²R^{2b})(CH₂)ₜ—, —C(O)—, —C(═NR^{1b}), —CR²(NR^{1b}R²)—, —CR²(OR²)—, —CR²(SR²)—, —C(O)CR²R^{2a}—, —CR²R^{2a}C(O), —S(O)—, —S(O)₂—, —SCR²R^{2a}—, —S(O)CR²R^{2a}—, —S(O)₂CR²R^{2a}—, —CR²R^{2a}S(O)—, —CR²R^{2a}S(O)₂—, —S(O)₂NR²CR²R^{2a}—, —NR²S(O)₂—, —CR²R^{2a}NR²S(O)₂—, —NR²S(O)₂CR²R^{2a}—, —NR²C(O)—, —C(O)NR²CR²R^{2a}—, —NR²C(O)CR²R^{2a}—, —CR²R^{2a}NR²C(O)—, —NR²CR²R^{2a}—, and —OCR²R^{2a}—;

Z² is selected from H, S(O)₂NHR^{3b}, C(O)R^{3b}, C(O)NHR^{3b}, C(O)OR^{3f}, S(O)R^{3f}, S(O)₂R^{3f}, C₁₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-C₃₋₁₀ carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;

R^{1a}, at each occurrence, is selected from H, —(CR³R^{3a})ᵣ—R^{1b}, —(CR³R^{3a})ᵣ—CR³R^{1b}R^{1b}, —(CR³R^{3a})ᵣ—O—(CR³R^{3a})ᵣ—R^{1b}, —(CR³R^{3a})ᵣ—NR²—(CR³R^{3a})ᵣ—R^{1b}, —(CR³R^{3a})ᵣS(O)ₚ—(CR³R^{3a})ᵣ—R^{1b}, —(CR³R^{3a})ᵣ—CO₂—(CR³R^{3a})ᵣ—R^{1b}, —(CR³R^{3a})ᵣ—C(O)NR²—(CR³R^{3a})ᵣ—R^{1b}, —(CR³R^{3a})ᵣ—C(O)—(CR³R^{3a})ᵣ—R^{1b}, —C₂₋₆ alkenylene-R^{1b}, —C₂₋₆ alkynylene-R^{1b}, and —(CR³R^{3a})ᵣ—C(═NR^{1b})NR³R^{1b}, provided that R^{1a} forms other than an N-halo, N—S, O—O, or N—CN bond;

alternatively, when two R^{1a} groups are attached to adjacent atoms, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;

R^{1b} is selected from H, C₁₋₃ alkyl, F, Cl, Br, I, —CN, —NO₂, —CHO, (CF₂)ᵣCF₃, (CR³R^{3a})ᵣOR², NR²R^{2a}, C(O)R^{2b}, CO₂R^{2b}, OC(O)R², CH(CH₂OR²)₂, (CF₂)ᵣCO₂R^{2a}, S(O)ₚR^{2b}, NR²(CH₂)ᵣOR², C(═NR^{2c})NR²R^{2a}, NR²C(O)R^{2b}, NR²C(O)NR²R^{2a}, NR²C(O)₂R^{2a}, OC(O)NR²R^{2a}, C(O)NR²R^{2a}, C(O)NR²(CH₂)ᵣOR², SO₂NR²R^{2a}, NR²SO₂R², C(O)NR²SO₂R², C₃₋₆ carbocycle substituted with 0-2 R^{4b}, and 5-10 membered heterocycle substituted with 0-2 R^{4b} and consisting of carbon atoms and from 1-4 heteroatoms selected from the group consisting of N, O, and S(O)ₚ, provided that R^{1b} forms other than an O—O, N-halo, N—S, or N—CN bond and provided that S(O)ₚR² forms other than S(O)₂H or S(O)H;

R², at each occurrence, is selected from H, CF₃, C₁₋₆ alkyl, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};

R^{2a}, at each occurrence, is selected from H, CF₃, C₁₋₆ alkyl, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};

alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;

R^{2b}, at each occurrence, is selected from CF₃, C₁₋₄ alkoxy substituted with 0-2 R^{4b}, C₁₋₆ alkyl substituted with 0-2 R^{4b}, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};

R^{2c}, at each occurrence, is selected from CF₃, OH, C₁₋₄ alkoxy, C₁₋₆ alkyl, —(CH₂)ᵣ—C₃-10 carbocycle substituted with 0-2 R^{4b}, and —(CH₂)ᵣ-5-10 membered heterocycle containing from 1-4 heteroatoms selected from the group consisting of N, O, and S(O)ₚ, and substituted with 0-2 R^{4b};

R^{2d}, at each occurrence, is selected from H, R^{4c}, C₁₋₆ alkyl substituted with 0-2 R^{4c}, —(CR³R^{3a})ᵣ—C₃₋₁₀ carbocycle substituted with 0-2 R^{4c}, and —(CR³R^{3a})ᵣ-5-10 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N-C-halo, S(O)ₚ-halo, O-halo, N—S, S—N, S(O)ₚ—S(O)ₚ, S—O, O—N, O—S, or O—O moiety;

alternatively, NR^{2d}R^{2d} forms a 5-10 membered saturated or unsaturated ring substituted with 0-2 R^{4b} and consisting of: 0-1 additional heteroatoms selected from the group consisting of N, O, and S(O)ₚ;

R^{2e}, at each occurrence, is selected from H, R^{4c}, C₁₋₆ alkyl substituted with 0-2 R^{4c}, —(CR³R^{3a})ᵣ—C₃₋₁₀ carbocycle substituted with 0-2 R^{4c}, and —(CR³R^{3a})ᵣ-5-10 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;

R^{2f}, at each occurrence, is selected from H, CF₃, C₁₋₄ alkoxy substituted with 0-2 R^{4b}, C₁₋₆ alkyl substituted with 0-2 R^{4b}, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ and substituted with 0-2 R^{4b};

alternatively, CR²R^{2f} forms a 5-8 membered ring consisting of: carbon atoms and 0-2 heteroatoms selected from N, O, and S(O)ₚ, and this ring is substituted with 0-2 R^{4b};

alternatively, NR²R^{2f} forms a 5-8 membered ring consisting of: carbon atoms and 0-2 additional heteroatoms selected from N, O, and S(O)ₚ, and this ring is substituted with 0-2 R^{4b};

alternatively, when B⁴ is SO₂R^{3b} and B⁵ is NR²R^{2f}, R^{3b} and R^{2f} combine to form a 5-8 membered ring consisting of: carbon atoms and 0-2 additional heteroatoms selected from N, O, and S(O)ₚ, and this ring is substituted with 0-2 R^{4b};

alternatively, when B⁴ is C(O)R^{3b} and B⁵ is NR²R^{2f}, R^{3b} and R^{2f} combine to form a 5-8 membered ring consisting of: carbon atoms and 0-2 additional heteroatoms selected from N, O, and S(O)ₚ, and this ring is substituted with 0-2 R^{4b};

alternatively, when B⁵ is NR²R^{2f}, B⁴ and R^{2f} combine to form a 5-8 membered ring consisting of: carbon atoms and 0-2 additional heteroatoms selected from N, O, and S(O)ₚ, and this ring is substituted with 0-2 R^{4b} and the R² group of NR²R^{2f}, in addition to the groups recited below, is selected from SO₂R^{3b}, C(O)R^{3b}, and —CN;

R³, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{3a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

alternatively, R³ and R^{3a}, together with the nitrogen atom to which they are attached, combine to form a 5 or 6 membered saturated, partially unsaturated, or unsaturated ring consisting of: carbon atoms, the nitrogen atom to which R³ and R^{3a} are attached, and 0-1 additional heteroatoms selected from the group consisting of N, O, and S(O)ₚ;

R^{3b}, at each occurrence, is selected from H, C₁₋₆ alkyl substituted with 0-2 R^{1a}, C₂₋₆ alkenyl substituted with 0-2 R^{1a}, C₂-₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;

R^{3c}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;

R^{3d}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C₁₋₄ alkyl-phenyl, and C(═O)R^{3c};

R^{3e}, at each occurrence, is selected from H, SO₂NHR³, SO₂NR³R³, C(O)R³, C(O)NHR³, C(O)OR^{3f}, S(O)R^{3f}, S(O)₂R^{3f}, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;

R^{3f}, at each occurrence, is selected from: C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;

R^{3g}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂)ᵣ-3-6 membered carbocycle, and —(CH₂)ᵣ-5-6 membered heterocycle consisting of: carbon atoms and 1-4 heteroatoms selected from the group consisting of N, O, and S(O)ₚ;

alternatively, CR³R^{3g} forms a cyclopropyl group;

R⁴, at each occurrence, is selected from ═O, CHO, (CR³R^{3a})ᵣOR², (CR³R^{3a})ᵣF, (CR³R^{3a})ᵣCl, (CR³R^{3a})ᵣBr, (CR³R^{3a})ᵣI, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣC(═NR²)NR²R^{2a}, (CR³R^{3a})ᵣC(═NS(O)₂R⁵)NR²R^{2a}, (CR³R^{3a})ᵣNR²C(═NR²)NR²R^{2a}, (CR³R^{3a})ᵣC(O)NR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣNR²SO₂R⁵, (CR³R^{3a})ᵣS(O)ₚR^{5a}, (CR³R^{3a})ᵣ(CF₂)ᵣCF₃, NHCH₂R^{1b}, OCH₂R^{1b}, SCH₂R^{1b}, NH(CH₂)₂(CH₂)ₜR^{1b}, O(CH₂)₂(CH₂)ₜR^{1b}, S(CH₂)₂(CH₂)ₜR^{1b}, (CR³R^{3a})ᵣ-5-6 membered carbocycle substituted with 0-1 R⁵, and a (CR³R^{3a})ᵣ-5-6 membered heterocycle consisting of: carbon atoms and 1-4 heteroatoms selected from the group consisting of N, O, and S(O)ₚ and substituted with 0-1 R⁵;

R^{4a} is selected from C₁₋₆ alkyl substituted with 0-2 R^{4c}, C₂₋₆ alkenyl substituted with 0-2 R^{4c}, C₂₋₆ alkynyl substituted with 0-2 R^{4c}, —(CR³R^{3g})ᵣ—C₅₋₁₀ membered carbocycle substituted with 0-3 R^{4c}, —(CR³R^{3g})ᵣ-5-10 membered heterocycle substituted with 0-3 R^{4c} and consisting of: carbon atoms and 1-4 heteroatoms selected from the group consisting of N, O, and S(O)ₚ, (CR³R^{3g})ᵣCN, (CR³R^{3g})ᵣC(═NR^{2d})NR^{2d}R^{2d}, (CR³R^{3g})ᵣNR^{2d}C(═NR^{2d})NR^{2d}R^{2d}, (CR³R^{3g})ᵣNR^{2d}C(R^{2e})(═NR^{2d}), (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣNR^{2d}C(O)R^{2e}, (CR³R^{3g})ᵣ—C(O)R^{2e}, (CR³R^{3g})ᵣ—OC(O)R^{2e}, (CR³R^{3g})ᵣ—C(O)NR^{2d}R^{2d}, (CR³R^{3g})ᵣ—C(O)OR^{2d}, (CR³R^{3g})ᵣ—NR^{2d}C(O)NR^{2d}R^{2d}, (CR³R^{3g})ᵣOC(O)NR^{2d}R^{2d}, (CR³R^{3g})ᵣ—NR^{2d}C(O)OR^{2d}, (CR³R^{3g})ᵣ—SO₂NR^{2d}R^{2d}, (CR³R^{3g})ᵣ—NR^{2d}SO₂NR^{2d}R^{2d}, (CR³R^{3g})ᵣ—C(O)NR^{2d}SO₂R^{2d}, (CR³R^{3g})ᵣNR^{2d}SO₂R^{2d}, and (CR³R^{3g})ᵣ—S(O)ₚR^{2d}, provided that S(O)ₚR^{2d} forms other than S(O)₂H or S(O)H and further provided that R^{4a} is other than a hydroxamic acid;

R^{4b}, at each occurrence, is selected from H, ═O, (CH₂)ᵣOR³, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣ—C(O)NR³R^{3a}, (CH₂)ᵣNR³C(O)NR³R^{3a}, (CH₂ᵣ—C(═NR³)NR³R^{3a}, (CH₂)ᵣNR³C(═NR³)NR³R^{3a}, (CH₂)ᵣSO₂NR³R^{3a}, (CH₂)ᵣNR³SO₂NR³R^{3a}, (CH₂)ᵣNR³SO₂—C₁₋₄ alkyl, (CH₂)ᵣNR³SO₂CF₃, (CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;

R^{4c}, at each occurrence, is selected from ═O, (CR³R^{3a})ᵣOR², (CR³R^{3a})ᵣF, (CR³R^{3a})ᵣBr, (CR³R^{3a})ᵣCl, (CR³R^{3a})ᵣCF₃, C₁₋₄ alkyl, C₂₋₆ alkenyl, C₂₋₆ alkynyl, (CR³R^{3a})ᵣCN, (CR³R^{3a})ᵣNO₂, (CR³R^{3a})ᵣNR²R^{2a}, (CR³R^{3a})ᵣN(→O)R²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NR²(CH₂)₂NR²R^{2a}, (CR³R^{3a})ᵣNR²C(O)NR²R^{2a}, (CR³R^{3a})ᵣC(═NR²)NR²R^{2a}, (CR³R^{3a})ᵣNR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣC(O)NR²SO₂—C₁₋₄ alkyl, (CR³R^{3a})ᵣNR²SO₂R^{5a}, (CR³R^{3a})ᵣC(O)NR²SO₂R^{5a}, (CR³R^{3a})ᵣS(O)ₚR^{5a}, (CF₂)ᵣCF₃, (CR³R^{3a})ᵣC₃₋₁₀ carbocycle substituted with 0-2 R^{4b}, and (CR³R^{3a})ᵣ4-10 membered heterocycle substituted with 0-2 R^{4b} and consisting of carbon atoms and from 1-4 heteroatoms selected from the group consisting of N, O, and S(O)ₚ;

R^{4d}, at each occurrence, is selected from H, (CR³R^{3a})ᵣOR², (CR³R^{3a})ᵣF, (CR³R^{3a})ᵣBr, (CR³R^{3a})ᵣCl, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NH(CH₂)₂NR²R^{2a}, (CR³R^{3a})ᵣNR²C(O)NR²R^{2a}, (CR³R^{3a})ᵣC(═NR²)NR²R^{2a}, (CR³R^{3a})ᵣNHC(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣC(O)NHSO₂—C₁₋₄ alkyl, (CR³R^{3a})NR²SO₂R⁵, (CR³R^{3a})ᵣS(O)ₚR^{5a}, (CR³R^{3a})ᵣ(CF₂)ᵣCF₃, (CR³R^{3a})ᵣ-5-6 membered carbocycle substituted with 0-1 R⁵, and a (CR³R^{3a})ᵣ-5-6 membered heterocycle consisting of: carbon atoms and 1-4 heteroatoms selected from the group consisting of N, O, and S(O)ₚ and substituted with 0-1 R⁵;

R⁵, at each occurrence, is selected from H, C₁₋₆ alkyl, ═O, (CH₂)ᵣOR³, F, Cl, Br, I, —CN, NO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣCH(═NOR^{3d}), (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³SO₂NR³R^{3a}, (CH₂)ᵣNR³SO₂—C₁₋₄ alkyl, (CH₂)ᵣNR³SO₂CF₃, (CH₂)ᵣNR³SO₂-phenyl, (CH₂)ᵣS(O)ₚCF3, (CH₂)ᵣS(O)ₚ-C₁₋₄ alkyl, (CH₂)ᵣS(O)ₚ-phenyl, (CF₂)ᵣCF₃, phenyl substituted with 0-2 R⁶, naphthyl substituted with 0-2 R⁶, and benzyl substituted with 0-2 R⁶;

R^{5a}, at each occurrence, is selected from C₁₋₆ alkyl, (CH₂)ᵣOR³, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CF₂)ᵣCF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond;

R⁶, at each occurrence, is selected from H, OH, (CH₂)ᵣOR², halo, C₁₋₄ alkyl, CN, NO₂, (CH₂)ᵣNR²R^{2a}, (CH₂)ᵣC(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl;

R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₄ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₁₋₄ alkyl-C(O)—;

R⁸, at each occurrence, is selected from H, C₁₋₆alkyl, and (CH₂)ₙ-phenyl;

alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;

R⁹, at each occurrence, is selected from H, C₁₋₆ alkyl, and (CH₂)ₙ-phenyl;

n, at each occurrence, is selected from 0, 1, 2, and 3;

p, at each occurrence, is selected from 0, 1, and 2;

r, at each occurrence, is selected from 0, 1, 2, 3, 4, 5, and 6; and

t, at each occurrence, is selected from 0, 1, 2, and 3.

In a second embodiment, the present invention provides a novel compound, or a pharmaceutically acceptable salt, solvate, or prodrug form thereof, wherein the compound is selected from:

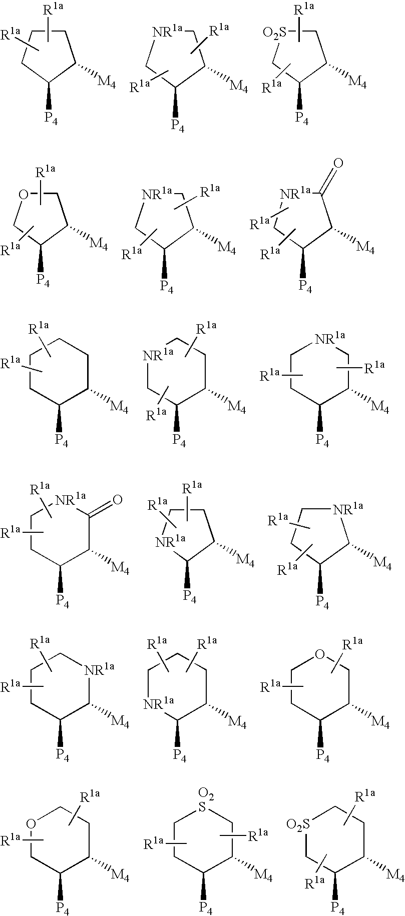

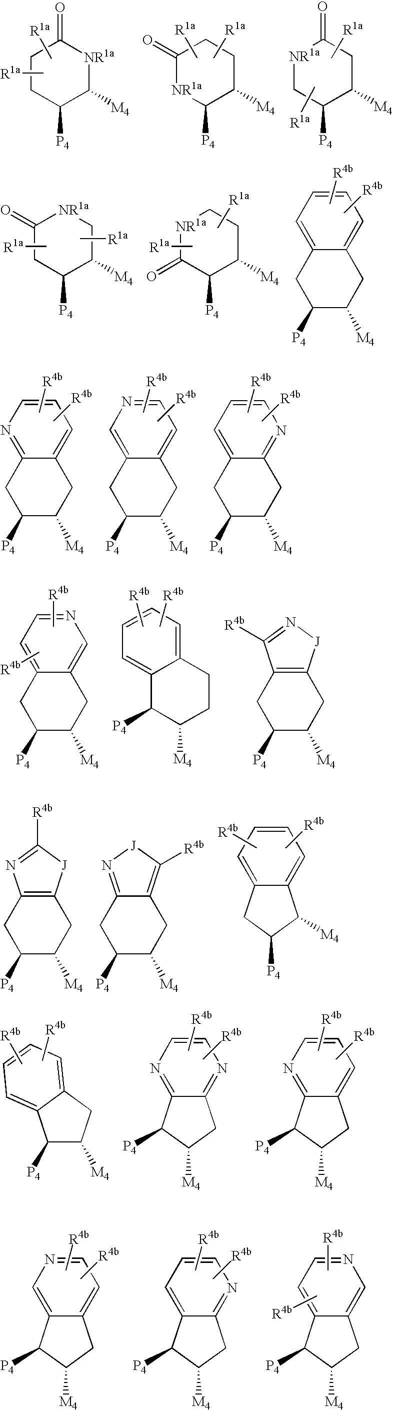

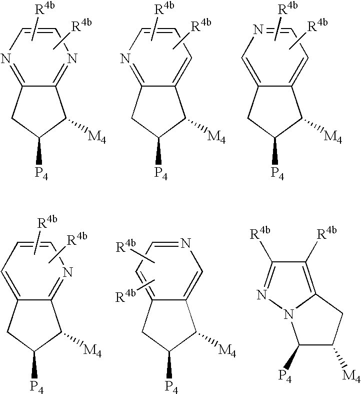

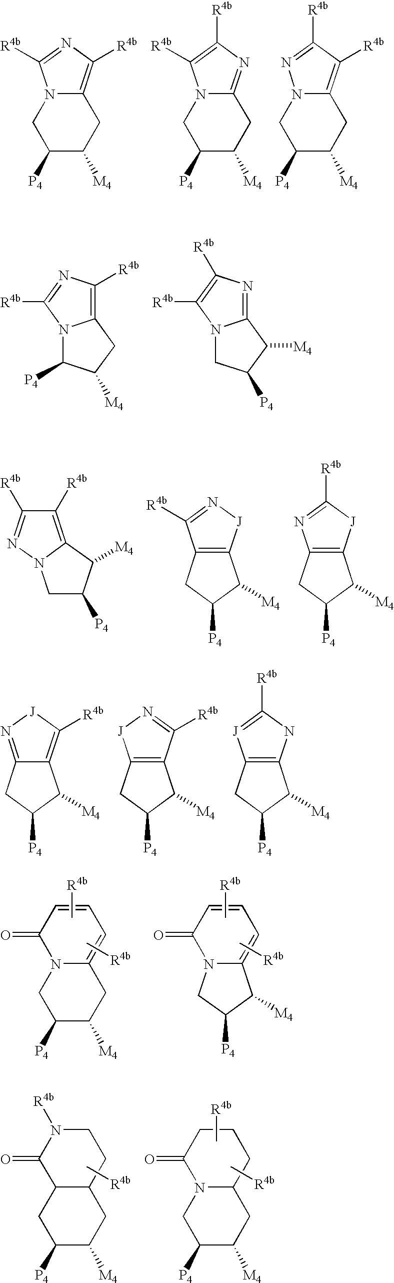

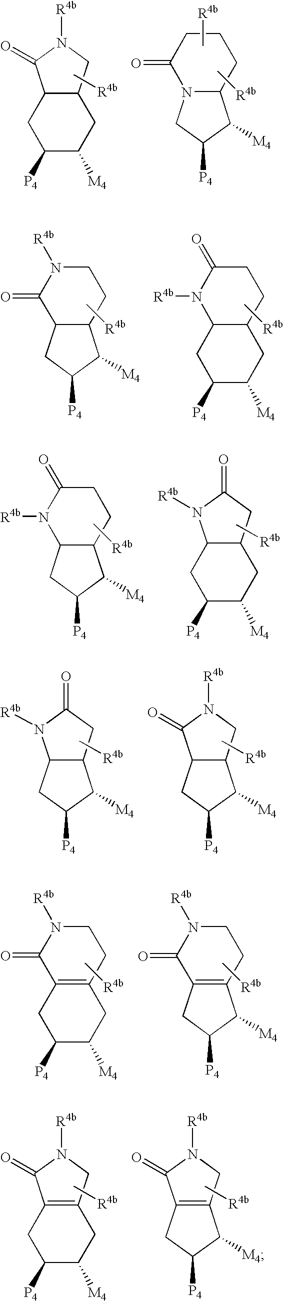

J is selected from O, S, S(O)₂, and NR^{1a};

G is selected from the group: 2-aminomethyl-4-chloro-phenyl, 2-aminosulfonyl-4-chloro-phenyl, 2-amido-4-chloro-phenyl, 4-chloro-2-methylsulfonyl-phenyl, 2-aminosulfonyl-4-fluoro-phenyl, 2-amido-4-fluoro-phenyl, 4-fluoro-2-methylsulfonyl-phenyl, 2-aminomethyl-4-bromo-phenyl, 2-aminosulfonyl-4-bromo-phenyl, 2-amido-4-bromo-phenyl, 4-bromo-2-methylsulfonyl-phenyl, 2-aminomethyl-4-methyl-phenyl, 2-aminosulfonyl-4-methyl-phenyl, 2-amido-4-methyl-phenyl, 2-methylsulfonyl-4-methyl-phenyl, 4-fluoro-pyrid-2-yl, 4-bromo-pyrid-2-yl, 4-methyl-pyrid-2-yl, 5-fluoro-thien-2-yl, 5-bromo-thien-2-yl, 5-methyl-thien-2-yl, 2-amido-4-methoxy-phenyl, 2-amido-phenyl, 2-aminomethyl-3-fluoro-phenyl, 2-aminomethyl-4-fluoro-phenyl, 2-aminomethyl-4-methoxy-phenyl, 2-aminomethyl-5-fluoro-phenyl, 2-aminomethyl-5-methoxy-phenyl, 2-aminomethyl-6-fluoro-phenyl, 2-aminomethyl-phenyl, 2-amino-pyrid-4-yl, 2-aminosulfonyl-4-methoxy-phenyl, 2-aminosulfonyl-phenyl, 2-methylsulfonyl-phenyl, 3-(N,N-dimethylamino)-4-chloro-phenyl, 3-(N,N-dimethylamino)-phenyl, 3-(N-methylamino)-4-chloro-phenyl, 3-(N-methylamino)-phenyl, 3-amido-phenyl, 3-amino-4-chloro-phenyl, 3-aminomethyl-phenyl, 3-amino-phenyl, 4-(N,N-dimethylamino)-5-chloro-thien-2-yl, 4-(N-methylamino)-5-chloro-thien-2-yl, 4-amino-5-chloro-thien-2-yl, 4-methoxy-2-methylsulfonyl-phenyl, 2-methoxy-pyrid-5-yl, 5-(N,N-dimethylamino)-4-chloro-thien-2-yl, 5-(N-methylamino)-4-chloro-thien-2-yl, 5-amino-4-chloro-thien-2-yl, 5-chloro-pyrid-2-yl, 5-chloro-thien-2-yl, 6-amino-5-chloro-pyrid-2-yl, 6-amino-pyrid-2-yl, 2-cyano-4-chloro-phenyl, 2-methoxy-4-chloro-phenyl, 2-fluoro-4-chloro-phenyl, 3-chloro-4-methyl-phenyl, 3-fluoro-4-chloro-phenyl, 3-methyl-4-chloro-phenyl, 3-fluoro-4-methyl-phenyl, 3,4-dimethyl-phenyl, 3-chloro-4-fluoro-phenyl, 3-methyl-4-fluoro-phenyl, 4-methylsulfanyl-phenyl, 2-chlorothiazol-5-yl, 5-chlorothiazol-2-yl, 3-pyridyl; 2-pyridyl, 5-fluoro-pyrid-2-yl, 5-methyl-pyrid-2-yl, 5-methoxy-pyrid-2-yl, 5-cyano-pyrid-2-yl, 2-pyrimidyl, 5-chloro-pyrimid-2-yl, 5-fluoro-pyrimid-2-yl, 5-methyl-pyrimid-2-yl, 5-methoxy-pyrimid-2-yl, 5-cyano-pyrimid-2-yl, phenyl, 3-chloro-phenyl, 3-fluoro-phenyl, 3-methyl-phenyl, 4-ethyl-phenyl, 3-methoxy-phenyl, 3-cyano-phenyl, 4-chloro-phenyl, 4-fluoro-phenyl, 4-methyl-phenyl, 4-methoxy-phenyl, 4-cyano-phenyl,

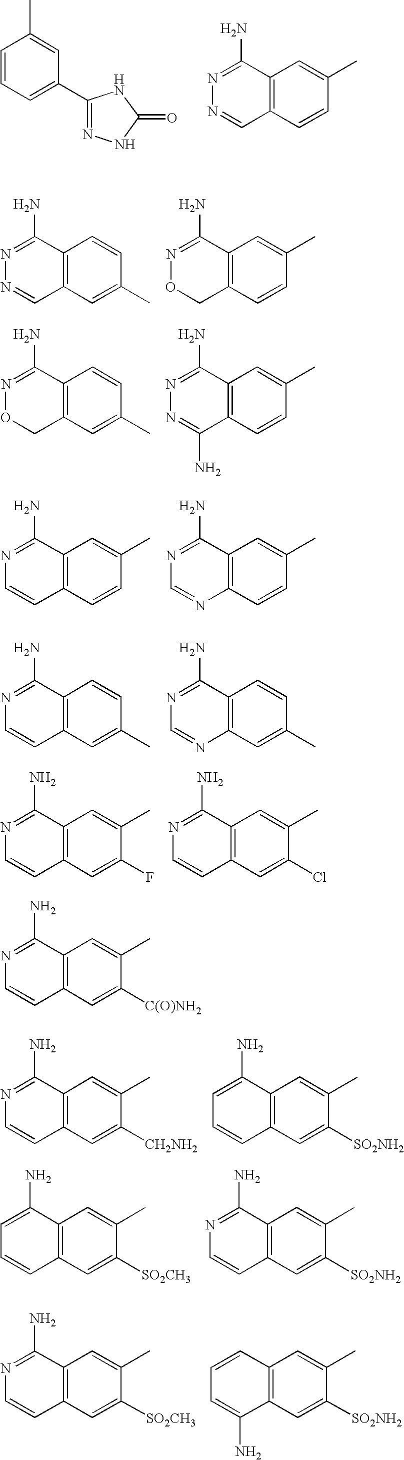

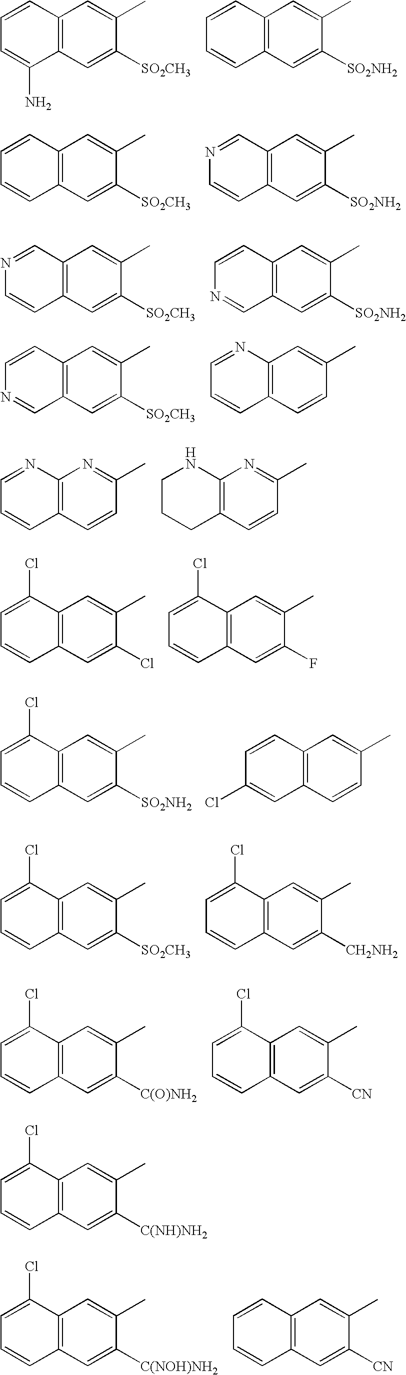

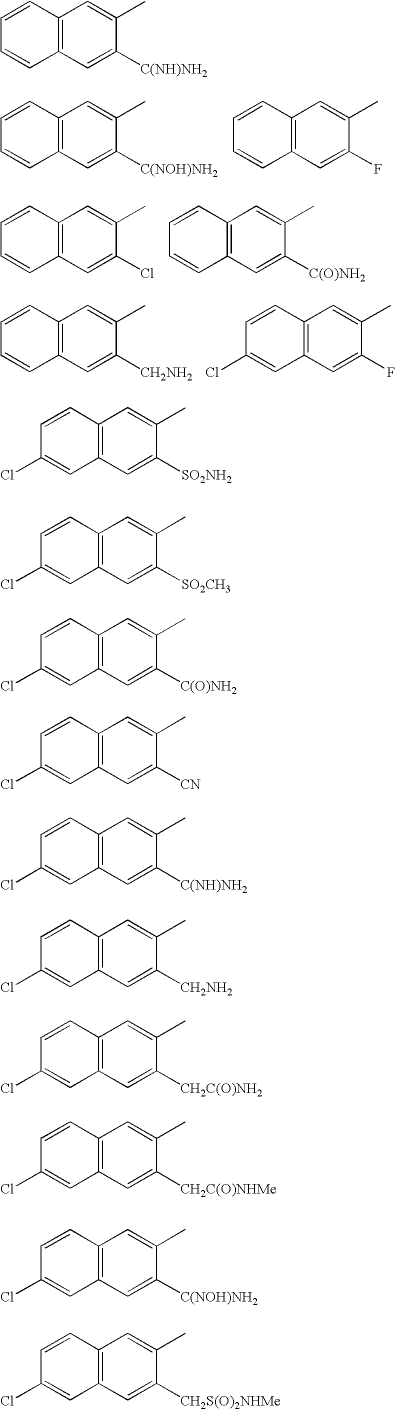

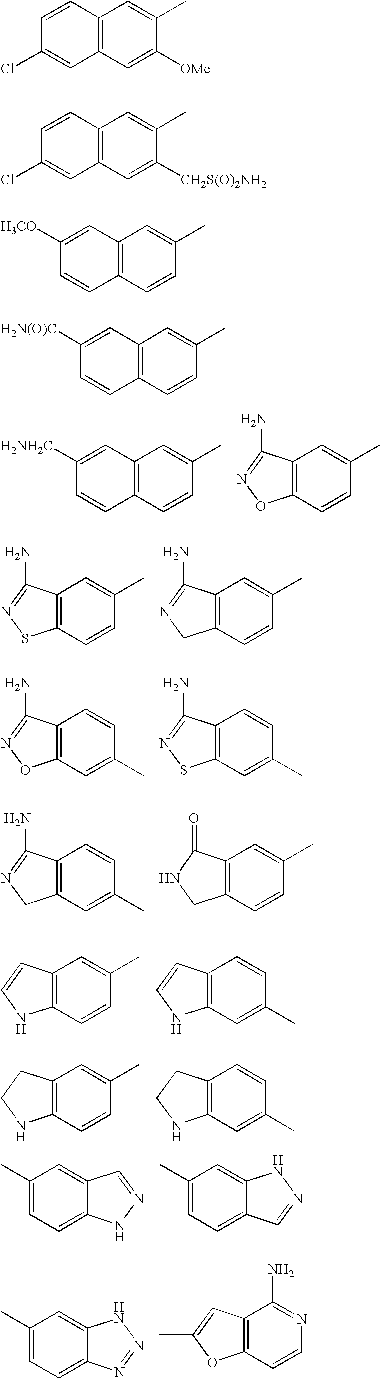

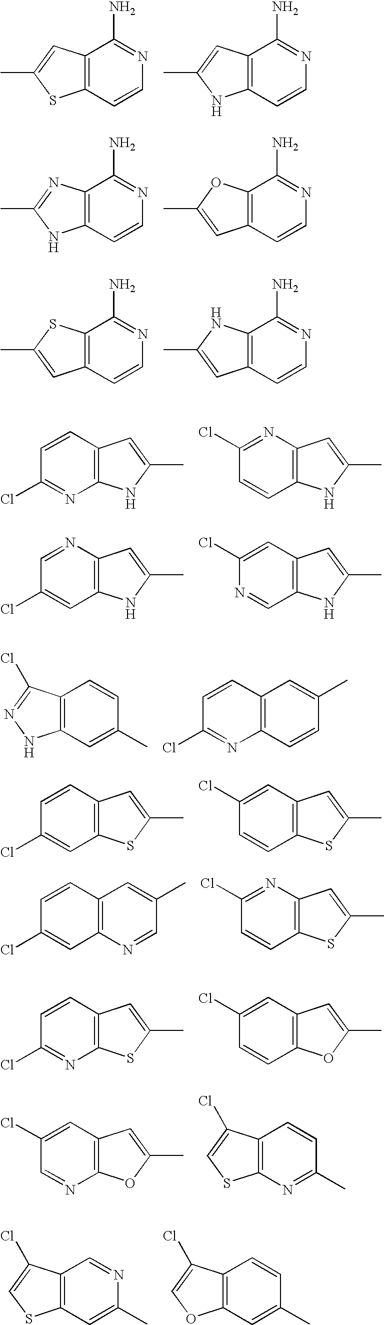

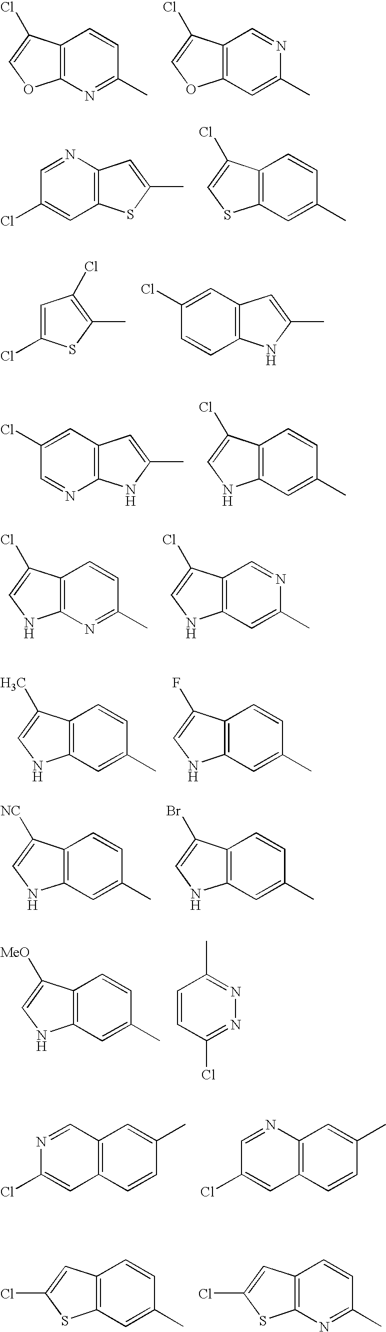

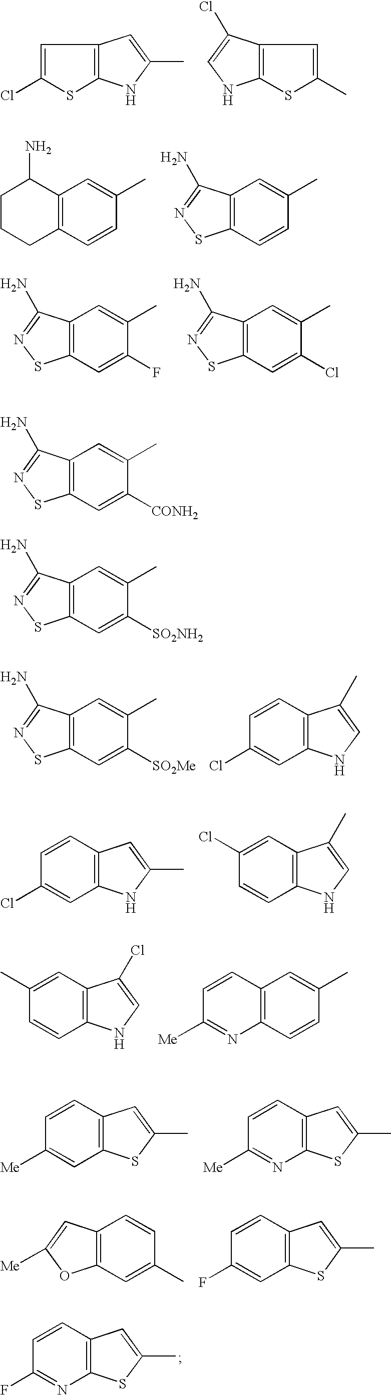

one of G₁ and Z is selected from CR³R^{3a}CR³R^{3a}, CR³R^{3a}C(O), C(O)CR³R^{3a}, C(O)O, OC(O), CR³R^{3a}O, OCR³R^{3a}, CR³R^{3a}NR^{3b}, NR^{3b}CR³R^{3a}, CR³R^{3a}NR^{3e}, NR^{3e}CR³R^{3a}, C(O)NR^{3b}, NR^{3b}C(O), CR³R^{3a}S(O), S(O)CR³R^{3a}, CR³R^{3a}S(O)₂, S(O)₂CR³R^{3a}, S(O)NR^{3b}, NR^{3b}S(O)₂, and S(O)₂NR^{3b}, and the other of G1 and Z is selected from CR³R^{3a}CR³R^{3a}, CR³R^{3a}C(O), C(O)CR³R^{3a}, C(O)O, OC(O), CR³R^{3a}O, OCR³R^{3a}, CR³R^{3a}NR^{3b}, CR³R^{3a}NR^{3e}, C(O)NR^{3b}, CR³R^{3a}S(O), S(O)CR³R^{3a}, CR³R^{3a}S(O)₂, S(O)₂CR³R^{3a}, S(O)NR^{3b}, and S(O)₂NR^{3b}, wherein the right side of Z/G₁ is attached to ring A/ring G, provided that neither Z nor G form an N—S, NCH₂N, NCH₂O, or NCH₂S bond with either group to which it is attached;

A is selected from: C₅₋₁₀ carbocycle substituted with 0-2 R⁴, and 5-10 membered heterocycle consisting of: carbon atoms and 1-4 heteroatoms selected from the group consisting of N, O, and S(O)ₚ and substituted with 0-2 R⁴;

B is selected from Y, X—Y, N(B¹)C(O)C(R³R^{3g})NB²B³, N(B¹)C(O)C(R³R^{3g})C(R³R^{3g})NB²B³, 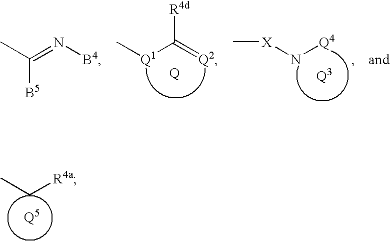
provided that Z and B are attached to different atoms on A, the R^{4d} shown is other than OH, and that the A-X—N moiety forms other than a N—N—N group;

B¹ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂₀₋₁—C₃₋₇ carbocycle substituted with 0-2 R^{4b}, and —(CH₂)₀₋₁-5-6 membered heterocycle consisting of: carbon atoms and 1-4 heteroatoms selected from the group consisting of N, O, and S(O)ₚ and substituted with 0-2 R^{4b};

B² is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, NR_{2d}R^{2d}, CH₂NR^{2d}R^{2d}, CH₂CH₂—NR^{2d}R^{2d}, C(O)R^{2e}, C(O)NR^{2d}R^{2d}, SO₂NR^{2d}R^{2d}, and S(O)ₚR^{5a};

B³ is selected from H, C₁₋₆ alkyl substituted with 0-1 R^{4c}, —(CH₂)₀₋₁-3-6 membered carbocycle substituted with 0-1 R⁵, and a —(CH₂)₀₋₁-5-6 membered heterocycle consisting of: carbon atoms and 1-4 heteroatoms selected from the group consisting of N, O, and S(O)ₚ and substituted with 0-1 R⁵;

B⁴ is selected from H, SO₂R^{3b}, C(O)R^{3b}, SO₂NR³R^{3b}, C(O)NR³R^{3b}, OR², and —CN;

B⁵ is NR²R^{2f} or CR³R²R^{2f};

ring Q is a 5-6 membered ring consisting of, in addition to the Q¹-CR^{4d}Q² group shown, carbon atoms and 0-2 heteroatoms selected from N, O, and S(O)ₚ, and the ring is substituted with an additional 0-2 R^{4d};

Q¹ and Q² are each N;

alternatively, Q¹ is CR³ and R^{4d} is NR²R^{2a} or NR^{3a}B⁴, provided that when Q¹ is CR³, then this R³ group optionally forms a ring with the R² group of R^{4d}, this ring is a 5-6 membered ring consisting of, in addition to the C—C—N shown, carbon atoms and from 0-1 additional heteroatoms selected from N, O, and S(O)ₚ, and this ring is substituted with 0-1 R⁵;

Q⁴ is selected from C═O and SO₂;

ring Q³ is a 4-7 membered monocyclic or tricyclic ring consisting of, in addition to the N-Q⁴ group shown, carbon atoms and 0-2 heteroatoms selected from NR^{4c}, O, S, S(O), and S(O)₂, wherein: 0-2 double bonds are present within the ring and the ring is substituted with 0-2 R⁴;

alternatively, ring Q³ is a 4-7 membered ring to which another ring is fused, wherein: the 4-7 membered ring consists of, in addition to the shown amide group, carbon atoms and 0-2 heteroatoms selected from NR^{4c}, O, S, S(O), and S(O)₂ and 0-1 double bonds are present within the ring; the fusion ring is phenyl or a 5-6 membered heteroaromatic consisting of carbon atoms and 1-2 heteroatoms selected from NR_{4c}, O, and S;

ring Q³, which includes the 4-7 membered ring and the fusion ring, is substituted with 0-3 R⁴;

ring Q⁵ is a C₃-₇ monocyclic carbocycle or 3-7 membered monocyclic heterocycle, wherein the carbocycle or heterocycle consists of: carbon atoms and 0-2 heteroatoms selected from N, O, and S(O)ₚ, the carbocycle or heterocycle further comprises 0-2 double bonds and 0-2 carbonyl groups, and the carbocycle or heterocycle is substituted with 0-2 R⁴;

X is selected from —(CR²R^{2a})₁₋₄—, —C(O)—, —C(═NR^{1c})—, —CR²(NR^{1b}R²)—, —C(O)CR²R^{2a}—, —CR²R^{2a}C(O), —C(O)NR²—, —NR²C(O)—, —C(O)NR²CR²R^{2a}—, —NR²C(O)CR²R^{2a}—, —CR²R^{2a}C(O)NR²—, —CR²R^{2a}NR²C(O)—, —NR²C(O)NR²—, —NR²—, —NR²CR²R^{2a}—, —CR²R^{2a}NR²—, —S(O)₂—, —NR²S(O)₂—, O, —CR²R^{2a}O—, and —OCR²R^{2a}—;

Y is selected from: CY¹Y²R^{4a}, NR³R^{3a}, and C(O)NR³R^{3a};

Y¹ and Y² are independently C₁₋₃ alkyl substituted with 0-2 R⁴;

alternatively, Y is selected from one of the following carbocyclic and heterocycles that are substituted with 1 R^{4a} and 0-2 R⁴: cyclopropyl, cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, isoxazolinyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolyl, benzimidazolyl, benzoxazolyl, benzthiazolyl, indazolyl, benzisoxazolyl, benzisothiazolyl, and isoindazolyl;

Z is selected from a bond, CH₂, CH₂CH₂, CH₂O, OCH₂, C(O), NH, CH₂NH, NHCH₂, CH₂C(O), C(O)CH₂, C(O)NH, NHC(O), NHC(O)CH₂C(O)NH, S(O)₂, CH₂S(O)₂, S(O)₂(CH₂), SO₂NH, and NHSO₂, wherein the right side of Z is attached to ring A, provided that Z does not form a N—S, NCH₂N, NCH₂O, or NCH₂S bond with either group to which it is attached;

Z² is selected from H, C₁₋₄ alkyl, phenyl, benzyl, C(O)R^{3b}, S(O)R^{3f}, and S(O)₂R^{3f};

R^{1a}, at each occurrence, is selected from H, —(CH₂)ᵣ—R^{1b}, —(CH(CH₃))ᵣ—R^{1b}, —(C(CH₃)₂)ᵣ—R^{1b}, —O—(CR³R^{3a})ᵣ—R^{1b}, —NR²—(CR³R^{3a})ᵣ—R^{1b}, and —S—(CR³R^{3a})ᵣ13 R^{1b}, provided that R^{1a} forms other than an N-halo, N—S, O—O, or N—CN bond;

alternatively, when two R^{1a} groups are attached to adjacent atoms, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;

R^{1b} is selected from H, CH₃, CH₂CH₃, CH₂CH₂CH₃, CH(CH₃)₂, F, Cl, Br, I, —CN, —CHO, CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, NR²C(O)NHR², NR²C(O)₂R^{2a}, OC(O)NR²R^{2a}, C(O)NR²R^{2a}, C(O)NR²(CH₂)ᵣOR², SO₂NR²R^{2a}, NR²SO₂R², C₅₋₆ carbocycle substituted with 0-2 R^{4b}, and 5-6 membered heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;

R², at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C5-6 carbocycle substituted with 0-2 R^{4b}, a C5-6 carbocyclic-CH₂-group substituted with 0-2 R^{4b}, and 5-6 membered heterocycle consisting of: carbon atoms and 1-4 heteroatoms selected from the group consisting of N, O, and S(O)ₚ, and substituted with 0-2 R^{4b};

R^{2a}, at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅-₆ carbocycle substituted with 0-2 R^{4b}, and 5-6 membered heterocycle consisting of: carbon atoms and 1-4 heteroatoms selected from the group consisting of N, O, and S(O)ₚ, and substituted with 0-2 R^{4b};

alternatively, NR²R^{2a}forms a 5 or 6 membered saturated, partially saturated or unsaturated ring substituted with 0-2 R^{4b} and consisting of: 0-1 additional heteroatoms selected from the group consisting of N, O, and S(O)ₚ;

R^{2b}, at each occurrence, is selected from CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅-₆ carbocycle substituted with 0-2 R ^{4b}, and 5-6 membered heterocycle consisting of: carbon atoms and 1-4 heteroatoms selected from the group consisting of N, O, and S(O)ₚ, and substituted with 0-2 R^{4b};

R^{2c}, at each occurrence, is selected from CF₃, OH, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅-₆ carbocycle substituted with 0-2 R^{4b}, and 5-6 membered heterocycle containing from 1-4 heteroatoms selected from the group consisting of N, O, and S(O)ₚ, and substituted with 0-2 R^{4b};

R^{2d}, at each occurrence, is selected from H, R^{4c}, C₁₋₄ alkyl substituted with 0-2 R^{4c}, —(CR³R^{3a})ᵣ—C₃₋₆ carbocycle substituted with 0-2 R^{4c}, and —(CR³R^{3a})ᵣ-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;

alternatively, NR^{2d}R^{2d} forms a 5 or 6 membered saturated, partially saturated or unsaturated ring substituted with 0-2 R^{4b} and consisting of: 0-1 additional heteroatoms selected from the group consisting of N, O, and S(O)ₚ;

R²e, at each occurrence, is selected from H, R^{4c}, C₁₋₄ alkyl substituted with

R^{2e}, at each occurrence, is selected from H, R^{4c}, C₁₄ alkyl substituted with 0-2 R^{4c}, —(CR³R^{3a})ᵣ—C₃-₆ carbocycle substituted with 0-2 R^{4c}, and —(CR³R^{3a})₄-5-6 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;

R^{2f}, at each occurrence, is selected from H, CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-1 R^{4b}, C₅-₆ carbocycle substituted with 0-2 R ^{4b}, and 5-6 membered heterocycle containing from 1-4 heteroatoms selected from the group consisting of N, O, and S(O)ₚ and substituted with 0-2 R^{4b};

alternatively, CR²R^{2f} forms a 5-6 membered ring consisting of: carbon atoms and 0-2 heteroatoms selected from N, O, and S(O)ₚ, and this ring is substituted with 0-2 R^{4b};

alternatively, NR²R^{2f} forms a 5-6 membered ring consisting of: carbon atoms and 0-2 additional heteroatoms selected from N, O, and S(O)ₚ, and this ring is substituted with 0-2 R^{4b};

alternatively, when B⁵ is NR²R^{2f}, B⁴ and R^{2f} combine to form a 5-6 membered ring consisting of: carbon atoms and 0-2 additional heteroatoms selected from N, O, and S(O)ₚ, and this ring is substituted with 0-2 R^{4b} and the R² group of NR²R^{2f}, in addition to the groups recited below, is selected from SO₂R^{3b} and C(O)R^{3b};

R³, at each occurrence, is selected from H, CH₃, CH₂CH₃, CH₂CH₂CH₃, CH(CH₃)₂, benzyl, and phenyl;

R^{3a}, at each occurrence, is selected from H, CH₃, CH₂CH₃, CH₂CH₂CH₃, CH(CH₃)₂, benzyl, and phenyl;

alternatively, R³ and R^{3a}, together with the nitrogen atom to which they are attached, combine to form a 5 or 6 membered saturated, partially unsaturated, or unsaturated ring consisting of: carbon atoms and the nitrogen atom to which R³ and R^{3a}are attached;

R^{3b}, at each occurrence, is selected from H, CF₃, CH₃, CH₂CH₃, CH₂CH₂CH₃, CH(CH₃)₂, —(C₀₋₁ alkyl)-5-6 membered carbocycle substituted with 0-1 R^{1a}, and —(C₀₋₁ alkyl)-5-6 membered heterocycle substituted with 0-1 R^{1a} and consisting of: carbon atoms and 1-4 heteroatoms selected from the group consisting of N, O, and S(O)ₚ;

R^{3c}, at each occurrence, is selected from CH₃, CH₂CH₃, CH₂CH₂CH₃, CH(CH₃)₂, benzyl, and phenyl;

R^{3d}, at each occurrence, is selected from H, CH₃, CH₂CH₃, CH₂CH₂CH₃, CH(CH₃)₂, CH₂-phenyl, CH₂CH₂-phenyl, and C(═O)R^{3c};

R⁴, at each occurrence, is selected from ═O, OR², CH₂OR², (CH₂)₂OR², F, Cl, Br, I, C₁₋₄ alkyl, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, NR²C(O)NR²R^{2a}, SO₂NR²R^{2a}, NR²SO₂NR²R^{2a}, S(O)ₚR^{5a}, NR²SO₂—C₁₋₄ alkyl, NR²SO₂R⁵, CF₃, CF₂CF₃, 5-6 membered carbocycle substituted with 0-1 R⁵, and a 5-6 membered heterocycle consisting of: carbon atoms and 1-4 heteroatoms selected from the group consisting of N, O, and S(O)ₚ, and substituted with 0-1 R⁵;

R^{4b}, at each occurrence, is selected from H, ═O, OR³, CH₂OR³, F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR³R^{3a}, CH₂NR³R^{3a}, C(O)R³, CH₂—C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, NR³C(O)NR³R^{3a}, CH₂NR³C(O)NR³R^{3a}, C(═NR³)NR³R^{3a}, CH₂C(═NR³)NR³R^{3a}, NR³C(═NR³)NR³R^{3a}, CH₂NR³C(═NR³)NR³R^{3a}, SO₂NR³R^{3a}, CH₂SO₂NR³R^{3a}, NR³SO₂NR³R^{3a}, CH₂NR³SO₂NR³R^{3a}, NR³SO₂—C₁₋₄ alkyl, CH₂NR³SO₂—C₁₋₄ alkyl, NR³SO₂CF₃, CH₂NR³SO₂CF₃, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, CF₃, and CH₂—CF₃;

R^{4c}, at each occurrence, is selected from ═O, (CR³R^{3a})ᵣOR², (CR³R^{3a})ᵣF, (CR³R^{3a})ᵣBr, (CR³R^{3a})ᵣCl, (CR³R^{3a})ᵣCF₃, C₁₋₄ alkyl, C₂₋₃ alkenyl, C₂₋₃ alkynyl, (CR³R^{3a})ᵣCN, (CR³R^{3a})ᵣNO₂, (CR³R^{3a})ᵣNR²R^{2a}, (CR³R^{3a})ᵣN(→O)R²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂R^{5a}, (CR³R^{3a})ᵣC(O)NR²SO₂R^{5a}, (CR³R^{3a})ᵣS(O)ₚR^{5a}, (CF₂)ᵣCF₃, (CR³R^{3a})ᵣC₃₋₁₀ carbocycle substituted with 0-2 R^{4b}, and (CR³R^{3a})ᵣ5-10 membered heterocycle consisting of carbon atoms and from 1-4 heteroatoms selected from the group consisting of N, O, and S(O)ₚ and substituted with 0-2 R^{4b};

R^{4d}, at each occurrence, is selected from H, CH₂OR², OR², C₁₋₄ alkyl, CH₂—CN, —CN, CH₂NO₂, NO₂, CH₂NR²R^{2a}, NR²R^{2a}, CH₂—C(O)R^{2c}, C(O)R^{2c}, NR²C(O)R^{2b}, (CH₂)ᵣC(O)NR²R^{2a}, NR²C(O)NR²R^{2a}, (CH₂)ᵣSO₂NR²R^{2a}, NR²SO₂NR²R^{2a}, NR²SO₂R⁵, (CH₂)ᵣS(O)ₚR^{5a}, CH₂CF₃, CF₃, CH₂-5-6 membered carbocycle substituted with 0-1 R⁵, 5-6 membered carbocycle substituted with 0-1 R⁵, a CH₂-5-6 membered heterocycle consisting of: carbon atoms and 1-4 heteroatoms selected from the group consisting of N, O, and S(O)ₚ and substituted with 0-1 R⁵, and a 5-6 membered heterocycle consisting of: carbon atoms and 1-4 heteroatoms selected from the group consisting of N, O, and S(O)ₚ and substituted with 0-1 R⁵;

R⁵, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, NR³C(O)NR³R^{3a}, CH(═NOR^{3d}), C(═NR³)NR³R^{3a}, NR³C(═NR³)NR³R^{3a}, SO₂NR³R^{3a}, NR³SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;

R^{5a}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, NR³R^{3a}, CH₂NR³R^{3a}, C(O)R³, CH₂C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, CF₃, CF₂CF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond; and

R⁶, at each occurrence, is selected from H, OH, OR², F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, CN, NO₂, NR²R^{2a}, CH₂NR²R^{2a}, C(O)R^{2b}, CH₂C(O)R^{2b}, NR²C(O)R^{2b}, NR²C(O)NR²R^{2a}, C(═NH)NH₂, NHC(═NH)NH₂, S0₂NR²R^{2a}, NR²SO₂NR²R^{2a}, and NR²SO₂C₁₋₄ alkyl.

In a third embodiment, the present invention provides a novel compound, or a pharmaceutically acceptable salt, solvate, or prodrug form thereof, wherein the compound is selected from:

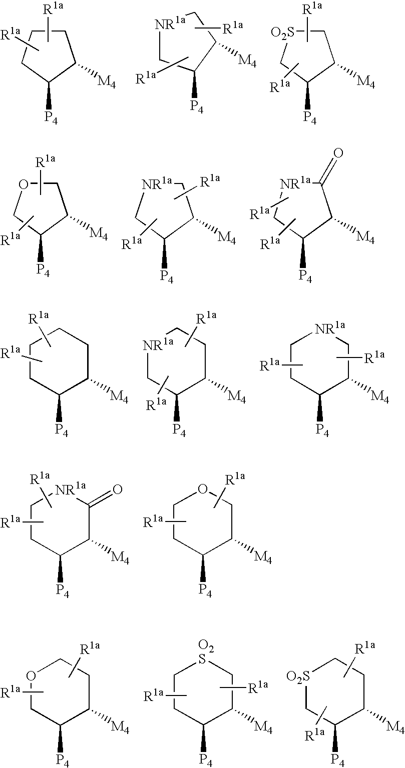

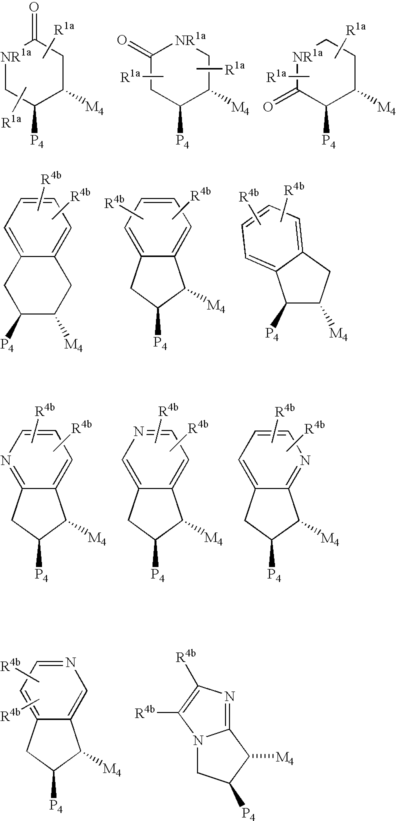

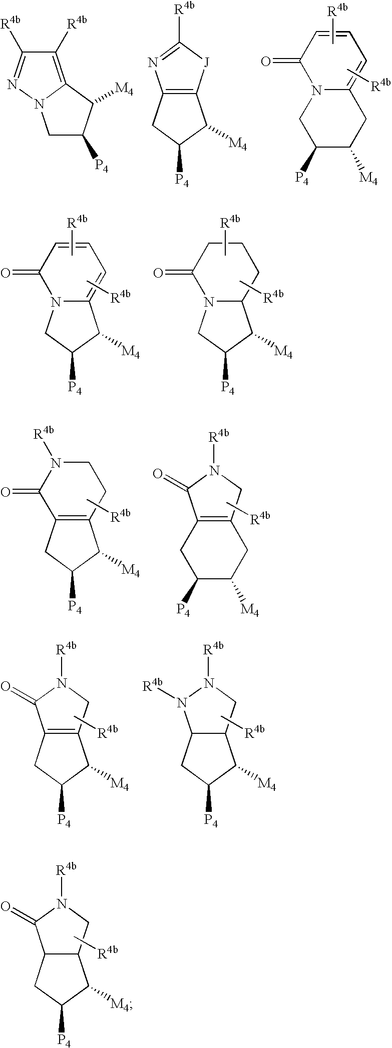

P₄ is G₁-G;

M₄ is Z-A-B;

G is selected from: 2-aminomethyl-4-chloro-phenyl, 2-aminosulfonyl-4-chloro-phenyl, 2-amido-4-chloro-phenyl, 4-chloro-2-methylsulfonyl-phenyl, 2-aminosulfonyl-4-fluoro-phenyl, 2-amido-4-fluoro-phenyl, 4-fluoro-2-methylsulfonyl-phenyl, 2-aminomethyl-4-bromo-phenyl, 2-aminosulfonyl-4-bromo-phenyl, 2-amido-4-bromo-phenyl, 4-bromo-2-methylsulfonyl-phenyl, 2-aminomethyl-4-methyl-phenyl, 2-aminosulfonyl-4-methyl-phenyl, 2-amido-4-methyl-phenyl, 2-methylsulfonyl-4-methyl-phenyl, 4-fluoro-pyrid-2-yl, 4-bromo-pyrid-2-yl, 4-methyl-pyrid-2-yl, 5-fluoro-thien-2-yl, 5-bromo-thien-2-yl, 5-methyl-thien-2-yl, 2-amido-4-methoxy-phenyl, 2-amido-phenyl, 2-aminomethyl-3-fluoro-phenyl, 2-aminomethyl-4-fluoro-phenyl, 2-amninomethyl-5-fluoro-phenyl, 2-aminomethyl-6-fluoro-phenyl, 2-aminomethyl-phenyl, 2-amino-pyrid-4-yl, 2-aminosulfonyl-4-methoxy-phenyl, 2-aminosulfonyl-phenyl, 3-amido-phenyl, 3-amino-4-chloro-phenyl, 3-aminomethyl-phenyl, 2-methoxy-pyrid-5-yl, 5-chloro-pyrid-2-yl, 5-chloro-thien-2-yl, 6-amino-5-chloro-pyrid-2-yl, 6-amino-pyrid-2-yl, 2-cyano-4-chloro-phenyl, 2-methoxy-4-chloro-phenyl, 2-fluoro-4-chloro-phenyl, 3-chloro-4-methyl-phenyl, 3-fluoro-4-chloro-phenyl, 3-methyl-4-chloro-phenyl, 3-fluoro-4-methyl-phenyl, 3,4-dimethyl-phenyl, 3-chloro-4-fluoro-phenyl, 3-methyl-4-fluoro-phenyl, 4-methylsulfanyl-phenyl, 2-chlorothiazol-5-yl, 5-chlorothiazol-2-yl, 3-pyridyl, 2-pyridyl, 5-fluoro-pyrid-2-yl, 5-methyl-pyrid-2-yl, 5-methoxy-pyrid-2-yl, 5-cyano-pyrid-2-yl, 2-pyrimidyl, 5-chloro-pyrimid-2-yl, 5-fluoro-pyrimid-2-yl, 5-methyl-pyrimid-2-yl, 5-methoxy-pyrimid-2-yl, 5-cyano-pyrimid-2-yl, phenyl, 3-chloro-phenyl, 3-fluoro-phenyl, 3-methyl-phenyl, 4-ethyl-phenyl, 3-methoxy-phenyl, 3-cyano-phenyl, 4-chloro-phenyl, 4-fluoro-phenyl, 4-methyl-phenyl, 4-methoxy-phenyl, 4-cyano-phenyl,

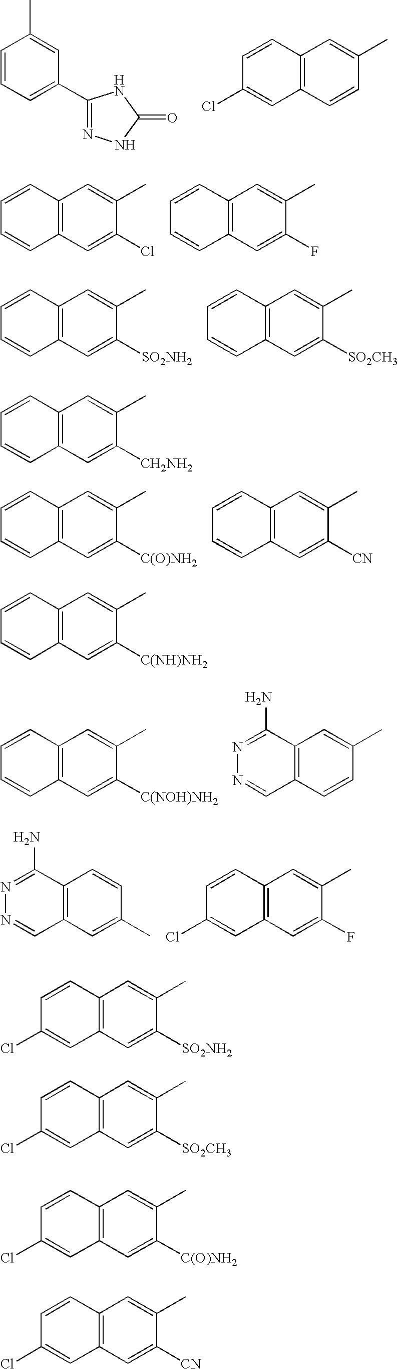

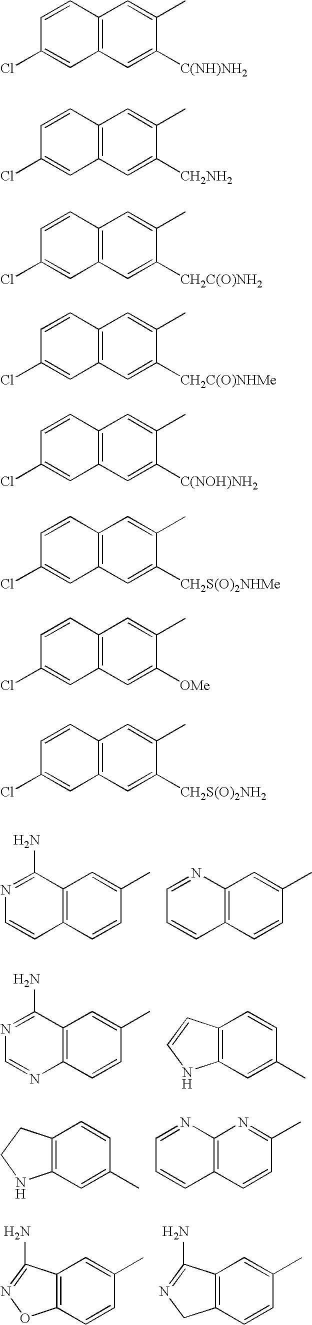

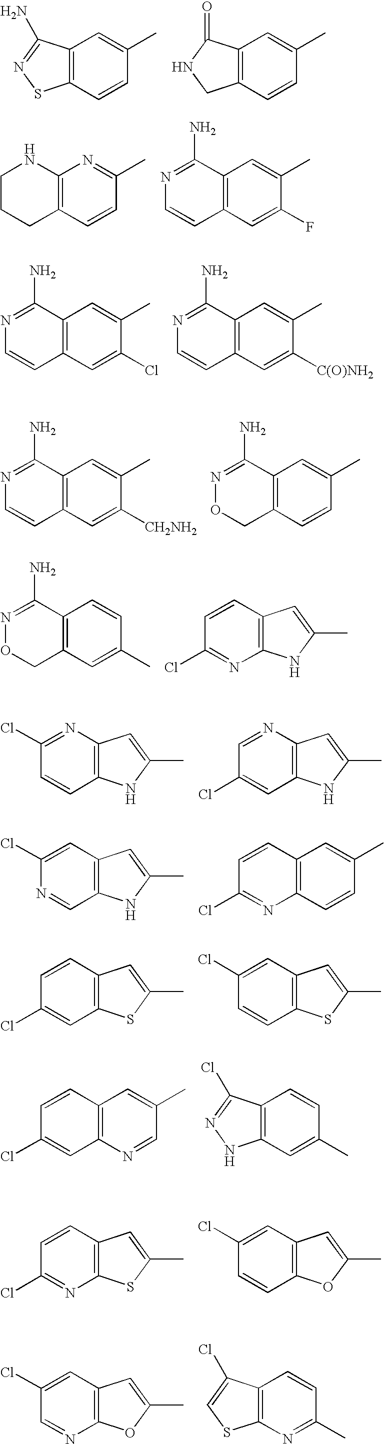

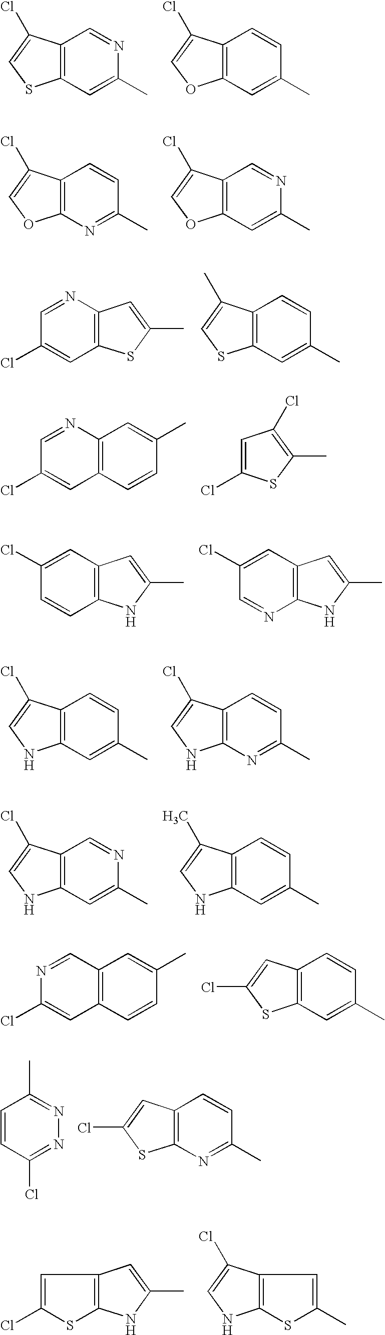

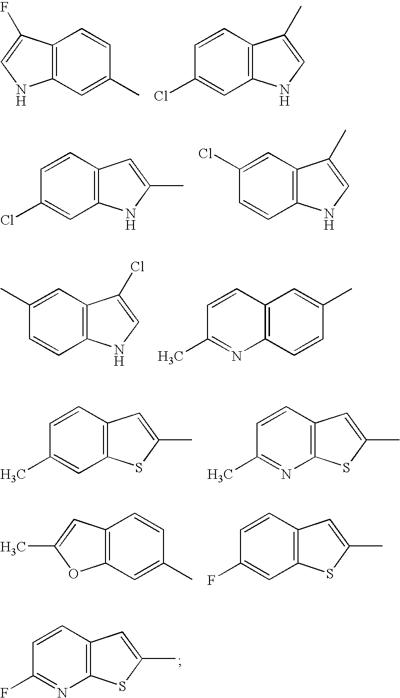

one of G₁ and Z is selected from CH₂CH₂, CH₂C(O), C(O)CH₂, C(O)O, OC(O), CH₂O, OCH₂, CH₂NH, NHCH₂, C(O)NH, NHC(O), CH₂S(O), S(O)CH₂, CH₂S(O)₂, S(O)₂CH₂, S(O)NH, NHS(O)₂, and S(O)₂NH, and the other of G₁ and Z is selected from CH₂CH₂, CH₂C(O), C(O)CH₂, C(O)O, OC(O), CH₂O, OCH₂, CH₂NH, C(O)NH, CH₂S(O), S(O)CH₂, CH₂S(O)₂, S(O)₂CH₂, S(O)NH, and S(O)₂NH, wherein the right side of Z/G₁ is attached to ring A/ring G, provided that neither Z nor G form an N—S, NCH₂N, NCH₂O, or NCH₂S bond with either group to which it is attached;

A is selected from one of the following carbocyclic and heterocyclic groups, which are substituted with 0-2 R⁴, cyclohexyl, phenyl, piperidinyl, piperazinyl, pyridyl, pyrimidyl, furanyl, morpholinyl, thienyl, pyrrolyl, pyrrolidinyl, oxazolyl, isoxazolyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolinyl, indolyl, benzimidazolyl, benzoxazolyl, benzthiazolyl, indazolyl, benzisoxazolyl, benzisothiazolyl, and isoindazolyl;

B¹ is selected from H, CH₃, CH₂CH₃, CH₂CH₂CH₃, CH(CH₃)₂, —(CH₂)₀₋₁—C₅₋₆ carbocycle substituted with 0-2 R^{4b}, and —(CH₂)₀₋₁-5-6 membered heterocycle consisting of: carbon atoms and 1-4 heteroatoms selected from the group consisting of N, O, and S(O)ₚ and substituted with 0-2 R^{4b};

B² is selected from H, CH₃, CH₂CH₃, CH₂CH₂CH₃, CH(CH₃)₂, C(O)R^{2e}, C(O)NR^{2d}R^{2d}, SO₂NR^{2d}R^{2d}, and S(O)ₚR^{5a};

B³ is selected from H, C₁₋₆ alkyl substituted with 0-1 R^{4c}, —(CH₂)₀₋₁-3-6 membered carbocycle substituted with 0-1 R⁵, and a -(CH₂)₀₋₁-5-6 membered heterocycle consisting of: carbon atoms and 1-4 heteroatoms selected from the group consisting of N, O, and S(O)ₚ and substituted with 0-1 R⁵;

B⁴ is selected from H, SO₂R^{3b} and OR²;

B⁵ is NR²R^{2f};

ring Q is a 5-6 membered ring consisting of, in addition to the N-CR^{4d}═N group shown, carbon atoms and 0-2 heteroatoms selected from N, O, and S(O)ₚ, and the ring is substituted with an additional 0-2 R^{4d};

Q⁴ is selected from C═O and SO₂;

ring Q³ is a 5-7 membered ring consisting of, in addition to the N-Q⁴ group shown, carbon atoms and 0-2 heteroatoms selected from NR^{4c}, O, S, S(O), and S(O)₂, wherein: 0-2 double bonds are present within the ring and the ring is substituted with 0-2 R^{4a};

alternatively, ring Q³ is a 5-7 membered ring to which another ring is fused, wherein: the 5-7 membered ring consists of, in addition to the shown amide group, carbon atoms and 0-2 heteroatoms selected from NR^{4c}, O, S, S(O), and S(O)₂, and 0-1 double bonds are present within the ring; the fusion ring is phenyl or a 5-6 membered heteroaromatic consisting of carbon atoms and 1-2 heteroatoms selected from NR^{4c}, O, and S;

ring Q³, which includes the 5-7 membered ring and the fusion ring, is substituted with 0-3 R^{4a};

ring Q⁵, is a C₃-₆ monocyclic carbocycle or 5-6 membered monocyclic heterocycle, wherein the carobocycle or heterocycle consists of carbon atoms and 0-2 heteroatoms selected from N, O, and S(O)p, the carbocycle or heterocycle further comprises 0-1 double bonds and 0-1 carbonyl groups, and the carbocycle or heterocycle is substituted with 0-2 R⁴;

X is selected from —(CR²R^{2a})₁₋₂—, —C(═NR^{1b})—, —C(O)—, —S(O)₂—, —NR²S(O)₂—, —NR²S(O)₂—, —NR²C(O)—, —C(O)NR²—, —NR²C(O)CR²R^{2a}—, —NR²C(O)NR²—, NR², —NR²CR²R^{2a}—, —CR²R^{2a}NR²—, O, —OCR²R^{2a}—, and —CR²R^{2a}O—;

Y is selected from: CY¹Y²R^{4a}, NR³R^{3a}, and C(O)NR³R^{3a};

Y¹ and Y² are independently C₁₋₂ alkyl substituted with 0-2 R⁴;

alternatively, Y is selected from one of the following carbocyclic and heterocycles that are substituted with 1 R^{4a} and 0-1 R⁴: cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, thiazolyl, isothiazolyl, pyrazolyl, imidazolyl, oxadiazole, thiadiazole, triazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,5-triazole, 1,3,4-triazole, benzofuran, benzothiofuran, indole, benzimidazole, benzimidazolone, benzoxazole, benzthiazole, indazole, benzisoxazole, benzisothiazole, and isoindazole;

R^{1a} is selected from H, R^{1b}, CH(CH₃)R^{1b}, C(CH₃)₂R^{1b}, CH₂R^{1b}, and CH₂CH₂R^{1b}, provided that R^{1a} forms other than an N-halo, N—S, or N—CN bond;

alternatively, when two R^{1a} groups are attached to adjacent atoms, together with the atoms to which they are attached they form a 5-6 membered ring consisting of: carbon atoms and 0-2 heteroatoms selected from the group consisting of N, O, and S(O)ₚ, this ring being substituted with 0-2 R^{4b} and 0-3 ring double bonds;

R^{1b} is selected from H, CH₃, CH₂CH₃, F, Cl, Br, —CN, —CHO, CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, C(O)NR²R^{2a}, SO₂NR²R^{2a}, NR²SO₂R², phenyl substituted with 0-2 R^{4b}, and 5-6 membered aromatic heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;

R², at each occurrence, is selected from H, CF₃, CH₃, CH₂CH₃, CH₂CH₂CH₃, CH(CH₃)₂, phenyl substituted with 0-2 R^{4b}, a benzyl substituted with 0-2 R^{4b}, and a 5-6 membered aromatic heterocycle consisting of: carbon atoms and 1-4 heteroatoms selected from the group consisting of N, O, and S(O)ₚ, and substituted with 0-2 R^{4b};

R^{2a}, at each occurrence, is selected from H, CF₃, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle consisting of: carbon atoms and 1-4 heteroatoms selected from the group consisting of N, O, and S(O)ₚ, and substituted with 0-2 R^{4b};

alternatively, NR²R^{2a}forms a 5 or 6 membered saturated, partially saturated or unsaturated ring substituted with 0-2 R^{4b} and consisting of: 0-1 additional heteroatoms selected from the group consisting of N, O, and S(O)ₚ;

R^{2b}, at each occurrence, is selected from CF₃, C₁₋₄ alkoxy, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle consisting of: carbon atoms and 1-4 heteroatoms selected from the group consisting of N, O, and S(O)ₚ, and substituted with 0-2 R^{4b};

R^{2c}, at each occurrence, is selected from CF₃, OH, OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle containing from 1-4 heteroatoms selected from the group consisting of N, O, and S(O)ₚ, and substituted with 0-2 R^{4b};

R^{2d}, at each occurrence, is selected from H, R^{4c}, C₁₋₄ alkyl substituted with 0-2 R^{4c}, C₃₋₆ carbocycle substituted with 0-2 R^{4c}, —(CR³R^{3a})—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CR³R^{3a})-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N-C-halo, S(O)ₚ-halo, O-halo, N—S, S—N, S(O)ₚ—S(O)ₚ, S—O, O—N, O—S, or O—O moiety;

R^{2e}, at each occurrence, is selected from H, R^{4c}, C₁₋₄ alkyl substituted with 0-2 R^{4c}, C₃₋₆ carbocycle substituted with 0-2 R^{4c}, —(CR³R^{3a})—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CR³R^{3a})-5-6 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;

R^{2f} at each occurrence, is selected from H, CF₃, CH₃, CH₂CH₃, CH₂CH₂CH₃, CH(CH₃)₂, OCH₃, and benzyl;

alternatively, NR²R^{2f} forms a 5-6 membered ring consisting of: carbon atoms and 0-2 additional heteroatoms selected from N, O, and S(O)ₚ, and this ring is substituted with 0-2 R^{4b};

alternatively, B⁴ and R^{2f} combine to form a 5-6 membered ring consisting of: carbon atoms and 0-1 additional heteroatoms selected from N, O, and S(O)ₚ, and this ring is substituted with 0-2 R^{4b} and the R² group of NR²R^{2f}, in addition to the groups recited below, can be SO₂R^{3b};

R^{3b}, at each occurrence, is selected from H, CF₃, CH₃, CH₂CH₃, CH₂CH₂CH₃, and CH(CH₃)₂;

R⁴, at each occurrence, is selected from H, ═O, CH₂OR², (CH₂)₂OR², OR², F, Cl, Br, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, NR²C(O)NR²R^{2a}, SO₂NR²R^{2a}, CF₃, and CF₂CF₃;

R^{4a} is selected from —(CR³R^{3g})ᵣ-5-6 membered carbocycle substituted with 0-3 R^{4c}, —(CR³R^{3g})ᵣ-5-6 membered heterocycle substituted with 0-3 R^{4c} and consisting of: carbon atoms and 1-4 heteroatoms selected from the group consisting of N, O, and S(O)ₚ, (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣNR^{2d}C(O)R^{2e}, (CR³R^{3g})ᵣ—C(O)R^{2e}, (CR³R^{3g})ᵣ—OC(O)R^{2e}, (CR³R^{3g})ᵣ—C(O)NR^{2d}R^{2d}, (CR³R^{3g})ᵣ—C(O)OR^{2d}, (CR³R^{3g})ᵣ—NR^{2d}C(O)NR^{2d}R^{2d}, (CR³R^{3g})ᵣNR^{2d}C(O)OR^{2d}, (CR³R^{3g})ᵣ—SO₂NR^{2d}R^{2d}, (CR³R^{3g})ᵣ—NR^{2d}SO₂R^{2d}, and (CR³R^{3g})ᵣ—S(O)ₚR^{2d}, provided that S(O)ₚR^{2d} forms other than S(O)₂H or S(O)H;

R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, CH₂—C(O)R³, C(O)OR^{3c}, CH₂—C(O)OR^{3c}, NR³C(O)R^{3a}, CH₂NR³C(O)R^{3a}, C(O)NR³R^{3a}, CH₂—C(O)NR³R^{3a}, SO₂NR³R^{3a}, CH₂SO₂NR³R^{3a}, NR³SO₂—C₁₋₄ alkyl, CH₂NR³SO₂—C₁₋₄ alkyl, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, and CF₃;

R^{4c}, at each occurrence, is selected from ═O, OR², (CR³R^{3a})OR², F, (CR³R^{3a})F, Br, (CR³R^{3a})Br, Cl, (CR³R^{3a})CI, CF₃, (CR³R^{3a})CF₃, C₂₋₃ alkenyl, C₂₋₃ alkynyl, C₁₋₄ alkyl, —CN, (CR³R^{3a})CN, NO₂, (CR³R^{3a})NO₂, NR²R^{2a}, (CR³R^{3a})NR²R^{2a}, N(→O)R²R^{2a}, (CR³R^{3a})N(→O)R²R^{2a}, C(O)R^{2c}, (CR³R^{3a})C(O)R^{2c}, NR²C(O)R^{2b}, (CR³R^{3a})NR²C(O)R^{2b}, C(O)NR²R^{2a}, (CR³R^{3a})C(O)NR²R^{2a}, NR²C(O)NR²R^{2a}, (CR³R^{3a})NR²C(O)NR²R^{2a}, SO₂NR²R^{2a}, (CR³R^{3a})SO₂NR²R^{2a}, NR²SO₂NR²R^{2a}, (CR³R^{3a})NR²SO₂NR²R^{2a}, NR²SO₂R^{5a}, (CR³R^{3a})NR²SO₂R^{5a}, S(O)ₚR^{5a}, (CR³R^{3a})S(O)ₚR^{5a}, CF₃, CF₂CF₃, C₃₋₁₀ carbocycle substituted with 0-2 R^{4b}, (CR³R^{3a})C₃₋₁₀ carbocycle substituted with 0-2 R^{4b}, 5-10 membered heterocycle consisting of carbon atoms and from 1-4 heteroatoms selected from the group consisting of N, O, and S(O)ₚ and substituted with 0-2 R^{4b}, and (CR³R^{3a})-5-10 membered heterocycle consisting of carbon atoms and from 1-4 heteroatoms selected from the group consisting of N, O, and S(O)ₚ and substituted with 0-2 R^{4b};

R^{4d}, at each occurrence, is selected from H, CH₂OR², OR², CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, CH₂NR²R^{2a}, NR²R^{2a}, C(O)R^{2c}, NR²C(O)R^{2c}, C(O)NR²R^{2a}, NR²C(O)NR²R^{2a}, NR²SO₂R⁵, SO₂NR²R^{2a}, 6 membered carbocycle substituted with 0-1 R⁵, and a 5-6 membered heterocycle consisting of: carbon atoms and 1-2 heteroatoms selected from the group consisting of N, O, and S(O)ₚ and substituted with 0-1 R⁵;

R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, SO₂NR³R^{3a}, NR³SO₂—C₁₋₄ alkyl, NR³SO₂CF₃, NR³SO₂-phenyl, S(O)ₚCF₃, S(O)ₚ-C₁₋₄ alky S(O)ₚ-phenyl, CF₃, phenyl substituted with 0-2 R⁶, naphthyl substituted with 0-2 R⁶, and benzyl substituted with 0-2 R⁶; and

R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, SO₂NR²R^{2a}, and NR²SO₂C₁₋₄ alkyl.

In a fourth embodiment, the present invention provides a novel compound or a pharmaceutically acceptable salt, solvate, or prodrug form thereof, wherein the compound is selected from:

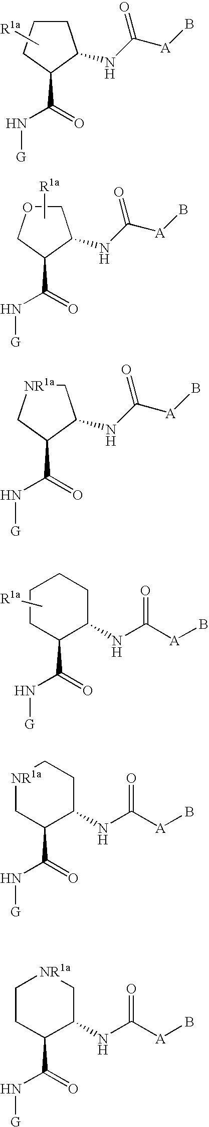

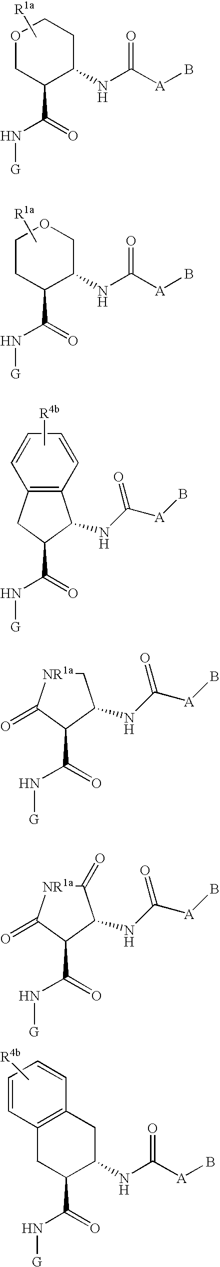

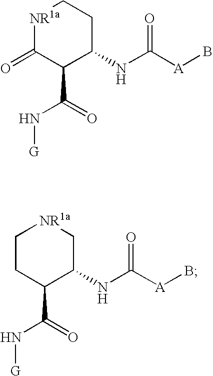

G is selected from:

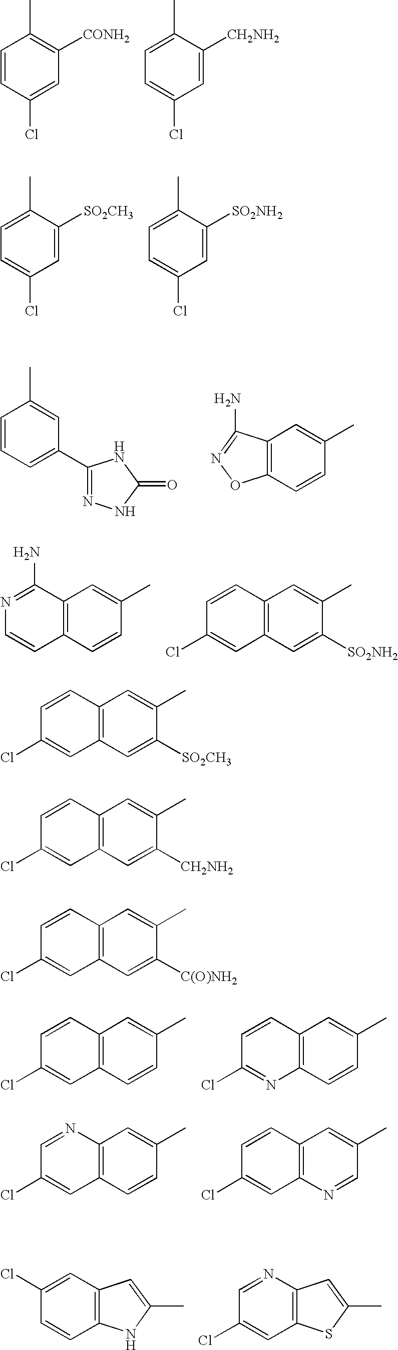

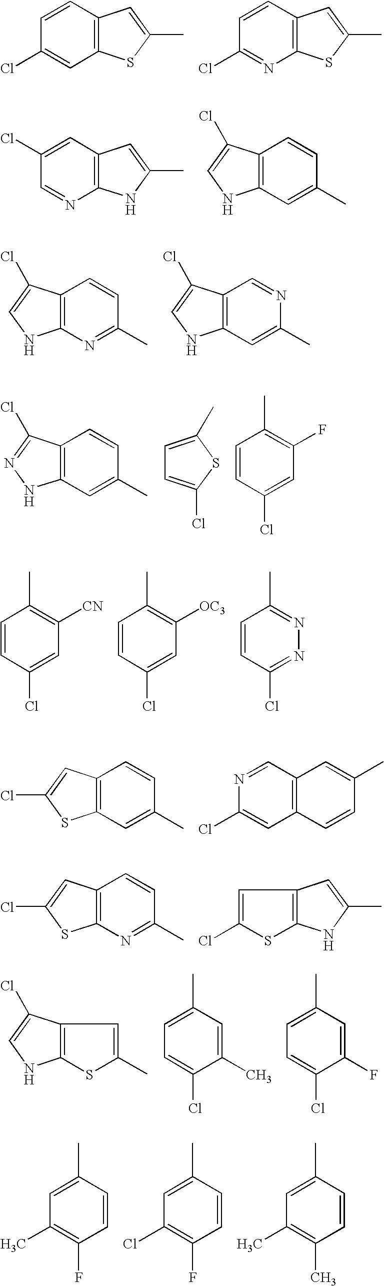

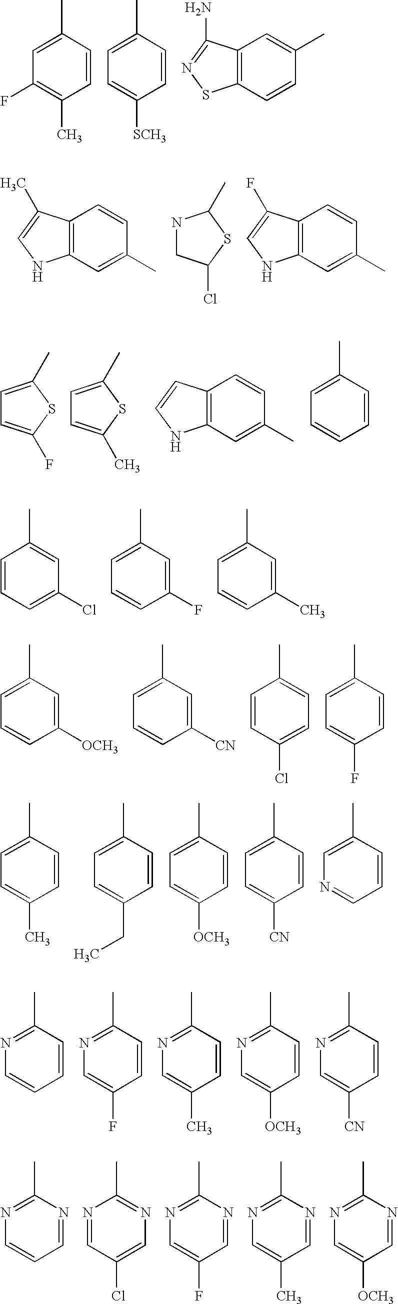

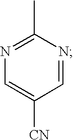

one of G₁ and Z is selected from NHCH₂, NHC(O), CH₂S(O), CH₂S(O)₂, and NHS(O)₂, and the other of G₁ and Z is selected from C(O)CH₂, C(O)O, C(O)NH, S(O)CH₂, S(O)₂CH₂, S(O)NH, and S(O)₂NH, wherein the right side of Z/G₁ is attached to ring A/ring G, provided that neither Z nor G form an N—S, NCH₂N, NCH₂O, or NCH₂S bond with either group to which it is attached;

A is selected from cyclohexyl, piperidinyl, piperazinyl, phenyl, pyridyl, and pyrimidyl, and is substituted with 0-2 R⁴;

B is selected from Y, N(B¹)C(O)C(R³R^{3g})NB²B³, 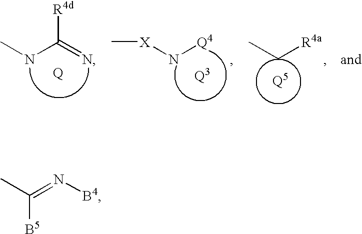
provided that Z and B are attached to different atoms on A, the R^{4d} shown is other than OH, and that the A-X—N moiety forms other than a N—N—N group;

B¹ is selected from H, CH₃, CH₂CH₃, CH₂CH₂CH₃, and CH(CH₃)₂;

B² is selected from H, CH₃, CH₂CH₃, CH₂CH₂CH₃, and CH(CH₃)₂;

B³ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, C₂₋₅ alkyl substituted with 1 R^{4c}, —(CH₂)₀₋₁-3-6 membered carbocycle substituted with 0-1 R⁵, and a —(CH₂)₀₋₁-5-6 membered heterocycle consisting of: carbon atoms and 1-4 heteroatoms selected from the group consisting of N, O, and S(O)ₚ and substituted with 0-1 R⁵;

B⁴ is selected from H, SO₂R^{3b}, and OR²;

B⁵ is NR²R^{2f};

ring Q is a 5-6 membered ring consisting of, in addition to the N—CR^{4d}═N group shown, carbon atoms and 0-1 heteroatoms selected from N, O, and S(O)ₚ, and the ring is substituted with an additional 0-2 R^{4d};

Q⁴ is selected from C═O and SO₂;

ring Q³ is a 6-7 membered ring consisting of, in addition to the N-Q⁴ group shown, carbon atoms and 0-1 heteroatoms selected from NR^{4c}, O, S, S(O), and S(O)₂, wherein: 0-2 double bonds are present within the ring and the ring is substituted with 0-2 R⁴;

alternatively, ring Q³ is a 5-7 membered ring to which another ring is fused, wherein: the 5-7 membered ring consists of, in addition to the shown amide group, carbon atoms and 0-1 heteroatoms selected from NR^{4c}, O, S, S(O), and S(O)₂, and 0-1 double bonds are present within the ring; the fusion ring is phenyl;

ring Q³, which includes the 5-7 membered ring and the fusion ring, is substituted with 0-2 R⁴;

ring Q⁵ is substituted with 0-1 R⁴ and is selected from cyclopropyl, cyclobutyl, cyclopentyl, cyclopentanonyl, cyclohexyl, cyclohexanonyl, pyrrolidinyl, pyrrolidinonyl, piperidinyl, piperidinonyl, tetrahydrofuranyl, and tetrahydropyranyl;

X is selected from CH₂, C(O), —S(O)₂—, —NHC(O)—, —C(O)NH—, —CH₂NH—, O, and —CH₂O—,

Y is selected from N(CH₃)₂, C(O)(CH₃)₂, C(CH₃)₂R^{4a} and C(CH₂CH₃)₂R^{4a};

altneratively, Y is selected from phenyl, pyridyl, pyrrolidino, N-pyrrolidino-carbonyl, morpholino, N-morpholino-carbonyl, 1,2,3-triazolyl, imidazolyl, and benzimidazolyl, and is substituted with 1 R^{4a} and 0-1 R⁴;

R^{1a}, at each occurrence, is selected from H, R^{1b}, CH(CH₃)R^{1b}, C(CH₃)₂R^{1b}, and CH₂R^{1b}, provided that R^{1a} forms other than an N-halo, N—S, or N—CN bond;

R^{1b} is selected from CH₃, CH₂CH₃, F, Cl, Br, —CN, CF₃, OR², NR²R^{2a}, C(O)R^{2b}, CO₂R^{2b}, CO₂R^{2a}, S(O)ₚR², C(O)NR²R^{2a}, SO₂NR²R^{2a}, NR²SO₂R², and 5-6 membered aromatic heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;

R², at each occurrence, is selected from H, CH₃, CH₂CH₃, CH₂CH₂CH₃, —CH(CH₃)₂, phenyl substituted with 0-1 R^{4b}, benzyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};

R^{2a}, at each occurrence, is selected from H, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};

alternatively, NR²R^{2a}forms a 5 or 6 membered saturated, partially saturated or unsaturated ring substituted with 0-1 R^{4b} and consisting of: 0-1 additional heteroatoms selected from the group consisting of N, O, and S(O)ₚ;

R^{2b}, at each occurrence, is selected from OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};

R^{2c}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle containing from 1-4 heteroatoms selected from the group consisting of N, O, and S(O)ₚ, and substituted with 0-1 R^{4b};

R^{2d}, at each occurrence, is selected from H, R^{4c}, C₁₋₄ alkyl substituted with 0-2 R^{4c}, C₃₋₆ carbocycle substituted with 0-2 R^{4c}, —(CH₂)—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;

R^{2e}, at each occurrence, is selected from H, R^{4c}, C₁₋₄ alkyl substituted with 0-2 R^{4c}, C₃₋₆ carbocycle substituted with 0-2 R^{4c}, —(CH₂)—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)-5-6 membered heterocycle and consisting of: carbon atoms and 1-4 heteroatoms selected from the group consisting of N, O, and S(O)ₚ, provided that R^{2e} forms other than a C(O)-halo or C(O)—S(O)ₚ moiety;

R^{2f}, at each occurrence, is selected from H, CH₃, CH₂CH₃, OCH₃, and benzyl;

alternatively, NR²R^{2f} forms a 5-6 membered ring consisting of: carbon atoms and 0-1 additional heteroatoms selected from N, O, and S(O)ₚ, and this ring is substituted with 0-1 R^{4b};

alternatively, B⁴ and R^{2f} combine to form a 5 membered ring consisting of: carbon atoms and 0-1 additional heteroatoms selected from N, O, and S(O)ₚ, and this ring is substituted with 0-2 R^{4b} and the R² group of NR²R^{2f}, in addition to the groups recited below, can be SO₂R^{3b};

R^{3b}, at each occurrence, is selected from H and CH₃;

R⁴, at each occurrence, is selected from H, ═O, OH, OR², CH₂OR², (CH₂)₂OR², F, Br, Cl, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, CF₃, and CF₂CF₃;

R^{4a} is selected from —(CR³R^{3g})ᵣ-5-6 membered carbocycle substituted with 0-3 R^{4c}, —(CR³R^{3g})ᵣ-5-6 membered heterocycle substituted with 0-3 R^{4c} and consisting of: carbon atoms and 1-4 heteroatoms selected from the group consisting of N, O, and S(O)ₚ, (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣ—C(O)NR^{2d}R^{2d}, (CR³R^{3g})ᵣ—NR^{2d}C(O)R^{2e}, (CR³R^{3g})ᵣ—C(O)R^{2e}, (CR³R^{3g})ᵣ—NR^{2d}C(O)NR^{2d}R^{2d}, (CR³R^{3g})ᵣ—NR^{2d}C(O)OR^{2d}, (CR³R^{3g})ᵣ—NR^{2d}SO₂R^{2d}, and (CR³R^{3g})ᵣ—S(O)ₚR^{2d}, provided that S(O)ₚR^{2d} forms other than S(O)₂H or S(O)H;

R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, and CF₃;

R^{4c}, at each occurrence, is selected from ═O, OR², CH₂OR², F, Br, Cl, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, C₂₋₃ alkenyl, C₂₋₃ alkynyl, —CN, NO₂, NR²R^{2a}, CH₂NR²R^{2a}, N(→O)R²R^{2a}, CH₂N(→O)R²R^{2a}, C(O)R^{2c}, CH₂C(O)R^{2c}, NR²C(O)R^{2b}, CH₂NR²C(O)R^{2b}, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, CH₂S(O)ₚR^{5a}, CF₃, CF₂CF₃, C₃₋₆ carbocycle substituted with 0-2 R^{4b}, (CH₂)C₃₋₆ carbocycle substitute with 0-2 R^{4b}, 5-6 membered heterocycle consisting of carbon atoms and from 1-4 heteroatoms selected from the group consisting of N, O, and S(O)ₚ and substituted with 0-2 R^{4b}, and (CH₂)-5-6 membered heterocycle consisting of carbon atoms and from 1-4 heteroatoms selected from the group consisting of N, O, and S(O)ₚ and substituted with 0-2 R^{4b};

R^{4d}, at each occurrence, is selected from H, CH₂OR², OR², CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, CH₂NR²R^{2a}, NR²R^{2a}, C(O)R^{2c}, NR²C(O)R^{2b}, C(O)NR²R^{2a}, SO₂NR²R^{2a}, NR²SO₂R⁵, phenyl substituted with 0-1 R⁵, and a 5-6 membered heterocycle consisting of: carbon atoms and 1 heteroatom selected from the group consisting of N, O, and S(O)ₚ and substituted with 0-1 R⁵;

R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶; and

R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, and SO₂NR²R^{2a}.

In a fifth embodiment, the present invention provides a novel compound or a pharmaceutically acceptable salt, solvate, or prodrug form thereof, wherein the compound is selected from:

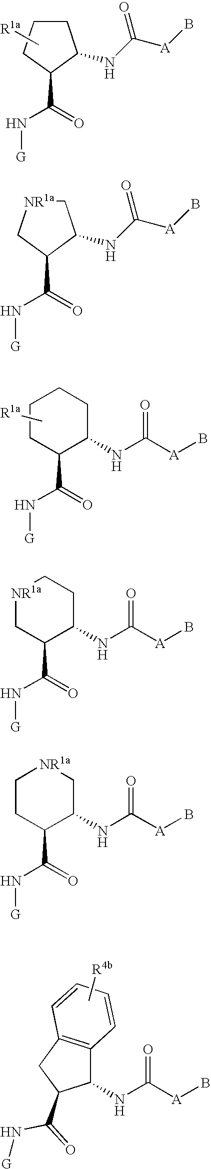

G is selected from:

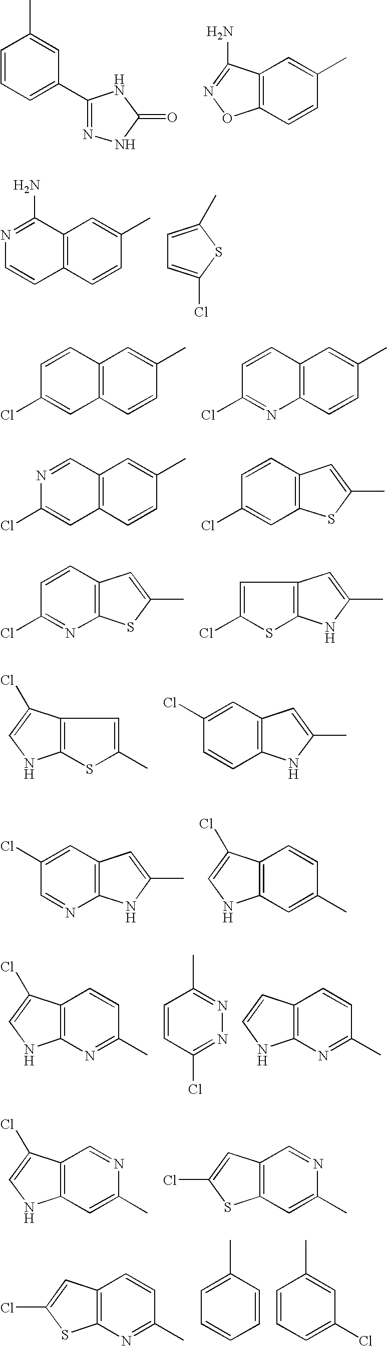

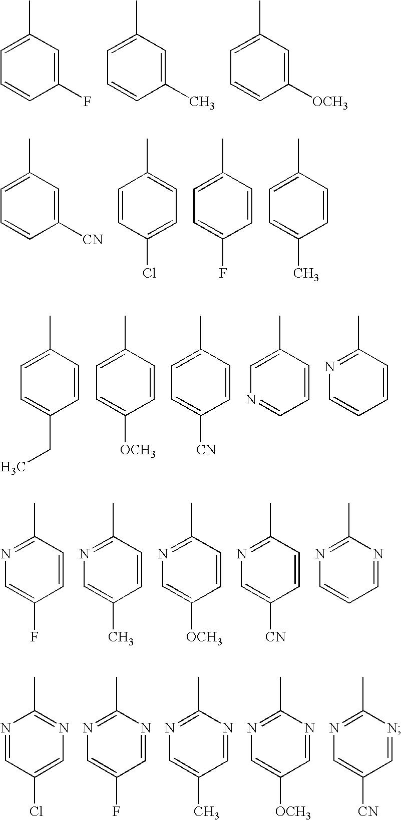

A is selected from the group: cyclohexyl, phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;

B is selected from Y, N(B¹)C(O)C(R³R^{3g})NB²B³, 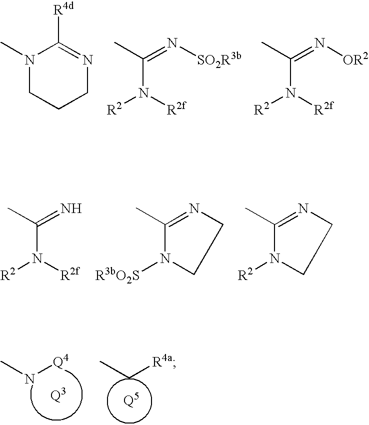
provided that Z and B are attached to different atoms on A and that the R^{4d} shown is other than OH;

B¹ is selected from H, CH₃, CH₂CH₃, and CH₂CH₂CH₃;

B² is selected from H, CH₃, and CH₂CH₃;

B³ is selected from CH₃, CH₂CH₃, CH₂CH₂CH₃, CH(CH₃)₂, CH₂CH₂CH₂CH₃, C(CH₃)₃, CH(CH₃)CH₂CH(CH₃)₂, CH₂CH₂OH, CH(CH₃)CH₂OH, CH(phenyl)CH₂CH₃, cyclopropyl, cyclobutyl, cyclopentyl, cyclohexyl, and CH₂-cyclopropyl; 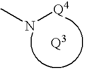
is attached to a different atom on A than M and is selected from the group:

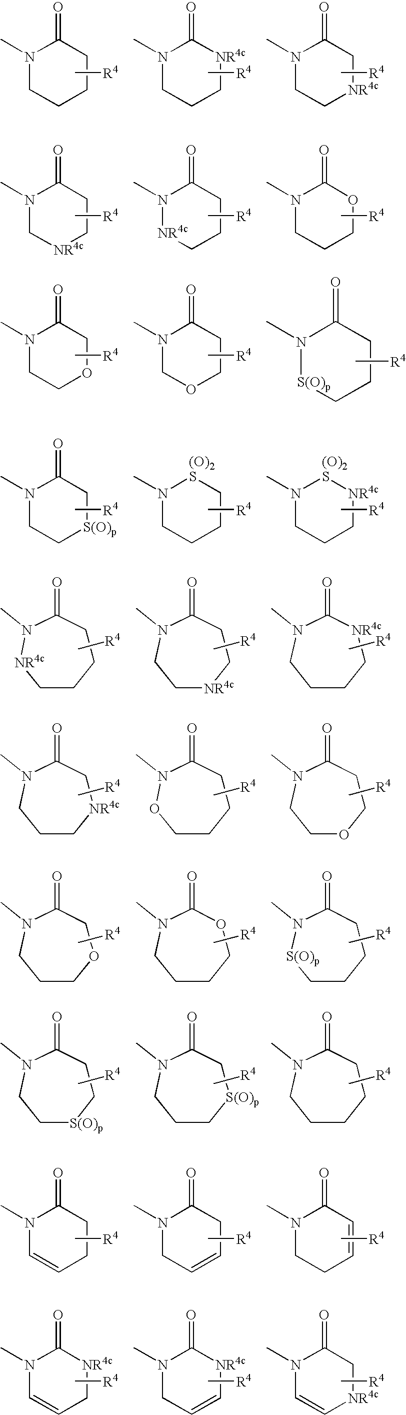

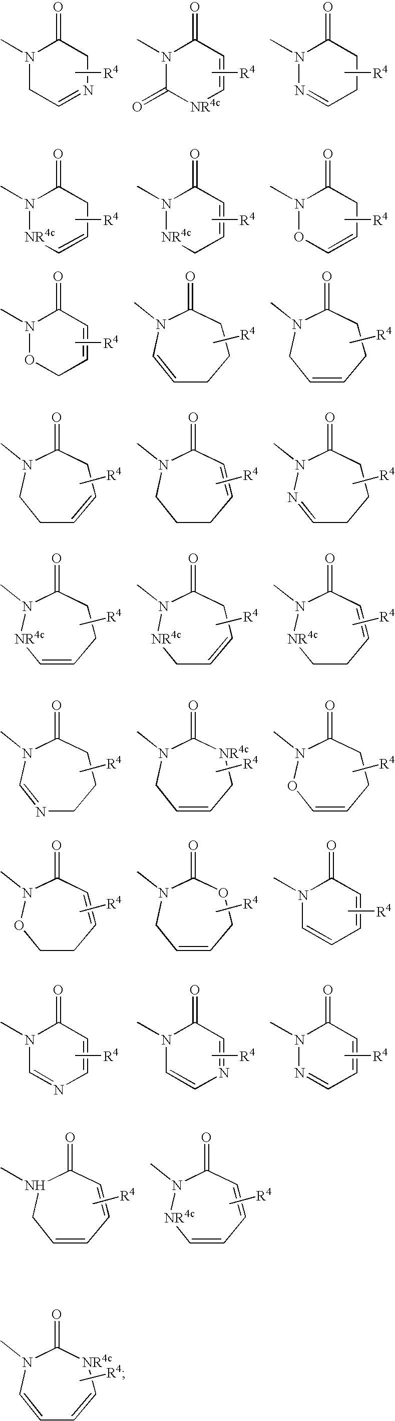

ring Q⁵ is selected from cyclopropyl, cyclobutyl, cyclopentyl, 2-cyclopentanonyl, cyclohexyl, 2-cyclohexanonyl, pyrrolidinyl (attached to A and R^{4a} at the 2-position), pyrrolidinyl (attached to A and R^{4a} at the 3-position), 2-pyrrolidinonyl (attached to A and R^{4a} at the 3-position), piperidinyl (attached to A and R^{4a} at the 4-position), 4-piperdinonyl (attached to A and R^{4a} at the 3-position), tetrahydrofuranyl, and tetrahydropyranyl (attached to A and R^{4a} at the 4-position);

Y is selected from N(CH₃)₂, C(O)(CH₃)₂, C(CH₃)₂R^{4a} and C(CH₂CH₃)₂R^{4a};

alternatively, Y is selected from phenyl, pyridyl, 1,2,3-triazolyl, imidazolyl, and benzimidazolyl, and is substituted with 1 R^{4a};

R^{1a}, at each occurrence, is selected from H, CH₃, CH₂CH₃, CH₂CH₂CH₃, CH₂F, CH₂Cl, Br, CH₂Br, —CN, CH₂CN, CF₃, CH₂CF₃, OCH₃, CH₂OH, C(CH₃)₂OH, CH₂OCH₃, NH₂, CH₂NH₂, NHCH₃, CH₂NHCH₃, N(CH₃)₂, CH₂N(CH₃)₂, CO₂H, COCH₃, CO₂CH₃, CH₂CO₂CH₃, SCH₃, CH₂SCH₃, S(O)CH₃, CH₂S(O)CH₃, S(O)₂CH₃, CH₂S(O)₂CH₃, C(O)NH₂, CH₂C(O)NH₂, SO₂NH₂, CH₂SO₂NH₂, NHSO₂CH₃, CH₂NHSO₂CH₃, pyridin-2-yl, pyridin-3-yl, pyridin-4-yl, pyridin-2-yl-N-oxide, pyridin-3-yl-N-oxide, pyridin-4-yl-N-oxide, imidazol-1-yl, CH₂-imidazol-1-yl, 4-methyl-oxazol-2-yl, 4-N,N-dimethylaminomethyl-oxazol-2-yl, 1,2,3,4-tetrazol-1-yl, 1,2,3,4-tetrazol-5-yl, CH₂-1,2,3,4-tetrazol-1-yl, and CH₂-1,2,3,4-tetrazol-5-yl, provided that R^{1a} forms other than an N-halo, N—S, or N—CN bond;

R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5 membered aromatic heterocycle consisting of: carbon atoms and 1-4 heteroatoms selected from the group consisting of N, O, and S(O)ₚ, and substituted with 0-1 R^{4b};

R^{2a}, at each occurrence, is selected from H, CH₃, and CH₂CH₃;

alternatively, NR²R^{2a}forms a 5 or 6 membered saturated, partially saturated or unsaturated ring substituted with 0-1 R^{4b} and consisting of: 0-1 additional heteroatoms selected from the group consisting of N, O, and S(O)ₚ;

R^{2b}, at each occurrence, is selected from OCH₃, OCH₂CH₃, CH₃, and CH₂CH₃;

R^{2c}, at each occurrence, is selected from OH, OCH₃, OCH₂CH₃, CH₃, and CH₂CH₃;

R^{2d}, at each occurrence, is selected from H, R^{4c}, C₁₋₄ alkyl substituted with 0-2 R^{4c}, C₃-₆ cycloalkyl substituted with 0-2 R^{4c}, phenyl substituted with 0-2 and 5-6 membered aromatic heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)p, S—O, O—N, O—S, or O—O moiety;

R^{2e}, at each occurrence, is selected from H, R^{4c}, C₁₋₄ alkyl substituted with 0-2 R^{4c}, C₃₋₆ cycloalkyl substituted with 0-2 R^{4c}, phenyl substituted with 0-2 R^{4c}, and 5-6 membered aromatic heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;

R^{2f}, at each occurrence, is selected from H, CH₃, CH₂CH₃, and OCH₃;

alternatively, NR²R^{2f} forms a ring selected from morpholine, piperazine, piperidine, and pyrrolidine;

R⁴, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, and C(CH₃)₃;

R^{4a} is selected from —(CH₂)ᵣ-5-6 membered carbocycle substituted with 0-3 R^{4c}, —(CH₂)ᵣ-5-6 membered heterocycle substituted with 0-3 R^{4c} and consisting of: carbon atoms and 1-4 heteroatoms selected from the group consisting of N, O, and S(O)ₚ, (CH₂)ᵣNR^{2d}R^{2d}, (CH₂)ᵣN(→O)R^{2d}R^{2d}, (CH₂)ᵣOR^{2d}, (CH₂)ᵣ—C(O)NR^{2d}R^{2d}, (CH₂)ᵣ—NR^{2d}C(O)R^{2e}, (CH₂)ᵣ—C(O)R^{2e}, (CH₂)ᵣ—NR^{2d}C(O)NR^{2d}R^{2d}, (CH₂)ᵣ—NR^{2d}C(O)OR^{2d}, (CH₂)ᵣ—NR^{2d}SO₂R^{2d}, and (CH₂)ᵣ—S(O)ₚR^{2d}, provided that S(O)ₚR^{2d} forms other than S(O)₂H or S(O)H;

R^{4b}, at each occurrence, is selected from H, ═O, OR³, CH₂OR³, F, Cl, CH₃, CH₂CH₃, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂-phenyl, S(O)₂CH₃, S(O)₂-phenyl, and CF₃;

R^{4c}, at each occurrence, is selected from ═O, OH, OCH₃, OCH₂CH₃, OCH₂CH₂CH₃, OCH(CH₃)₂, CH₃, CH₂CH₃, CH₂CH₂CH₃, CH(CH₃)₂, C₂₋₃ alkenyl, C₂₋₃ alkynyl, CH₂OH, CH₂OCH₃, CH₂OCH₂CH₃, CH₂OCH₂CH₂CH₃, CH₂OCH(CH₃)₂, F, Br, Cl, CF₃, NR²R^{2a}, CH₂NR²R^{2a}, N(→O)R²R^{2a}, CH₂N(→O)R²R^{2a}, C(O)R^{2c}, CH₂C(O)R^{2c}, NR²C(O)R^{2b}, CH₂NR²C(O)R^{2b}, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, CH₂S(O)ₚR^{5a}, CF₃, cyclopropyl substituted with 0-1 R^{4b}, cyclobutyl substituted with 0-1 R^{4b}, cyclopentyl substituted with 0-1 R^{4b}, phenyl substituted with 0-1 R^{4b}, —CH₂-cyclopropyl substituted with 0-1 R^{4b}, —CH₂-cyclobutyl substituted with 0-1 R^{4b}, —CH₂-cyclopentyl substituted with 0-1 R^{4b}, benzyl substituted with 0-2 R^{4b}, 5-6 membered aromatic heterocycle consisting of carbon atoms and from 1-4 heteroatoms selected from the group consisting of N, O, and S(O)ₚ and substituted with 0-2 R^{4b}, and (CH₂)-5-6 membered aromatic heterocycle consisting of carbon atoms and from 1-4 heteroatoms selected from the group consisting of N, O, and S(O)ₚ and substituted with 0-2 R^{4b};

R^{4d}, at each occurrence, is selected from H, OCH₃, CH₃, CH₂CH₃, CH₂CH₂CH₃, CH(CH₃)₂, NR²R^{2a}, NR²C(O)R^{2b}, NR²SO₂R⁵, phenyl, 2-oxo-pyrrolidinyl, and 2-oxo-piperidinyl;

R⁵, at each occurrence, is selected from H, ═O, CH₃, CH₂CH₃, OR³, CH₂OR³, F, Cl, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)₂—CH₃, S(O)₂-phenyl, CF₃, phenyl substituted with 0-2 R⁶, naphthyl substituted with 0-2 R⁶, and benzyl substituted with 0-2 R⁶; and

R⁶, at each occurrence, is selected from H, OH, OR², F, Cl, CH₃, CH₂CH₃, NR²R^{2a}, CH₂NR²R^{2a}, C(O)R^{2b}, CH₂C(O)R^{2b}, NR²C(O)R^{2b}, and SO₂NR²R^{2a},

In a sixth embodiment, the present invention provides a novel compound or a pharmaceutically acceptable salt, solvate, or prodrug form thereof, wherein the compound is selected from:

A is selected from the group: phenyl, 2-pyridyl, 2-pyrimidyl, and 2-F-phenyl, wherein B is substituted at the 4-position of A;

B is selected from:

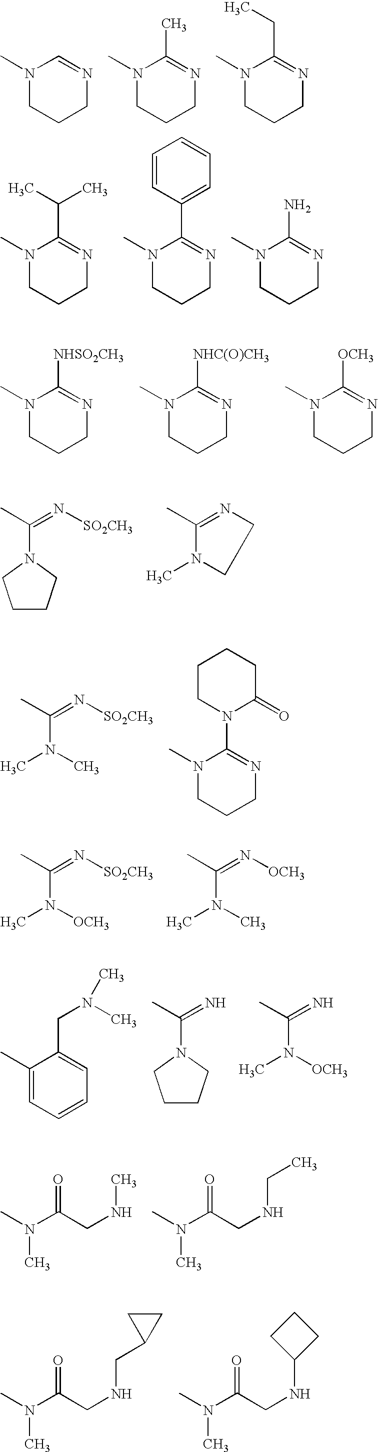

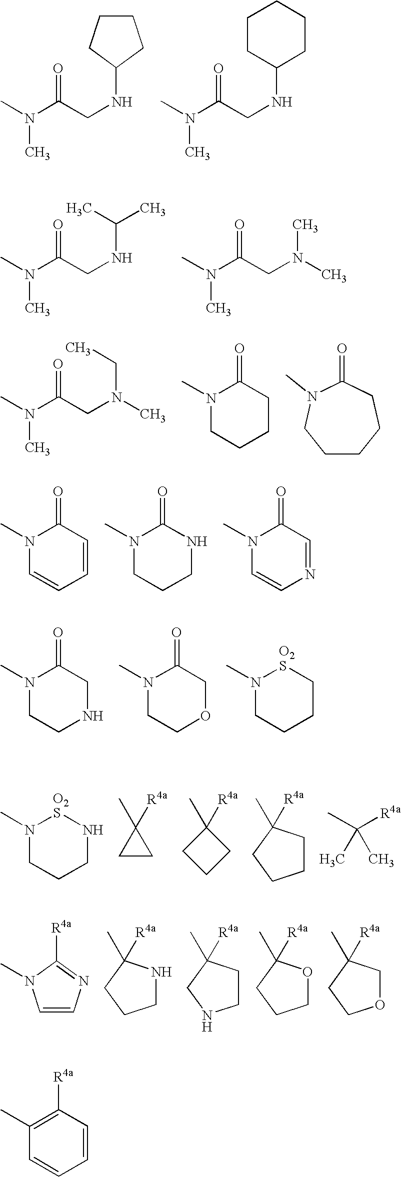

R^{2d}, at each occurrence, is selected from H, C₁₋₄ alkyl substituted with 0-1 R^{4c}, C₃₋₆ cycloalkyl substituted with 0-2 R^{4c}, phenyl substituted with 0-2 R^{4c}, and a 5-6 membered aromatic heterocycle consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety ;

R^{2e}, at each occurrence, is selected from H, C₁₋₄ alkyl substituted with 0-1 R^{4c}, C₃₋₆ cycloalkyl substituted with 0-2 R^{4c}, phenyl, substituted with 0-2 R^{4c}, and 5-6 membered aromatic heterocycle consisting of: carbon atoms and 1-4 heteroatoms selected from the group consisting of N, O, and S(O)ₚ, provided that R^{2e} forms other than a C(O)-halo or C(O)—S(O)ₚ moiety;

R^{4a} is selected from NR^{2d}R^{2d}, CH₂NR^{2d}R^{2d}, CH₂CH₂NR^{2d}R^{2d}, N(→O)R^{2d}R^{2d}, CH₂N(→O)R^{2d}R^{2d}, CH₂OR^{2d}, C(O)R^{2e}, C(O)NR^{2d}R^{2d}, CH₂C(O)NR^{2d}R^{2d}, NR^{2d}C(O)R^{2e}, CH₂NR^{2d}C(O)R^{2e}, NR^{2d}C(O)NR^{2d}R^{2d}, CH₂NR^{2d}C(O)NR^{2d}R^{2d}, NR^{2d}C(O)OR^{2d}, CH₂NR^{2d}C(O)OR^{2d}, NR^{2d}SO₂R^{2d}, CH₂NR^{2d}SO₂R^{2d}, S(O)ₚR^{2d}, CH₂S(O)ₚR^{2d}, 5-6 membered carbocycle substituted with 0-2 R^{4c}, —(CH₂)-5-6 membered carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)-5-6 membered heterocycle substituted with 0-2 R^{4c} and consisting of: carbon atoms and 1-4 heteroatoms selected from the group consisting of N, O, and S(O)ₚ provided that S(O)ₚR^{2d} forms other than S(O)₂H or S(O)H; and

R^{4c} is selected from ═O, OH, OCH₃, OCH₂CH₃, OCH₂CH₂CH₃, OCH(CH₃)₂, CH₃, CH₂CH₃, CH₂CH₂CH₃, CH(CH₃)₂, CH═CH₂, CH≡CH, CH₂OH, CH₂OCH₃, CH₂OCH₂CH₃, CH₂OCH₂CH₂CH₃, CH₂OCH(CH₃)₂, F, Br, Cl, CF₃, NR²R^{2a}, CH₂NR²R^{2a}, C(O)R^{2c}, CH₂C(O)R^{2c}, NR²C(O)R^{2b}, CH₂NR²C(O)R^{2b}, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, and CH₂S(O)ₚR^{5a}.

In a seventh embodiment, the present invention provides a novel compound or a pharmaceutically acceptable salt, solvate, or prodrug form thereof, wherein the compound is selected from:

A-B is selected from:

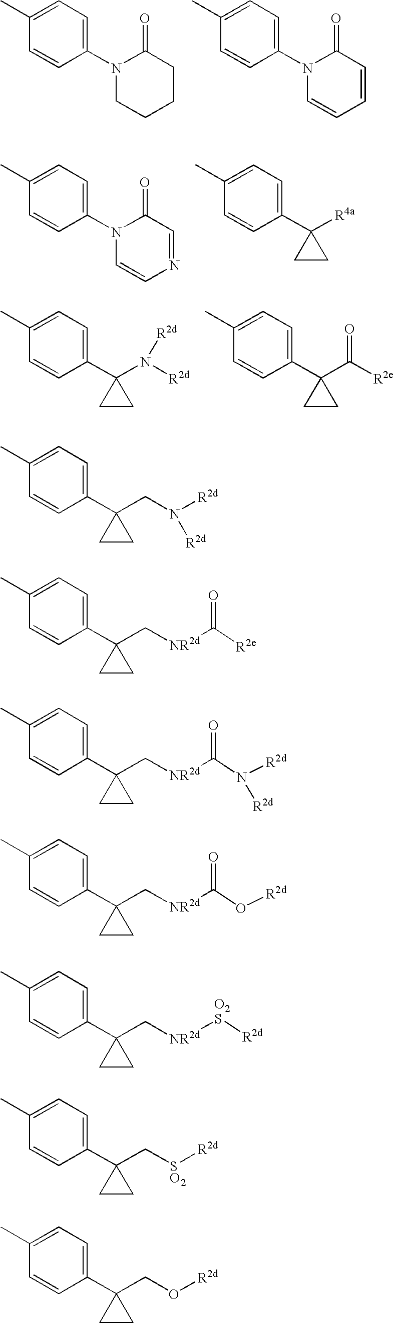

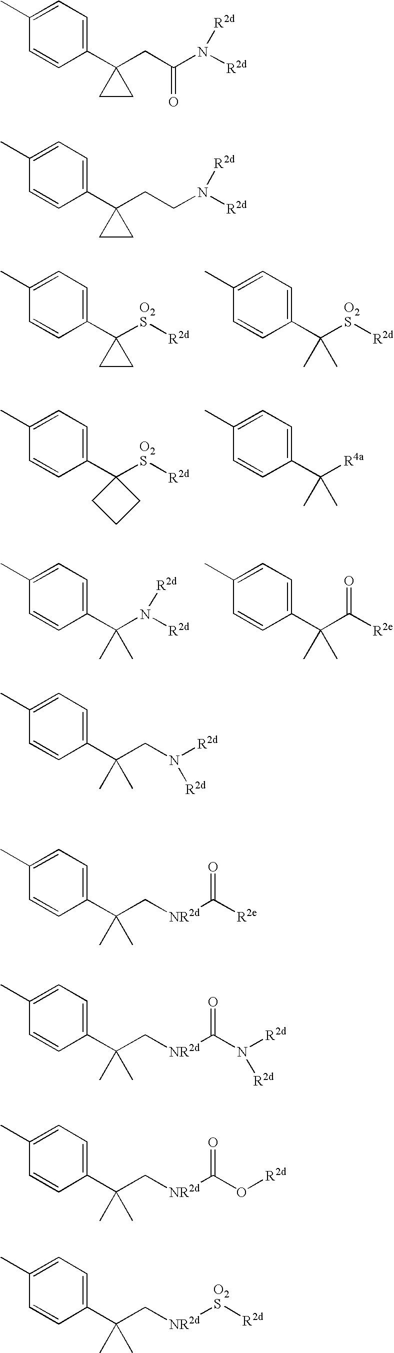

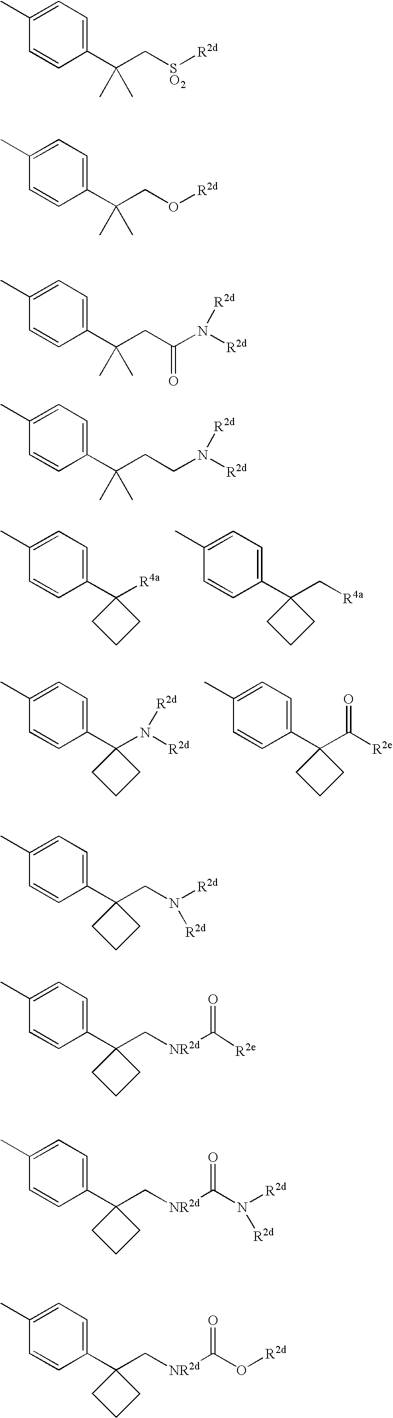

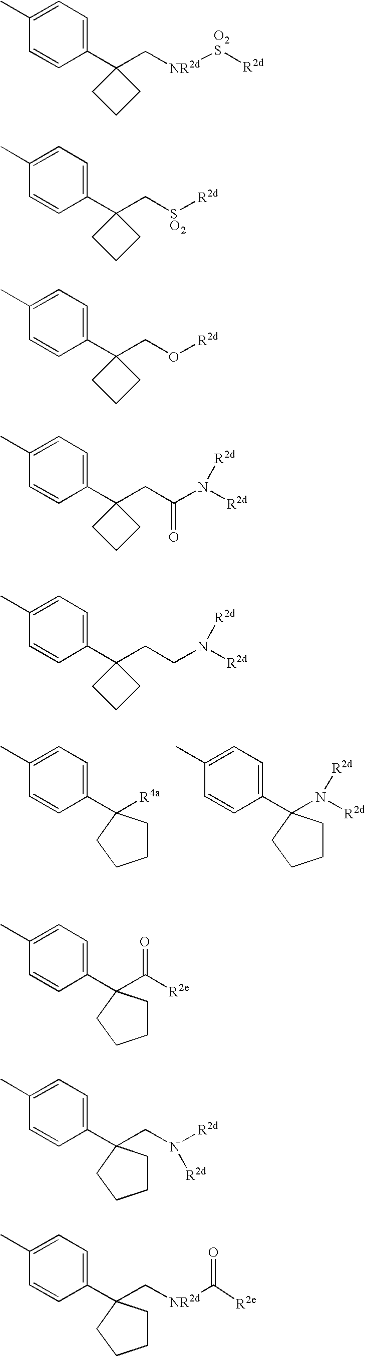

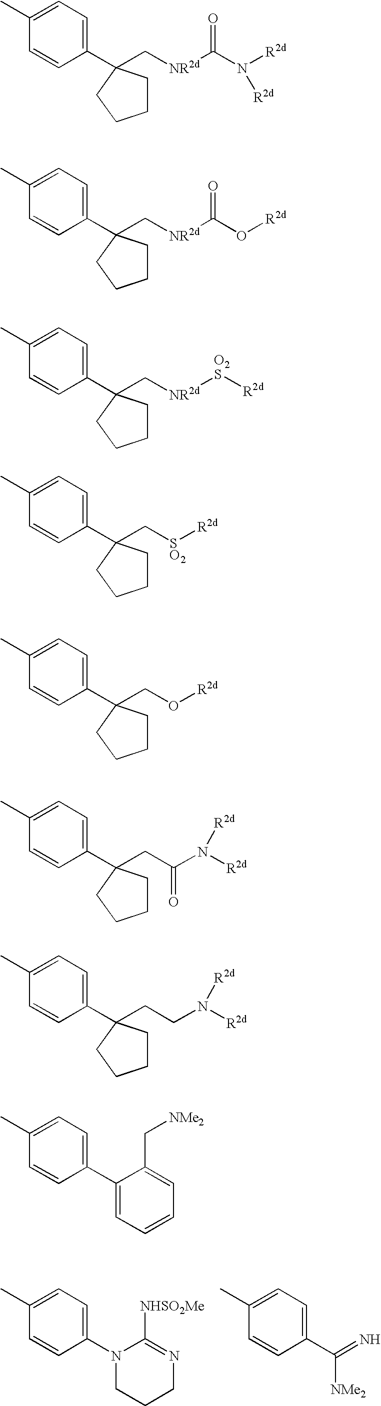

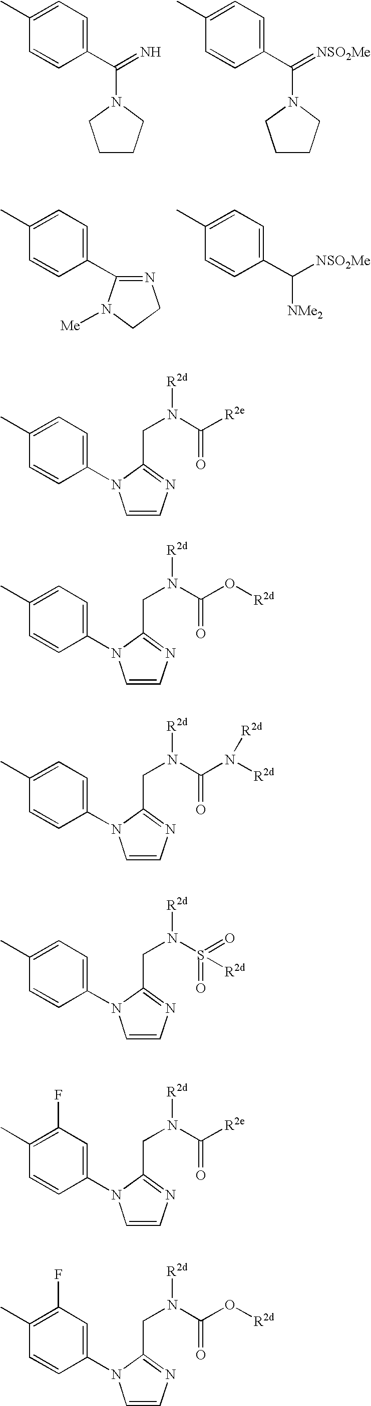

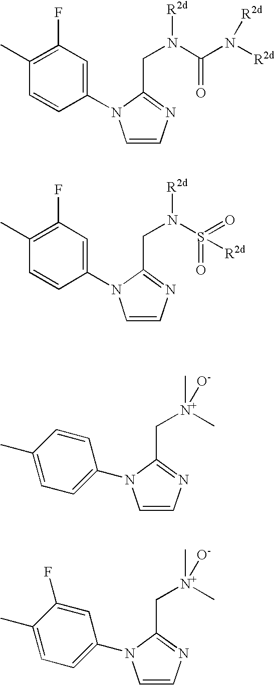

R^{2d}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;

R^{2e}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;

R^{4a} is substituted with 0-2 R^{4c} and selected from morpholine, 1,1-dioxo-thiomorpholine, dihydropyridine, piperidine, piperazine, pyrrolidine, imidazole, imidazoline, imidazolidine, oxazoline, and thiazoline; and

R^{4c} is selected from ═O, OH, OCH₃, and CH₃.

In an eighth embodiment, the present invention provides a novel compound selected from Examples 1-2 or a pharmaceutically acceptable salt, solvate, or prodrug form thereof.

In a ninth embodiment, the present invention provides a novel compound selected from Examples 1-200 of Table 1.

In another embodiment, the present invention provides a novel pharmaceutical composition, comprising: a pharmaceutically acceptable carrier and a therapeutically effective amount of a compound of the present invention or a pharmaceutically acceptable salt, solvate, or prodrug form thereof.

In another embodiment, the present invention provides a novel method for treating a thromboembolic disorder, comprising: administering to a patient in need thereof a therapeutically effective amount of a compound of the present invention or a pharmaceutically acceptable salt, solvate, or prodrug form thereof.

In another preferred embodiment, the present invention provides a novel method, wherein the thromboembolic disorder is selected from the group consisting of arterial cardiovascular thromboembolic disorders, venous cardiovascular thromboembolic disorders, and thromboembolic disorders in the chambers of the heart.

In another preferred embodiment, the present invention provides a novel method, wherein the thromboembolic disorder is selected from unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another embodiment, the present invention provides a novel method of treating a patient in need of thromboembolic disorder treatment, comprising:

administering a compound of the present invention or a pharmaceutically acceptable salt form thereof in an amount effective to treat a thromboembolic disorder

In another embodiment, the present invention provides a novel method, comprising: administering a compound of the present invention or a pharmaceutically acceptable salt form thereof in an amount effective to treat a thromboembolic disorder.

In another embodiment, the present invention provides a novel method for treating a thromboembolic disorder, comprising: administering to a patient in need thereof a therapeutically effective amount of a first and second therapeutic agent, wherein the first therapeutic agent is compound of the present invention or a pharmaceutically acceptable salt thereof and the second therapeutic agent is at least one agent selected from a second factor Xa inhibitor, an anti-coagulant agent, an anti-platelet agent, a thrombin inhibiting agent, a thrombolytic agent, and a fibrinolytic agent.

In another preferred embodiment, the present invention provides a novel method, wherein the second therapeutic agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

In another preferred embodiment, the present invention provides a novel method, wherein the second therapeutic agent is at least one anti-platelet agent.

In another preferred embodiment, the present invention provides a novel method, wherein the anti-platelet agent is aspirin and clopidogrel.

In another preferred embodiment, the present invention provides a novel method, wherein the anti-platelet agent is clopidogrel.

In another embodiment, the present invention provides a novel article of manufacture, comprising:
(a) a first container;
(b) a pharmaceutical composition located within the first container, wherein the composition, comprises: a first therapeutic agent, comprising: a compound of the present invention or a pharmaceutically acceptable salt form thereof; and,
(c) a package insert stating that the pharmaceutical composition can be used for the treatment of a thromboembolic disorder.

In another preferred embodiment, the present invention provides a novel article of manufacture, further comprising:
(d) a second container;

wherein components (a) and (b) are located within the second container and component (c) is located within or outside of the second container.

In another embodiment, the present invention provides a novel article of manufacture, comprising:

(a) a first container;

(b) a pharmaceutical composition located within the first container, wherein the composition, comprises: a first therapeutic agent, comprising: a compound of the present invention or a pharmaceutically acceptable salt form thereof; and,

(c) a package insert stating that the pharmaceutical composition can be used in combination with a second therapeutic agent to treat a thromboembolic disorder.

In another preferred embodiment, the present invention provides a novel article of manufacture, further comprising:

(d) a second container; wherein components (a) and (b) are located within the second container and component (c) is located within or outside of the second container.

In another embodiment, the present invention provides novel compounds as described above for use in therapy.

In another embodiment, the present invention provides the use of novel compounds as described above for the manufacture of a medicament for the treatment of a thromboembolic disorder.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### DEFINITIONS

The compounds herein described have asymmetric centers and are shown as being trans-substituted. The trans-substitution pattern shown and its mirror image are both encompassed by the presently claimed invention. In one embodiment, the shown absolute stereochemistry is preferred. In another embodiment, the mirror image of the shown stereochemistry is preferred.

Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention. All tautomers of shown or described compounds are also considered to be part of the present invention.

Preferably, the molecular weight of compounds of the present invention is less than about 500, 550, 600, 650, 700, 750, or 800 grams per mole. Preferably, the molecular weight is less than about 800 grams per mole. More preferably, the molecular weight is less than about 750 grams per mole. Even more preferably, the molecular weight is less than about 700 grams per mole.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that-are formed between two adjacent ring atoms (e.g., C═C, C═N, or N═N). The present invention, in general, does not cover groups such as N-halo, S(O)H, and SO₂H.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R⁶) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

As used herein, “alkyl” is intended to include both branched and straight-chain saturated aliphatic hydrocarbon groups having the specified number of carbon atoms. C₁₋₆alkyl, is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. C₁₋₆ alkoxy, is intended to include C₁, C₂, C₃, C₄, C₅, and C₆ alkoxy groups. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. C₃₋₇ cycloalkyl is intended to include C₃, C₄, C₅, C₆, and C₇ cycloalkyl groups. Alkenyl” is intended to include hydrocarbon chains of either straight or branched configuration and one or more unsaturated carbon-carbon bonds that may occur in any stable point along the chain, such as ethenyl and propenyl. C₂-₆ alkenyl is intended to include C₂, C₃, C₄, C₅, and C₆ alkenyl groups. “Alkynyl” is intended to include hydrocarbon chains of either straight or branched configuration and one or more triple carbon-carbon bonds that may occur in any stable point along the chain, such as ethynyl and propynyl. C₂₋₆ Alkynyl is intended to include C₂, C₃, C₄, C₅, and C₆ alkynyl groups.

“Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3, 4, 5, 6, or 7-membered monocyclic or bicyclic or 7, 8, 9, 10, 11, 12, or 13-membered bicyclic or tricyclic ring, any of which may be saturated, partially unsaturated, or unsaturated (aromatic). Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl. As shown above, bridged rings are also included in the definition of carbocycle (e.g., [2.2.2]bicyclooctane). When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a trycyclic ring. When a ring is bridged, the substituents recited for the ring may also be present on the bridge.

As used-herein, the term “heterocycle” or “heterocyclic group” is intended to mean a stable 3, 4, 5, 6, or 7-membered monocyclic or 7, 8, 9, 10, 11, or 12-membered bicyclic or tricyclic heterocyclic ring which is saturated, partially unsaturated or unsaturated (aromatic), and which consists of carbon atoms and 1, 2, 3, 4, or 5 ring heteroatoms independently selected from the group consisting of N, O and S. Heterocycle includes any bicyclic group in which one heterocyclic ring is fused to a second ring, which may be carbocyclic (e.g. benzo fusion) or heterocyclic. When a heterocycle is referred to as an “aromatic heterocycle” or “heteroaryl,” this means that a fully unsaturated, i.e., aromatic, ring is present in the heterocycle. An aromatic heterocycle only requires one ring to be aromatic, if more than one ring is present. The aromatic portion of the aromatic heterocycle can be a carbocycle or heterocycle. The nitrogen and sulfur heteroatoms in the heterocycle may optionally be oxidized (i.e., N→O and S(O)p). The nitrogen atom may be unsubstituted (i.e., N or NH) or substituted (i.e., NR wherein R is a substituent) and may optionally be quaternized. The heterocyclic ring may be attached to its pendant group at any heteroatom or carbon atom that results in a stable structure. The heterocyclic rings described herein may be substituted on a carbon or on a nitrogen atom, if the resulting compound is stable. It is preferred that when the total number of S and O atoms in the heterocycle exceeds 1, then these heteroatoms are not adjacent to one another. It is preferred that the total number of S and O atoms in the heterocycle is not more than 1. It is to be noted that total number of S and O atoms in the aromatic heterocycle is not more than 1. Bridged and spiro rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a trycyclic ring. When a ring is bridged, the substituents recited for the ring may also be present on the bridge. Spiro rings are formed when to or more atoms (i.e., C, O, N, or S) of a chain are attached to the same carbon atom of a heterocycle (or carbocycle if fused to a heterocycle). When a spiro ring is present, the substituents recited for the ring may also be present on the spiro. When the term “heterocycle” is used, it is intended to include heteroaryl.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p 1445, the disclosure of which is hereby incorporated by reference.

In addition, compounds of formula I may have prodrug forms. Any compound that will be converted in vivo to provide the bioactive agent (i.e., a compound of formula I) is a prodrug within the scope and spirit of the invention.

Various forms of prodrugs are well known in the art. For examples of such prodrug derivatives, see:
a) Design of Prodrugs, edited by H. Bundgaard, (Elsevier, 1985), and Methods in Enzymology, Vol. 42, at pp. 309-396, edited by K. Widder, et. al. (Academic Press, 1985);
b) A Textbook of Drug Design and Development, edited by Krosgaard-Larsen and H. Bundgaard, Chapter 5, “Design and Application of Prodrugs,” by H. Bundgaard, at pp. 113-191 (1991);
c) H. Bundgaard, Advanced Drug Delivery Reviews, Vol. 8, p. 1-38 (1992);
d) H. Bundgaard, et al, Journal of Pharmaceutical Sciences, Vol. 77, p. 285 (1988); and
e) N. Kakeya, et. al., Chem Phar Bull., Vol. 32, p. 692 (1984).

Preparation of prodrugs is well known in the art and described in, for example, Medicinal Chemistry: Principles and Practice, ed. F. D. King, The Royal Society of Chemistry, Cambridge, UK, 1994, which is incorporated herein by reference in its entirety.

Radiolabelled compounds of the present invention, i.e., wherein one or more of the atoms described are replaced by a radioactive isotope of that atom (e.g., C replaced by ¹³C or by ¹⁴C; and isotopes of hydrogen include tritium and deuterium), are also provided herein. Such compounds have a variety of potential uses, e.g., as standards and reagents in determining the ability of a potential pharmaceutical to bind to target proteins or receptors, or for imaging compounds of this invention bound to biological receptors in vivo or in vitro.

“Stable compound” and “stable structure” are meant to indicate a compound that is sufficiently robust to survive isolation to a useful degree of purity from a reaction mixture, and formulation into an efficacious therapeutic agent. It is preferred that there presently recited compounds do not contain a N-halo, S(O)₂H, or S(O)H group.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention that is effective when administered alone or in combination to inhibit factor Xa. “Therapeutically effective amount” is also intended to include an amount of the combination of compounds claimed that is effective to inhibit factor Xa. The combination of compounds is preferably a synergistic combination. Synergy, as described, for example, by Chou and Talalay, Adv. Enzyme Regul. 1984, 22:27-55, occurs when the effect (in this case, inhibition of factor Xa) of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antithrombotic effect, or some other beneficial effect of the combination compared with the individual components.

The present invention further includes compositions comprising one or more compounds of the present invention and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., Mack Publishing Company, Easton, Pa., 1985, which is incorporated herein by reference in its entirety.

Synthesis

All references cited herein are hereby incorporated in their entirety herein by reference.

The compounds of the present invention can be prepared in a-number of ways known to one skilled in the art of organic synthesis. The compounds of the present invention can be synthesized using the methods described below, together with synthetic methods known in the art of synthetic organic chemistry, or by variations thereon as appreciated by those skilled in the art. Preferred methods include, but are not limited to, those described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process Scheme over another in order to obtain a desired compound of the invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Wiley and Sons, 1999).

The compounds of the present invention as represented as Formula I in Scheme 1 can be prepared as outlined in the following Schemes and via standard methods known to those skilled in the art. M in Formula I corresponds to the central ring of the present compounds.

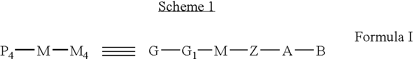

The A-B group for compounds of the present invention can be obtained via the Ullman reaction or Buchwald modified Ullman reaction (J. Am. Chem. Soc. 2001, 123, 7727) using CuI and 1,2-cyclohexyldiamine or 1,10-phenanthroline as the catalyst as outlined in Scheme 2.

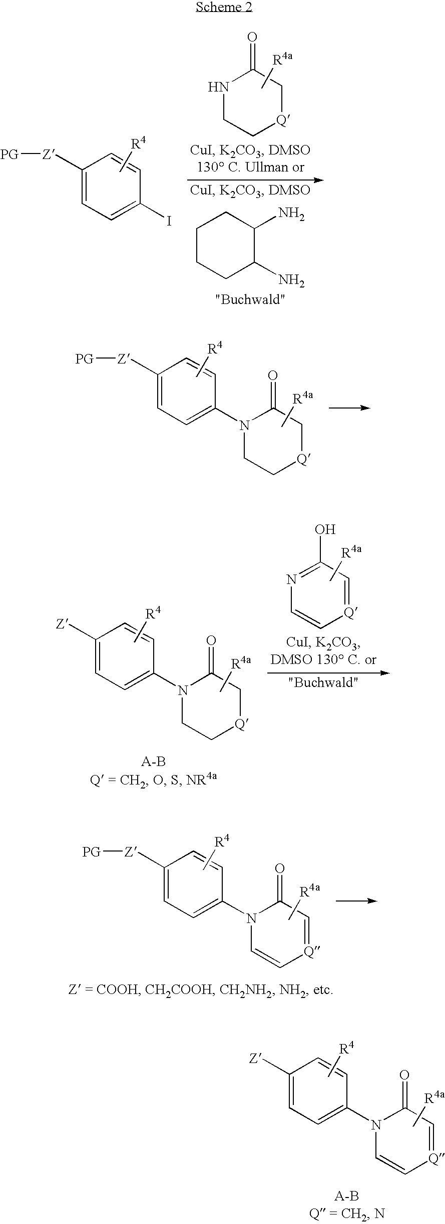

A-B groups wherein the B group contains an oxidizable group can be obtained by oxidation, e.g., S to SO and SO₂. The Ullman coupling methodology can also be applied to prepare the cyclic urea or cyclic carbamate analogs shown in Scheme 3.

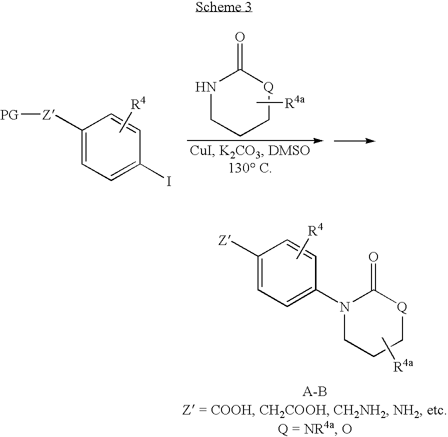

The A-B group can also be prepared via aromatic nucleophile displacement of substituted halo-benzenes followed by saponification as shown in Scheme 4.

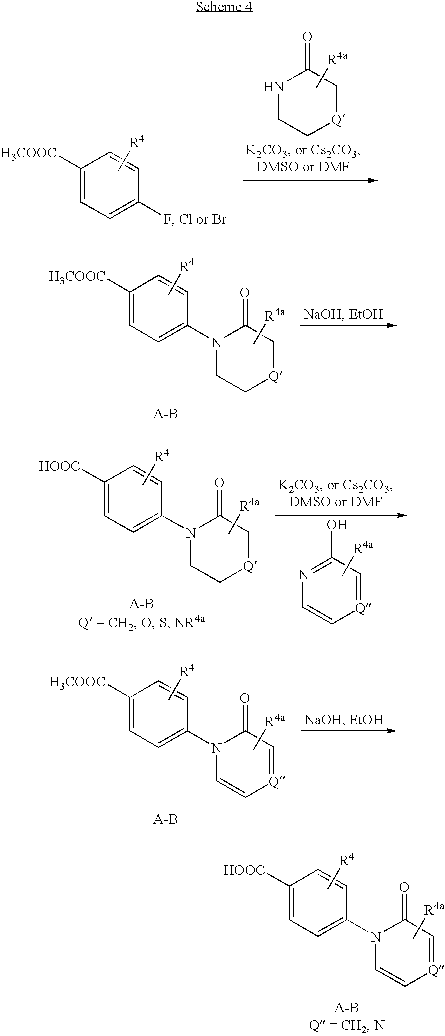

A-B groups can also be prepared via aromatic nucleophilic substitution of fluoronitrobenzenes with the 5-7 membered bases followed by α-carbon oxidation with KMnO₄ as shown in Scheme 5.

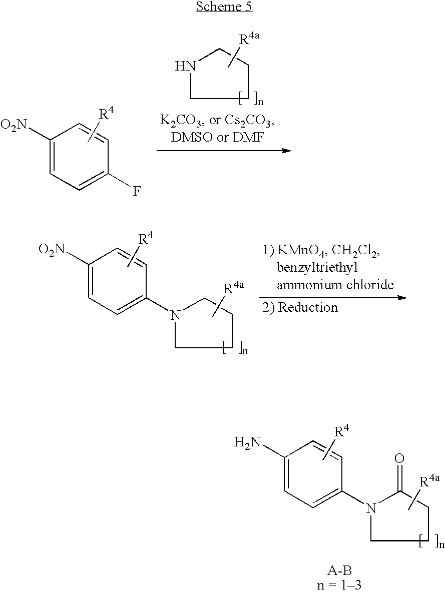

Lactam, cyclic sulfonamide, cyclic urea, and cyclic carbamate A-B analogs can also be prepared via the method outlined in Scheme 6.

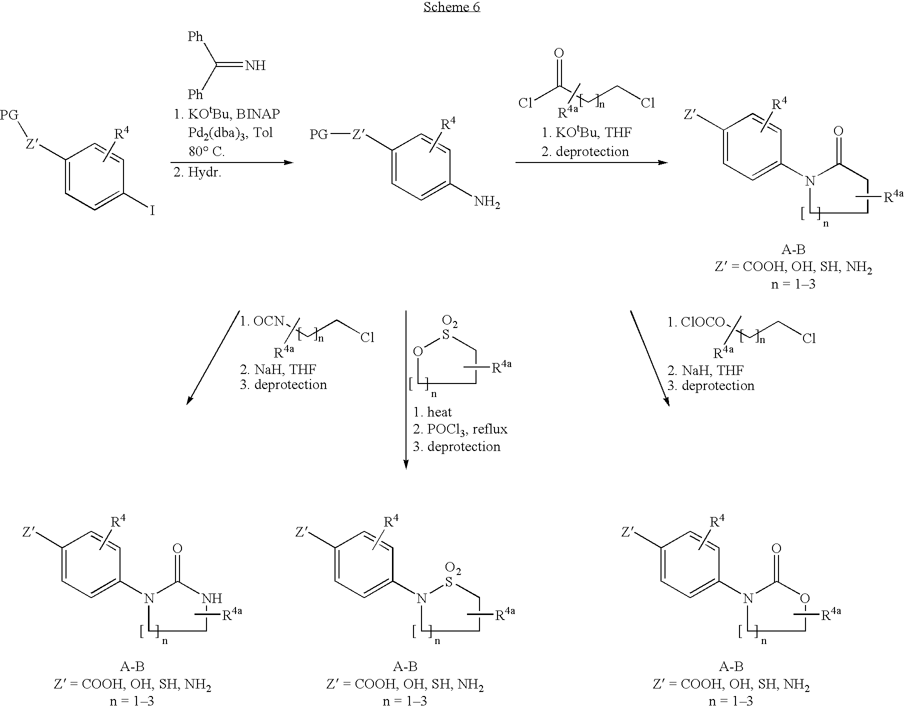

The piperidone A-B groups shown above can also be further elaborated to afford other compounds of the present invention by numerous methods known to those skilled in the art (e.g., see Scheme 7).

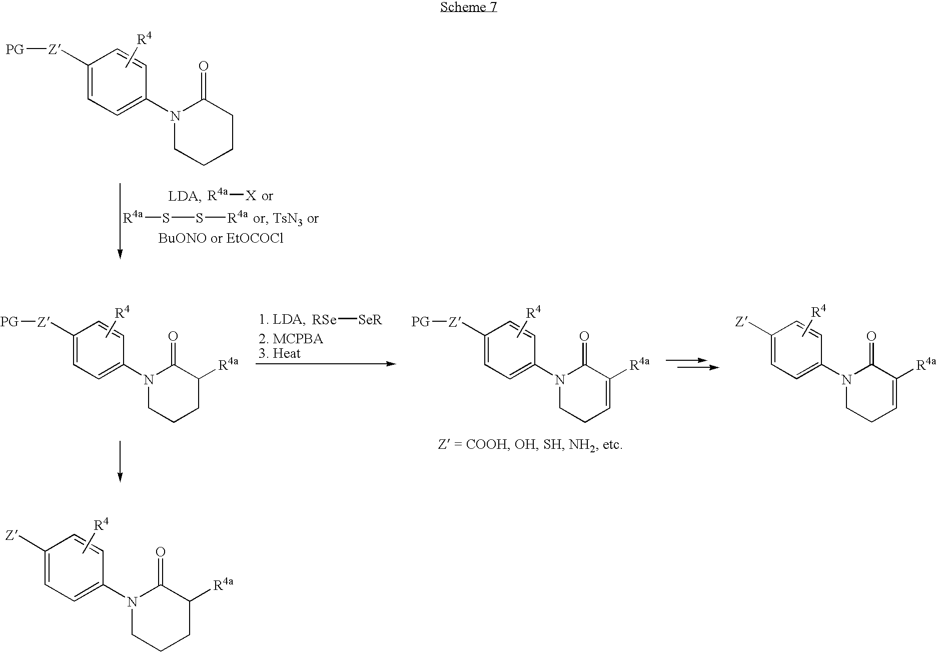

Additional A-B intermediates can be synthesized by chemical manipulation of the amino functionality of the compounds described above (see Scheme 8).

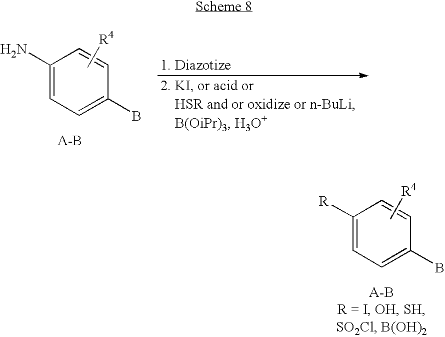

Other possible A-B groups can be synthesized from the carboxylic ester intermediates that can be homologated via the Arndt Eistert methodology to afford other A-B intermediates (see Scheme 9). Alternatively, the ester functionality can be reduced to the alcohol that in turn can be converted to a variety of A-B groups by procedures known to those skilled in the art.

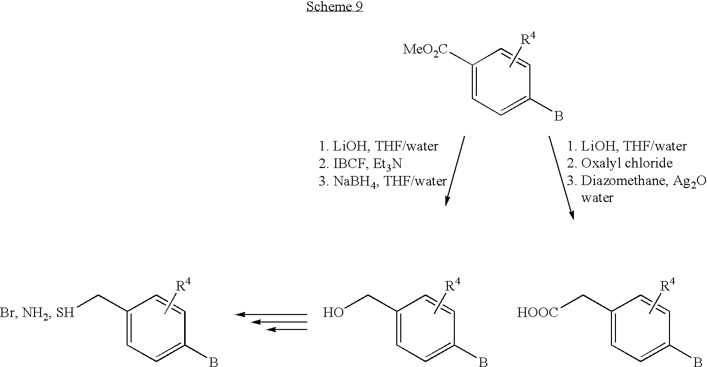

Substituted pyridyl and pyrimidyl A-B analogs (see structures below) can also be prepared using routes similar to those of Schemes 2-9.

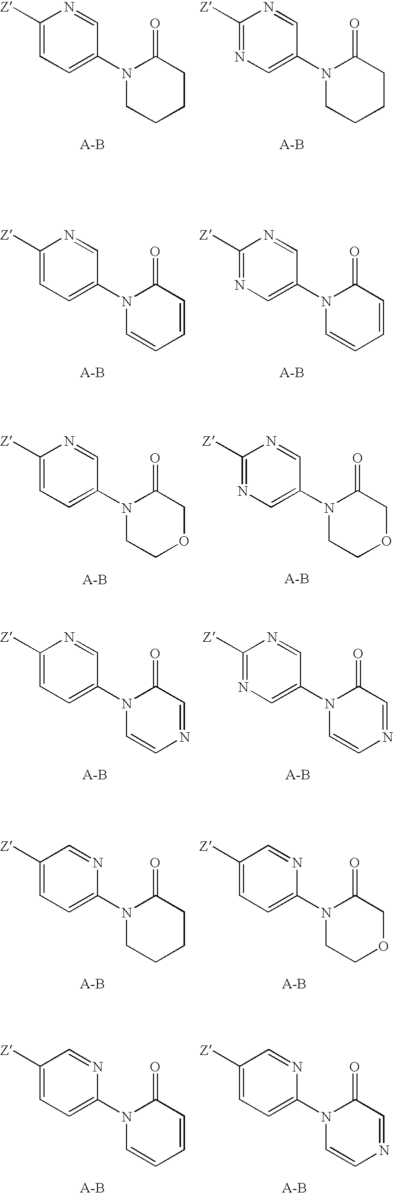

The non-aromatic intermediates in Scheme 10 can be synthesized via procedures known to those skilled in the art. These intermediates can than be further manipulated to incorporate substituent R^{4a} via procedures previously described.

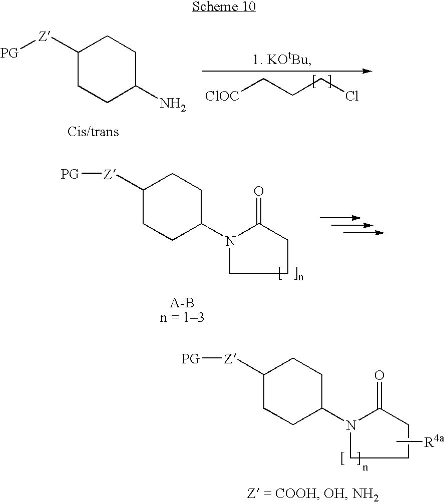

Alternative non-aromatic intermediates can be synthesized via procedures known to those skilled in the art, e.g., see Scheme 11. These intermediates can also be further manipulated to incorporate substituent R^{4a} via procedures described previously. Further modifications of the ester functionality can be done via procedures described above.

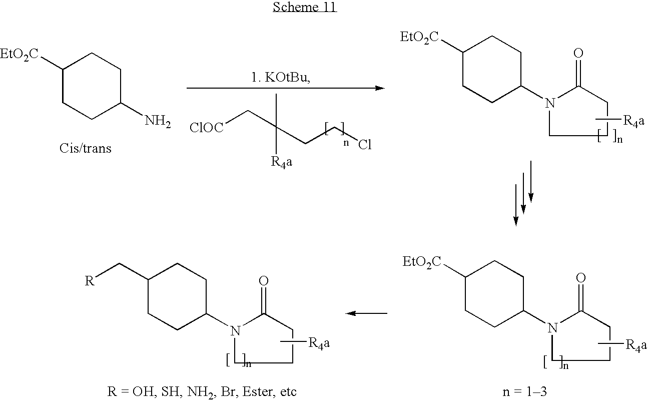

A-B groups wherein A is indoline can be prepared as shown in Scheme 12. Alternatively, the indoline can be attached to the other half of the desired compound prior to formation of the lactam ring.

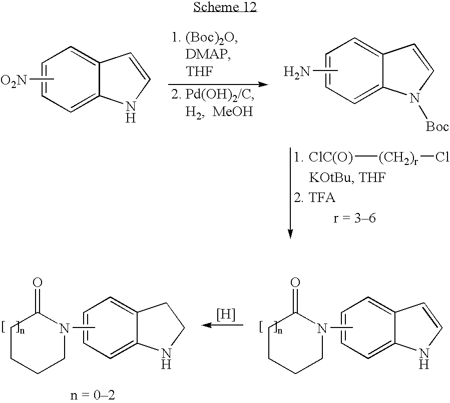

Schemes 2-12 describe how to make the A-B moieties of the present invention. For compounds wherein A is an indoline or similar bicycle, one of ordinary skill in the art can look to U.S. Pat. No. 6,429,205 for starting materials and intermediates to which the present B group can be coupled or from which the present A-B groups can be formed.

The functionalized G moiety of the present invention can be prepared using methods known to those of ordinary skill in the art. All of the following patents and publications are incorporated herein by reference. For compounds wherein G is a ring substituted with a basic moiety, one of ordinary skill in the art can look to U.S. Pat. No. 5,939,418, U.S. Pat. No. 5,925,635, U.S. Pat. No. 6,057,342, U.S. Pat. No. 6,187,797, U.S. Pat. No. 6,020,357, U.S. Pat. No. 6,060,491, U.S. Pat. No. 6,191,159, U.S. Pat. No. 6,339,099, U.S. Pat. No. 6,271,237 U.S. Pat. No. 6,399,644, U.S. Pat. No. 6,407,256, U.S. Pat. No. 6,413,980, WO02/00651, WO02/102380, WO02/094197, U.S. Pat. No. 2003/78,255, and U.S. Pat. No. 2003/18,023 for starting materials. For compounds wherein G is a ring substituted with a non-basic group, one of ordinary skill in the art can look to U.S. Pat. No. 5,998,424, U.S. Pat. No. 6,413,980, U.S. Pat. No. 6,399,644, U.S. Pat. No. 6,407,256, WO02/00651, WO02/102380, WO02/094197, US 2003/78,255, and US 2003/18,023 for starting materials. For compounds wherein G is a bicyclic moiety, one of ordinary skill in the art can look to U.S. Pat. No. 6,339,099 U.S. Pat. No. 6,369,227, U.S. Pat. No. 6,413,980, WO02/00651, WO02/102380, WO02/094197, US 2003/78,255, and US 2003/18,023 for starting materials.

For compounds wherein ring M is a 5- or 6-membered ring, one of ordinary skill in the art can look to WO01/70673 and WO02/2525 for starting materials and intermediates to which the present B and/or A-B groups and G and/or G₁-G groups can be coupled.

Compounds of the present invention wherein ring M is a non-aromatic carbocycle or heterocycle can be prepared by using the methods known to those skilled in the art. Scheme 13 illustrates some of the monocyclic/heterocyclic M intermediates that can be used to prepared compounds of the present invention (R^{z} is the point of attachment of Z-A-B and can be a protecting group; a group modifiable to Z or A-Z; or Z, A-Z, or A; R^{g} is the point of attachment for G₁-G and can be a protecting group, a group modifiable to G₁, or G₁-G). These intermediates can be prepared using methods known to those of ordinary skill in the art.

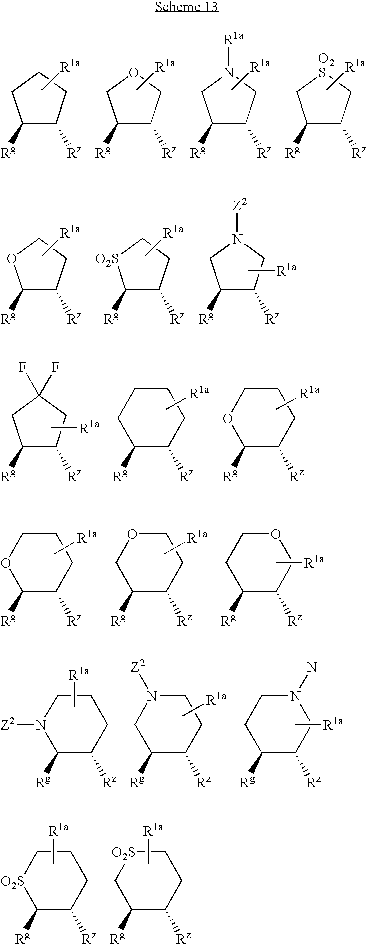

Scheme 14 describes general methods of converting the M rings shown in Scheme 13 to compounds of the present invention wherein linker Z can be —NHCO—, —NHCH₂—, and —NHSO₂—. The cyclic β-amino acid intermediate 1 can be acylated, sulfonylated or reductively aminated (or alternatively alkylated) to give 2-4, respectively.

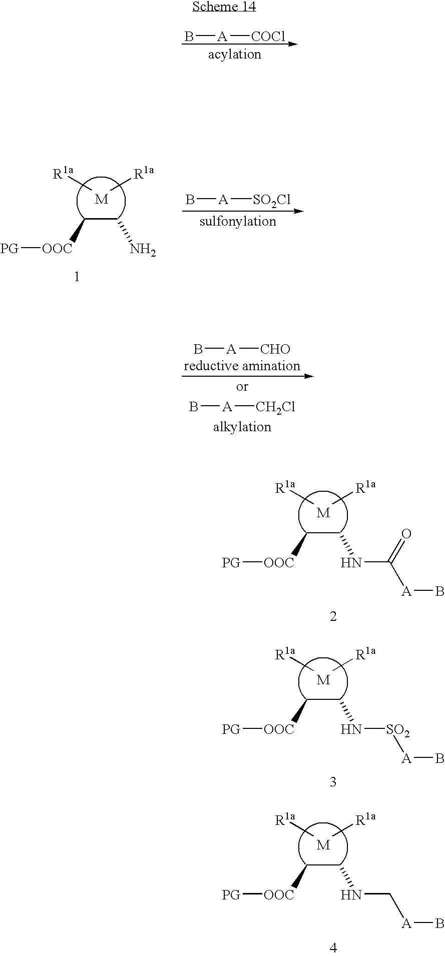

Scheme 15 describes general methods of converting the elaborated M rings shown in Scheme 14 to compounds of the present invention wherein linker G₁ can be —CONH— and —CH₂NH—. Deprotection of 5, followed by standard coupling gives 6 where G1 is —CONH—. Alternatively the ester in 5 can be converted directly to the amide 6 according to Weinreb's conditions with trimethylaluminum. When G₁ is —CH₂NH—, amide 7 is reduced to amine 8 which is converted to 9 according to Scheme 14. Alternatively, the ester can be converted to aldehyde 11. Following reductive amination of 11 with G-NH₂, deprotection, and subjection to Scheme 14 gives 9.

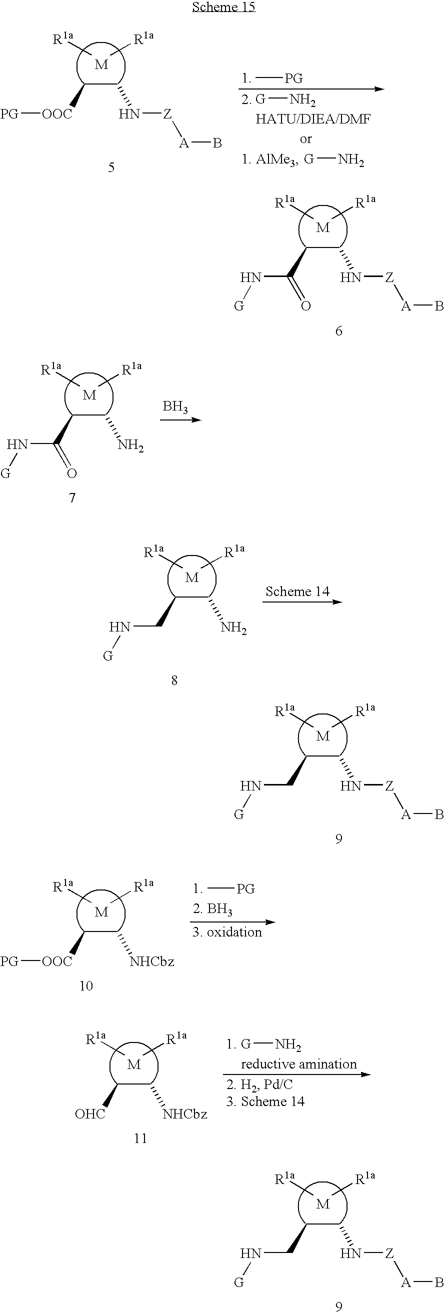

Alternatively, the linkers can be reversed such that B-A-COCl, B-A-CHO, B-A-CH₂Cl, and B-A-SO₂Cl in Scheme 14 can be replaced by G-COCl, G-CHO, G-CH₂Cl, and G-SO₂Cl, respectively. Similarly, G-NH₂ in Scheme 15 can be replaced by B-A-NH₂.

Schemes 16-26 describe the synthesis of a variety of monocyclic M cores. The β-amino ester moiety can be synthesized following a variety of literature routes as reviewed in “Enantioselective Synthesis of β-Amino Acids” (E. Juaristi, Ed. Wiley-VCH, 1997). A series of compounds where M is a carbocycle can be prepared as described in Schemes 16-20. One approach using Davies protocol is summarized in Scheme 16 (Davies, S. G. et. al. J. Chem. Soc. Perkin Tran I, 1994, 1411). Michael addition of lithium amide 13 to 14 gives cis-product 15. Epimerization of 15 with KHMDS in tert-butanol gives the trans-isomer 16. Hydrogenolysis over palladium on carbon provides 17. The stereochemical configuration of 15 is governed by the chirality of 13. Since both amine enantiomers of 13 are commercially available, this approach provides ready access to both enantiomers.

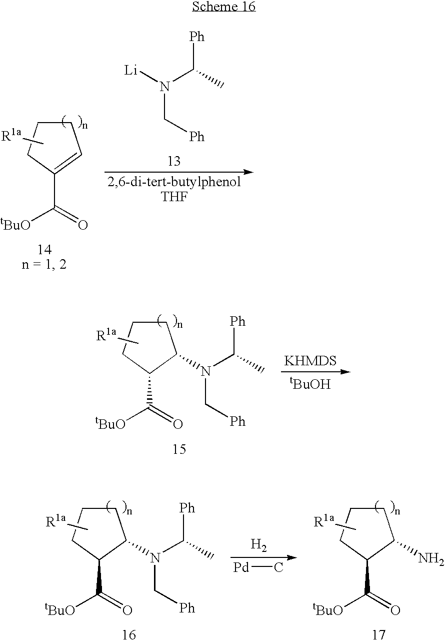

Alternatively, the carbocyclic β-amino acid can be prepared according to Perlmutter's protocol which is summarized in Scheme 17 (Perlmutter, P. Eur. J. Org. Chem. 2003, 756.) Diastereoselective condensation of pyridyl thioester 18 with enantiopure imine 19 gives lactam 20. Opening of the lactam with benzyl alcohol and chlorotrimethylsilane followed by ring closing metathesis with Grubb's catalyst gives the carbocyclic β-amino ester 21. The olefin provides a handle for introducing additional functional groups (see Wipf, P. et al.; Tetrahedron Letters, 2000, 41, 8747). Hydrogenolysis gives amino acid 22. (For a similar ring-closing metathesis approach see: Davies, S. Tetrahedron, 2003, 59, 3253).

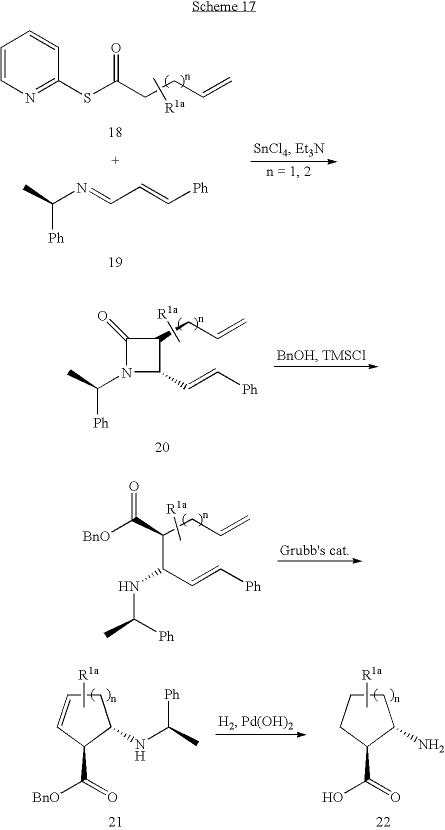

Alternatively, these carbocyclic β-amino acids can be prepared from the corresponding dicarboxylate derivatives (Scheme 18) The dicarboxylate derivatives 23 can be desymmetrized either through enzymatic resolution (for example with lipase, see Gais, H. J. et al. J. Am. Chem. Soc. 1989, 54, 5115) or through chemical resolution (for example with TADOLates, see Seebach, D. et al., Angew. Chem. Int. Ed. Engl. 1995, 34, 2395 or Knochel, P. et al. Tetrahedron: Asymmetry, 1997, 8, 987.).

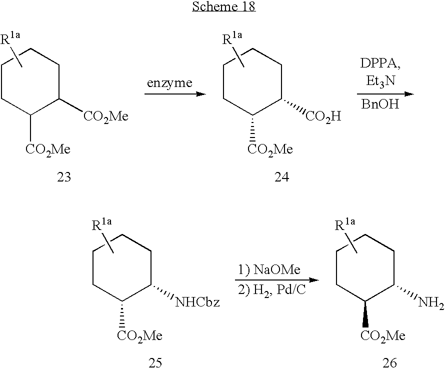

The optically pure mono-ester 24 is converted to Cbz protected β-amino acid ester 25 through Curtius rearrangement (for a related example, see Ohno, M. et al, Tetrahedron Lett. 1984, 25, 2557). Epimerization under basic conditions gives the trans-isomer and removal of the Cbz protecting provides amino ester 26. In a similar method, the trans-dicarboxylate can be converted in one pot procedure according to Berkessel to give the β-amino acid 27. (Scheme 19) (Berkessel, A. et al., Eur. J. Org. Chem. 2002, 2948). The dicarboxylates can be synthesized via Diels-Alder chemistry (see Wipf, P. et al.; Tetrahedron Letters, 2000, 41, 8747).

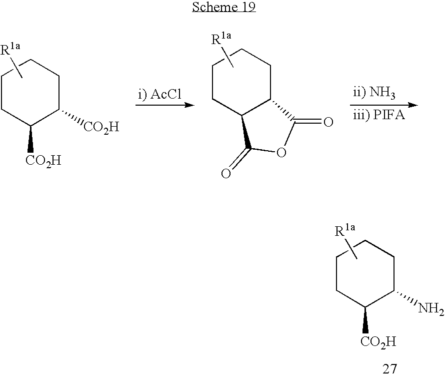

Another procedure for the synthesis of cyclic β-amino acids employs a [2+2] cycloaddition of chlorosulfonyl isocyanate and alkenes to give the β-lactam 28 (Scheme 20, Dhar, D. N.; Murthy, K. S. K. Synthesis 1986, 437-449 and Fulop, F. et al. Tetrahedron: Asymmetry, 2003, 14, 3965). The β-lactam can be opened with a variety of reagent such as chlorotrimethylsilane/alcohol solvent to give the cis-β-amino ester. The trans-β-amino acids 29 are available by equilibration under basic conditions.

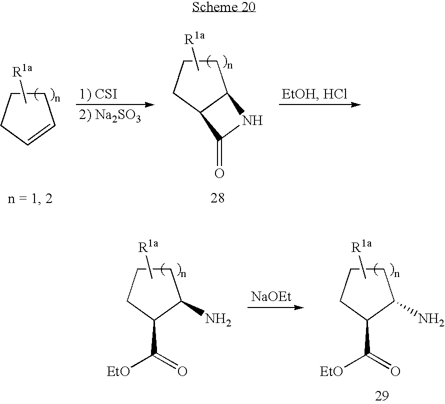

A series of compounds where M is pyrrolidine can be prepared as described in Scheme 21. 1,3-Dipolar cycloaddition of in situ generated non-stabilized azomethine ylide with benzyl methyl fumerate, according to Joucla's procedure, gives pyrrolidine 30 (Joucla, M.; Mortier, J., J. Chem. Commun. 1985, 1566). Protecting group manipulations and Curtius rearrangement gives the β-amino ester 31. Hydrogenolysis provides amino acid ester 32.

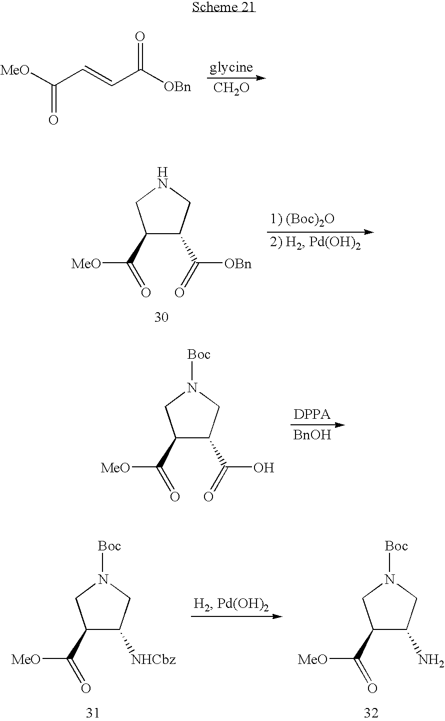

Following the introduction of the desired G-G, and Z-A-B groups as described in Schemes 14 and 15, the pyrrolidine nitrogen is unmasked and, functionalized to various tertiary amines, amides, ureas, sulfonamides, and sulfonyl ureas 34 following procedures well known in the literature (Scheme 22). Alternatively, following the introduction of the desired Z-A-B groups as described in Scheme 14, the pyrrolidine nitrogen is unmasked to give 35. Functionalized to various tertiary amines, amides, ureas, sulfonamides, and sulfonyl ureas following procedures well known in the literature to give 36. The desired G-G₁ groups can be installed in 36 to give 34 according to Scheme 15.

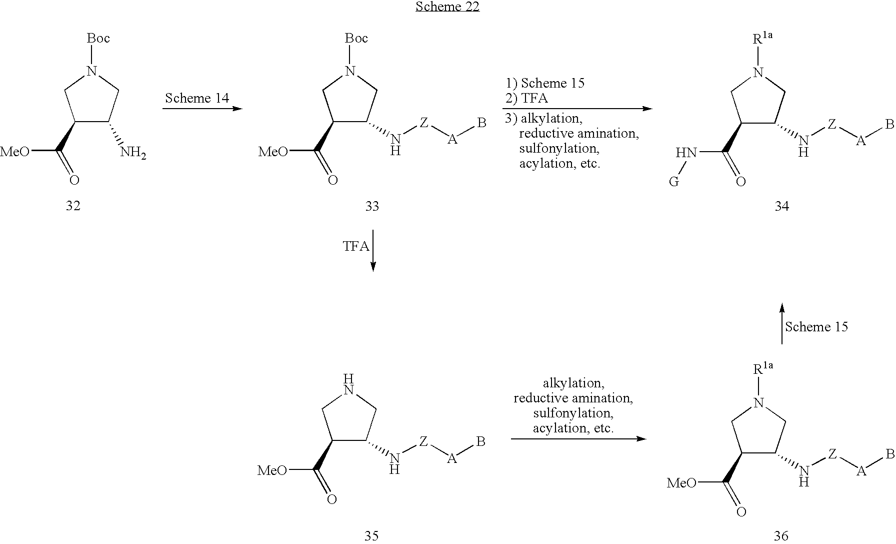

A series of compounds where ring M is piperidine can be prepared following the sequence outlined in Scheme 23 (Gellman, S. H. Eur. J. Org. Chem., 2003, 721). Stereoselective reduction of β-enamino ester 37 according to Palmieri gives the β-amino ester moiety with cis-stereochemistry (Palmieri, G. et. al., J. Org. Chem. 1996, 61, 5557). The trans-isomer is prepared by epimerization under basic conditions (eg. DBU, toluene, reflux or with alkoxide) to give 38. Hydrogenolysis gives amino acid ester 39. Installation of the desired G-G₁ and Z-A-B groups as well as functionalization of the piperidine nitrogen can be achieved as described for the pyrrolidine series in Scheme 22.

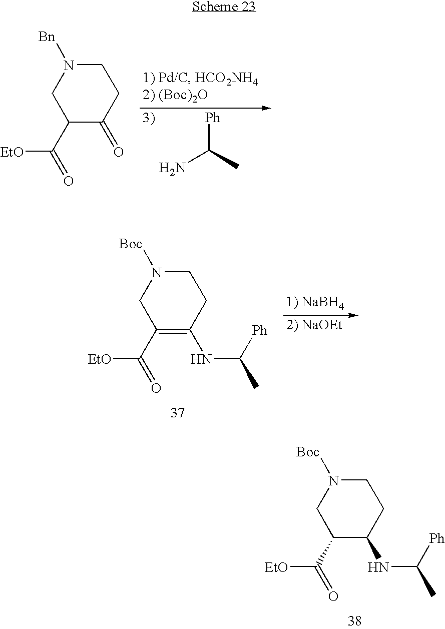

In a very similar manner, the other 3,4-disubstituted piperidine regioisomer can be converted to 40 as shown in Scheme 24. Installation of the desired G-G₁ and Z-A-B groups as well as functionalization of the piperidine nitrogen can be achieved as described for the pyrrolidine series in Scheme 22.

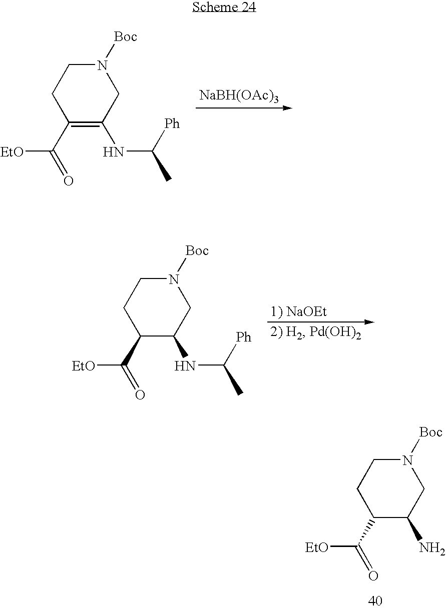

Scheme 25 describes the synthesis of 3,4-disubstituted tetrahydropyrans. Coupling of oxazolinone 41 with cinnamoyl chloride and subsequent boron-mediated aldol condensation (Galatsis, P.; Millan, S. D.; Ferguson, G.; J. Org. Chem. 1997, 62(15), 5048) with aldehyde 42 can give alcohol 43. Lithium borohydride auxiliary removal, protection of the primary alcohol with TBSCl, mesylate formation of the secondary alcohol, displacement of the mesylate with azide and reduction of the azide followed by protection gives 44. Ozonolysis followed by reductive workup, mesylate formation of the alcohol, selective fluoride deprotection of the TBMPS (Guindon, Y.; Fortin, R.; Yaokim, C.; Gillard, J. W.; Tetrahedron Letters, 1984, 25, 4717), and basic cyclization can provide tetrahydropyrans 45. Fluoride deprotection followed by Swern oxidation can produce aldehyde 46 for reductive amination. Alternatively, the alcohol can be oxidized with PDC (Corey, E. J.; Schmidt, G. Tetrahedron Letters, 1979, 5, 399) to acid 47.

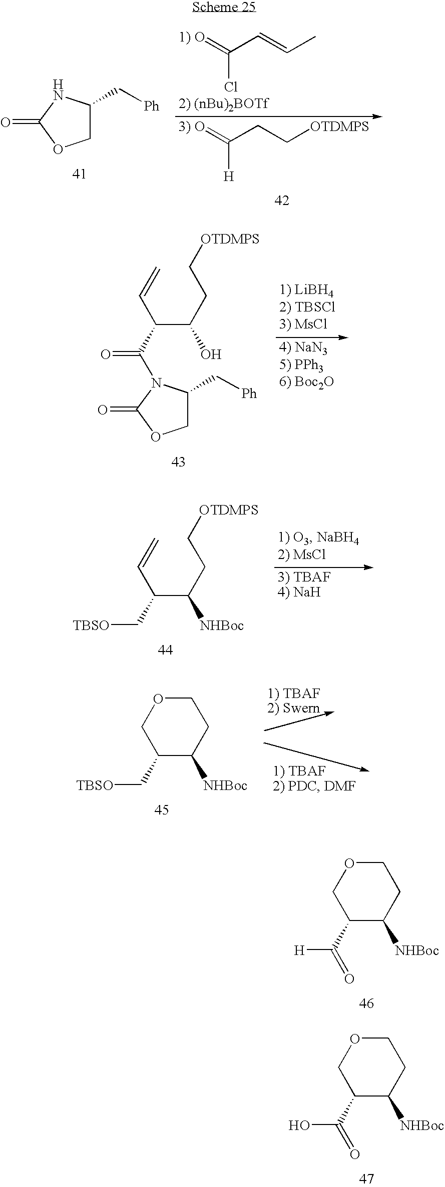

The preparation of the regioisomeric 3,4-disubstituted tetrahydropyran is shown in Scheme 26. One of the key differences is the aldol reaction with the truncated aldehyde 48. Furthermore, instead of ozonolysis, the olefin 49 is hydroborated, the resulting alcohol mesylated, and after deprotection, undergoes ring closure to give the desired tetrahydropyran 50. Oxidation can give either 51 or 52.

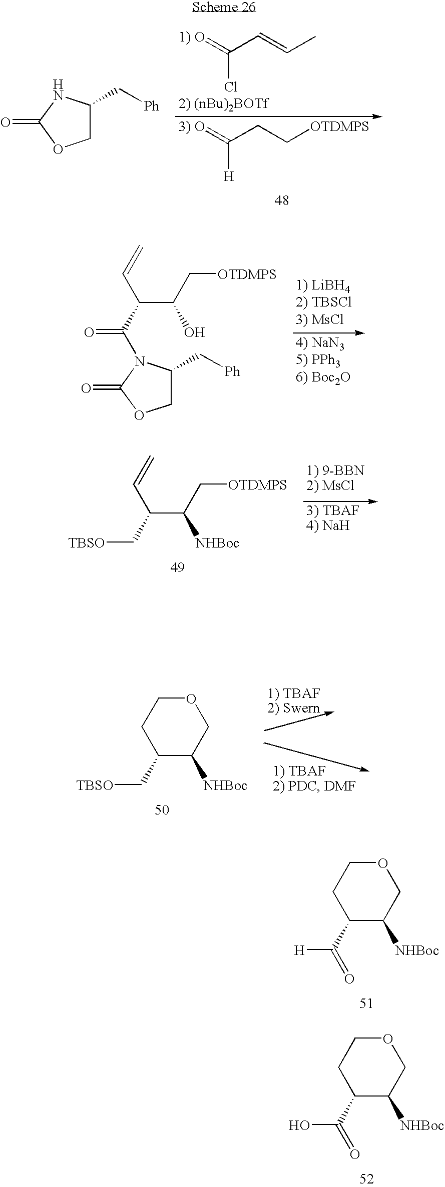

Scheme 27 illustrates numerous bicyclic M intermediates that can be used to prepare compounds of the present invention. These intermediates can be prepared using methods known to those of ordinary skill in the art and using similar methods described previously.

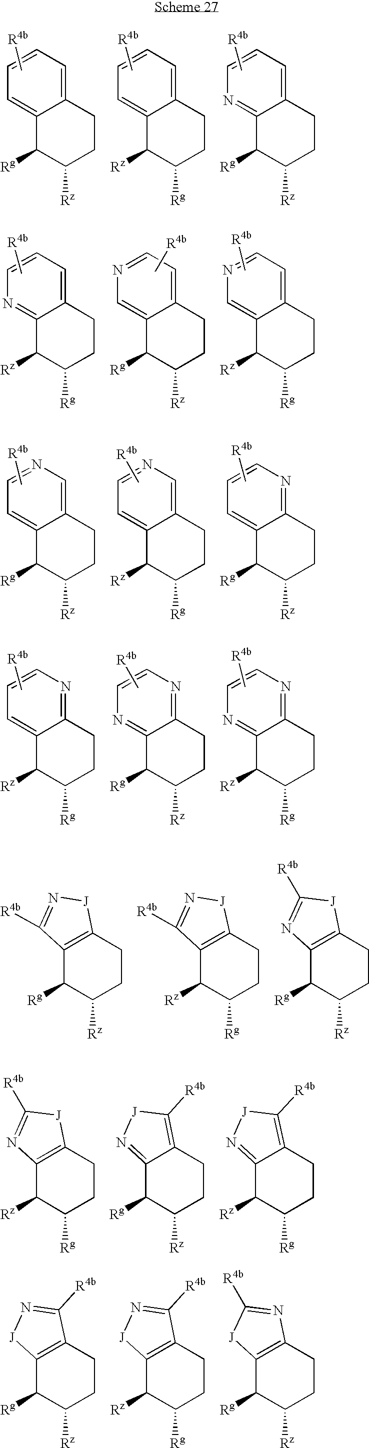

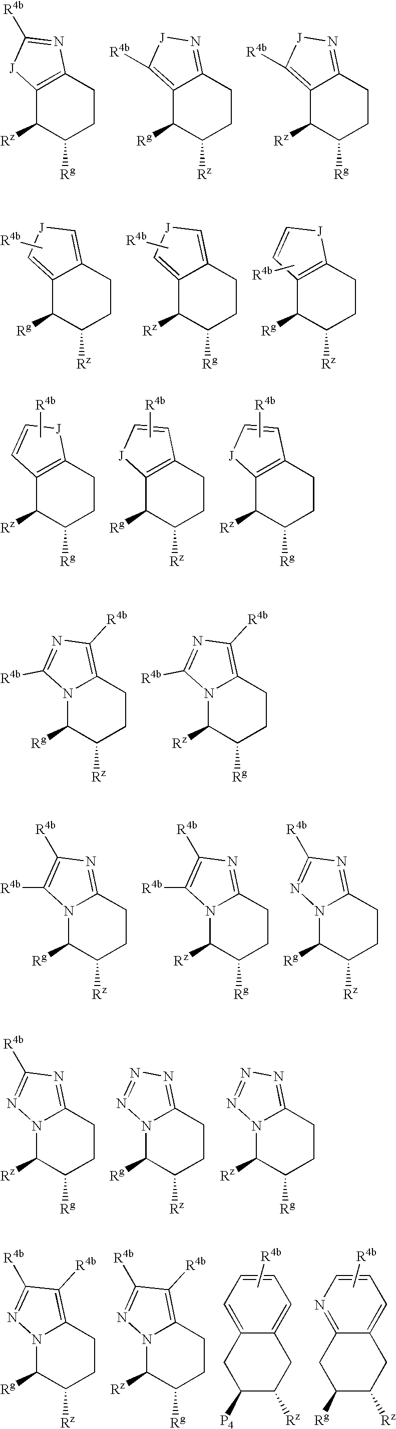

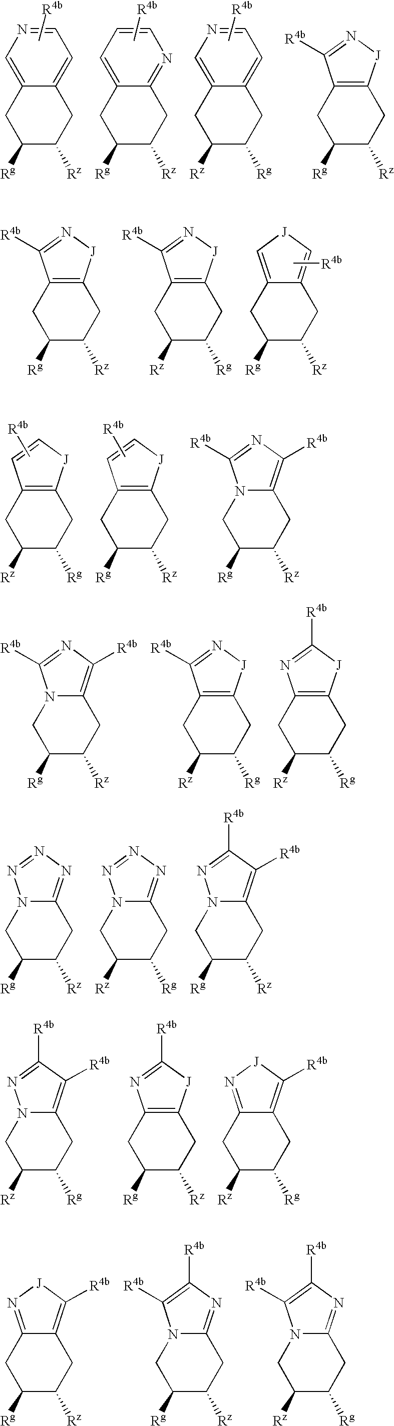

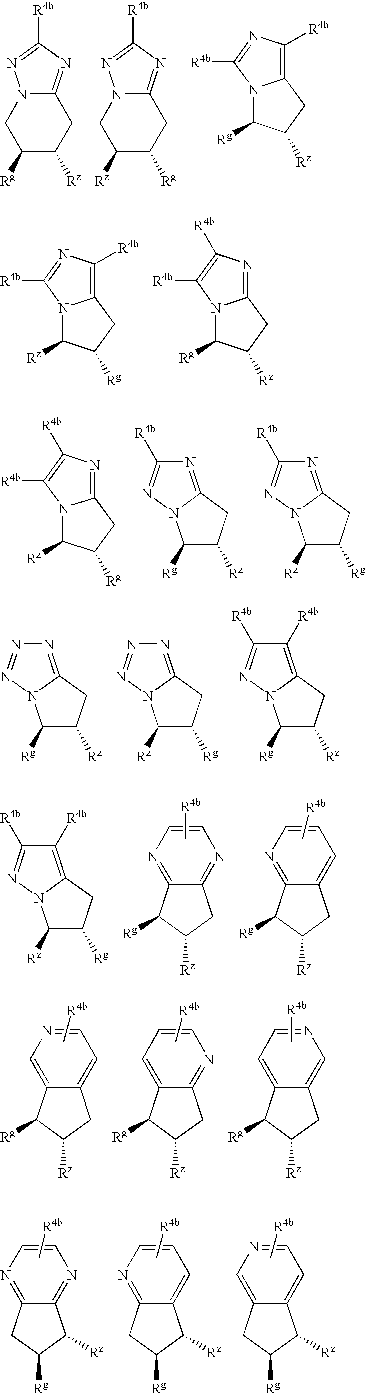

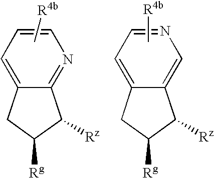

A series of compounds where M is indane can be prepared as described in Scheme 28. [2+2] Cycloaddition of chlorosulfonyl isocyanate and indene gives the β-lactam 53. Ring opening and epimerization gives the 1-aminoindane-2-carboxylic ester 54. (Fulop, F. et. al. Tetrahedron: Asymmetry, 2000, 11, 4179.)

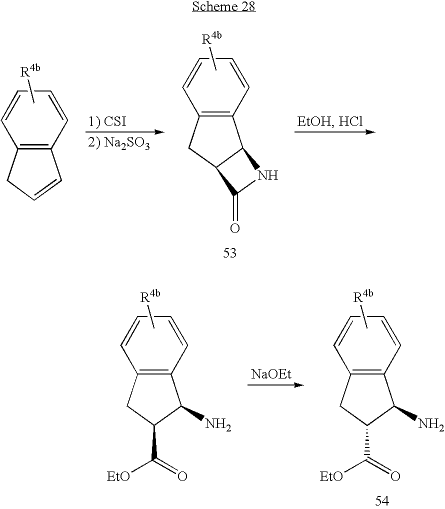

Alternatively, Scheme 29 describes direct preparation of the trans-1-aminoindane-2-carboxylic ester 56. Conjugate addition of lithium methyl benzyl amide 13 followed by intramolecular alkylation gives indane skeleton 55 possessing the trans-stereochemistry. (Price, D. A. Synlett, 1999, 12, 1919.) Transesterification and hydrogenolysis via transfer hydrogenation gives 56.

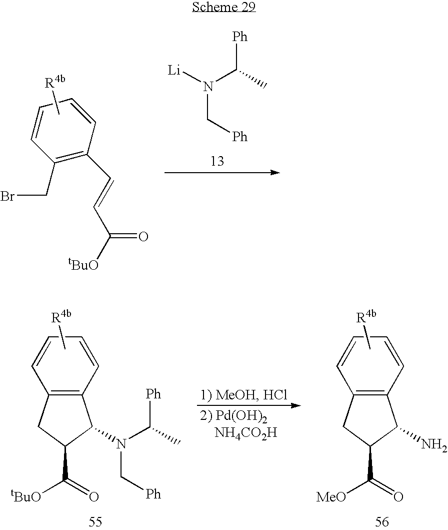

Scheme 30 depicts numerous spiro and bridged M intermediates that can be used to prepare compounds of the present invention. These intermediates can be prepared using methods known to those of ordinary skill in the art and using the methods described previously.

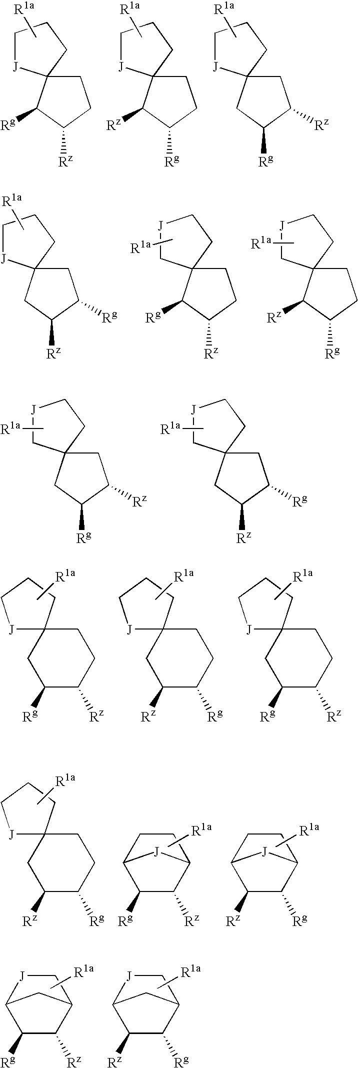

One enantiomer of a compound of formula I may display superior activity compared with the other (Scheme 31). Thus the following absolute stereochemistries are considered to be part of the present invention. Asymmetric syntheses as described above can be used to synthesize chiral, nonracemic material. When racemic syntheses are performed the enantiomers can be separated by HPLC using a chiral column or by resolution using a resolving agent.

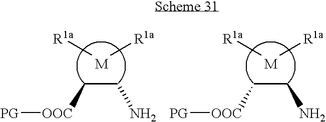

Representative cyclohexane β-amino acid derivatives of the invention can be prepared as shown in Scheme 32 and 33. Esterification of the trans-2-amino-1-cyclohexane carboxylic acid in an acidic alcohol solvent such as methanol at elevated temperatures provides the amino ester which is acetylated with an acid chloride, such as 57, in a solvent such as dichloromethane and with a base such as pyridine to give 58. Coupling of methyl ester 58 and an aniline, such as 5-chloro-2-amino pyridine, with trimethylaluminum in a solvent such as dichloroethane at elevated temperatures gives the bis-amide 59. (Weinreb, S. M., Tetrahedron Lett., 1977, 48, 4171.)

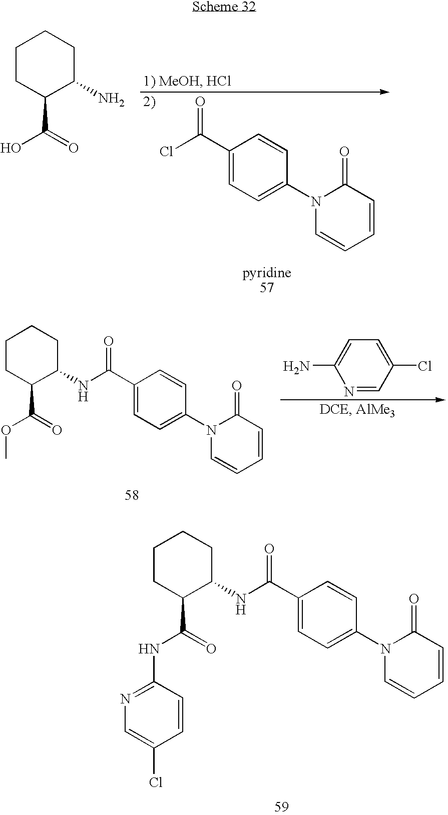

Amino ester 60 is acetylated with an acid chloride, such as 4-chlorobenzoylchloride, in a solvent such as dichloromethane and with a base such as pyridine to give 61. Coupling of methyl ester 61 and an aniline, such as 62, with trimethylaluminum in a solvent such as dichloroethane at elevated temperatures gives the bis-amide 63. (Weinreb, S. M., Tetrahedron Lett., 1977, 48, 4171.) 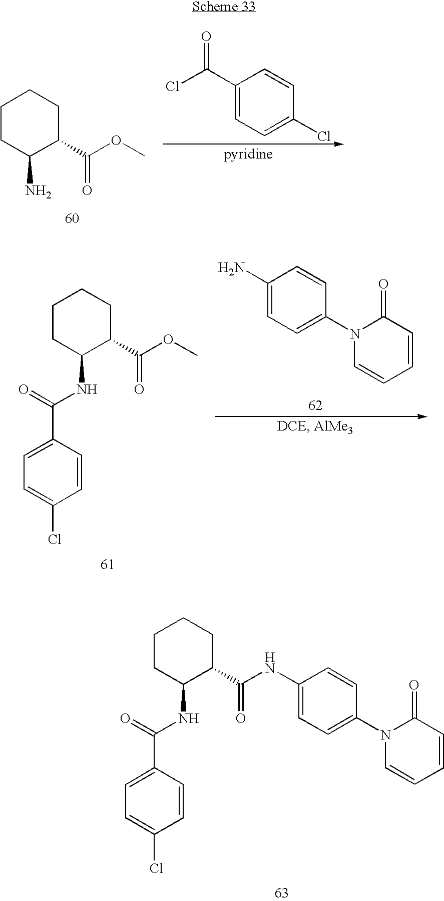
Utility

The compounds of this invention are inhibitors of factor Xa and are useful as anticoagulants for the treatment or prevention of thromboembolic disorders in mammals (i.e., factor Xa-associated disorders). In general, a thromboembolic disorder is a circulatory disease caused by blood clots (i.e., diseases involving fibrin formation, platelet activation, and/or platelet aggregation). The term “thromboembolic disorders” as used herein includes arterial cardiovascular thromboembolic disorders, venous cardiovascular or cerebrovascular thromboembolic disorders, and thromboembolic disorders in the chambers of the heart. The term “thromboembolic disorders” as used herein also includes specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g. after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy. The anticoagulant effect of compounds of the present invention is believed to be due to inhibition of factor Xa or thrombin.

The effectiveness of compounds of the present invention as inhibitors of factor Xa was determined using purified human factor Xa and synthetic substrate. The rate of factor Xa hydrolysis of chromogenic substrate S2222 (Diapharma/Chromogenix, West Chester, Ohio) was measured both in the absence and presence of compounds of the present invention. Hydrolysis of the substrate resulted in the release of pNA, which was monitored spectrophotometrically by measuring the increase in absorbance at 405 nm. A decrease in the rate of absorbance change at 405 nm in the presence of inhibitor is indicative of enzyme inhibition. The results of this assay are expressed as inhibitory constant, Kᵢ.

Factor Xa determinations were made in 0.10 M sodium phosphate buffer, pH 7.5, containing 0.20 M NaCl, and 0.5% PEG 8000. The Michaelis constant, Kₘ, for substrate hydrolysis was determined at 25° C. using the method of Lineweaver and Burk. Values of Kᵢ were determined by allowing 0.2-0.5 nM human factor Xa (Enzyme Research Laboratories, South Bend, Ind.) to react with the substrate (0.20 mM−1 mM) in the presence of inhibitor. Reactions were allowed to go for 30 min and the velocities (rate of absorbance change vs. time) were measured in the time frame of 25-30 min. The following relationship was used to calculate Kᵢ values:
(vₒ−vₛ)vₛ=I/(Kᵢ(1+S/Kₘ))
where:
vₒ is the velocity of the control in the absence of inhibitor;
vₛ is the velocity in the presence of inhibitor;
I is the concentration of inhibitor;
Kᵢ is the dissociation constant of the enzyme:inhibitor complex;
S is the concentration of substrate;
Kₘ is the Michaelis constant.

Compounds tested in the above assay are considered to be active if they exhibit a Kᵢ of ≦10 μM. Preferred compounds of the present invention have Kᵢ's of ≦1 μM. More preferred compounds of the present invention have Kᵢ's of ≦0.1 μM. Even more preferred compounds of the present invention have Kᵢ's of ≦0.01 μM. Still more preferred compounds of the present invention have Kᵢ's of ≦0.001 μM. Using the methodology described above, a number of compounds of the present invention were found to exhibit Kᵢ's of ≦10 μM, thereby confirming the utility of the compounds of the present invention as effective Xa inhibitors.

The antithrombotic effect of compounds of the present invention can be demonstrated in a rabbit arterio-venous (AV) shunt thrombosis model. In this model, rabbits weighing 2-3 kg anesthetized with a mixture of xylazine (10 mg/kg i.m.) and ketamine (50 mg/kg i.m.) are used. A saline-filled AV shunt device is connected between the femoral arterial and the femoral venous cannulae. The AV shunt device consists of a piece of 6-cm tygon tubing that contains a piece of silk thread. Blood will flow from the femoral artery via the AV-shunt into the femoral vein. The exposure of flowing blood to a silk thread will induce the formation of a significant thrombus. After 40 min, the shunt is disconnected and the silk thread covered with thrombus is weighed. Test agents or vehicle will be given (i.v., i.p., s.c., or orally) prior to the opening of the AV shunt. The percentage inhibition of thrombus formation is determined for each treatment group. The ID₅₀ values (dose which produces 50% inhibition of thrombus formation) are estimated by linear regression.

The compounds of the present invention may also be useful as inhibitors of serine proteases, notably human thrombin, Factor VIIa, Factor IXa, Factor XIa, urokinase, plasma kallikrein, and plasmin. Because of their inhibitory action, these compounds are indicated for use in the prevention or treatment of physiological reactions, blood coagulation and inflammation, catalyzed by the aforesaid class of enzymes. Specifically, the compounds have utility as drugs for the treatment of diseases arising from elevated thrombin activity such as myocardial infarction, and as reagents used as anticoagulants in the processing of blood to plasma for diagnostic and other commercial purposes.

Some compounds of the present invention were shown to be direct acting inhibitors of the serine protease thrombin by their ability to inhibit the cleavage of small molecule substrates by thrombin in a purified system. In vitro inhibition constants were determined by the method described by Kettner et al. in J. Biol. Chem. 265, 18289-18297 (1990), herein incorporated by reference. In these assays, thrombin-mediated hydrolysis of the chromogenic substrate S2238 (Helena Laboratories, Beaumont, Tex.) was monitored spectrophotometrically. Addition of an inhibitor to the assay mixture results in decreased absorbance and is indicative of thrombin inhibition. Human thrombin (Enzyme Research Laboratories, Inc., South Bend, Ind.) at a concentration of 0.2 nM in 0.10 M sodium phosphate buffer, pH 7.5, 0.20 M NaCl, and 0.5% PEG 6000, was incubated with various substrate concentrations ranging from 0.20 to 0.02 mM. After 25 to 30 min of incubation, thrombin activity was assayed by monitoring the rate of increase in absorbance at 405 nm that arises owing to substrate hydrolysis. Inhibition constants were derived from reciprocal plots of the reaction velocity as a function of substrate concentration using the standard method of Lineweaver and Burk. Using the methodology described above, some compounds of this invention were evaluated and found to exhibit a Kᵢ of less than 10 μm, thereby confirming the utility of the compounds of the present invention as effective thrombin inhibitors.

The compounds are administered to a mammal in a therapeutically effective amount. By “therapeutically effective amount” it is meant an amount of a compound of the present invention that, when administered alone or in combination with an additional therapeutic agent to a mammal, is effective to treat a thromboembolic condition or disease.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

Additional therapeutic agents include other anti-coagulant or coagulation inhibitory agents, anti-platelet or platelet inhibitory agents, thrombin inhibitors, thrombolytic or fibrinolytic agents, anti-arrythmic agents, anti-hypertensive agents, calcium channel blockers (L-type and T-type), cardiac glycosides, diruetics, mineralocorticoid receptor antagonists, phospodiesterase inhibitors, cholesterol/lipid lowering agents and lipid profile therapies, anti-diabetic agents, anti-depressants, anti-inflammatory agents (steroidal and non-steroidal), anti-osteoporosis agents, hormone replacement therapies, oral contraceptives, anti-obesity agents, anti-anxiety agents, anti-proliferative agents, anti-tumor agents, anti-ulcer and gastroesophageal reflux disease agents, growth hormone and/or growth hormone secretagogues, thyroid mimetics (including thyroid receptor antagonist), anti-infective agents, anti-viral agents, anti-bacterial agents, and anti-fungal agents.

Other anticoagulant agents (or coagulation inhibitory agents) that may be used in combination with the compounds of this invention include warfarin and heparin (either unfractionated heparin or any commercially available low molecular weight heparin, for example LOVENOX™), synthetic pentasaccharide, direct acting thrombin inhibitors including hirudin and argatrobanas, factor VIIa, IXa, XIa inhibitors, well as other factor Xa inhibitors such as those described in the publications identified above under Background of the Invention.

The term anti-platelet agents (or platelet inhibitory agents), as used herein, denotes agents that inhibit platelet function, for example by inhibiting the aggregation, adhesion or granular secretion of platelets. Agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. Of the NSAIDS, aspirin (acetylsalicyclic acid or ASA) and piroxicam are preferred. Other suitable platelet inhibitory agents include IIb/IIIa antagonists (e.g., tirofiban, eptifibatide, and abciximab), thromboxane-A2-receptor antagonists (e.g., ifetroban), thromboxane-A2-synthetase inhibitors, phosphodiesterase-III (PDE-III) inhibitors (e.g., dipyridamole, cilostazol), and PDE V inhibitors (such as sildenafil), and pharmaceutically acceptable salts or prodrugs thereof.

The term anti-platelet agents (or platelet inhibitory agents), as used herein, is also intended to include ADP (adenosine diphosphate) receptor antagonists, preferably antagonists of the purinergic receptors P₂Y₁ and P₂Y₁₂, with P₂Y₁₂ being even more preferred. Preferred P₂Y₁₂ receptor antagonists include ticlopidine and clopidogrel, including pharmaceutically acceptable salts or prodrugs thereof. Clopidogrel is an even more preferred agent. Ticlopidine and clopidogrel are also preferred compounds since they are known to be gentle on the gastro-intestinal tract in use.

The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets, and/or the granular secretion of plasminogen activator inhibitor-1 and/or serotonin) and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, and melagatran, including pharmaceutically acceptable salts and prodrugs thereof. Boroarginine derivatives and boropeptides include N-acetyl and peptide derivatives of boronic acid, such as C-terminal α-aminoboronic acid derivatives of lysine, ornithine, arginine, homoarginine and corresponding isothiouronium analogs thereof The term hirudin, as used herein, includes suitable derivatives or analogs of hirudin, referred to herein as hirulogs, such as disulfatohirudin.

The term thrombolytics or fibrinolytic agents (or thrombolytics or fibrinolytics), as used herein, denote agents that lyse blood clots (thrombi). Such agents include tissue plasminogen activator (TPA, natural or recombinant) and modified forms thereof, anistreplase, urokinase, streptokinase, tenecteplase (TNK), lanoteplase (nPA), factor VIIa inhibitors, PAI-1 inhibitors (i.e., inactivators of tissue plasminogen activator inhibitors), alpha2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in EP 028,489, the disclosure of which is hereby incorporated herein by reference herein. The term urokinase, as used herein, is intended to denote both dual and single chain urokinase, the latter also being referred to herein as prourokinase.

Examples of suitable anti-arrythmic agents for use in combination with the present compounds include: Class I agents (such as propafenone); Class II agents (such as carvadiol and propranolol); Class III agents (such as sotalol, dofetilide, amiodarone, azimilide and ibutilide); Class IV agents (such as ditiazem and verapamil); K⁺ channel openers such as I_{Ach} inhibitors, and I_{Kur} inhibitors (e.g., compounds such as those disclosed in WO01/40231).

Examples of suitable anti-hypertensive agents for use in combination with the compounds of the present invention include: alpha adrenergic blockers; beta adrenergic blockers; calcium channel blockers (e.g., diltiazem, verapamil, nifedipine, amlodipine and mybefradil); diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., omapatrilat, gemopatrilat and nitrates); and β-blockers (e.g., propanolol, nadolo, or carvedilol).

Examples of suitable cardiac glycosides for use in combination with the compounds of the present invention include digitalis and ouabain.

Examples of suitable mineralocorticoid receptor antagonists for use in combination with the compounds of the present invention include sprionolactone and eplirinone.

Examples of suitable cholesterol/lipid lowering agents and lipid profile therapies for use in combination with the compounds of the present invention include: HMG-CoA reductase inhibitors (e.g., pravastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin)); squalene synthetase inhibitors; fibrates; bile acid sequestrants (such as questran); ACAT inhibitors; MTP inhibitors; lipooxygenase inhibitors; choesterol absorption inhibitors; and cholesterol ester transfer protein inhibitors (e.g., CP-529414).

Examples of suitable anti-diabetic agents for use in combination with the compounds of the present invention include: biguanides (e.g., metformin); glucosidase inhibitors (e.g., acarbose); insulins (including insulin secretagogues or insulin sensitizers); meglitinides (e.g., repaglinide); sulfonylureas (e.g., glimepiride, glyburide and glipizide); biguanide/glyburide combinations (e.g., glucovance), thiozolidinediones (e.g., troglitazone, rosiglitazone and pioglitazone), PPAR-alpha agonists, PPAR-gamma agonists, PPAR alpha/gamma dual agonists, SGLT2 inhibitors, inhibitors of fatty acid binding protein (aP2) such as those disclosed in WO00/59506, glucagon-like peptide-1 (GLP-1), and dipeptidyl peptidase IV (DP4) inhibitors.

Examples of suitable anti-depressant agents for use in combination with the compounds of the present invention include nefazodone and sertraline.

Examples of suitable anti-inflammatory agents for use in combination with the compounds of the present invention include: prednisone; dexamethasone; enbrel; protien tyrosine kinase (PTK) inhibitors; cyclooxygenase inhibitors (including NSAIDs, and COX-1 and/or COX-2 inhibitors); aspirin; indomethacin; ibuprofen; prioxicam; naproxen; celecoxib; and/or rofecoxib.

Examples of suitable anti-osteoporosis agents for use in combination with the compounds of the present invention include alendronate and raloxifene.

Examples of suitable hormone replacement therapies for use in combination with the compounds of the present invention include estrogen (e.g., congugated estrogens) and estradiol.

Examples of suitable anti-coagulants for use in combination with the compounds of the present invention include heparins (e.g., unfractioned and low molecular weight heparins such as enoxaparin and dalteparin).

Examples of suitable anti-obesity agents for use in combination with the compounds of the present invention include orlistat and aP2 inhibitors (such as those disclosed in WO00/59506).

Examples of suitable anti-anxiety agents for use in combination with the compounds of the present invention include diazepam, lorazepam, buspirone, and hydroxyzine pamoate.

Examples of suitable anti-proliferative agents for use in combination with the compounds of the present invention include cyclosporin A, paclitaxel, adriamycin; epithilones, cisplatin, and carboplatin.

Examples of suitable anti-ulcer and gastroesophageal reflux disease agents for use in combination with the compounds of the present invention include famotidine, ranitidine, and omeprazole.

Administration of the compounds of the present invention (i.e., a first therapeutic agent) in combination with at least one additional therapeutic agent (i.e., a second therapeutic agent), preferably affords an efficacy advantage over the compounds and agents alone, preferably while permitting the use of lower doses of each (i.e., a synergistic combination). A lower dosage minimizes the potential of side effects, thereby providing an increased margin of safety. It is preferred that at least one of the therapeutic agents is administered in a sub-therapeutic dose. It is even more preferred that all of the therapeutic agents be administered in sub-therapeutic doses. Sub-therapeutic is intended to mean an amount of a therapeutic agent that by itself does not give the desired therapeutic effect for the condition or disease being treated. Synergistic combination is intended to mean that the observed effect of the combination is greater than the sum of the individual agents administered alone.

The compounds of the present invention are also useful as standard or reference compounds, for example as a quality standard or control, in tests or assays involving the inhibition of factor Xa. Such compounds may be provided in a commercial kit, for example, for use in pharmaceutical research involving factor Xa. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving factor Xa. For example, the presence of factor Xa in an unknown sample could be determined by addition of chromogenic substrate S2222 to a series of solutions containing test sample and optionally one of the compounds of the present invention. If production of pNA is observed in the solutions containing test sample, but not in the presence of a compound of the present invention, then one would conclude factor Xa was present.

Compounds of the present invention may further be useful as diagnostic agents and adjuncts. For example, the present compounds may be useful in maintaining whole and fractionated blood in the fluid phase such as required for analytical and biological testing.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof; and, (c) a package insert stating that the pharmaceutical composition can be used for the treatment of a thromboembolic disorder (as defined previously). In another embodiment, the package insert states that the pharmaceutical composition can be used in combination (as defined previously) with a second therapeutic agent to treat a thromboembolic disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

Dosage and Formulation

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

By way of general guidance, the daily oral dosage of each active ingredient, when used for the indicated effects, will range between about 0.001 to 1000 mg/kg of body weight, preferably between about 0.01 to 100 mg/kg of body weight per day, and most preferably between about 1.0 to 20 mg/kg/day. Intravenously, the most preferred doses will range from about 1 to about 10 mg/kg/min during a constant rate infusion. Compounds of this invention may be administered in a single daily dose, or the total daily dosage may be administered in divided doses of two, three, or four times daily.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl-or propyl-paraben, and chlorobutanol.

Suitable pharmaceutical carriers are described in Remington's Pharmaceutical Sciences, 18th ed., Mack Publishing Company, Easton, Pa., 1990, a standard reference text in this field.

Where the compounds of this invention are combined with other anticoagulant agents, for example, a daily dosage may be about 0.1 to 100 milligrams of the compound of The present invention and about 1 to 7.5 milligrams of the second anticoagulant, per kilogram of patient body weight. For a tablet dosage form, the compounds of this invention generally may be present in an amount of about 5 to 10 milligrams per dosage unit, and the second anti-coagulant in an amount of about 1 to 5 milligrams per dosage unit.

Where the compounds of the present invention are administered in combination with an anti-platelet agent, by way of general guidance, typically a daily dosage may be about 0.01 to 25 milligrams of the compound of The present invention and about 50 to 150 milligrams of the anti-platelet agent, preferably about 0.1 to 1 milligrams of the compound of The present invention and about 1 to 3 milligrams of antiplatelet agents, per kilogram of patient body weight.

Where the compounds of The present invention are administered in combination with thrombolytic agent, typically a daily dosage may be about 0.1 to 1 milligrams of the compound of The present invention, per kilogram of patient body weight and, in the case of the thrombolytic agents, the usual dosage of the thrombolyic agent when administered alone may be reduced by about 70-80% when administered with a compound of The present invention.

Where two or more of the foregoing second therapeutic agents are administered with the compound of The present invention, generally the amount of each component in a typical daily dosage and typical dosage form may be reduced relative to the usual dosage of the agent when administered alone, in view of the additive or synergistic effect of the therapeutic agents when administered in combination.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a lowviscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

Other features of the invention will become apparent in the course of the following descriptions of exemplary embodiments that are afforded for illustration of the invention and are not intended to be limiting thereof.

### EXAMPLES

Abbreviations used in the examples may include:
(a) Brine=saturated aqueous sodium chloride
(b) h or hr=hour(s)
(c) g=gram(s)
(d) mg=milligram(s)
(e) L=liter(s)
(f) mL=milliliter(s)
(g) μL=microliter(s)
(h) mmol=millimolar
(i) M=molar
(j) meq=milliequivalent(s)
(k) min=minute(s)
(l) N=normal
(m) Rochelle's salt=potassium sodium tartrate
(n) rt or RT=room temperature
(o) sat or sat'd=saturated
(p) ESI=electrospray ionization mass spectroscopy
(q) HPLC=high performance liquid chromatography
(r) MS=mass spectrometry
(s) LC/MS=liquid chromatography mass spectrometry
(t) HRMS=high resolution mass spectrometry
(u) NMR=nuclear magnetic resonance spectroscopy

### Example 1

### trans-N-2-((5-chloropyridin-2-yl)carbamoyl)cyclohexyl)4-(2-oxopyridin-1(2H)-yl)benzamide

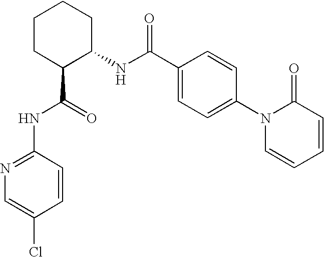

Part A: Hydrogen chloride gas was bubbled through a cooled (0° C.) suspension of trans-2-amino-1-cyclohexane carboxylic acid (3.0 g, 20.9 mmol) in methanol (200 mL) for several minutes to give a clear, colorless solution. The reaction was placed in a preheated oil bath (78° C.). After 3 h, the reaction was cooled to rt and concentrated to give a white solid. The solid was dissolved in water and basified with sat. Na₂CO₃ and extracted with dichloromethane (3×50 mL). The combined organic layers were washed with brine, dried over MgSO₄, filtered, and concentrated to give 1.95 g (59%) of β-amino ester as a clear, colorless liquid. The material was used without further purification. ¹H NMR (400 MHz, CDCl₃) δ: 3.70 (s, 3H), 2.89-2.86 (m, 1H), 2.09-2.06 (m, 1H), 1.96-1.86 (m, 2H), 1.77-1.72 (m, 2H), 1.46-1.08 (m, 4H). ¹³C NMR (125 MHz, CDCl₃) δ: 175.94, 53.04, 51.62, 51.57, 35.28, 29.07, 25.17, 25.03.

Part B: To a cooled (0° C.) solution of the product from Part A (0.331 g, 2.11 mmol) in pyridine (7.0 mL) was added 4-(2-oxopyridin-1(2H)-yl)benzoyl chloride (WO 2003/026652, 0.542 g, 2.32 mmol). The resulting orange-red solution was stirred at 0° C. for 30 min. and then warmed to rt for 2 h. The reaction was concentrated to give an orange residue, which was dissolved in dichloromethane, washed with sat. NaHCO₃, brine, dried over MgSO₄, filtered, and concentrated to give an orange solid. Column chromatography (gradient 0 to 100% EtOAc in hexane, then 100% EtOAc) gave 0.150 g (20%) of a yellow solid. LC/MS [M+H]⁺=355.09.

¹H NMR (400 MHz, CDCl₃) δ: 7.83 (d, J=8.8 Hz, 2H), 7.44-7.40 (m, 3H), 7.31 (dd, J=7.0, 1.8 Hz, 1H), 6.66 (d, J=9.2 Hz, 1H), 6.53-6.48 (m, 1H), 6.28 (t, J=7.0 Hz, 1H), 4.22-4.13 (m, 1H), 2.43 (dt, J=11.0, 3.5 Hz, 1H), 2.16-2.13 (m, 1H), 2.04-1.96 (m, 1H), 1.84-1.60 (m, 3H), 1.51-1.41 (m, 1H), 1.35-1.22 (m, 2H). ¹³C NMR (125 MHz, CDCl₃) δ: 174.32, 165.70, 162.18, 143.09, 140.22, 137.52, 134.95, 128.15, 126.63, 121.94, 106.40, 51.96, 50.63, 49.67, 32.64, 28.56, 24.70, 24.52.

Part C: To a cooled suspension (0° C.) of 2-amino-5-chloropyridine (0.472 g, 0.3.67 mmol) in dichloromethane (7.3 mL) was added dropwise a 2 M solution of trimethylaluminum in hexane (1.8 mL, 3.6 mmol). The reaction was warmed to rt and stirred for 15 min. to give a yellow suspension. Next a solution of the compound from Part B (0.130 g, 0.367 mmol) in dichloromethane (2.5 mL) was added and the resulting solution was placed in a preheated oil bath (40° C.). After 7 h., the reaction was cooled to rt and quenched with a 10% aqueous Rochelle's salt, and the reaction was diluted with dichloromethane. The biphasic mixture was stirred vigorously for several minutes. The layers were separated, and the aqueous layer was extracted with dichloromethane. The combined organic layers were washed with 1.0 N HCl, brine, dried over MgSO₄, filtered, and concentrated to give a pale yellow solid weighing 0.0831 g. Recrystallization from dichloroethane gave 0.0181 g (9%) of the title compound as a flaky, shiny solid. LC/MS [M+H]⁺=451.11. ¹H NMR (500 MHz, DMSO-d₆) δ: 10.38 (s, 1H), 8.34 (d, J=2.2 Hz, 1H), 8.28 (d, J=8.2 Hz, 1H), 8.06 (d, J=8.8 Hz, 1H), 7.84-7.82 (m, 3H), 7.62 (dd, J=7.2, 1.6 Hz, 1H), 7.53-7.49 (m, 1H), 7.45 (d, J=8.2 Hz, 2H), 6.48 (d, J=9.4 Hz, 1H), 6.32 (t, J=6.6 Hz, 1H), 4.08-4.06 (m, 1H), 2.75-2.71 (m, 1H), 1.96-1.93 (m, 2H), 1.74-1.72 (m, 2H); 1.56-1.53 (m, 1H), 1.37-1.22 (m, 3H). HRMS m/z calc'd for C₂₄H₂₄N₄O₃Cl [M+H]⁺=451.1537. Found 451.1536. m.p. 301-303° C.

### Example 2

### trans-N-((1S,2S)-2-((4-(2-oxopyridin-1(2H)-yl)phenyl)carbamoyl)cyclohexyl)4-chlorobenzamide

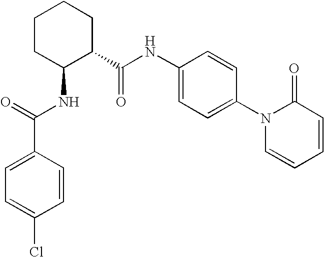

Part A: To a cooled (0° C.) solution of the compound from Part A, Example 1 (0.300 g, 1.90 mmol) in pyridine (6.3 mL) was added dropwise 4-chlorobenzoyl chloride (0.25 mL, 2.00 mmol). The resulting clear, bright yellow solution was stirred at 0° C. for 30 min. and then warmed to rt for 1 h. The reaction was diluted with dichloromethane, washed with sat. NaHCO₃, brine, dried over MgSO₄, filtered, and concentrated to give a pale yellow solid weighing 0.528 g. Column chromatography on silica gel (0 to 50% EtOAc in hexane) provided 0.494 g (88%) of the compound as a white solid. LC/MS [M +H]⁺=295.98. ¹H NMR (500 MHz, CDCl₃) δ: 7.64 (d, J=8.2 Hz, 2H), 7.36 (d, J=8.8 Hz, 2H), 6.18-6.16 (m, 1H), 4.16-4.09 (m, 1H), 3.62 (s, 3H), 2.39 (dt, J=11.6, 3.3 Hz, 1H), 2.18-2.14 (m, 1H), 1.96-1.95 (m, 1H), 1.78-1.76 (m, 2H), 1.70-1.62 (m, 1H), 1.47-1.39 (m, 1H), 1.31-1.17(m, 2H). ¹³C NMR (125 MHz, CDCl₃) δ: 174.23, 165.73, 137.61, 133.14, 128.77, 128.32, 51.97, 50.79, 49.80, 32.78, 28.39, 24.63, 24.47.

Part B: To a cooled suspension (0° C.) of 1-(4-aminophenyl)pyridin-2(1H)-one (WO 2004/031145, 0.142 g, 0.760 mmol) in dichloroethane (5 mL) was added dropwise a 2 M solution of trimethylaluminum in hexane (0.35 mL, 0.708 mmol). The reaction was warmed to rt and stirred for 15 min. to give a yellow suspension. Next a solution of the compound from Part A (0.075 g, 0.253 mmol) in dichloroethane (2.5 mL) was added, and the resulting solution was placed in a preheated oil bath (85° C.). After 6 h, the reaction was cooled to rt and quenched with sat. Rochelle's salt. The biphasic mixture was stirred vigorously for several minutes. The layers were separated, and the aqueous layer was extracted with dichloromethane. The combined organic layers were washed with brine, dried over MgSO₄, filtered, and concentrated to give a brown solid weighing 0.149 g. Recrystallization from dichlorethane gave 0.026 g (23%) of the title compound as an off-white solid. LC/MS [M+H]⁺=447.88. ¹H NMR (500 MHz, DMSO-d₆) δ: 9.86 (s, 1H), 8.31 (d, J=8.2 Hz, 1H), 7.76 (d, J=8.8 Hz, 2H), 7.62 (d, J=8.8 Hz, 2H), 7.56 (dd, J=6.8, 1.9 Hz, 1H), 7.48 (d, J=8.2 Hz, 2H), 7.47-7.44 (m, 1H), 7.26 (d, J=8.8 Hz, 2H), 6.43 (d, J=8.8 Hz, 1H), 6.26 (dt, J=6.6, 1.1 Hz, 1H), 4.08-4.04 (m, 1H), 2.60 (dt, J=11.5, 3.3 Hz, 1H), 1.96-1.92 (m, 2H), 1.75-1.73 (m, 2H), 1.58-1.51 (m, 1H), 1.35-1.22 (m, 3H). HRMS m/z calc'd for C₂₅H₂₅N₃O₃Cl [M+H]⁺=450.1584. Found 450.1573.

Examples shown in Table 1 below can be prepared by following the procedures of Examples 1-2 and schemes 1-33 and by using appropriate commercially available starting materials. Table 1 contains representative species of the present invention. Examples 1-200 recite a G group, central ring, and an A-B group that are specifically shown in the legend at the top of the table. Thus, each of Examples 1-200 represent a specific compound.

TABLE 1
G Groups

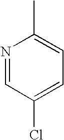

G-1:

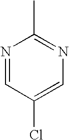

G-2:

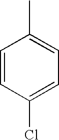

G-3:

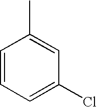

G-4:

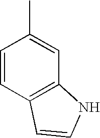

G-5:
A-B Groups

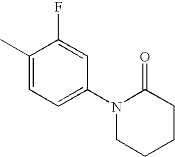

A-1:

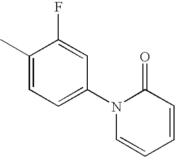

A-2:

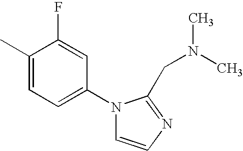

A-3:

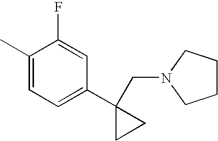

A-4:

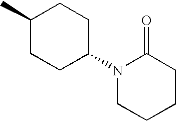

A-5:

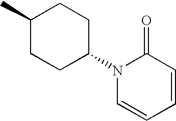

A-6:

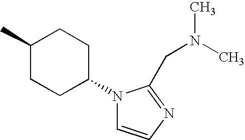

A-7:

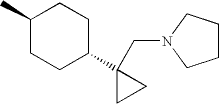

A-8:
Central Rings

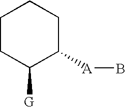

R-1:

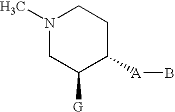

R-2:

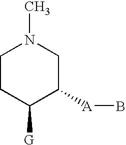

R-3:

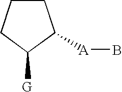

R-4:

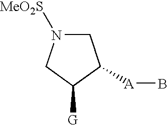

R-5:

| Ex. # | G group | A-B group | Central Ring |
|---|---|---|---|
| 1. | G-1 | A-1 | R-1 |
| 2. | G-2 | A-1 | R-1 |
| 3. | G-3 | A-1 | R-1 |
| 4. | G-4 | A-1 | R-1 |
| 5. | G-5 | A-1 | R-1 |
| 6. | G-1 | A-2 | R-1 |
| 7. | G-2 | A-2 | R-1 |
| 8. | G-3 | A-2 | R-1 |
| 9. | G-4 | A-2 | R-1 |
| 10. | G-5 | A-2 | R-1 |
| 11. | G-1 | A-3 | R-1 |
| 12. | G-2 | A-3 | R-1 |
| 13. | G-3 | A-3 | R-1 |
| 14. | G-4 | A-3 | R-1 |
| 15. | G-5 | A-3 | R-1 |
| 16. | G-1 | A-4 | R-1 |
| 17. | G-2 | A-4 | R-1 |
| 18. | G-3 | A-4 | R-1 |
| 19. | G-4 | A-4 | R-1 |
| 20. | G-5 | A-4 | R-1 |
| 21. | G-1 | A-5 | R-1 |
| 22. | G-2 | A-5 | R-1 |
| 23. | G-3 | A-5 | R-1 |
| 24. | G-4 | A-5 | R-1 |
| 25. | G-5 | A-5 | R-1 |
| 26. | G-1 | A-6 | R-1 |
| 27. | G-2 | A-6 | R-1 |
| 28. | G-3 | A-6 | R-1 |
| 29. | G-4 | A-6 | R-1 |
| 30. | G-5 | A-6 | R-1 |
| 31. | G-1 | A-7 | R-1 |
| 32. | G-2 | A-7 | R-1 |
| 33. | G-3 | A-7 | R-1 |
| 34. | G-4 | A-7 | R-1 |
| 35. | G-5 | A-7 | R-1 |
| 36. | G-1 | A-8 | R-1 |
| 37. | G-2 | A-8 | R-1 |
| 38. | G-3 | A-8 | R-1 |
| 39. | G-4 | A-8 | R-1 |
| 40. | G-5 | A-8 | R-1 |
| 41. | G-1 | A-1 | R-2 |
| 42. | G-2 | A-1 | R-2 |
| 43. | G-3 | A-1 | R-2 |
| 44. | G-4 | A-1 | R-2 |
| 45. | G-5 | A-1 | R-2 |
| 46. | G-1 | A-2 | R-2 |
| 47. | G-2 | A-2 | R-2 |
| 48. | G-3 | A-2 | R-2 |
| 49. | G-4 | A-2 | R-2 |
| 50. | G-5 | A-2 | R-2 |
| 51. | G-1 | A-3 | R-2 |
| 52. | G-2 | A-3 | R-2 |
| 53. | G-3 | A-3 | R-2 |
| 54. | G-4 | A-3 | R-2 |
| 55. | G-5 | A-3 | R-2 |
| 56. | G-1 | A-4 | R-2 |
| 57. | G-2 | A-4 | R-2 |
| 58. | G-3 | A-4 | R-2 |
| 59. | G-4 | A-4 | R-2 |
| 60. | G-5 | A-4 | R-2 |
| 61. | G-1 | A-5 | R-2 |
| 62. | G-2 | A-5 | R-2 |
| 63. | G-3 | A-5 | R-2 |
| 64. | G-4 | A-5 | R-2 |
| 65. | G-5 | A-5 | R-2 |
| 66. | G-1 | A-6 | R-2 |
| 67. | G-2 | A-6 | R-2 |
| 68. | G-3 | A-6 | R-2 |
| 69. | G-4 | A-6 | R-2 |
| 70. | G-5 | A-6 | R-2 |
| 71. | G-1 | A-7 | R-2 |
| 72. | G-2 | A-7 | R-2 |
| 73. | G-3 | A-7 | R-2 |
| 74. | G-4 | A-7 | R-2 |
| 75. | G-5 | A-7 | R-2 |
| 76. | G-1 | A-8 | R-2 |
| 77. | G-2 | A-8 | R-2 |
| 78. | G-3 | A-8 | R-2 |
| 79. | G-4 | A-8 | R-2 |
| 80. | G-5 | A-8 | R-2 |
| 81. | G-1 | A-1 | R-3 |
| 82. | G-2 | A-1 | R-3 |
| 83. | G-3 | A-1 | R-3 |
| 84. | G-4 | A-1 | R-3 |
| 85. | G-5 | A-1 | R-3 |
| 86. | G-1 | A-2 | R-3 |
| 87. | G-2 | A-2 | R-3 |
| 88. | G-3 | A-2 | R-3 |
| 89. | G-4 | A-2 | R-3 |
| 90. | G-5 | A-2 | R-3 |
| 91. | G-1 | A-3 | R-3 |
| 92. | G-2 | A-3 | R-3 |
| 93. | G-3 | A-3 | R-3 |
| 94. | G-4 | A-3 | R-3 |
| 95. | G-5 | A-3 | R-3 |
| 96. | G-1 | A-4 | R-3 |
| 97. | G-2 | A-4 | R-3 |
| 98. | G-3 | A-4 | R-3 |
| 99. | G-4 | A-4 | R-3 |
| 100. | G-5 | A-4 | R-3 |
| 101. | G-1 | A-5 | R-3 |
| 102. | G-2 | A-5 | R-3 |
| 103. | G-3 | A-5 | R-3 |
| 104. | G-4 | A-5 | R-3 |
| 105. | G-5 | A-5 | R-3 |
| 106. | G-1 | A-6 | R-3 |
| 107. | G-2 | A-6 | R-3 |
| 108. | G-3 | A-6 | R-3 |
| 109. | G-4 | A-6 | R-3 |
| 110. | G-5 | A-6 | R-3 |
| 111. | G-1 | A-7 | R-3 |
| 112. | G-2 | A-7 | R-3 |
| 113. | G-3 | A-7 | R-3 |
| 114. | G-4 | A-7 | R-3 |
| 115. | G-5 | A-7 | R-3 |
| 116. | G-1 | A-8 | R-3 |
| 117. | G-2 | A-8 | R-3 |
| 118. | G-3 | A-8 | R-3 |
| 119. | G-4 | A-8 | R-3 |
| 120. | G-5 | A-8 | R-3 |
| 121. | G-1 | A-1 | R-4 |
| 122. | G-2 | A-1 | R-4 |
| 123. | G-3 | A-1 | R-4 |
| 124. | G-4 | A-1 | R-4 |
| 125. | G-5 | A-1 | R-4 |
| 126. | G-1 | A-2 | R-4 |
| 127. | G-2 | A-2 | R-4 |
| 128. | G-3 | A-2 | R-4 |
| 129. | G-4 | A-2 | R-4 |
| 130. | G-5 | A-2 | R-4 |
| 131. | G-1 | A-3 | R-4 |
| 132. | G-2 | A-3 | R-4 |
| 133. | G-3 | A-3 | R-4 |
| 134. | G-4 | A-3 | R-4 |
| 135. | G-5 | A-3 | R-4 |
| 136. | G-1 | A-4 | R-4 |
| 137. | G-2 | A-4 | R-4 |
| 138. | G-3 | A-4 | R-4 |
| 139. | G-4 | A-4 | R-4 |
| 140. | G-5 | A-4 | R-4 |
| 141. | G-1 | A-5 | R-4 |
| 142. | G-2 | A-5 | R-4 |
| 143. | G-3 | A-5 | R-4 |
| 144. | G-4 | A-5 | R-4 |
| 145. | G-5 | A-5 | R-4 |
| 146. | G-1 | A-6 | R-4 |
| 147. | G-2 | A-6 | R-4 |
| 148. | G-3 | A-6 | R-4 |
| 149. | G-4 | A-6 | R-4 |
| 150. | G-5 | A-6 | R-4 |
| 151. | G-1 | A-7 | R-4 |
| 152. | G-2 | A-7 | R-4 |
| 153. | G-3 | A-7 | R-4 |
| 154. | G-4 | A-7 | R-4 |
| 155. | G-5 | A-7 | R-4 |
| 156. | G-1 | A-8 | R-4 |
| 157. | G-2 | A-8 | R-4 |
| 158. | G-3 | A-8 | R-4 |
| 159. | G-4 | A-8 | R-4 |
| 160. | G-5 | A-8 | R-4 |
| 161. | G-1 | A-1 | R-5 |
| 162. | G-2 | A-1 | R-5 |
| 163. | G-3 | A-1 | R-5 |
| 164. | G-4 | A-1 | R-5 |
| 165. | G-5 | A-1 | R-5 |
| 166. | G-1 | A-2 | R-5 |
| 167. | G-2 | A-2 | R-5 |
| 168. | G-3 | A-2 | R-5 |
| 169. | G-4 | A-2 | R-5 |
| 170. | G-5 | A-2 | R-5 |
| 171. | G-1 | A-3 | R-5 |
| 172. | G-2 | A-3 | R-5 |
| 173. | G-3 | A-3 | R-5 |
| 174. | G-4 | A-3 | R-5 |
| 175. | G-5 | A-3 | R-5 |
| 176. | G-1 | A-4 | R-5 |
| 177. | G-2 | A-4 | R-5 |
| 178. | G-3 | A-4 | R-5 |
| 179. | G-4 | A-4 | R-5 |
| 180. | G-5 | A-4 | R-5 |
| 181. | G-1 | A-5 | R-5 |
| 182. | G-2 | A-5 | R-5 |
| 183. | G-3 | A-5 | R-5 |
| 184. | G-4 | A-5 | R-5 |
| 185. | G-5 | A-5 | R-5 |
| 186. | G-1 | A-6 | R-5 |
| 187. | G-2 | A-6 | R-5 |
| 188. | G-3 | A-6 | R-5 |
| 189. | G-4 | A-6 | R-5 |
| 190. | G-5 | A-6 | R-5 |
| 191. | G-1 | A-7 | R-5 |
| 192. | G-2 | A-7 | R-5 |
| 193. | G-3 | A-7 | R-5 |
| 194. | G-4 | A-7 | R-5 |
| 195. | G-5 | A-7 | R-5 |
| 196. | G-1 | A-8 | R-5 |
| 197. | G-2 | A-8 | R-5 |
| 198. | G-3 | A-8 | R-5 |
| 199. | G-4 | A-8 | R-5 |
| 200. | G-5 | A-8 | R-5 |

Numerous modifications and variations of the present invention are possible in light of the above teachings. It is therefore to be understood that within the scope of the appended claims, the invention may be practiced otherwise that as specifically described herein.

What is claimed is:

1. A compound or a pharmaceutically acceptable salt or solvate form thereof, wherein the compound is selected from: 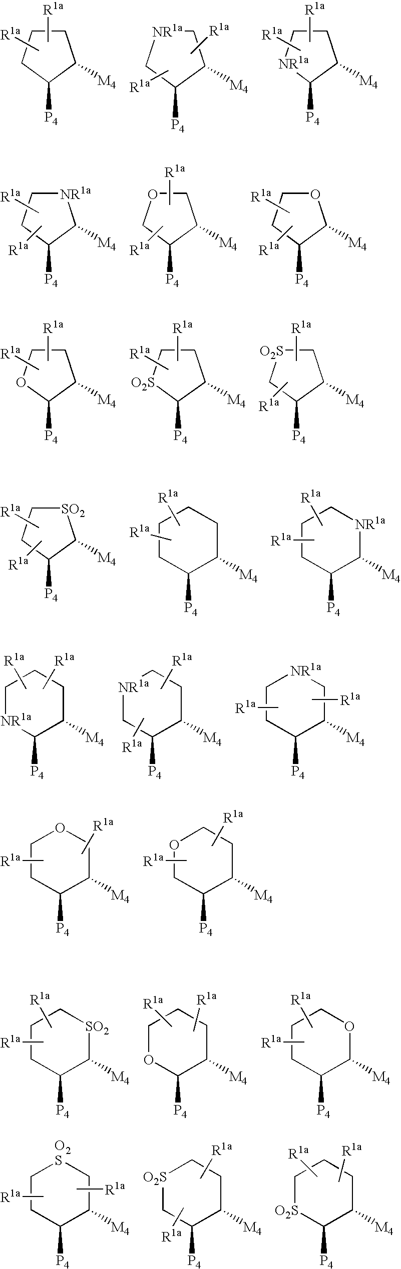 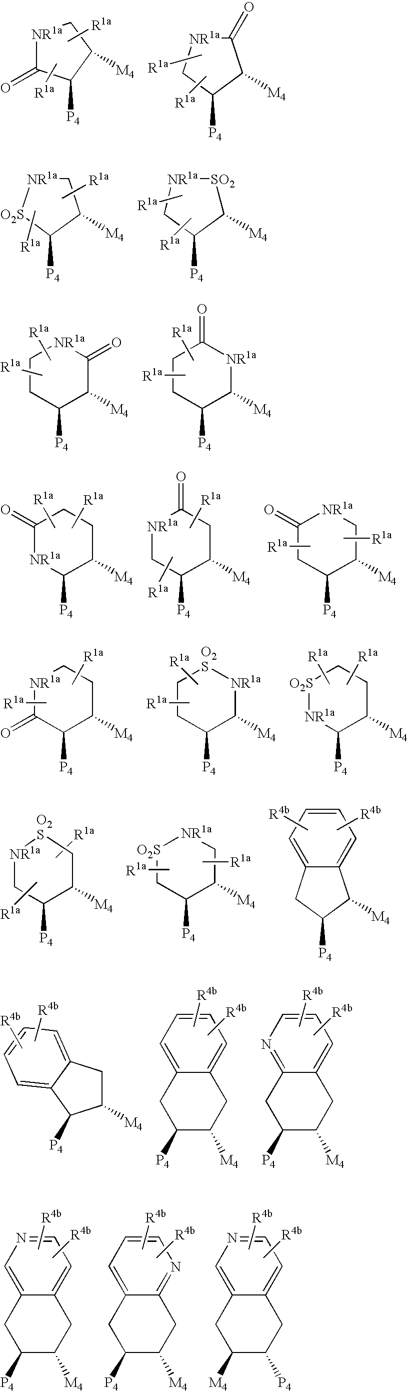 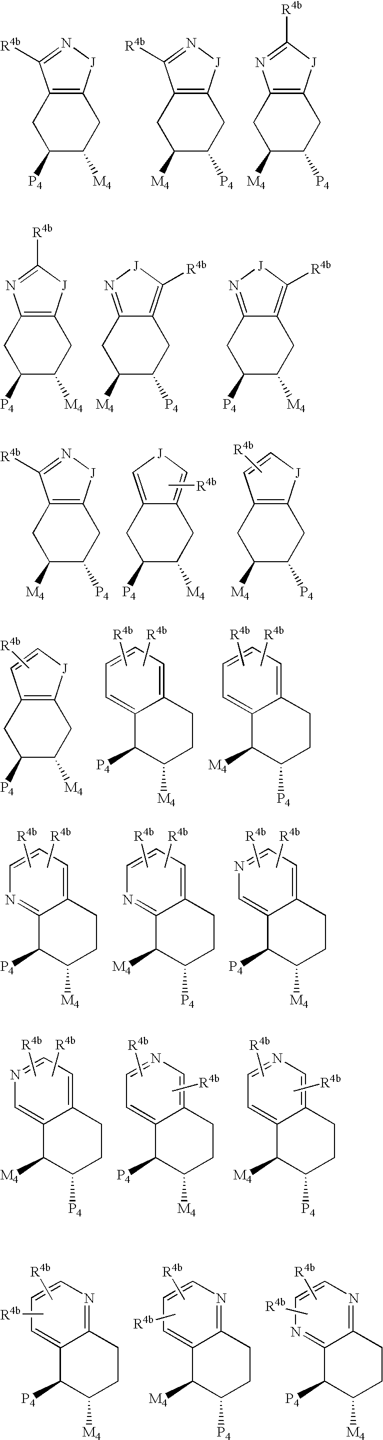 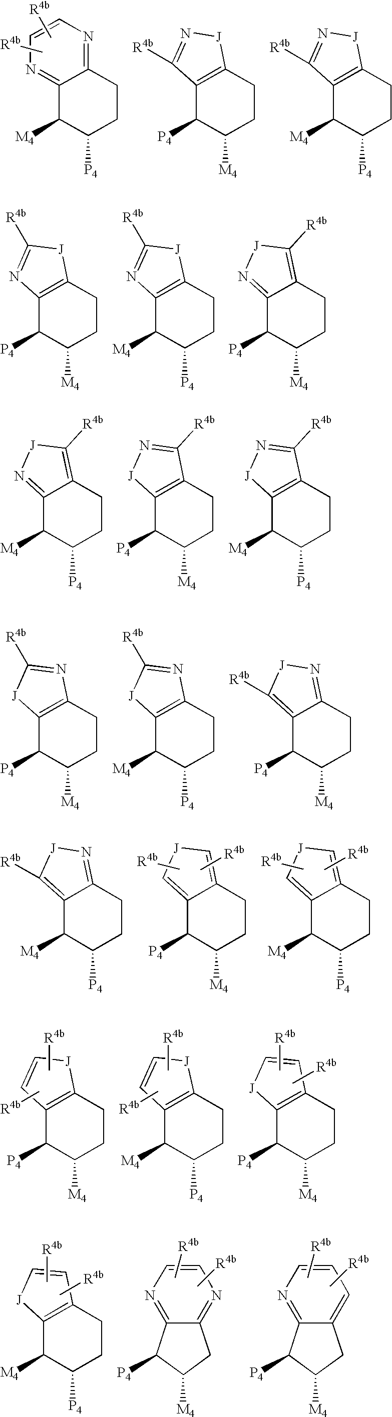 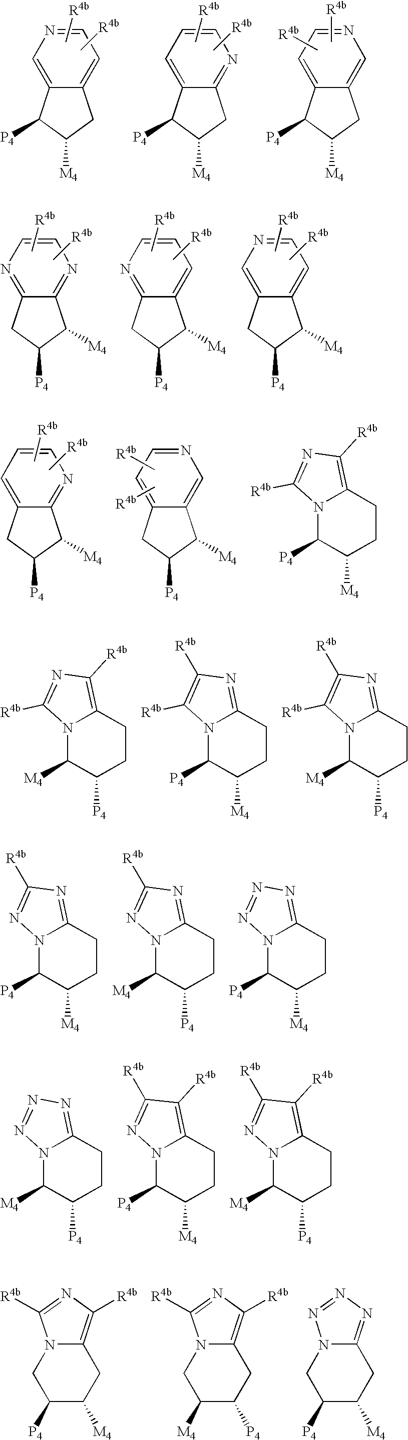 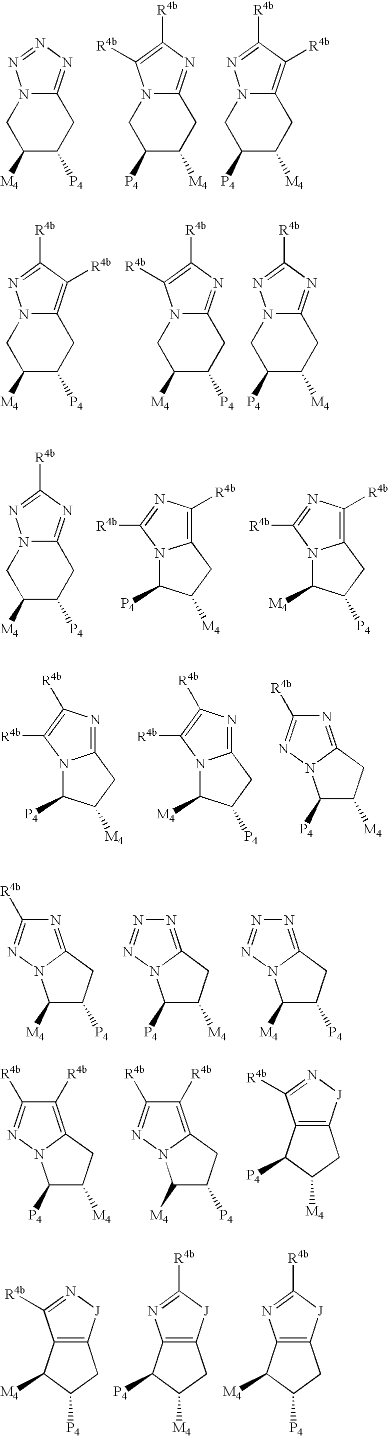 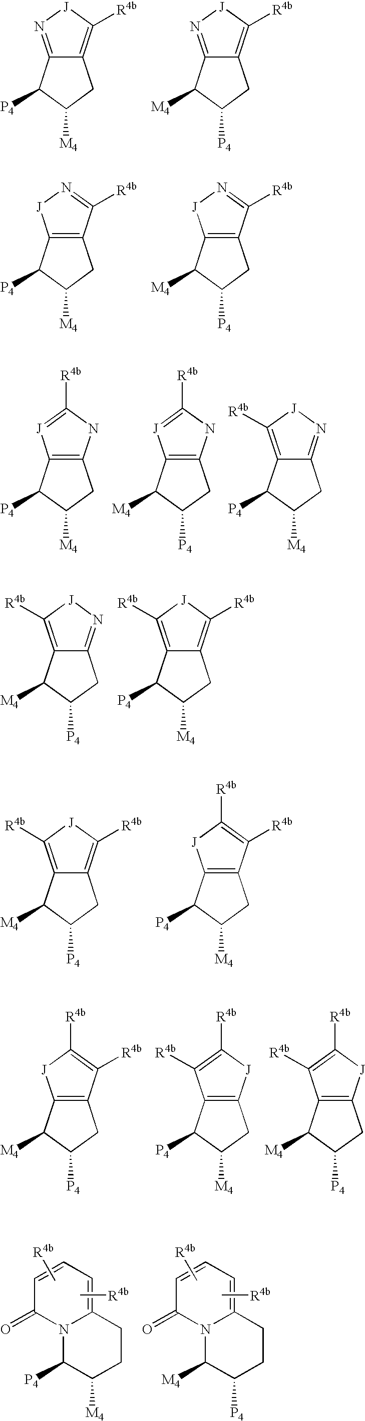 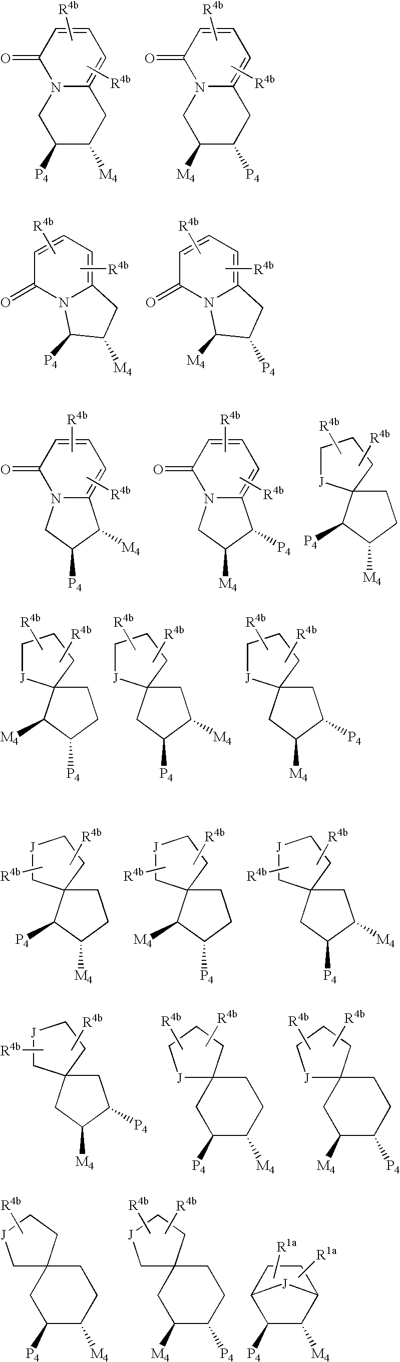 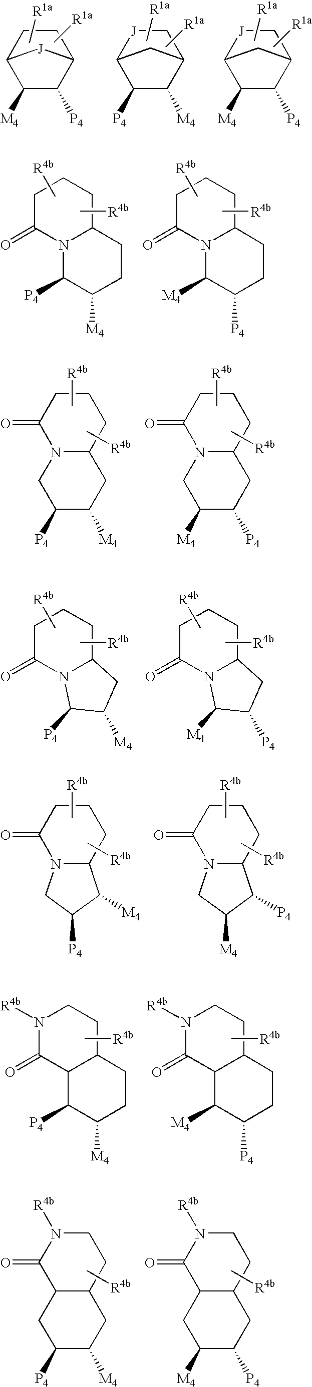 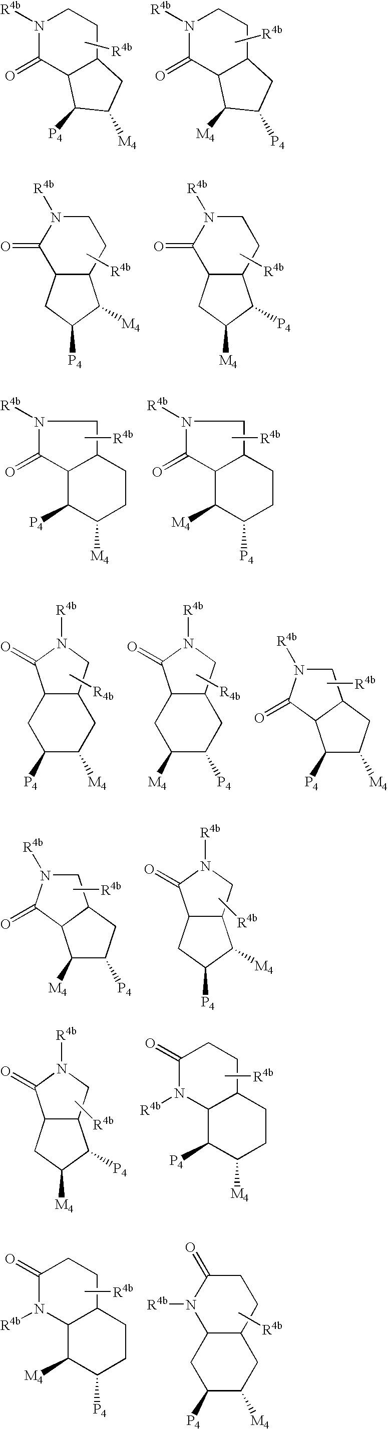 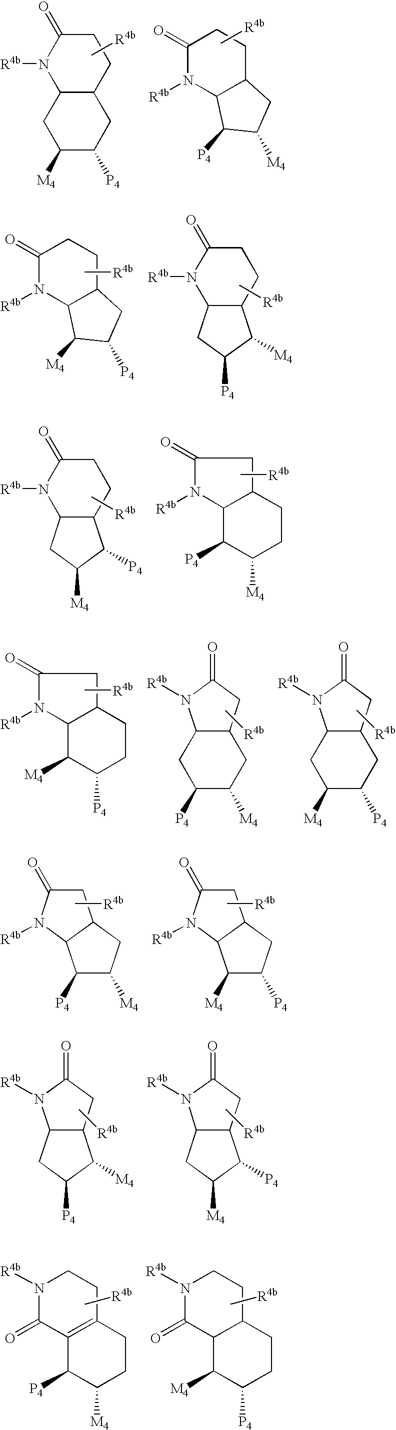 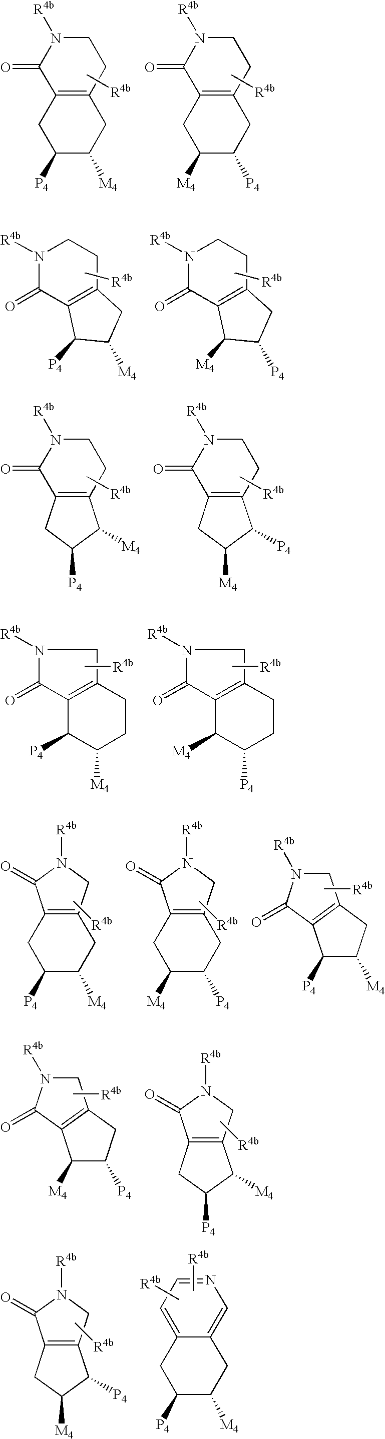 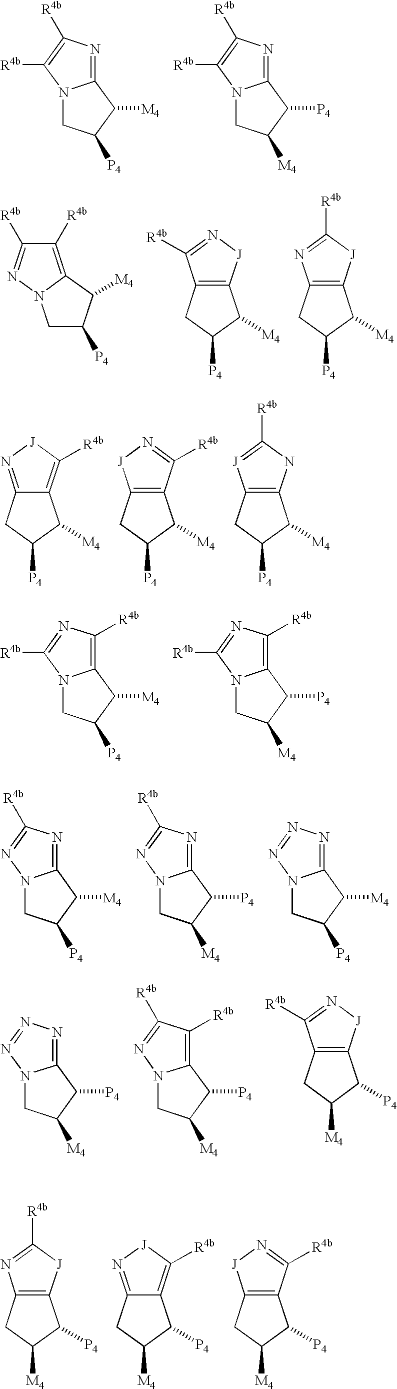 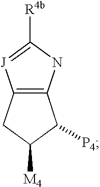
or a pharmaceutically acceptable salt thereof, wherein:
J is selected from O, S, S(O)₂, CR^{1a}, and NR^{1a};
one of P₄ and M₄ is -Z-A-B and the other -G₁-G;
G is a group of formula IIa or IIb: 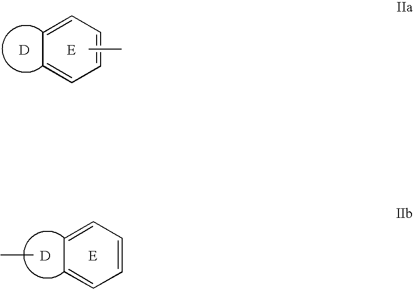
ring D, including the two atoms of Ring E to which it is attached, is a 5-6 membered ring consisting of carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-3 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-3 R;
alternatively, ring D is absent and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and with a 5-6 membered heterocycle consisting of: carbon atoms and 1-4 heteroatoms selected from the group consisting of N, O, and S(O)ₚ, wherein the 5-6 membered heterocycle is substituted with 0-2 carbonyls and 1-3 R and there are 0-3 ring double bonds;
R is selected from H, C₁₋₄ alkyl, F, Cl, Br, I, OH, OCH₃, OCH₂CH₃, OCH(CH₃)₂, OCH₂CH₂CH₃, —CN, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, C(═NR⁸)NR⁷R⁹, NHC(═NR⁸)NR⁷R⁹, ONHC(═NR⁸)NR⁷R⁹, NR⁸CH(═NR⁷), (CR⁸R⁹)ₜC(O)H, (CR⁸R⁹)ₜC(O)R^{2c}, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜOR³, (CR⁸R⁹)ₜS(O)ₚNR⁷R⁸, (CR⁸R⁹)ₜNR⁷S(O)ₚR⁷, (CR⁸R⁹)ₜSR³, (CR⁸R⁹)ₜS(O)R³, (CR⁸R⁹)ₜS(O)₂R³, and OCF₃, provided that S(O)ₚR⁷ and S(O)₂R³ form other than S(O)₂H or S(O)H;
alternatively, when 2 R groups are attached to adjacent atoms, they combine to form methylenedioxy or ethylenedioxy;
one of G₁ and Z is selected from CR³R^{3a}CR³R^{3a}, CR³═CR³, C≡C, CR³R^{3a}C(O), C(O)CR³R^{3a}, C(O)O, OC(O), CR³R^{3a}O, OCR³R^{3a}, CR³R^{3a}NR^{3b}, NR^{3b}CR³R^{3a}, CR³R^{3a}NR^{3e}, NR^{3e}CR³R^{3a}, C(O)NR^{3b}, NR^{3b}C(O), CR³R^{3a}S, SCR³R^{3a}, CR³R^{3a}S(O), S(O)CR³R^{3a}, CR³R^{3a}S(O)₂, S(O)₂CR³R^{3a}, S(O)NR^{3b}, NR^{3b}S(O)₂, and S(O)₂NR^{3b}, and the other of G₁ and Z is selected from CR³R^{3a}CR³R^{3a}, CR³═CR³, C≡C, CR³R^{3a}C(O), C(O)CR³R^{3a}, C(O)O, OC(O), CR³R^{3a}O, OCR³R^{3a}, CR³R^{3a}NR^{3b}, CR³R^{3a}NR^{3e}, C(O)NR^{3b}, CR³R^{3a}S, SCR³R^{3a}, CR³R^{3a}S(O), S(O)CR³R^{3a}, CR³R^{3a}S(O)₂, S(O)₂CR³R^{3a}, S(O)NR^{3b}, and S(O)₂NR^{3b}, wherein the right side of Z/G₁ is attached to ring A/ring G, provided that neither Z nor G form an N—S, NCH₂N, NCH₂O, or NCH₂S bond with either group to which it is attached;
A is selected from: C₃₋₁₀ carbocycle substituted with 0-2 R⁴, and 5-12 membered heterocycle consisting of: carbon atoms and 1-4 heteroatoms selected from the group consisting of N, O, and S(O)ₚ and substituted with 0-2 R⁴;
B is selected from Y, X—Y, N(B¹)C(O)C(R³R^{3g})₁₋₄NB²B³,C(B⁵)═NB⁴, and 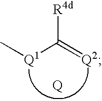
provided that Z and B are attached to different atoms on A and that the R^{4d} shown is other than OH;
B¹ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂)₀₋₂—C₃₋₇ carbocycle substituted with 0-2 R^{4b}, and —(CH₂)₀₋₂-5-6 membered heterocycle consisting of: carbon atoms and 1-4 heteroatoms selected from the group consisting of N, O, and S(O)ₚ and substituted with 0-2 R^{4b};
B² is selected from H, C₁₋₆alkyl substituted with 0-2 R^{4c}, C(O)R^{2e}, C(O)OR^{2d}, C(O)NR^{2d}R^{2d}, C(O)NH(CH₂)₂NR^{2d}R^{2d}, SO₂NR^{2d}R^{2d}, C(O)NHSO₂—C₁₋₄ alkyl, and S(O)ₚR^{5a};
B³ is selected from H, C₁₋₆ alkyl substituted with 0-2 R^{4c}, —(CH₂)₀₋₂-3-6 membered carbocycle substituted with 0-2 R⁵, and a —(CH₂)₀₋₂ -4-6 membered heterocycle consisting of: carbon atoms and 1-4 heteroatoms selected from the group consisting of N, O, and S(O)ₚ and substituted with 0-2 R⁵;
B⁴ is selected from H, SO₂R^{3b}, C(O)R^{3b}, SO₂NR³R^{3b}, C(O)NR³R^{3b}, OR², SR², —CN, and NO₂;
B⁵ is NR²R^{2f} or CR³R²R^{2f};
Q¹ and Q² are each N;
alternatively, Q¹ is CR³ and R^{4d} is NR²R^{2a} or NR^{3a}B⁴, provided that when Q¹ is CR³, then this R³ group optionally forms a ring with the R² group of R^{4d}, this ring is a 5-6 membered ring consisting of, in addition to the C—C—N shown, carbon atoms and from 0-1 additional heteroatoms selected from N, O, and S(O)ₚ, and this ring is substituted with 0-1 R⁵;
ring Q is a 5-8 membered ring consisting of, in addition to the Q¹-CR^{4d}=Q² group shown, carbon atoms and 0-2 heteroatoms selected from N, O, and S(O)ₚ, and the ring is substituted with an additional 0-2 R^{4d};
Y is selected from: CY¹Y²R^{4a}, NR³R^{3a}, C(O)NR³R^{3a}, C3-10 carbocycle substituted 0-2 R⁴ and 0-1 R^{4a}, and, 3-10 membered heterocycle consisting of: carbon atoms and 1-4 heteroatoms selected from the group consisting of N, O, and S(O)ₚ and substituted with 0-2 R⁴ and 0-1 R^{4a};
Y¹ and Y² are independently C₁₋₄ alkyl substituted with 0-2 R⁴;
X is absent or is selected from —(CR²R^{2a})₁₋₄—, —CR²(CR²R^{2b})(CH₂)ₜ—, —C(O)—, —C(═NR^{1b})—, —CR²(NR^{1b}R²)—, —CR²(OR²)—, —CR²(SR²)—, —C(O)CR²R^{2a}—, —CR²R^{2a}C(O), —S(O)—, —S(O)₂—, —SCR²R^{2a}—, —S(O)CR²R^{2a}—, —S(O)₂CR²R^{2a}, —CR²R^{2a}S(O)—, —CR²R^{2a}S(O)₂—, —S(O)₂NR²CR²R^{2a}—, —NR²S(O)₂—, —CR²R^{2a}NR²S(O)₂—, —NR²S(O)₂CR²R^{2a}—, —NR²C(O)—, —C(O)NR²CR²R^{2a}—, —NR²C(O)CR²R^{2a}—, —CR²R^{2a}NR²C(O)—, —NR²CR²R^{2a}—, and —OCR²R^{2a}—;
Z² is selected from H, S(O)₂NHR^{3b}, C(O)R^{3b}, C(O)NHR^{3b}, C(O)OR^{3f}, S(O)R^{3f}, S(O)₂R^{3f}, C₁₋₆alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-C₃₋₁₀ carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{1a}, at each occurrence, is selected from H, —(CR³R^{3a})ᵣ—R^{1b}, —(CR³R^{3a})ᵣ—CR³R^{1b}R^{1b}, —(CR³R^{3a})ᵣ—O—(CR³R^{3a})ᵣ—R^{1b}, —(CR³R^{3a})ᵣ—NR²—(CR³R^{3a})ᵣ—R^{1b}, —(CR³R^{3a})ᵣ—S(O)ₚ—(CR³R^{3a})ᵣ—R^{1b}, —(CR³R^{3a})ᵣ—CO₂—(CR³R^{3a})ᵣ—R^{1b}, —(CR³R^{3a})ᵣ—C(O)NR²—(CR³R^{3a})ᵣ—R^{1b}, —(CR³R^{3a})ᵣ—C(O)—(CR³R^{3a})ᵣ—R^{1b}, —C₂₋₆ alkenylene-R^{1b}, —C₂₋₆ alkynylene-R^{1b}, and —(CR³R^{3a})ᵣ—C(═NR^{1b})NR³R^{1b}, provided that R^{1a} forms other than an N-halo, N—S, O—O, or N—CN bond;
alternatively, when two R^{1a} groups are attached to adjacent atoms, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;
R^{1b} is selected from H, C₁₋₃ alkyl, F, Cl, Br, I, —CN, —NO₂, —CHO, (CF₂)ᵣCF₃, (CR³R^{3a})ᵣOR², NR²R^{2a}, C(O)R^{2b}, CO₂R^{2b}, OC(O)R², CH(CH₂OR²)₂, (CF₂)ᵣCO₂R^{2a}, S(O)ₚR^{2b}, NR²(CH₂)ᵣOR², C(═NR^{2c})NR²R^{2a}, NR²C(O)R^{2b}, NR²C(O)NR²R^{2a}, NR²C(O)₂R^{2a}, OC(O)NR²R^{2a}, C(O)NR²R^{2a}, C(O)NR²(CH₂)ᵣOR², SO₂NR²R^{2a}, NR²SO₂R², C(O)NR²SO₂R², C₃₋₆ carbocycle substituted with 0-2 R^{4b}, and 5-10 membered heterocycle substituted with 0-2 R^{4b} and consisting of carbon atoms and from 1-4 heteroatoms selected from the group consisting of N, O, and S(O)ₚ, provided that R^{1b} forms other than an O—O, N-halo, N—S, or N—CN bond and provided that S(O)ₚR² forms other than S(O)₂H or S(O)H;
R², at each occurrence, is selected from H, CF₃, C₁₋₆ alkyl, —(CH₂)ᵣC₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};
R^{2a}, at each occurrence, is selected from H, CF₃, C₁₋₆ alkyl, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};
alternatively, NR²R^{2a}forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy substituted with 0-2 R^{4b}, C₁₋₆alkyl substituted with 0-2 R^{4b}, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ, and substituted with 0-2 R^{4b};
R^{2c}, at each occurrence, is selected from CF₃, OH, C₁₋₄ alkoxy, C₁₋₆ alkyl, —(CH₂)ᵣ—C₃₋₁₀ carbocycle substituted with 0-2 R^{4b}, and —(CH₂)ᵣ-5-10 membered heterocycle containing from 1-4 heteroatoms selected from the group consisting of N, O, and S(O)ₚ, and substituted with 0-2 R^{4b};
R^{2d}, at each occurrence, is selected from H, R^{4c}, C₁₋₆ alkyl substituted with 0-2 R^{4c}, —(CR³R^{3a})ᵣ—C₃₋₁₀ carbocycle substituted with 0-2 R^{4c}, and —(CR³R^{3a})ᵣ-5-10 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;
alternatively, NR^{2d}R^{2d} forms a 5-10 membered saturated, partially saturated or unsaturated ring substituted with 0-2 R^{4b} and consisting of: 0-1 additional heteroatoms selected from the group consisting of N, O, and S(O)ₚ;
R^{2e}, at each occurrence, is selected from H, R^{4c}, C₁₋₆alkyl substituted with 0-2 R^{4c}, —(CR³R^{3a})ᵣ—C₃₋₁₀ carbocycle substituted with 0-2 R^{4c}, and —(CR³R^{3a})ᵣ-5-10 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;
R^{2f}, at each occurrence, is selected from H, CF₃, C₁₋₄ alkoxy substituted with 0-2 R^{4b}, C₁₋₆ alkyl substituted with 0-2 R^{4b}, —(CH₂)ᵣ—C₃₋₁₀ carbocycle substituted with 0-2 R^{4b}, and —(CH₂)ᵣ-5-10 membered heterocycle consisting of: carbon atoms and 1-4 heteroatoms selected from the group consisting of N, O, and S(O)ₚ and substituted with 0-2 R^{4b};
alternatively, CR²R^{2f} forms a 5-8 membered ring consisting of: carbon atoms and 0-2 heteroatoms selected from N, O, and S(O)ₚ, and this ring is substituted with 0-2 R^{4b};
alternatively, NR²R^{2f} forms a 5-8 membered ring consisting of: carbon atoms and 0-2 additional heteroatoms selected from N, O, and S(O)ₚ, and this ring is substituted with 0-2 R^{4b};
alternatively, when B⁴ is SO₂R^{3b} and B⁵ is NR²R^{2f}, R^{3b} and R^{2f} combine to form a 5-8 membered ring consisting of: carbon atoms and 0-2 additional heteroatoms selected from N, O, and S(O)ₚ, and this ring is substituted with 0-2 R^{4b};
alternatively, when B⁴ is C(O)R^{3b} and B⁵ is NR²R^{2f}, R^{3b} and R^{2f} combine to form a 5-8 membered ring consisting of: carbon atoms and 0-2 additional heteroatoms selected from N, O, and S(O)ₚ, and this ring is substituted with 0-2 R^{4b};
alternatively, when B⁵ is NR²R^{2f}, B⁴ and R^{2f} combine to form a 5-8 membered ring consisting of: carbon atoms and 0-2 additional heteroatoms selected from N, O, and S(O)ₚ, and this ring is substituted with 0-2 R^{4b} and the R² group of NR²R^{2f}, in addition to the groups recited below, is selected from SO₂R^{3b}, C(O)R^{3b}, and —CN;
R³, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, R³ and R^{3a}, together with the nitrogen atom to which they are attached, combine to form a 5 or 6 membered saturated, partially unsaturated, or unsaturated ring consisting of: carbon atoms, the nitrogen atom to which R³ and R^{3a}are attached, and 0-1 additional heteroatoms selected from the group consisting of N, O, and S(O)ₚ;
R^{3b}, at each occurrence, is selected from H, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3c}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3d}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C₁₋₄ alkyl-phenyl, and C(═O)R^{3c};
R^{3e}, at each occurrence, is selected from H, SO₂NHR³, SO₂NR³R³, C(O)R³, C(O)NHR³, C(O)OR^{3f}, S(O)R^{3f}, S(O)₂R^{3f}, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3f}, at each occurrence, is selected from: C₁₋₆alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3g}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂)ᵣ-3-6 membered carbocycle, and —(CH₂)ᵣ-5-6 membered heterocycle consisting of: carbon atoms and 1-4 heteroatoms selected from the group consisting of N, O, and S(O)ₚ;
alternatively, CR³R^{3g} forms a cyclopropyl group;
R⁴, at each occurrence, is selected from ═O, CHO, (CR³R^{3a})ᵣOR², (CR³R^{3a})ᵣF, (CR³R^{3a})ᵣCl, (CR³R^{3a})ᵣBr, (CR³R^{3a})ᵣ, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣC(═NR²)NR²R^{2a}, (CR³R^{3a})ᵣC(═NS(O)₂R⁵)NR²R^{2a}, (CR³R^{3a})ᵣNR²C(═NR²)NR²R^{2a}, (CR³R^{3a})ᵣC(O)NR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣNR²SOₛR⁵, (CR³R^{3a})ᵣS(O)ₚR^{5a}, (CR³R^{3a})ᵣ(CF₂)ᵣCF₃, NHCH₂R^{1b}, OCH₂R^{1b}, SCH₂R^{1b}, NH(CH₂)₂(CH₂)ₜR^{1b}, O(CH₂)₂(CH₂)ₜR^{1b}, S(CH₂)₂(CH₂)ₜR^{1b}, (CR³R^{3a})ᵣ-5-6 membered carbocycle substituted with 0-1 R⁵, and a (CR³R^{3a})ᵣ-5-6 membered heterocycle consisting of: carbon atoms and 1-4 heteroatoms selected from the group consisting of N, O, and S(O)ₚ and substituted with 0-1 R⁵;
R^{4a} is selected from C₁₋₆ alkyl substituted with 0-2 R^{4c}, C₂₋₆ alkenyl substituted with 0-2 R^{4c}, C₂₋₆ alkynyl substituted with 0-2 R^{4c}, —(CR³R^{3g})ᵣ—C₅₋₁₀ membered carbocycle substituted with 0-3 R^{4c}, —(CR³R^{3g})ᵣ-5-10 membered heterocycle substituted with 0-3 R^{4c} and consisting of: carbon atoms and 1-4 heteroatoms selected from the group consisting of N, O, and S(O)ₚ, (CR³R^{3g})ᵣCN, (CR³R^{3g})ᵣC(═NR^{2d})NR^{2d}R^{2d}, (CR³R^{3g})ᵣNR^{2d}C(═NR^{2d})NR^{2d}R^{2d}, (CR³R^{3g})ᵣNR^{2d}C(R^{2e})(═NR^{2d}), (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣ—NR^{2d}C(O)R^{2e}, (CR³R^{3g})ᵣ—C(O)R^{2e}, (CR³R^{3g})ᵣOC(O)R^{2e}, (CR³R^{3g})ᵣ—C(O)NR^{2d}R^{2d}, (CR³R^{3g})ᵣ—C(O)OR^{2d}, (CR³R^{3g})ᵣNR^{2d}C(O)OR^{2d}, (CR³R^{3g})ᵣ—OC(O)NR^{2d}R^{2d}, (CR³R^{3g})ᵣ—NR^{2d}(C(O)OR^{2d}, (CR³R^{3g})₅—SO₂NR^{2d}R^{2d}, (CR³R^{3g})ᵣ—NR^{2d}SO₂NR^{2d}R^{2d}, (CR³R^{3g})ᵣ—C(O)NR^{2d}SO₂R^{2d}, (CR³R^{3g})ᵣ—NR^{2d}SO₂R^{2d}, and (CR³R^{3g})ᵣ—S(O)ₚR^{2d}, provided that S(O)ₚR^{2d} forms other than S(O)₂H or S(O)H and further provided that R^{4a} is other than a hydroxamic acid;
R^{4b}, at each occurrence, is selected from H, ═O, (CH₂)ᵣOR³, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR³R^{3a}, (CH₂ᵣC(O)R³, (CH₂)ᵣC(O)OR^{3c}, (CH₂)ᵣNR³C(O)R^{3a}, (CH₂)ᵣ—C(O)NR³R^{3a}, (CH₂)ᵣNR³C(O)NR³R^{3a}, (CH₂)ᵣ—C(═NR³)NR³R^{3a}, (CH₂)ᵣNR³C(═NR³)NR³R^{3a}, (CH₂)ᵣSO₂NR³R^{3a}, (CH₂)ᵣNR³ SO₂NR³R^{3a}, (CH₂)ᵣNR³SO₂—C₁₋₄ alkyl, (CH₂)ᵣNR³SO₂CF₃, (CH₂)ᵣNR³ SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R^{4c}, at each occurrence, is selected from ═O, (CR³R^{3a})ᵣOR², (CR³R^{3a})ᵣF, (CR³R^{3a})ᵣBr, (CR³R^{3a})ᵣCl, (CR³R^{3a})ᵣCF₃, C₁₋₄ alkyl, C₂₋₆ alkenyl, C₂₋₆ alkynyl, (CR³R^{3a})ᵣCN, (CR³R^{3a})ᵣNO₂, (CR³R^{3a})ᵣNR²R^{2a}, (CR³R^{3a})ᵣN(→O)R²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NR²(CH₂)₂NR²R^{2a}, (CR³R^{3a})ᵣNR²C(O)NR²R^{2a}, (CR³R^{3a})ᵣC(═NR²)NR²R^{2a}, (CR³R^{3a})ᵣNR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣC(O)NR²SO₂—C₁₋₄ alkyl, (CR³R^{3a})ᵣNR²SO₂R^{5a}, (CR³R^{3a})ᵣC(O)NR²SO₂R^{5a}, (CR³R^{3a})ᵣS(O)ₚR^{5a}, (CF₂)ᵣCF₃, (CR³R^{3a})ᵣC₃₋₁₀ carbocycle substituted with 0-2 R^{4b}, and (CR³R^{3a})ᵣ4-10 membered heterocycle substituted with 0-2 R^{4b} and consisting of carbon atoms and from 1-4 heteroatoms selected from the group consisting of N, O, and S(O)ₚ;
R^{4d}, at each occurrence, is selected from H, (CR³R^{3a})ᵣOR², (CR³R^{3a})ᵣF, (CR³R^{3a})ᵣBr, (CR³R^{3a})ᵣCl, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NH(CH₂)₂NR²R^{2a}, (CR³R^{3a})ᵣNR²C(O)NR²R^{2a}, (CR³R^{3a})ᵣC(═NR²)NR²R^{2a}, (CR³R^{3a})ᵣNHC(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣC(O)NHSO₂—C₁₋₄ alkyl, (CR³R^{3a})NR²SO₂R⁵, (CR³R^{3a})ᵣS(O)ₚR^{5a}, (CR³R^{3a})ᵣ(CF₂)ᵣCF₃, (CR³R^{3a})ᵣ-5-6 membered carbocycle substituted with 0-1 R⁵, and a (CR³R^{3a})ᵣ-5-6 membered heterocycle consisting of: carbon atoms and 1-4 heteroatoms selected from the group consisting of N, O, and S(O)ₚ and substituted with 0-1 R⁵;
R⁵, at each occurrence, is selected from H, C₁₋₆ alkyl, ═O, (CH₂)ᵣOR³, F, Cl, Br, I, —CN, NO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣCH(═NOR^{3d}), (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³ SO₂NR³R^{3a}, (CH₂)ᵣNR³SO₂—C₁₋₄ alkyl, (CH₂)ᵣNR³SO₂CF₃, (CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, (CF₂)ᵣCF₃, phenyl substituted with 0-2 R⁶, naphthyl substituted with 0-2 R⁶, and benzyl substituted with 0-2 R⁶;
R^{5a}, at each occurrence, is selected from C₁₋₆alkyl, (CH₂)ᵣOR³, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CF₂)ᵣCF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond;
R⁶, at each occurrence, is selected from H, OH, (CH₂)ᵣOR², halo, C₁₋₄ alkyl, CN, NO₂, (CH₂)ᵣNR²R^{2a}, (CH₂)ᵣC(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl;
R⁷, at each occurrence, is selected from H, OH, C₁₋₆ alkyl, C₁₋₆alkyl-C(O)—, C₁₋₆ alkyl-O—, (CH₂)ₙ-phenyl, C₁₋₄ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl-C₁₋₄ alkyl-C(O)—;
R⁸, at each occurrence, is selected from H, C₁₋₆ alkyl, and (CH₂)ₙ-phenyl;
alternatively, R⁷ and R⁸, when attached to the same nitrogen, combine to form a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)n-phenyl;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

2. A compound according to claim 1, wherein the compound is selected from: 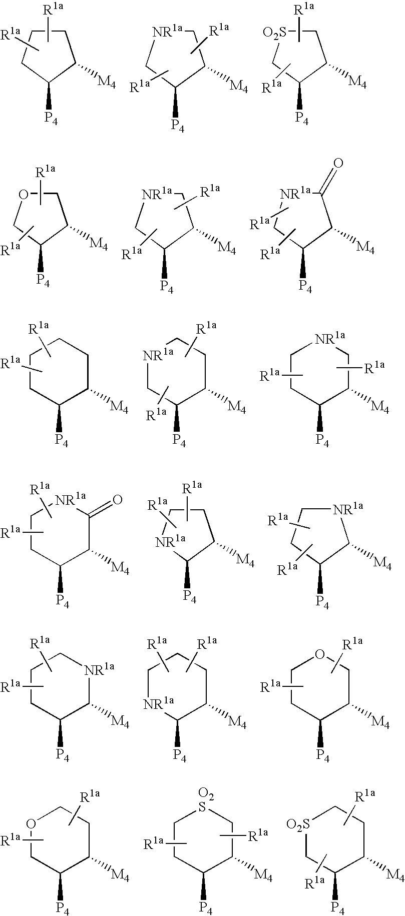 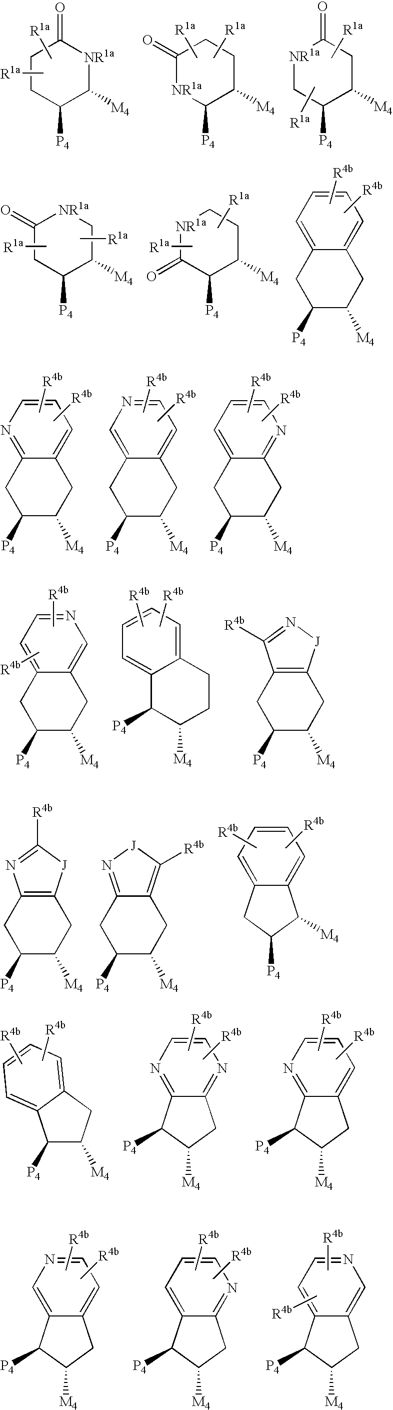 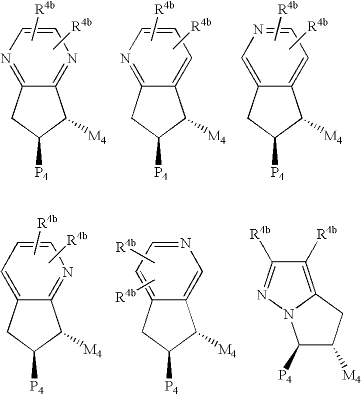 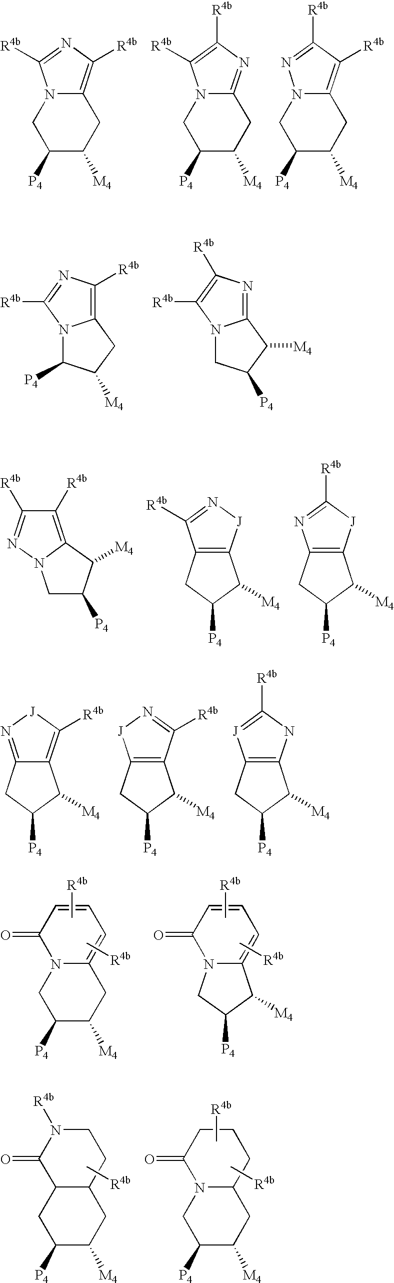 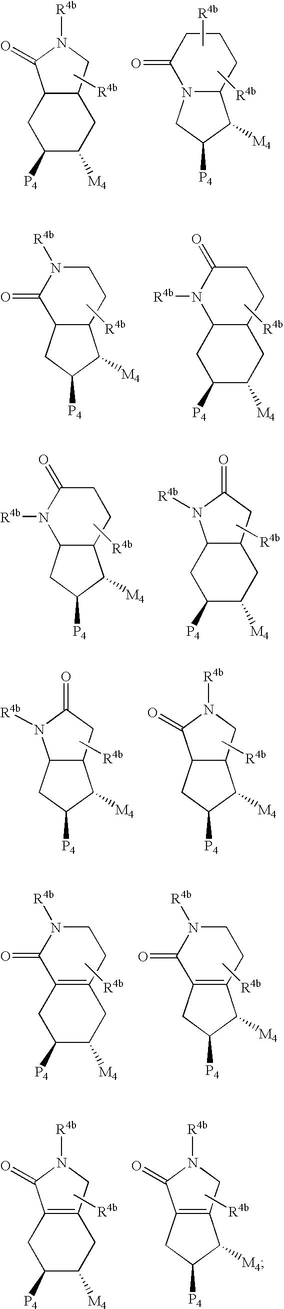
J is selected from O, S, S(O)₂, and NR^{1a};
G is selected from the group: 2-aminomethyl-4-chloro-phenyl, 2-aminosulfonyl-4-chloro-phenyl, 2-amido-4-chloro-phenyl, 4-chloro-2-methylsulfonyl-phenyl, 2-aminosulfonyl-4-fluoro-phenyl, 2-amido-4-fluoro-phenyl, 4-fluoro-2-methylsulfonyl-phenyl, 2-aminomethyl-4-bromo-phenyl, 2-aminosulfonyl-4-bromo-phenyl, 2-amido-4-bromo-phenyl, 4-bromo-2-methylsulfonyl-phenyl, 2-aminomethyl-4-methyl-phenyl, 2-aminosulfonyl-4-methyl-phenyl, 2-amido-4-methyl-phenyl, 2-methylsulfonyl-4-methyl-phenyl, 4-fluoro-pyrid-2-yl, 4-bromo-pyrid-2-yl, 4-methyl-pyrid-2-yl, 5-fluoro-thien-2-yl, 5-bromo-thien-2-yl, 5-methyl-thien-2-yl, 2-amido-4-methoxy-phenyl, 2-amido-phenyl, 2-aminomethyl-3-fluoro-phenyl, 2-aminomethyl-4-fluoro-phenyl, 2-aminomethyl-4-methoxy-phenyl, 2-aminomethyl-5-fluoro-phenyl, 2-aminomethyl-5-methoxy-phenyl, 2-aminomethyl-6-fluoro-phenyl, 2-aminomethyl-phenyl, 2-amino-pyrid-4-yl, 2-aminosulfonyl-4-methoxy-phenyl, 2-aminosulfonyl-phenyl, 2-methylsulfonyl-phenyl, 3-(N,N-dimethylamino)-4-chloro-phenyl, 3-(N,N-dimethylamino)-phenyl, 3-(N-methylamino)-4-chloro-phenyl, 3-(N-methylamino)-phenyl, 3-amido-phenyl, 3-amino-4-chloro-phenyl, 3-aminomethyl-phenyl, 3-amino-phenyl, 4-(N,N-dimethylamino)-5-chloro-thien-2-yl, 4-(N-methylamino)-5-chloro-thien-2-yl, 4-amino-5-chloro-thien-2-yl, 4-methoxy-2-methylsulfonyl-phenyl, 2-methoxy-pyrid-5-yl, 5-(N,N-dimethylamino)-4-chloro-thien-2-yl, 5-(N-methylamino)-4-chloro-thien-2-yl, 5-amino-4-chloro-thien-2-yl, 5-chloro-pyrid-2-yl, 5-chloro-thien-2-yl, 6-amino-5-chloro-pyrid-2-yl, 6-amino-pyrid-2-yl, 2-cyano-4-chloro-phenyl, 2-methoxy-4-chloro-phenyl, 2-fluoro-4-chloro-phenyl, 3-chloro-4-methyl-phenyl, 3-fluoro-4-chloro-phenyl, 3-methyl-4-chloro-phenyl, 3-fluoro-4-methyl-phenyl, 3,4-dimethyl-phenyl, 3-chloro-4-fluoro-phenyl, 3-methyl-4-fluoro-phenyl, 4-methylsulfanyl-phenyl, 2-chlorothiazol-5-yl, 5-chlorothiazol-2-yl, 3-pyridyl; 2-pyridyl, 5-fluoro-pyrid-2-yl, 5-methyl-pyrid-2-yl, 5-methoxy-pyrid-2-yl, 5-cyano-pyrid-2-yl, 2-pyrimidyl, 5-chloro-pyrimid-2-yl, 5-fluoro-pyrimid-2-yl, 5-methyl-pyrimid-2-yl, 5-methoxy-pyrimid-2-yl, 5-cyano-pyrimid-2-yl, phenyl, 3-chloro-phenyl, 3-fluoro-phenyl, 3-methyl-phenyl, 4-ethyl-phenyl, 3-methoxy-phenyl, 3-cyano-phenyl, 4-chloro-phenyl, 4-fluoro-phenyl, 4-methyl-phenyl, 4-methoxy-phenyl, 4-cyano-phenyl, 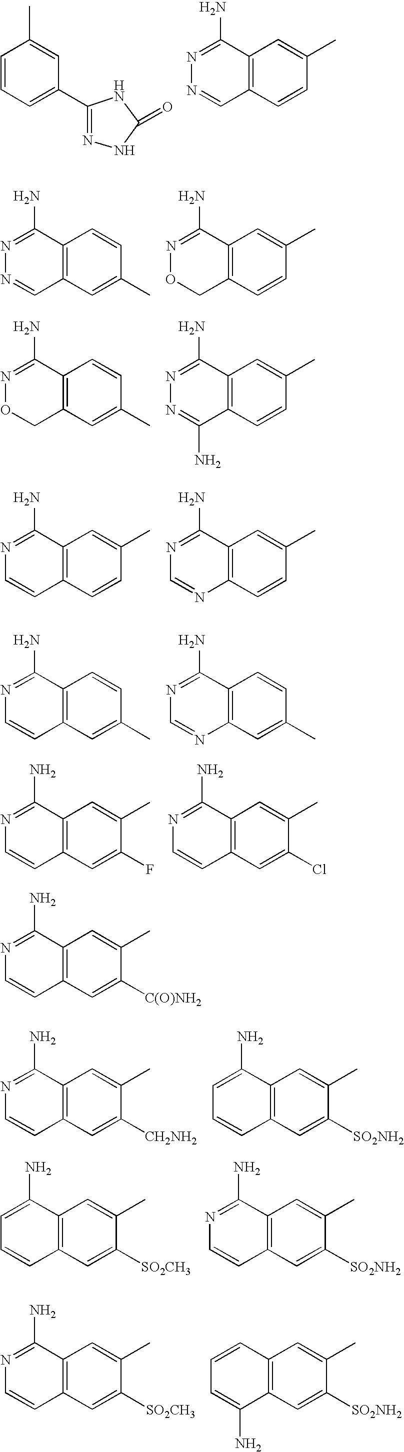 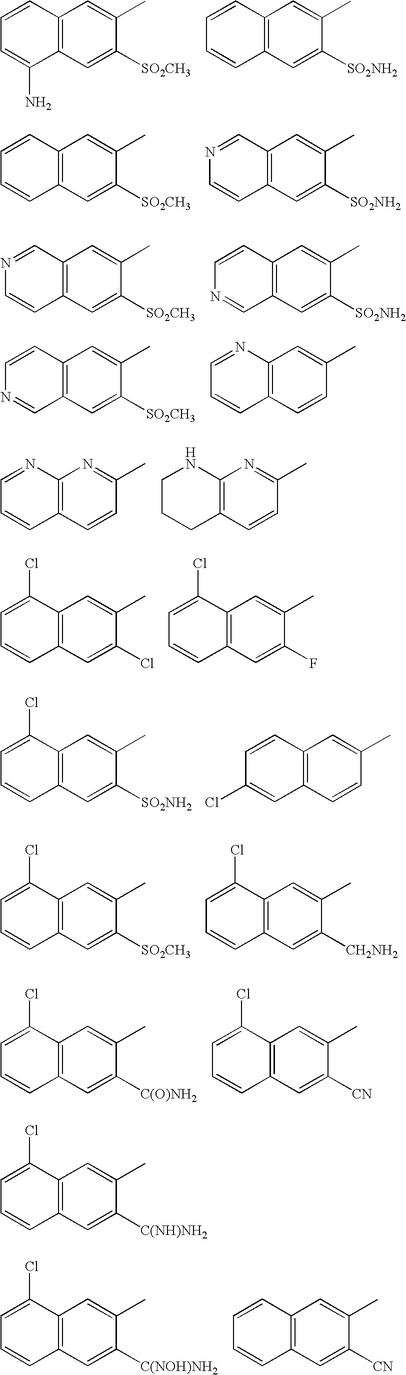 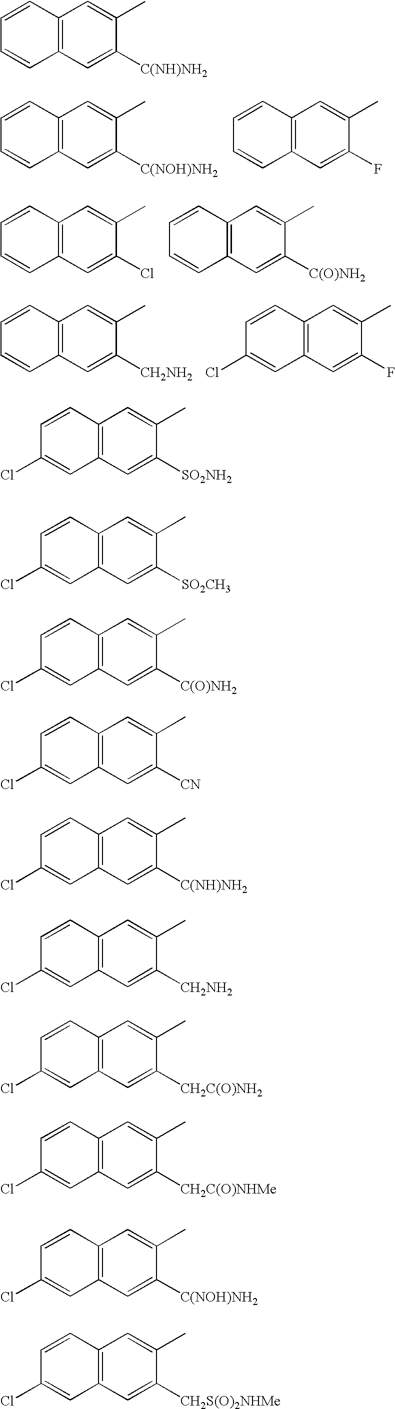 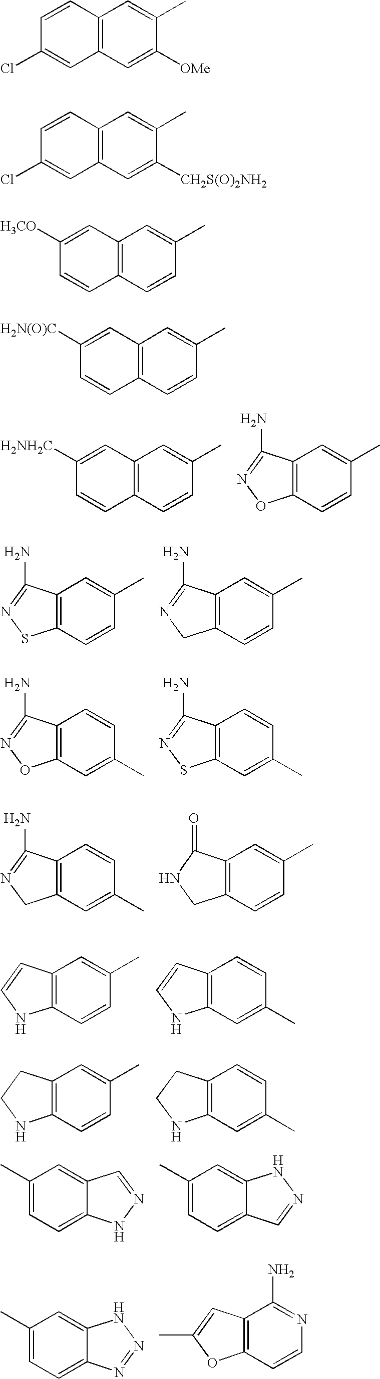 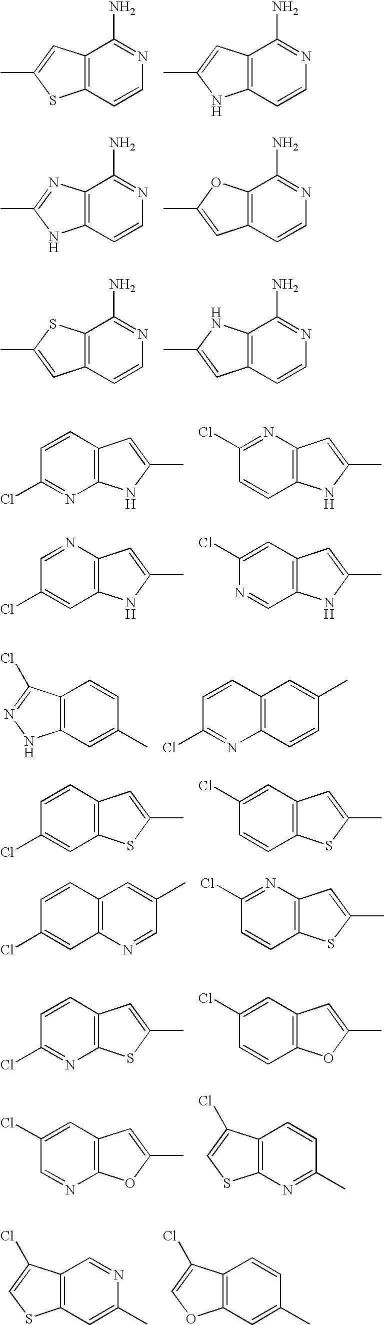 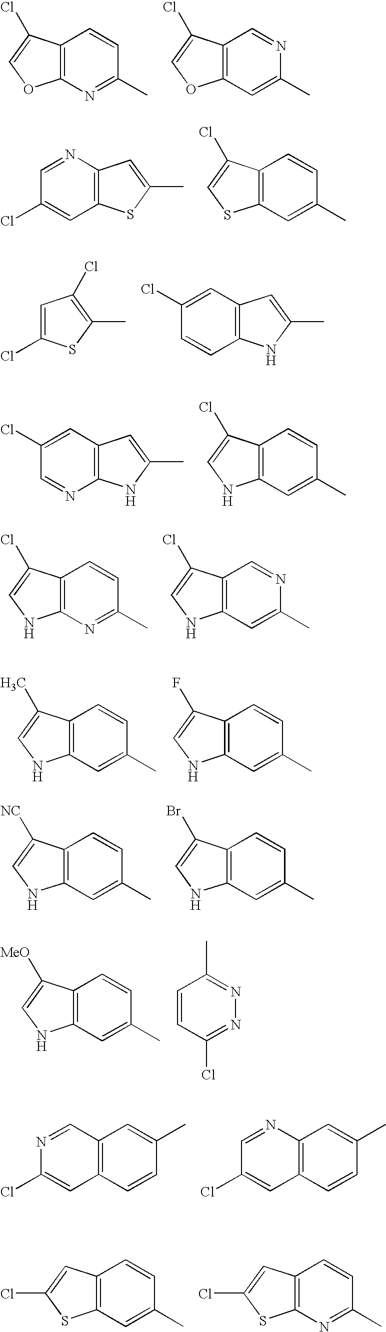 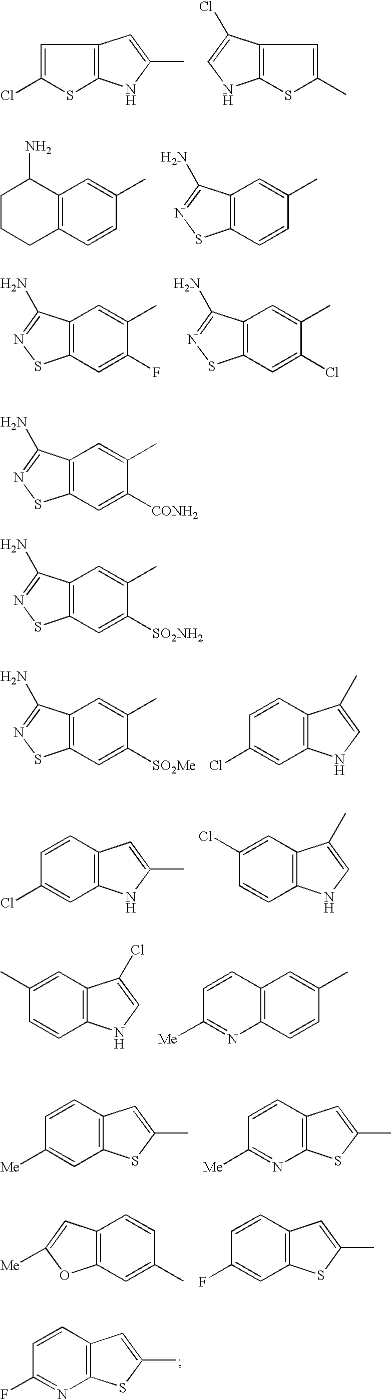
one of G₁ and Z is selected from CR³R^{3a}CR³R^{3a}, CR³R^{3a}C(O), C(O)CR³R^{3a}, C(O)O, OC(O), CR³R^{3a}O, OCR³R^{3a}, CR³R^{3a}NR^{3b}, NR^{3b}CR³R^{3a}, CR³R^{3a}NR^{3e}, NR^{3e}CR³R^{3a}, C(O)NR^{3b}, NR^{3b}C(O), CR³R^{3a}S(O), S(O)CR³R^{3a}, CR³R^{3a}S(O)₂, S(O)₂CR³R^{3a}, S(O)NR^{3b}, NR^{3b}S(O)₂, and S(O)₂NR^{3b}, and the other of G₁ and Z is selected from CR³R^{3a}CR³R^{3a}, CR³R^{3a}C(O), C(O)CR³R^{3a}, C(O)O, OC(O), CR³R^{3a}O, OCR³R^{3a}, CR³R^{3a}NR^{3b}, CR³R^{3a}NR^{3e}, C(O)NR^{3b}, CR³R^{3a}S(O), S(O)CR³R^{3a}, CR³R^{3a}S(O)₂, S(O)₂CR³R^{3a}, S(O)NR^{3b}, and S(O)₂NR^{3b}, wherein the right side of Z/G₁ is attached to ring A/ring G, provided that neither Z nor G form an N—S, NCH₂N, NCH₂O, or NCH₂S bond with either group to which it is attached;
A is selected from: C₅₋₁₀ carbocycle substituted with 0-2 R⁴, and 5-10 membered heterocycle consisting of: carbon atoms and 1-4 heteroatoms selected from the group consisting of N, O, and S(O)ₚ and substituted with 0-2 R⁴;
B is selected from Y, X—Y, N(B¹)C(O)C(R³R^{3g})NB²B³, N(B¹)C(O)C(R³R^{3g})C(R³R^{3g})NB²B³, 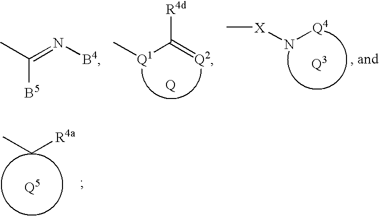
provided that Z and B are attached to different atoms on A, the R^{4d} shown is other than OH, and that the A-X—N moiety forms other than a N—N—N group;
B¹ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —(CH₂)₀₋₁—C₃₋₇ carbocycle substituted with 0-2 R^{4b}, and —(CH₂)₀₋₁-5-6 membered heterocycle consisting of: carbon atoms and 1-4 heteroatoms selected from the group consisting of N, O, and S(O)ₚ and substituted with 0-2 R^{4b};
B² is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, NR^{2d}R^{2d}, CH₂NR^{2d}R^{2d}, CH₂CH₂—NR^{2d}R^{2d}, C(O)R^{2e}, C(O)NR^{2d}R^{2d}, SO₂NR^{2d}R^{2d}, and S(O)ₚR^{5a};
B³ is selected from H, C₁₋₆ alkyl substituted with 0-1 R^{4c}, —(CH₂)₀₋₁-3-6 membered carbocycle substituted with 0-1 R⁵, and a —(CH₂)₀₋₁-5-6 membered heterocycle consisting of: carbon atoms and 1-4 heteroatoms selected from the group consisting of N, O, and S(O)ₚ and substituted with 0-1 R⁵;
B⁴ is selected from H, SO₂R^{3b}, C(O)R^{3b}, SO₂NR³R^{3b}, C(O)NR³R^{3b}, OR², and —CN;
B⁵ is NR²R^{2f} or CR³R²R^{2f};
ring Q is a 5-6 membered ring consisting of, in addition to the Q¹-CR-^{4d}=Q² group shown, carbon atoms and 0-2 heteroatoms selected from N, O, and S(O)ₚ, and the ring is substituted with an additional 0-2 R^{4d};
Q¹ and Q² are each N;
alternatively, Q¹ is CR³ and R^{4d} is NR²R^{2a} or NR^{3a}B⁴, provided that when Q¹ is CR³, then this R³ group optionally forms a ring with the R² group of R^{4d}, this ring is a 5-6 membered ring consisting of, in addition to the C—C—N shown, carbon atoms and from 0-1 additional heteroatoms selected from N, O, and S(O)ₚ, and this ring is substituted with 0-1 R⁵;
Q⁴ is selected from C═O and SO₂;
ring Q³ is a 4-7 membered monocyclic or tricyclic ring consisting of, in addition to the N-Q⁴ group shown, carbon atoms and 0-2 heteroatoms selected from NR^{4c}, O, S, S(O), and S(O)₂, wherein: 0-2 double bonds are present within the ring and the ring is substituted with 0-2 R⁴;
alternatively, ring Q³ is a 4-7 membered ring to which another ring is fused, wherein: the 4-7 membered ring consists of, in addition to the shown amide group, carbon atoms and 0-2 heteroatoms selected from NR^{4c}, O, S, S(O), and S(O)₂ and 0-1 double bonds are present within the ring; the fusion ring is phenyl or a 5-6 membered heteroaromatic consisting of carbon atoms and 1-2 heteroatoms selected from NR^{4c}, O, and S;
ring Q³, which includes the 4-7 membered ring and the fusion ring, is substituted with 0-3 R⁴;
ring Q⁵ is a C₃-₇ monocyclic carbocycle or 3-7 membered monocyclic heterocycle, wherein the carbocycle or heterocycle consists of: carbon atoms and 0-2 heteroatoms selected from N, O, and S(O)p, the carbocycle or heterocycle further comprises 0-2 double bonds and 0-2 carbonyl groups, and the carbocycle or heterocycle is substituted with 0-2 R⁴;
X is selected from —CR²R^{2a})₁₋₄—, —C(O)—, C(═NR^{1c})—, CR²(NR^{1b}R²)—, —C(O)CR²R^{2a}—, —CR²R^{2a}C(O), —C(O)NR²—, —NR²C(O)—, —C(O)NR²CR²R^{2a}—, —NR²C(O)CR²R^{2a}—, —CR²R^{2a}C(O)NR²—, —CR²R^{2a}NR²C(O)—, —NR²C(O)NR²—, —NR²—, —NR²CR²R^{2a}—, —CR²R^{2a}NR²—, —S(O)₂—, —NR²S(O)₂—, O, —CR²R^{2a}O—, and —OCR²R^{2a}—;
Y is selected from: CY¹Y²R^{4a}, NR³R^{3a}, and C(O)NR³R^{3a};
Y¹ and Y² are independently C₁₋₃ alkyl substituted with 0-2 R⁴;
alternatively, Y is selected from one of the following carbocyclic and heterocycles that are substituted with 1 R^{4a} and 0-2 R⁴: cyclopropyl, cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, isoxazolinyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolyl, benzimidazolyl, benzoxazolyl, benzthiazolyl, indazolyl, benzisoxazolyl, benzisothiazolyl, and isoindazolyl;
Z is selected from a bond, CH₂, CH₂CH₂, CH₂O, OCH₂, C(O), NH, CH₂NH, NHCH₂, CH₂C(O), C(O)CH₂, C(O)NH, NHC(O), NHC(O)CH₂C(O)NH, S(O)₂, CH₂S(O)₂, S(O)₂(CH₂), SO₂NH, and NHSO₂, wherein the right side of Z is attached to ring A, provided that Z does not form a N—S, NCH₂N, NCH₂O, or NCH₂S bond with either group to which it is attached;
Z² is selected from H, C₁₋₄ alkyl, phenyl, benzyl, C(O)R^{3b}, S(O)R^{3f}, and S(O)₂R^{3f}.
R^{1a}, at each occurrence, is selected from H, —(CH₂)ᵣ—R^{1b}, —(CH(CH₃))ᵣ—R^{1b}, —(C(CH₃)₂)ᵣ—R^{1b}, —O—(CR³R^{3a})ᵣ—R^{1b}, —NR²—(CR³R^{3a})ᵣ—R^{1b}, and —S—(CR³R^{3a})ᵣ—R^{1b}, provided that R^{1a} forms other than an N-halo, N—S, O—O, or N—CN bond;
alternatively, when two R^{1a} groups are attached to adjacent atoms, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;
R^{1b} is selected from H, CH₃, CH₂CH₃, CH₂CH₂CH₃, CH(CH₃)₂, F, Cl, Br, I, —CN, —CHO, CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, NR²C(O)NHR², NR²C(O)₂R^{2a}, OC(O)NR²R^{2a}, C(O)NR²R^{2a}, C(O)NR²(CH₂)ᵣOR², SO₂NR²R^{2a}, NR²SO₂R², C₅₋₆ carbocycle substituted with 0-2 R^{4b}, and 5-6 membered heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅₋₆ carbocycle substituted with 0-2 R^{4b}, a C₅₋₆ carbocyclic-CH₂-group substituted with 0-2 R^{4b}, and 5-6 membered heterocycle consisting of: carbon atoms and 1-4 heteroatoms selected from the group consisting of N, O, and S(O)ₚ, and substituted with 0-2 R^{4b};
R^{2a}, at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅₋₆ carbocycle substituted with 0-2 R^{4b}, and 5-6 membered heterocycle consisting of: carbon atoms and 1-4 heteroatoms selected from the group consisting of N, O, and S(O)ₚ, and substituted with 0-2 R^{4b};
alternatively, NR²R^{2a}forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅₋₆ carbocycle substituted with 0-2 R^{4b}, and 5-6 membered heterocycle consisting of: carbon atoms and 1-4 heteroatoms selected from the group consisting of N, O, and S(O)ₚ, and substituted with 0-2 R^{4b};
R^{2c}, at each occurrence, is selected from CF₃, OH, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-2 R^{4b}, C₅₋₆ carbocycle substituted with 0-2 R^{4b}, and 5-6 membered heterocycle containing from 1-4 heteroatoms selected from the group consisting of N, O, and S(O)ₚ, and substituted with 0-2 R^{4b};
R^{2d}, at each occurrence, is selected from H, R^{4c}, C₁₋₄ alkyl substituted with 0-2 R^{4c}, —(CR³R^{3a})ᵣ—C₃₋₆ carbocycle substituted with 0-2 R^{4c}, and —(CR³R^{3a})ᵣ-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety ;
alternatively, NR^{2d}R^{2d} forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2e}, at each occurrence, is selected from H, R^{4c}, C₁₋₄ alkyl substituted with
R^{2e}, at each occurrence, is selected from H, R^{4c}, C_{1 4} alkyl substituted with 0-2 R^{4c}, —(CR³R^{3a})ᵣ—C₃₋₆ carbocycle substituted with 0-2 R^{4c}, and —(CR³R^{3a})ᵣ-5-6 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;
R^{2f}, at each occurrence, is selected from H, CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl substituted with 0-1 R^{4b}, C₅₋₆ carbocycle substituted with 0-2 R^{4b}, and 5-6 membered heterocycle containing from 1-4 heteroatoms selected from the group consisting of N, O, and S(O)ₚ and substituted with 0-2 R^{4b};
alternatively, CR²R^{2f} forms a 5-6 membered ring consisting of: carbon atoms and 0-2 heteroatoms selected from N, O, and S(O)ₚ, and this ring is substituted with 0-2 R^{4b};
alternatively, NR²R^{2f} forms a 5-6 membered ring consisting of: carbon atoms and 0-2 additional heteroatoms selected from N, O, and S(O)ₚ, and this ring is substituted with 0-2 R^{4b};
alternatively, when B⁵ is NR²R^{2f}, B⁴ and R^{2f} combine to form a 5-6 membered ring consisting of: carbon atoms and 0-2 additional heteroatoms selected from N, O, and S(O)ₚ, and this ring is substituted with 0-2 R^{4b} and the R² group of NR²R^{2f}, in addition to the groups recited below, is selected from SO₂R^{3b} and C(O)R^{3b};
R³, at each occurrence, is selected from H, CH₃, CH₂CH₃, CH₂CH₂CH₃, CH(CH₃)₂, benzyl, and phenyl;
R^{3a}, at each occurrence, is selected from H, CH₃, CH₂CH₃, CH₂CH₂CH₃, CH(CH₃)₂, benzyl, and phenyl;
alternatively, R³ and R^{3a}, together with the nitrogen atom to which they are attached, combine to form a 5 or 6 membered saturated, partially unsaturated, or unsaturated ring consisting of: carbon atoms and the nitrogen atom to which R³ and R^{3a}are attached;
R^{3b}, at each occurrence, is selected from H, CF₃, CH₃, CH₂CH₃, CH₂CH₂CH₃, CH(CH₃)₂, —(CO₀₋₁ alkyl)-5-6 membered carbocycle substituted with 0-1 R^{1a}, and —(C₀₋₁ alkyl)-5-6 membered heterocycle substituted with 0-1 R^{1a} and consisting of: carbon atoms and 1-4 heteroatoms selected from the group consisting of N, O, and S(O)ₚ;
R^{3c}, at each occurrence, is selected from CH₃, CH₂CH₃, CH₂CH₂CH₃, CH(CH₃)₂, benzyl, and phenyl;
R^{3d}, at each occurrence, is selected from H, CH₃, CH₂CH₃, CH₂CH₂CH₃, CH(CH₃)₂, CH₂-phenyl, CH₂CH₂-phenyl, and C(═O)R^{3c};
R⁴, at each occurrence, is selected from ═O, OR², CH₂OR², (CH₂)₂OR², F, Cl, Br, I, C₁₋₄ alkyl, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, NR²C(O)NR²R^{2a}, SO₂NR²R^{2a}, NR²SO₂NR²R^{2a},S(O)ₚR^{5a}, NR²SO₂—C₁₋₄ alkyl, NR²SO₂R⁵, CF₃, CF₂CF₄, 5-6 membered carbocycle substituted with 0-1 R⁵, and a 5-6 membered heterocycle consisting of: carbon atoms and 1-4 heteroatoms selected from the group consisting of N, O, and S(O)ₚ, and substituted with 0-1 R⁵;
R^{4b}, at each occurrence, is selected from H, ═O, OR³, CH₂OR³, F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR³R^{3a}, CH₂NR³R^{3a}, C(O)R³, CH₂—C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, NR³C(O)NR³R^{3a}, CH₂NR³C(O)NR³R^{3a}, C(═NR³)NR³R^{3a}, CH₂C(═NR³)NR³R^{3a}, NR³C(═NR³)NR³R^{3a}, CH₂NR³C(═NR³)NR³R^{3a}, SO₂NR³R^{3a}, CH₂SO₂NR³R^{3a}, NR³SO₂NR³R^{3a}, CH₂NR³SO₂NR³R^{3a}, NR³SO₂—C₁₋₄ alkyl, CH₂NR³SO₂—C₁₋₄ alkyl, NR³SO₂CF₃, CH₂NR³SO₂CF₃, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, CF₃, and CH₂—CF₃;
R^{4c}, at each occurrence, is selected from ═O, (CR³R^{3a})ᵣOR², (CR³R^{3a})ᵣF, (CR³R^{3a})ᵣBr, (CR³R^{3a})ᵣCl, (CR³R^{3a})ᵣCF₃, C₁₋₄ alkyl, C₂₋₃ alkenyl, C₂₋₃ alkynyl, (CR³R^{3a})ᵣCN, (CR³R^{3a})ᵣNO₂, (CR³R^{3a})ᵣNR²R^{2a}, (CR³R^{3a})ᵣN(→O)R²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂R^{5a}, (CR³ R^{3a})ᵣC(O)NR²SO₂R^{5a}, (CR³R^{3a})ᵣS(O)ₚR^{5a}, (CF₂)ᵣCF₃, (CR³R^{3a})ᵣC₃₋₁₀ carbocycle substituted with 0-2 R^{4b}, and (CR³R^{3a})ᵣ5-10 membered heterocycle consisting of carbon atoms and from 1-4 heteroatoms selected from the group consisting of N, O, and S(O)ₚ and substituted with 0-2 R^{4b};
R^{4d}, at each occurrence, is selected from H, CH₂OR², OR², C₁₋₄ alkyl, CH₂—CN, —CN, CH₂NO₂, NO₂, CH₂NR²R^{2a}, NR²R^{2a}, CH₂—C(O)R^{2c}, C(O)R^{2c}, NR²C(O)R^{2b}, (CH₂)ᵣC(O)NR²R^{2a}, NR²C(O)NR²R^{2a}, (CH₂)ᵣCO₂NR²R^{2a}, NR²SO₂NR²R^{2a}, NR²SO₂R⁵, (CH₂)ᵣS(O)ₚR^{5a}, CH₂CF₃, CF₃, CH₂-5-6 membered carbocycle substituted with 0-1 R⁵, 5-6 membered carbocycle substituted with 0-1 R⁵, a CH₂-5-6 membered heterocycle consisting of: carbon atoms and 1-4 heteroatoms selected from the group consisting of N, O, and S(O)ₚ and substituted with 0-1 R⁵, and a 5-6 membered heterocycle consisting of: carbon atoms and 1-4 heteroatoms selected from the group consisting of N, O, and S(O)ₚ and substituted with 0-1 R⁵;
R⁵, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, NR³C(O)NR³R^{3a}, CH(═NOR^{3d}), C(═NR³)NR³R^{3a}, NR³C(═NR³)NR³R^{3a}, SO₂NR³R^{3a}, NR³SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;
R^{5a}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, NR³R^{3a}, CH₂NR³R^{3a}, C(O)R³, CH₂C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, CF₃, CF₂CF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond; and
R⁶, at each occurrence, is selected from H, OH, OR², F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, CN, NO₂, NR²R^{2a}, CH₂NR²R^{2a}, C(O)R^{2b}, CH₂C(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl.

3. A compound according to claim 2, wherein the compound is selected from: 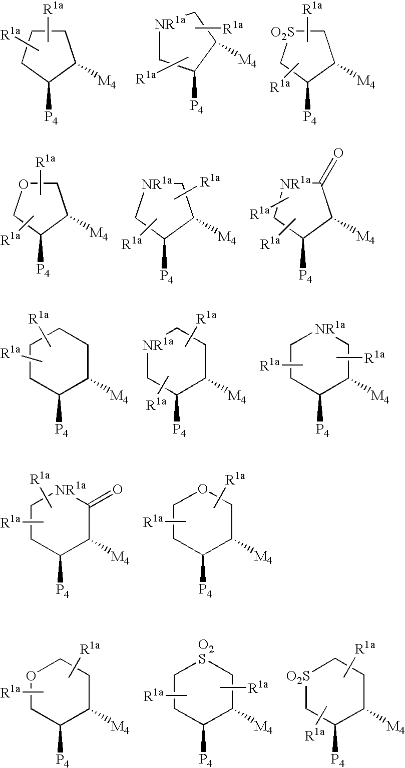 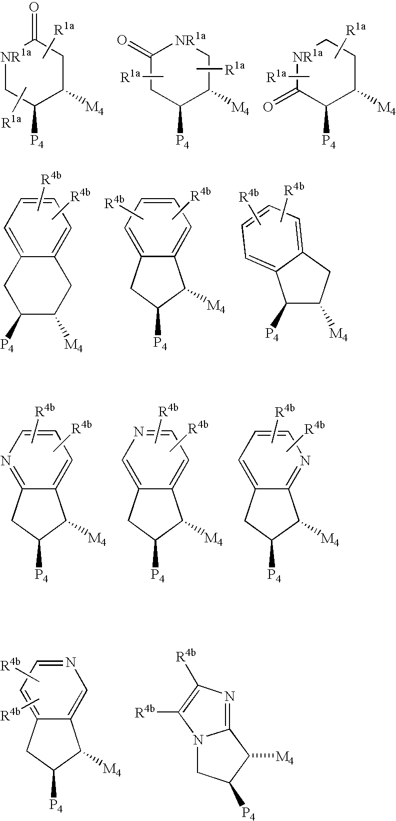 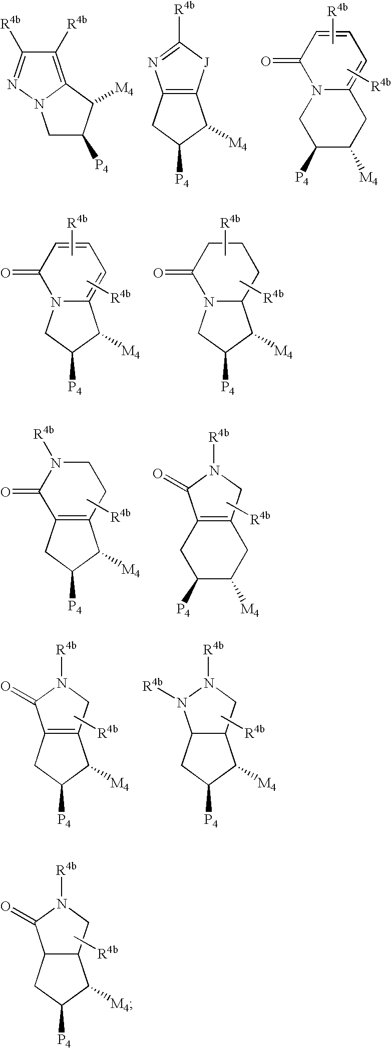
P₄ is G₁-G;
M₄ is Z-A-B;
G is selected from: 2-aminomethyl-4-chloro-phenyl, 2-aminosulfonyl-4-chloro-phenyl, 2-amido-4-chloro-phenyl, 4-chloro-2-methylsulfonyl-phenyl, 2-aminosulfonyl-4-fluoro-phenyl, 2-amido-4-fluoro-phenyl, 4-fluoro-2-methylsulfonyl-phenyl, 2-aminomethyl-4-bromo-phenyl, 2-aminosulfonyl-4-bromo-phenyl, 2-amido-4-bromo-phenyl, 4-bromo-2-methylsulfonyl-phenyl, 2-aminomethyl-4-methyl-phenyl, 2-aminosulfonyl-4-methyl-phenyl, 2-amido-4-methyl-phenyl, 2-methylsulfonyl-4-methyl-phenyl, 4-fluoro-pyrid-2-yl, 4-bromo-pyrid-2-yl, 4-methyl-pyrid-2-yl, 5-fluoro-thien-2-yl, 5-bromo-thien-2-yl, 5-methyl-thien-2-yl, 2-amido-4-methoxy-phenyl, 2-amido-phenyl, 2-aminomethyl-3-fluoro-phenyl, 2-aminomethyl-4-fluoro-phenyl, 2-aminomethyl-5-fluoro-phenyl, 2-aminomethyl-6-fluoro-phenyl, 2-aminomethyl-phenyl, 2-amino-pyrid-4-yl, 2-aminosulfonyl-4-methoxy-phenyl, 2-aminosulfonyl-phenyl, 3-amido-phenyl, 3-amino-4-chloro-phenyl, 3-aminomethyl-phenyl, 2-methoxy-pyrid-5-yl, 5-chloro-pyrid-2-yl, 5-chloro-thien-2-yl, 6-amino-5-chloro-pyrid-2-yl, 6-amino-pyrid-2-yl, 2-cyano-4-chloro-phenyl, 2-methoxy-4-chloro-phenyl, 2-fluoro-4-chloro-phenyl, 3-chloro-4-methyl-phenyl, 3-fluoro-4-chloro-phenyl, 3-methyl-4-chloro-phenyl, 3-fluoro-4-methyl-phenyl, 3,4-dimethyl-phenyl, 3-chloro-4-fluoro-phenyl, 3-methyl-4-fluoro-phenyl, 4-methylsulfanyl-phenyl, 2-chlorothiazol-5-yl, 5-chlorothiazol-2-yl, 3-pyridyl, 2-pyridyl, 5-fluoro-pyrid-2-yl, 5-methyl-pyrid-2-yl, 5-methoxy-pyrid-2-yl, 5-cyano-pyrid-2-yl, 2-pyrimidyl, 5-chloro-pyrimid-2-yl, 5-fluoro-pyrimid-2-yl, 5-methyl-pyrimid-2-yl, 5-methoxy-pyrimid-2-yl, 5-cyano-pyrimid-2-yl, phenyl, 3-chloro-phenyl, 3-fluoro-phenyl, 3-methyl-phenyl, 4-ethyl-phenyl, 3-methoxy-phenyl, 3-cyano-phenyl, 4-chloro-phenyl, 4-fluoro-phenyl, 4-methyl-phenyl, 4-methoxy-phenyl, 4-cyano-phenyl, 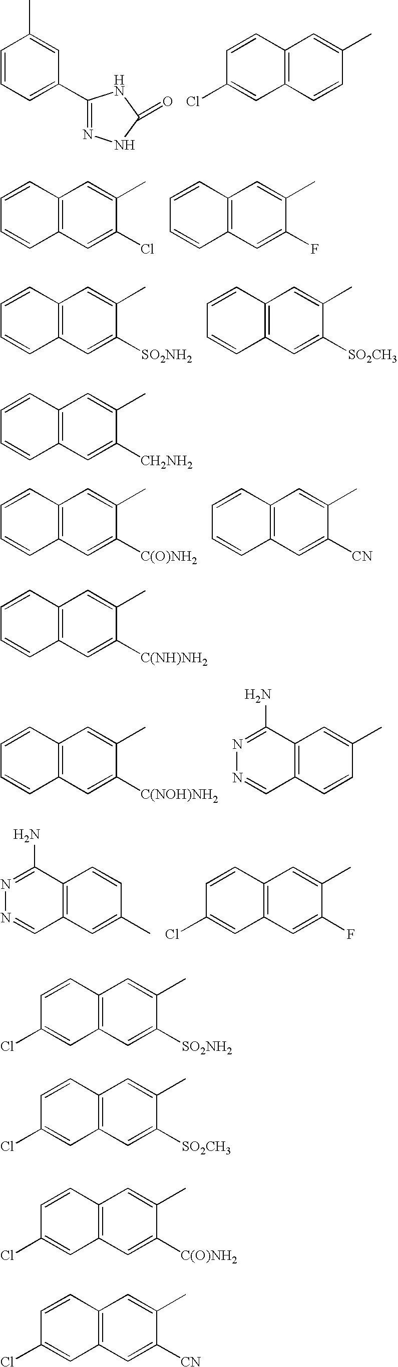 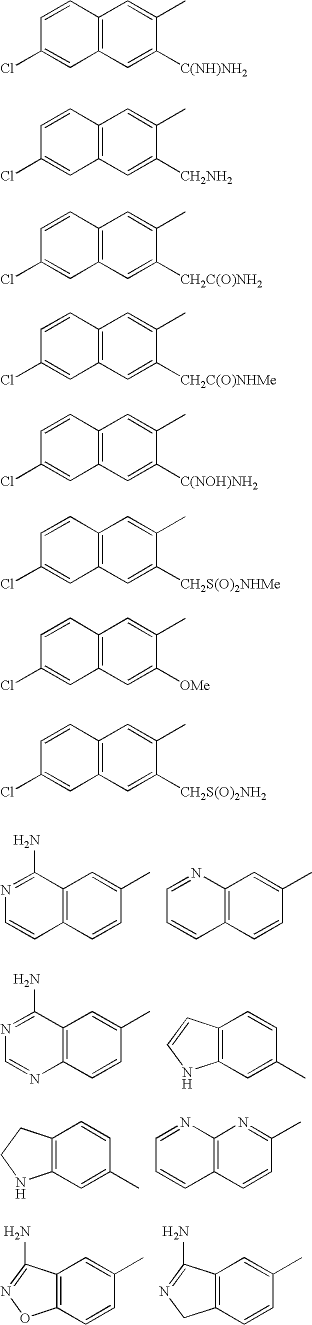 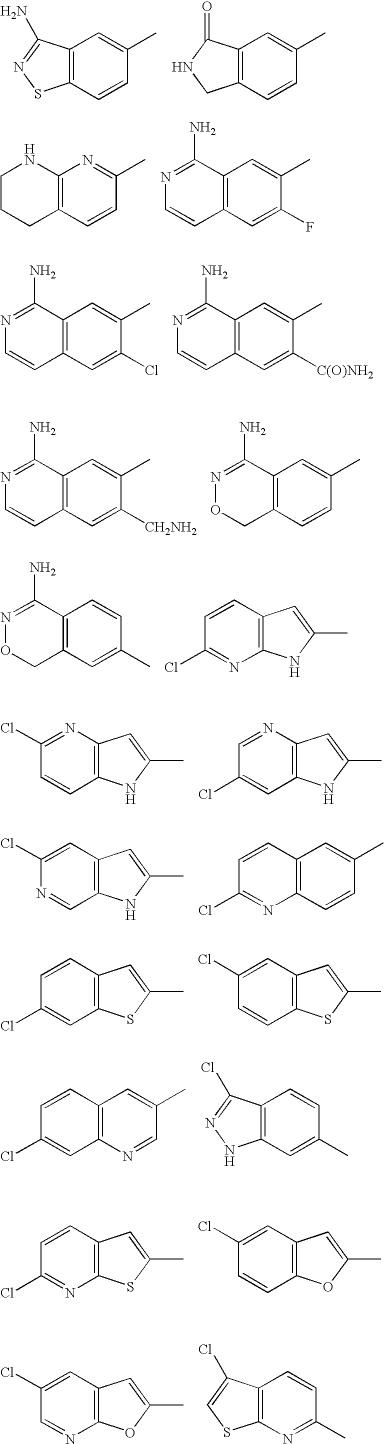 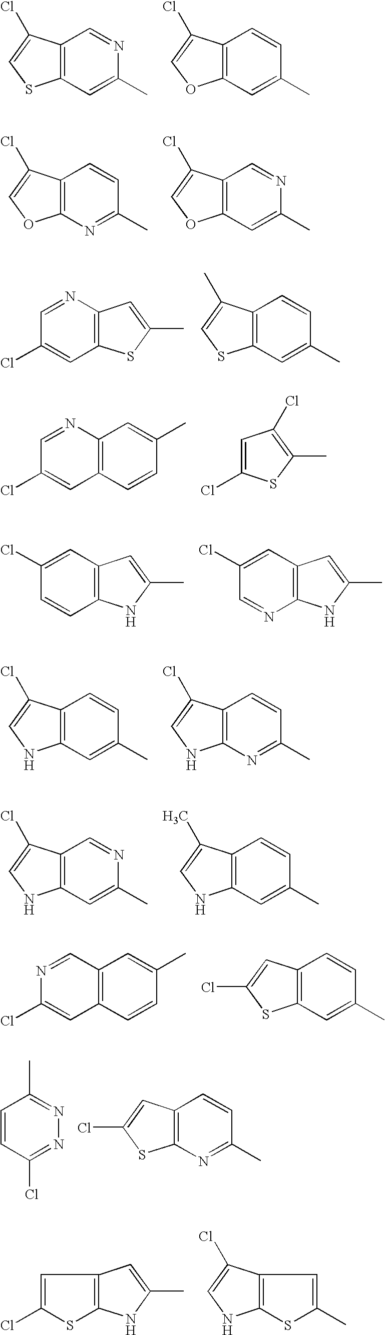 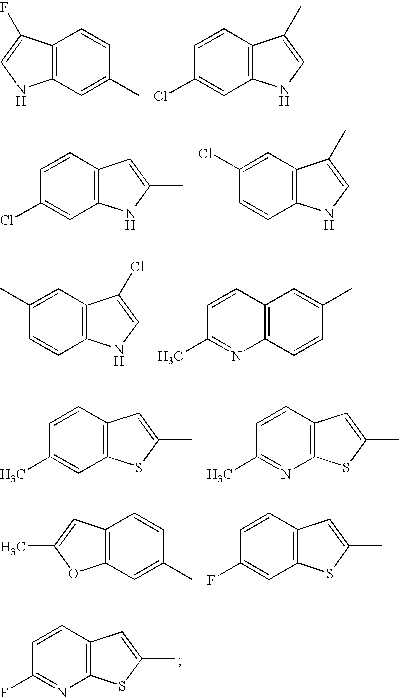
one of G₁ and Z is selected from CH₂CH₂, CH₂C(O), C(O)CH₂, C(O)O, OC(O), CH₂O, OCH₂, CH₂NH, NHCH₂, C(O)NH, NHC(O), CH₂S(O), S(O)CH₂, CH₂S(O)₂, S(O)₂CH₂, S(O)NH, NHS(O)₂, and S(O)₂NH, and the other of G₁ and Z is selected from CH₂CH₂, CH₂C(O), C(O)CH₂, C(O)O, OC(O), CH₂O, OCH₂, CH₂NH, C(O)NH, CH₂S(O), S(O)CH₂, CH₂S(O)₂, S(O)₂CH₂, S(O)NH, and S(O)₂NH, wherein the right side of Z/G₁ is attached to ring A/ring G, provided that neither Z nor G form an N—S, NCH₂N, NCH₂O, or NCH₂S bond with either group to which it is attached;
A is selected from one of the following carbocyclic and heterocyclic groups, which are substituted with 0-2 R⁴, cyclohexyl, phenyl, piperidinyl, piperazinyl, pyridyl, pyrimidyl, furanyl, morpholinyl, thienyl, pyrrolyl, pyrrolidinyl, oxazolyl, isoxazolyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolinyl, indolyl, benzimidazolyl, benzoxazolyl, benzthiazolyl, indazolyl, benzisoxazolyl, benzisothiazolyl, and isoindazolyl;
B¹ is selected from H, CH₃, CH₂CH₃, CH₂CH₂CH₃, CH(CH₃)₂, —(CH₂)₀₋₁—C₅₋₆ carbocycle substituted with 0-2 R^{4b}, and —(CH₂)₀₋₁-5-6 membered heterocycle consisting of: carbon atoms and 1-4 heteroatoms selected from the group consisting of N, O, and S(O)ₚ and substituted with 0-2 R^{4b};
B² is selected from H, CH₃, CH₂CH₃, CH₂CH₂CH₃, CH(CH₃)₂, C(O)R^{2e}, C(O)NR^{2d}R^{2d}, SO₂NR^{2d}R^{2d}, and S(O)ₚR^{5a};
B³ is selected from H, C₁₋₆alkyl substituted with 0-1 R^{4c}, —(CH₂)₀₋₁-3-6 membered carbocycle substituted with 0-1 R⁵, and a —(CH₂)₀₋₁-5-6 membered heterocycle consisting of: carbon atoms and 1-4 heteroatoms selected from the group consisting of N, O, and S(O)ₚ and substituted with 0-1 R⁵;
B⁴ is selected from H, SO₂R^{3b} and OR²;
B⁵ is NR²R^{2f};
ring Q is a 5-6 membered ring consisting of, in addition to the N—CR^{4d}═N group shown, carbon atoms and 0-2 heteroatoms selected from N, O, and S(O)ₚ, and the ring is substituted with an additional 0-2 R^{4d};
Q⁴ is selected from C═O and SO₂;
ring Q³ is a 5-7 membered ring consisting of, in addition to the N-Q⁴ group shown, carbon atoms and 0-2 heteroatoms selected from NR^{4c}, O, S, S(O), and S(O)₂, wherein: 0-2 double bonds are present within the ring and the ring is substituted with 0-2 R^{4a};
alternatively, ring Q³ is a 5-7 membered ring to which another ring is fused, wherein: the 5-7 membered ring consists of, in addition to the shown amide group, carbon atoms and 0-2 heteroatoms selected from NR^{4c}, O, S, S(O), and S(O)₂, and 0-1 double bonds are present within the ring; the fusion ring is phenyl or a 5-6 membered heteroaromatic consisting of carbon atoms and 1-2 heteroatoms selected from NR^{4c}, O, and S;
ring Q³, which includes the 5-7 membered ring and the fusion ring, is substituted with 0-3 R^{4a};
ring Q⁵, is a C₃₋₆ monocyclic carbocycle or 5-6 membered monocyclic heterocycle, wherein the carobocycle or heterocycle consists of carbon atoms and 0-2 heteroatoms selected from N, O, and S(O)p, the carbocycle or heterocycle further comprises 0-1 double bonds and 0-1 carbonyl groups, and the carbocycle or heterocycle is substituted with 0-2 R⁴;
X is selected from —(CR²R^{2a})₁₋₂—, —C(═NR^{1b})—, —C(O)—, —S(O)₂—, —NR²S(O)₂—, —NR²S(O)₂—, —NR²C(O)—, —C(O)NR²—, —NR²C(O)CR²R^{2a}—, —NR²C(O)NR²—, NR²—NR²CR²R^{2a}—, —CR²R^{2a}NR²—, O, —OCR²R^{2a}—, and —CR²R^{2a}O—;
Y is selected from: CY¹Y²R^{4a}, NR³R^{3a}, and C(O)NR³R^{3a};
Y¹ and Y² are independently C₁₋₂ alkyl substituted with 0-2 R⁴;
alternatively, Y is selected from one of the following carbocyclic and heterocycles that are substituted with 1 R^{4a} and 0-1 R⁴: cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, thiazolyl, isothiazolyl, pyrazolyl, imidazolyl, oxadiazole, thiadiazole, triazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,5-triazole, 1,3,4-triazole, benzofuran, benzothiofuran, indole, benzimidazole, benzimidazolone, benzoxazole, benzthiazole, indazole, benzisoxazole, benzisothiazole, and isoindazole;
R^{1a} is selected from H, R^{1b}, CH(CH₃)R^{1b}, C(CH₃)₂R^{1b}, CH₂R^{1b}, and CH₂CH₂R^{1b}, provided that R^{1a} forms other than an N-halo, N—S, or N—CN bond;
alternatively, when two R^{1a} groups are attached to adjacent atoms, together with the atoms to which they are attached they form a 5-6 membered ring consisting of: carbon atoms and 0-2 heteroatoms selected from the group consisting of N, O, and S(O)ₚ, this ring being substituted with 0-2 R^{4b} and 0-3 ring double bonds;
R^{1b} is selected from H, CH₃, CH₂CH₃, F, Cl, Br, —CN, —CHO, CF₃, OR², NR²R^{2a}, C(O )R^{2b}, CO₂R^{2b}, OC(O)R², CO₂R^{2a}, S(O)ₚR², NR²(CH₂)ᵣOR^{2 NR}²C(O)R^{2b}, C(O)NR²R^{2a}, SO₂NR²R^{2a}, NR²SO₂R², phenyl substituted with 0-2 R^{4b}, and 5-6 membered aromatic heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, phenyl substituted with 0-2 R^{4b}, a benzyl substituted with 0-2 R^{4b}, and a 5-6 membered aromatic heterocycle consisting of: carbon atoms and 1-4 heteroatoms selected from the group consisting of N, O, and S(O)ₚ, and substituted with 0-2 R^{4b};
R^{2a}, at each occurrence, is selected from H, CF₃, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle consisting of: carbon atoms and 1-4 heteroatoms selected from the group consisting of N, O, and S(O)ₚ, and substituted with 0-2 R^{4b};
alternatively, NR²R^{2a}forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle consisting of: carbon atoms and 1-4 heteroatoms selected from the group consisting of N, O, and S(O)ₚ, and substituted with 0-2 R^{4b};
R^{2c}, at each occurrence, is selected from CF₃, OH, OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-2 R^{4b}, phenyl substituted with 0-2 R^{4b}, and 5-6 membered aromatic heterocycle containing from 1-4 heteroatoms selected from the group consisting of N, O, and S(O)ₚ, and substituted with 0-2 R^{4b};
R^{2d}, at each occurrence, is selected from H, R^{4c}, C₁₋₄ alkyl substituted with 0-2 R^{4c}, C₃₋₆ carbocycle substituted with 0-2 R^{4c}, —(CR³R^{3a})—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CR³R^{3a})-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;
R^{2e}, at each occurrence, is selected from H, R^{4c}, C₁₋₄ alkyl substituted with 0-2 R^{4c}, C₃₋₆ carbocycle substituted with 0-2 R^{4c}, —(CR³R^{3a})—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CR³R^{3a})-5-6 membered heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;
R^{2f}, at each occurrence, is selected from H, CF₃, CH₃, CH₂CH₃, CH₂CH₂CH₃, CH(CH₃)₂, OCH₃, and benzyl;
alternatively, NR²R^{2f} forms a 5-6 membered ring consisting of: carbon atoms and 0-2 additional heteroatoms selected from N, O, and S(O)ₚ, and this ring is substituted with 0-2 R^{4b};
alternatively, B⁴ and R^{2f} combine to form a 5-6 membered ring consisting of: carbon atoms and 0-1 additional heteroatoms selected from N, O, and S(O)ₚ, and this ring is substituted with 0-2 R^{4b} and the R² group of NR²R^{2f}, in addition to the groups recited below, can be SO₂R^{3b};
R^{3b}, at each occurrence, is selected from H, CF₃, CH₃, CH₂CH₃, CH₂CH₂CH₃, and CH(CH₃)₂;
R⁴, at each occurrence, is selected from H, ═O, CH₂OR², (CH₂)₂OR², OR², F, Cl, Br, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, NR²C(O)NR²R^{2a}, SO₂NR²R^{2a}, CF₃, and CF₂CF₃;
R^{4a} is selected from —(CR³R^{3g})ᵣ-5-6 membered carbocycle substituted with 0-3 R^{4c}, —(CR³R^{3g})ᵣ-5-6 membered heterocycle substituted with 0-3 R^{4c} and consisting of: carbon atoms and 1-4 heteroatoms selected from the group consisting of N, O, and S(O)ₚ, (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣ—NR^{2d}C(O)R^{2e}, (CR³R^{3g})ᵣ—C(O)R^{2e}, (CR³R^{3g})ᵣ—OC(O)R^{2e}, (CR³R^{3g})ᵣ—C(O)NR^{2d}R^{2d}, (CR³R^{3g})ᵣ—C(O)OR^{2d}, (CR³R^{3g})ᵣNR^{2d}C(O)NR^{2d}R^{2d}, (CR³R^{3g})ᵣ—NR^{2d}C(O)OR^{2d}, (CR³R^{3g})ᵣ—SO₂NR^{2d}R^{2d}, (CR³R^{3g})ᵣ—NR^{2d}SO₂R^{2d}, and (CR³R^{3g})ᵣ—S(O)ₚR^{2d}, provided that S(O)ₚR^{2d} forms other than S(O)₂H or S(O)H;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, CH₂—C(O)R³, C(O)OR^{3c}, CH₂—C(O)OR^{3c}, NR³C(O)R^{3a}, CH₂NR³C(O)R^{3a}, C(O)NR³R^{3a}, CH₂—C(O)NR³R^{3a}, SO₂NR³R^{3a}, CH₂SO₂NR³R^{3a}, NR³SO₂—C₁₋₄ alkyl, CH₂NR³SO₂—C₁₋₄ alkyl, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, and CF₃;
R^{4c}, at each occurrence, is selected from ═O, OR², (CR³R^{3a})OR², F, (CR³R^{3a})F, Br, (CR³R^{3a})Br, Cl, (CR³R^{3a})Cl, CF₃, (CR³R^{3a})CF₃, C₂₋₃ alkenyl, C₂₋₃ alkynyl, C₁₋₄ alkyl, —CN, (CR³R^{3a})CN, NO₂, (CR³R^{3a})NO₂, NR²R^{2a}, (CR³R^{3a})NR²R^{2a}, N(→O)R²R^{2a}, (CR³R^{3a})N(→O)R²R^{2a}, C(O)R^{2c}, (CR³R^{3a})C(O)R^{2c}, NR²C(O)R^{2b}, (CR³R^{3a})NR²C(O)R^{2b}, C(O)NR²R^{2a}, (CR³R^{3a})C(O)NR²R^{2a}, NR²C(O)NR²R^{2a}, (CR³R^{3a})NR²C(O)NR²R^{2a}, SO₂NR²R^{2a}, (CR³R^{3a})SO₂NR²R^{2a}, NR²SO₂NR²R^{2a}, (CR³R^{3a})NR²SO₂NR²R^{2a}, NR²SO₂R^{5a}, (CR³R^{3a})NR²SO₂R^{5a}, S(O)ₚR^{5a}, (CR³R^{3a})S(O)ₚR^{5a}, CF₃, CF₂CF₃, C₃₋₁₀ carbocycle substituted with 0-2 R^{4b}, (CR³R^{3a})C₃₋₁₀ carbocycle substituted with 0-2 R^{4b}, 5-10 membered heterocycle consisting of carbon atoms and from 1-4 heteroatoms selected from the group consisting of N, O, and S(O)ₚ and substituted with 0-2 R^{4b}, and (CR³R^{3a})-5-10 membered heterocycle consisting of carbon atoms and from 1-4 heteroatoms selected from the group consisting of N, O, and S(O)ₚ and substituted with 0-2 R^{4b};
R^{4d}, at each occurrence, is selected from H, CH₂OR², OR², CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, CH₂NR²R^{2a}, NR²R^{2a}, C(O)R^{2c}, NR²C(O)R^{2b}, C(O)NR²R^{2a}, NR²C(O)NR²R^{2a}, NR²SO₂R⁵, SO₂NR²R^{2a}, 6 membered carbocycle substituted with 0-1 R⁵, and a 5-6 membered heterocycle consisting of: carbon atoms and 1-2 heteroatoms selected from the group consisting of N, O, and S(O)ₚ and substituted with 0-1 R⁵;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, SO₂NR²R^{2a}, and NR²SO₂C₁₋₄ alkyl.

4. A compound according to claim 3, wherein the compound is selected from: 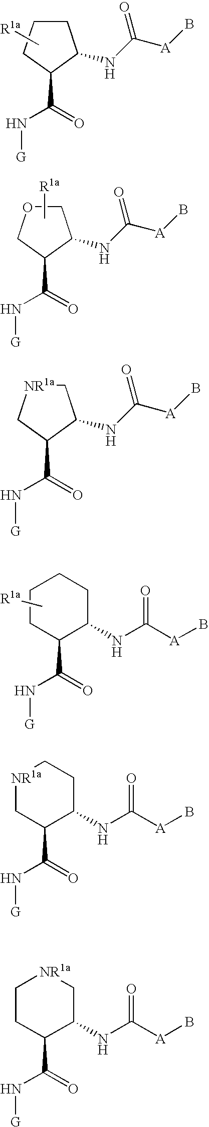 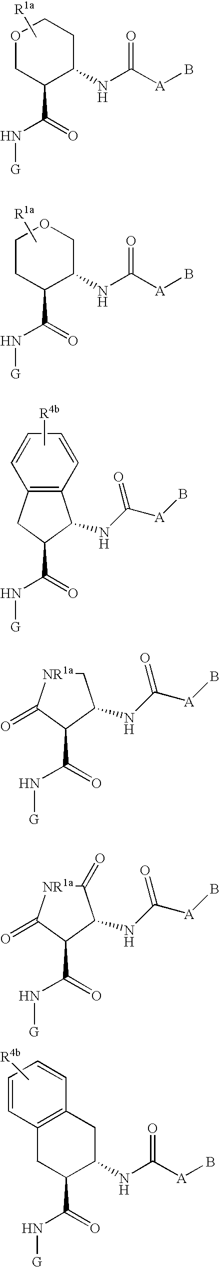 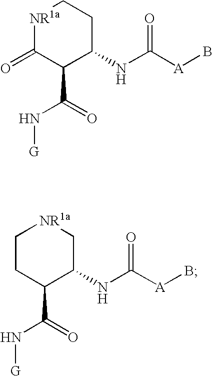
G is selected from: 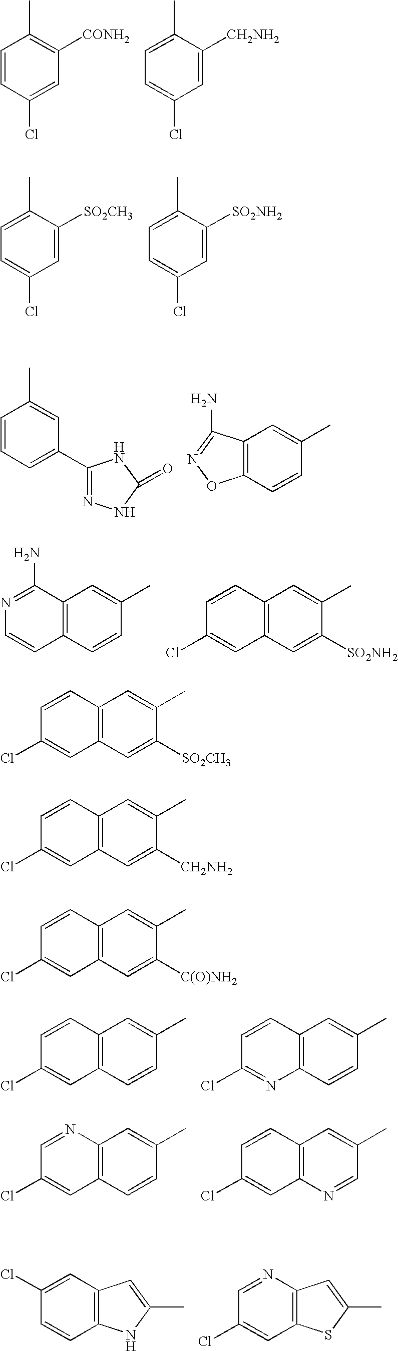 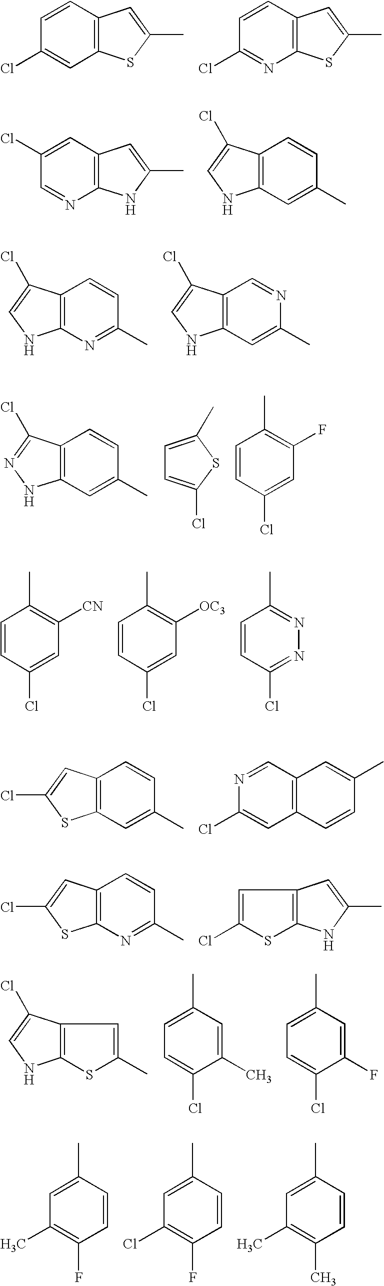 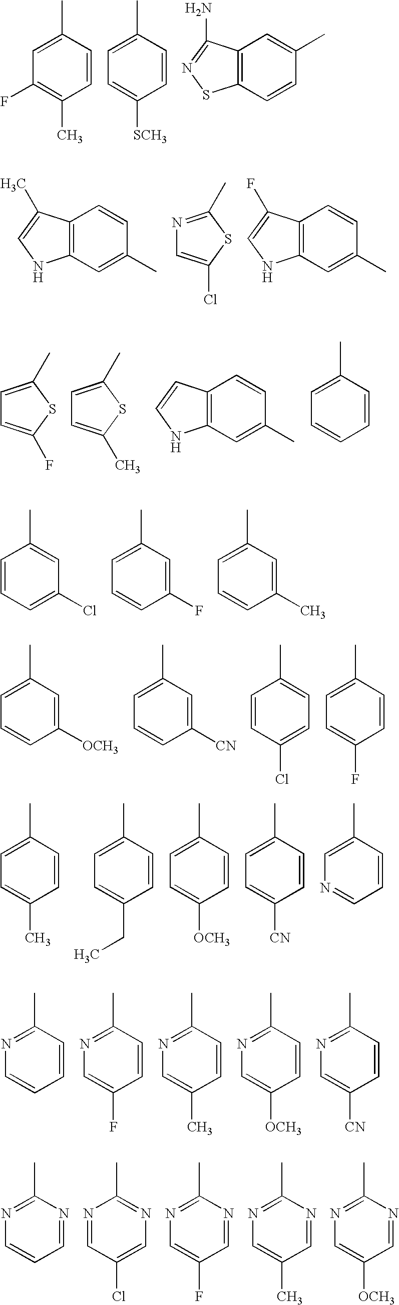 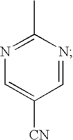
one of G₁ and Z is selected from NHCH₂, NHC(O), CH₂S(O), CH₂S(O)₂, and NHS(O)₂, and the other of GI and Z is selected from C(O)CH₂, C(O)O, C(O)NH, S(O)CH₂, S(O)₂CH₂, S(O)NH, and S(O)₂NH, wherein the right side of Z/G₁ is attached to ring A/ring G, provided that neither Z nor G form an N—S, NCH₂N, NCH₂O, or NCH₂S bond with either group to which it is attached;
A is selected from cyclohexyl, piperidinyl, piperazinyl, phenyl, pyridyl, and pyrimidyl, and is substituted with 0-2 R⁴;
B is selected from Y, N(B¹)C(O)C(R³R^{3g})NB²B³, 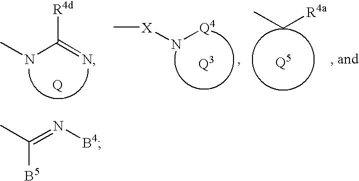
provided that Z and B are attached to different atoms on A, the R^{4d} shown is other than OH, and that the A-X—N moiety forms other than a N—N—N group;
B¹ is selected from H, CH₃, CH₂CH₃, CH₂CH₂CH₃, and CH(CH₃)₂;
B² is selected from H, CH₃, CH₂CH₃, CH₂CH₂CH₃, and CH(CH₃)₂;
B³ is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, C₂₋₅ alkyl substituted with 1 R^{4c}, —(CH₂)₀₋₁-3-6 membered carbocycle substituted with 0-1 R⁵, and a —(CH₂)₀₋₁-5-6 membered heterocycle consisting of: carbon atoms and 1-4 heteroatoms selected from the group consisting of N, O, and S(O)ₚ and substituted with 0-1 R⁵;
B⁴ is selected from H, SO₂R^{3b}, and OR²;
B⁵ is NR²R^{2f};
ring Q is a 5-6 membered ring consisting of, in addition to the N—CR^{4d}═N group shown, carbon atoms and 0-1 heteroatoms selected from N, O, and S(O)ₚ, and the ring is substituted with an additional 0-2 R^{4d};
Q⁴ is selected from C═O and SO₂;
ring Q³ is a 6-7 membered ring consisting of, in addition to the N-Q⁴ group shown, carbon atoms and 0-1 heteroatoms selected from NR^{4c}, O, S, S(O), and S(O)₂, wherein: 0-2 double bonds are present within the ring and the ring is substituted with 0-2 R⁴;
alternatively, ring Q³ is a 5-7 membered ring to which another ring is fused, wherein: the 5-7 membered ring consists of, in addition to the shown amide group, carbon atoms and 0-1 heteroatoms selected from NR^{4c}, O, S, S(O), and S(O)₂, and 0-1 double bonds are present within the ring; the fusion ring is phenyl;
ring Q³, which includes the 5-7 membered ring and the fusion ring, is substituted with 0-2 R⁴;
ring Q⁵ is substituted with 0-1 R⁴ and is selected from cyclopropyl, cyclobutyl, cyclopentyl, cyclopentanonyl, cyclohexyl, cyclohexanonyl, pyrrolidinyl, pyrrolidinonyl, piperidinyl, piperidinonyl, tetrahydrofuranyl, and tetrahydropyranyl;
X is selected from CH₂, C(O), —S(O)₂—, —NHC(O)—, —C(O)NH—, —CH₂NH—, O, and —CH₂O—;
Y is selected from N(CH₃)₂, C(O)(CH₃)₂, C(CH₃)₂R^{4a} and C(CH₂CH₃)₂R^{4a};
altneratively, Y is selected from phenyl, pyridyl, pyrrolidino, N-pyrrolidino-carbonyl, morpholino, N-morpholino-carbonyl, 1,2,3-triazolyl, imidazolyl, and benzimidazolyl, and is substituted with 1 R^{4a} and 0-1 R⁴;
R^{1a}, at each occurrence, is selected from H, R^{1b}, CH(CH₃)R^{1b}, C(CH₃)₂R^{1b}, and CH₂R^{1b}, provided that R^{1a} forms other than an N-halo, N—S, or N—CN bond;
R^{1b} is selected from CH₃, CH₂CH₃, F, Cl, Br, —CN, CF₃, OR², NR²R^{2a}, C(O)R^{2b}, CO₂R^{2b}, CO₂R^{2a}, S(O)ₚR², C(O)NR²R^{2a}, SO₂NR²R^{2a}, NR²SO₂R², and 5-6 membered aromatic heterocycle consisting of carbon atoms and from 1-4 heteroatoms selected from the group consisting of N, O, and S(O)ₚ, and substituted with 0-2 R^{4b}, provided that R^{1b} forms other than an O—O, N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};
R^{2a}, at each occurrence, is selected from H, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};
alternatively, NR²R^{2a}forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle consisting of: carbon atoms and 1-4 heteroatoms selected from the group consisting of N, O, and S(O)ₚ, and substituted with 0-1 R^{4b};
R^{2c}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl substituted with 0-1 R^{4b}, phenyl substituted with 0-1 R^{4b}, and 5-6 membered aromatic heterocycle containing from 1-4 heteroatoms selected from the group consisting of N, O, and S(O)ₚ, and substituted with 0-1 R^{4b};
R^{2d}, at each occurrence, is selected from H, R^{4c}, C₁₋₄ alkyl substituted with 0-2 R^{4c}, C₃₋₆ carbocycle substituted with 0-2 R^{4c}, —(CH₂)-C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)-5-6 membered heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ—S—O, O—N, O—S, or O—O moiety;
R^{2e}, at each occurrence, is selected from H, R^{4c}, C₁₋₄ alkyl substituted with 0-2 R^{4c}, C₃₋₆ carbocycle substituted with 0-2 R^{4c}, —(CH₂—C₃₋₆ carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)-5-6 membered heterocycle and consisting of: carbon atoms and 1-4 heteroatoms selected from the group consisting of N, O, and S(O)ₚ, provided that R^{2e} forms other than a C(O)-halo or C(O)—S(O)ₚ moiety;
R^{2f}, at each occurrence, is selected from H, CH₃, CH₂CH₃, OCH₃, and benzyl;
alternatively, NR²R^{2f} forms a 5-6 membered ring consisting of: carbon atoms and 0-1 additional heteroatoms selected from N, O, and S(O)ₚ, and this ring is substituted with 0-1 R^{4b};
alternatively, B⁴ and R^{2f} combine to form a 5 membered ring consisting of: carbon atoms and 0-1 additional heteroatoms selected from N, O, and S(O)ₚ, and this ring is substituted with 0-2 R^{4b} and the R² group of NR²R^{2f}, in addition to the groups recited below, can be SO₂R^{3b};
R^{3b}, at each occurrence, is selected from H and CH₃;
R⁴, at each occurrence, is selected from H, ═O, OH, OR², CH₂OR², (CH₂)₂OR², F, Br, Cl, I, CH₃, CH₂CH₃, CH₂CH₂CH₃, CH(CH₃)₂, CH₂CH₂CH₃, CH₂CH(CH₃)₂, CH(CH₃)CH₂CH₃, C(CH₃)₃, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, CF₃, and CF₂CF₃;
R^{4a} is selected from —(CR³R^{3g})ᵣ-5-6 membered carbocycle substituted with 0-3 R^{4c}, —(CR³R^{3g})ᵣ-5-6 membered heterocycle substituted with 0-3 R^{4c} and consisting of: carbon atoms and 1-4 heteroatoms selected from the group consisting of N, O, and S(O)ₚ, (CR³R^{3g})ᵣNR^{2d}R^{2d}, (CR³R^{3g})ᵣN(→O)R^{2d}R^{2d}, (CR³R^{3g})ᵣOR^{2d}, (CR³R^{3g})ᵣ—C(O)NR^{2d}R^{2d}, (CR³R^{3g})ᵣ—NR^{2d}C(O)R^{2e}, (CR³R^{3g})ᵣ—C(O)R^{2e}, (CR³R^{3g})ᵣ—NR^{2d}C(O)NR^{2d}R^{2d}, (CR³R^{3g})ᵣ—NR^{2d}C(O)OR^{2d}, (CR³R^{3g})ᵣ—NR^{2d}SO₂R^{2d}, and (CR³R^{3g})ᵣ—S(O)ₚR^{2d}, provided that S(O)ₚR^{2d} forms other than S(O)₂H or S(O)H;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, and CF₃;
R^{4c}, at each occurrence, is selected from ═O, OR², CH₂OR², F, Br, Cl, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, C₂₋₃ alkenyl, C₂₋₃ alkynyl, —CN, NO₂, NR²R^{2a}, CH₂NR²R^{2a}, N(→O)R²R^{2a}, CH₂N(→O)R²R^{2a}, C(O)R^{2c}, CH₂C(O)R^{2c}, NR²C(O)R^{2b}, CH₂NR²C(O)R^{2b}, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, CH₂S(O)ₚR^{5a}, CF₃, CF₂CF₃, C₃₋₆ carbocycle substituted with 0-2 R^{4b}, (CH₂)C₃₋₆ carbocycle substitute with 0-2 R^{4b}, 5-6 membered heterocycle consisting of carbon atoms and from 1-4 heteroatoms selected from the group consisting of N, O, and S(O)ₚ and substituted with 0-2 R^{4b}, and (CH₂)-5-6 membered heterocycle consisting of carbon atoms and from 1-4 heteroatoms selected from the group consisting of N, O, and S(O)ₚ and substituted with 0-2 R^{4b};
R^{4d}, at each occurrence, is selected from H, CH₂OR², OR², CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, CH₂NR²R^{2a}, NR²R^{2a}, C(O)R^{2c}, NR²C(O)R^{2b}, C(O)NR²R^{2a}, SO₂NR²R^{2a}, NR²SO₂R⁵, phenyl substituted with 0-1 R⁵, and a 5-6 membered heterocycle consisting of: carbon atoms and 1 heteroatom selected from the group consisting of N, O, and S(O)ₚ and substituted with 0-1 R⁵;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂-C₁₋₄ alkyl, NR³SO₂-phenyl, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, and SO₂NR²R^{2a}.

5. A compound according to claim 4, wherein the compound is selected from: 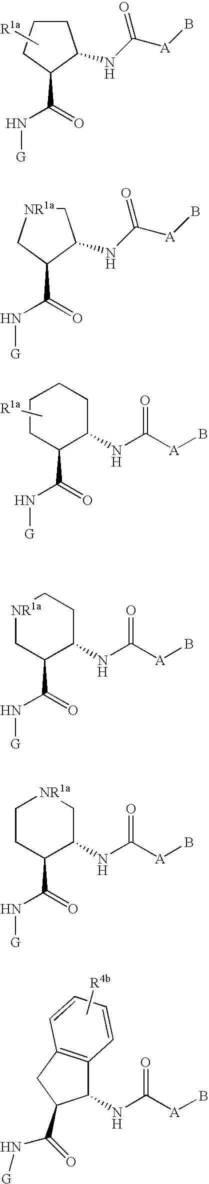
G is selected from: 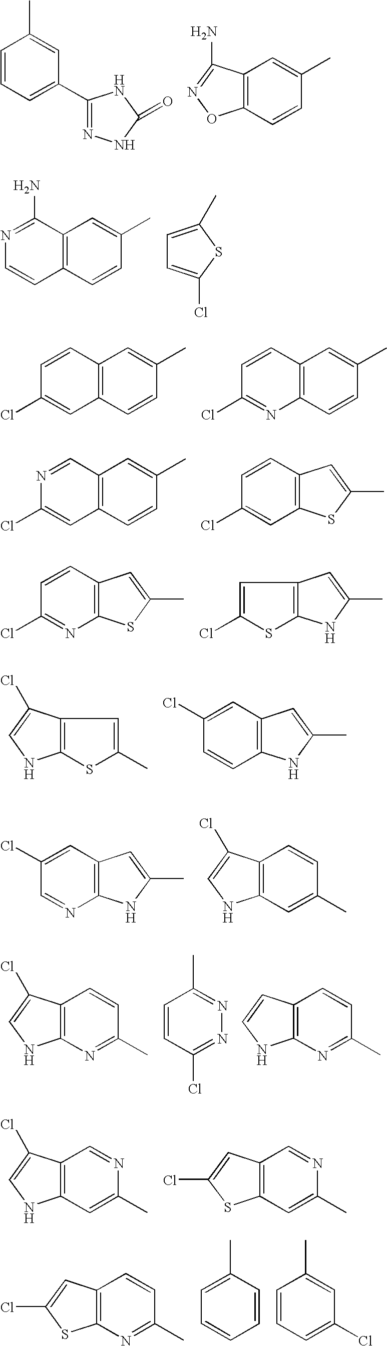 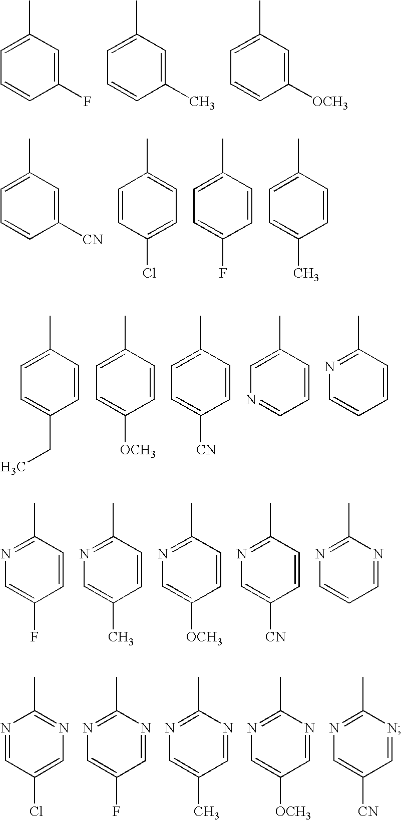
A is selected from the group: cyclohexyl, phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;
B is selected from Y, N(B¹)C(O)C(R³R^{3g})NB²B³, 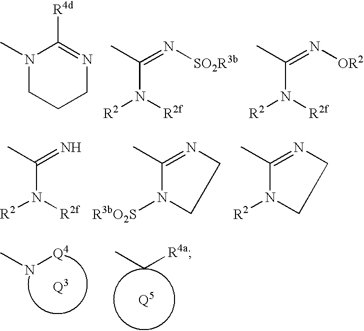
provided that Z and B are attached to different atoms on A and that the R^{4d} shown is other than OH;
B¹ is selected from H, CH₃, CH₂CH₃, and CH₂CH₂CH₃;
B² is selected from H, CH₃, and CH₂CH₃;
B³ is selected from CH₃, CH₂CH₃, CH₂CH₂CH₃, CH(CH₃)₂, CH₂CH₂CH₂CH₃, C(CH₃)₃, CH(CH₃)CH₂CH(CH₃)₂, CH₂CH₂OH, CH(CH₃)CH₂OH, CH(phenyl)CH₂CH₃, cyclopropyl, cyclobutyl, cyclopentyl, cyclohexyl, and CH₂-cyclopropyl; 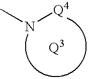
is attached to a different atom on A than M and is selected from the group: 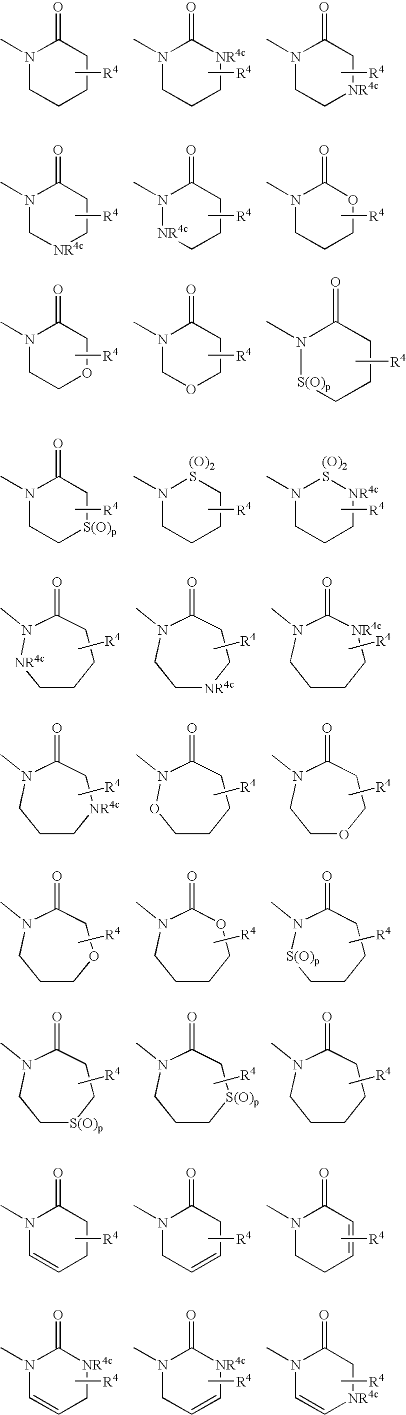 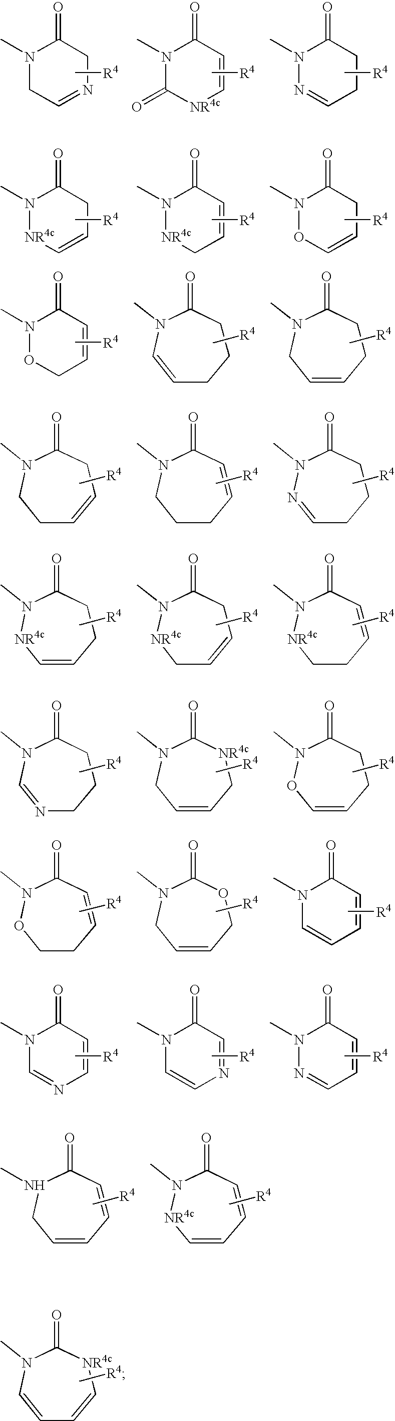
ring Q⁵ is selected from cyclopropyl, cyclobutyl, cyclopentyl, 2-cyclopentanonyl, cyclohexyl, 2-cyclohexanonyl, pyrrolidinyl (attached to A and R^{4a} at the 2-position), pyrrolidinyl (attached to A and R^{4a} at the 3-position), 2-pyrrolidinonyl (attached to A and R^{4a} at the 3-position), piperidinyl (attached to A and R^{4a} at the 4-position), 4-piperdinonyl (attached to A and R^{4a} at the 3-position), tetrahydrofuranyl, and tetrahydropyranyl (attached to A and R^{4a} at the 4-position);
Y is selected from N(CH₃)₂, C(O)(CH₃)₂, C(CH₃)₂R^{4a} and C(CH₂CH₃)₂R^{4a};
alternatively, Y is selected from phenyl, pyridyl, 1,2,3-triazolyl, imidazolyl, and benzimidazolyl, and is substituted with 1 R^{4a};
R^{1a}, at each occurrence, is selected from H, CH₃, CH₂CH₃, CH₂CH₂CH₃, CH₂F, CH₂Cl, Br, CH₂Br, —CN, CH₂CN, CF₃, CH₂CF₃, OCH₃, CH₂OH, C(CH₃)₂OH, CH₂OCH₃, NH₂, CH₂NH₂, NHCH₃, CH₂NHCH₃, N(CH₃)₂, CH₂N(CH₃)₂, CO₂H, COCH₃, CO₂CH₃, CH₂CO₂CH₃, SCH₃, CH₂SCH₃, S(O)CH₃, CH₂S(O)CH₃, S(O)₂CH₃, CH₂S(O)₂CH₃, C(O)NH₂, CH₂C(O)NH₂, SO₂NH₂, CH₂SO₂NH₂, NHSO₂CH₃, CH₂NHSO₂CH₃, pyridin-2-yl, pyridin-3-yl, pyridin-4yl, pyridin-2-yl-N-oxide, pyridin-3-yl-N-oxide, pyridin-4-yl-N-oxide, imidazol-1-yl, CH₂-imidazol-1-yl, 4-methyl-oxazol-2-yl, 4-N,N-dimethylaminomethyl-oxazol-2-yl, 1,2,3,4-tetrazol-1-yl, 1,2,3,4-tetrazol-5-yl, CH₂-1,2,3,4-tetrazol-1-yl, and CH₂-1,2,3,4-tetrazol-5-yl, provided that R^{1a} forms other than an N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5 membered aromatic heterocycle consisting of: carbon atoms and 1-4 heteroatoms selected from the group consisting of N, O, and S(O)ₚ, and substituted with 0-1 R^{4b};
R^{2a}, at each occurrence, is selected from H, CH₃, and CH₂CH₃;
alternatively, NR²R^{2a}forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OCH₃, OCH₂CH₃, CH₃, and CH₂CH₃;
R^{2c}, at each occurrence, is selected from OH, OCH₃, OCH₂CH₃, CH₃, and CH₂CH₃;
R^{2d}, at each occurrence, is selected from H, R^{4c}, C₁₋₄ alkyl substituted with 0-2 R^{4c}, C₃₋₆ cycloalkyl substituted with 0-2 R^{4c}, phenyl substituted with 0-2 R^{4c}, and 5-6 membered aromatic heterocycle substituted with 0-2 R^{4c} and consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;
R^{2e}, at each occurrence, is selected from H, R^{4c}, C₁₋₄ alkyl substituted with 0-2 R^{4c}, C₃₋₆ cycloalkyl substituted with 0-2 R^{4c}, phenyl substituted with 0-2 R^{4c}, and 5-6 membered aromatic heterocycle substituted with 0-2 R^{4c} and consisting of: carbon atoms and 1-4 heteroatoms selected from the group consisting of N, O, and S(O)ₚ, provided that R^{2e} forms other than a C(O)-halo or C(O)—S(O)ₚ moiety;
R^{2f}, at each occurrence, is selected from H, CH₃, CH₂CH₃, and OCH₃;
alternatively, NR²R^{2f} forms a ring selected from morpholine, piperazine, piperidine, and pyrrolidine;
R⁴, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, and C(CH₂)₃;
R^{4a} is selected from —(CH₂)ᵣ-5-6 membered carbocycle substituted with 0-3 R^{4c}, —(CH₂)ᵣ-5-6 membered heterocycle substituted with 0-3 R^{4c} and consisting of: carbon atoms and 1-4 heteroatoms selected from the group consisting of N, O, and S(O)ₚ, (CH₂)ᵣNR^{2d}R^{2d}, (CH₂)ᵣN(→O)R^{2d}R^{2d}, (CH₂)ᵣOR^{2d}, (CH₂)ᵣC(O)NR^{2d}R^{2d}, (CH₂)ᵣ—NR^{2d}C(O)R^{2e}, (CH₂)ᵣ—C(O)R^{2e}, (CH₂)ᵣ—NR^{2d}C(O)NR^{2d}R^{2d}, (CH₂)ᵣ—NR^{2d}C(O)OR^{2d}, (CH₂)ᵣ—NR^{2d}SO₂R^{2d}, and (CH₂)ᵣ—S(O)ₚR^{2d}, provided that S(O)ₚR^{2d} forms other than S(O)₂H or S(O)H;
R^{4b}, at each occurrence, is selected from H, ═O, OR³, CH₂OR³, F, Cl, CH₃, CH₂CH₃, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂-phenyl, S(O)₂CH₃, S(O)₂-phenyl, and CF₃;
R^{4c}, at each occurrence, is selected from ═O, OH, OCH₃, OCH₂CH₃, OCH₂CH₂CH₃, OCH(CH₃)₂, CH₃, CH₂CH₃, CH₂CH₂CH₃, CH(CH₃)₂, C₂₋₃ alkenyl, C₂₋₃ alkynyl, CH₂OH, CH₂OCH₃, CH₂OCH₂CH₃, CH₂OCH₂CH₂CH₃, CH₂OCH(CH₃)₂, F, Br, Cl, CF₃, NR²R^{2a}, CH₂NR²R^{2a}, N(→O)R²R^{2a}, CH₂N(→O)R²R^{2a}, C(O)R^{2c}, CH₂C(O)R^{2c}, NR²C(O)R^{2b}, CH₂NR²C(O)R²b, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, CH₂S(O)ₚR^{5a}, CF₃, cyclopropyl substituted with 0-1 R^{4b}, cyclobutyl substituted with 0-1 R^{4b}, cyclopentyl substituted with 0-1 R^{4b}, phenyl substituted with 0-1 R^{4b}, —CH₂-cyclopropyl substituted with β-l R^{4b}, —CH₂-cyclobutyl substituted with 0-1 R^{4b}, —CH₂-cyclopentyl substituted with 0-1 R^{4b}, benzyl substituted with 0-2 R^{4b}, 5-6 membered aromatic heterocycle consisting of carbon atoms and from 1-4 heteroatoms selected from the group consisting of N, O, and S(O)ₚ and substituted with 0-2 R^{4b}, and (CH₂)-5-6 membered aromatic heterocycle consisting of carbon atoms and from 1-4 heteroatoms selected from the group consisting of N, O, and S(O)ₚ and substituted with 0-2 R^{4b};
R^{4d}, at each occurrence, is selected from H, OCH₃, CH₃, CH₂CH₃, CH₂CH₂CH₃, CH(CH₃)₂, NR²R^{2a}, NR²C(O)R²b, NR²SO₂R⁵, phenyl, 2-oxo-pyrrolidinyl, and 2-oxo-piperidinyl;
R⁵, at each occurrence, is selected from H, ═O, CH₃, CH₂CH₃, OR³, CH₂OR³, F, Cl, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)₂—CH₃, S(O)₂-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, NR²R^{2a}, CH₂NR²R^{2a}, C(O)R^{2b}, CH₂C(O)R^{2b}, NR²C(O)R^{2b}, and SO₂NR²R^{2a}.

6. A compound according to claim 5, wherein the compound is selected from:
A is selected from the group: phenyl, 2-pyridyl, 2-pyrimidyl, and 2-F-phenyl, wherein B is substituted at the 4-position of A;
B is selected from: 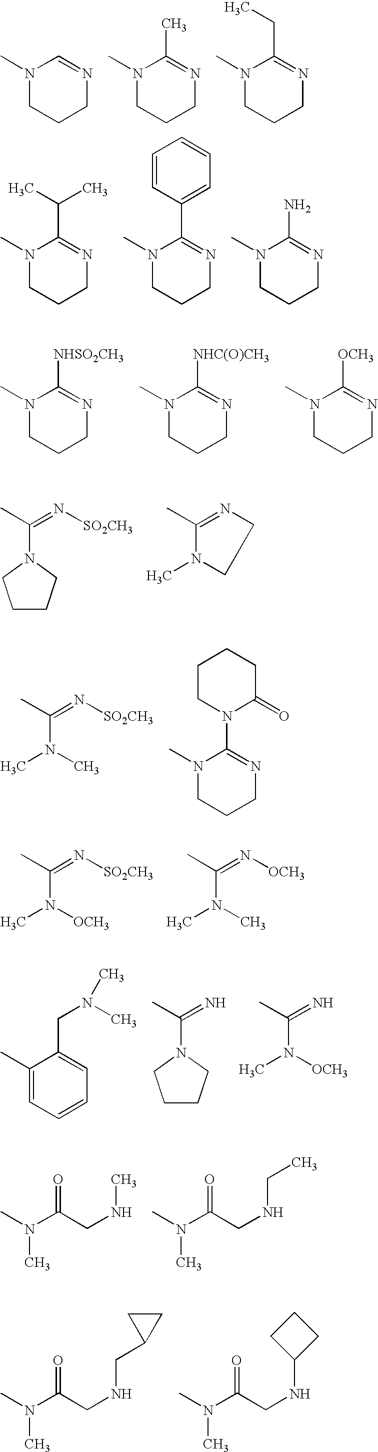 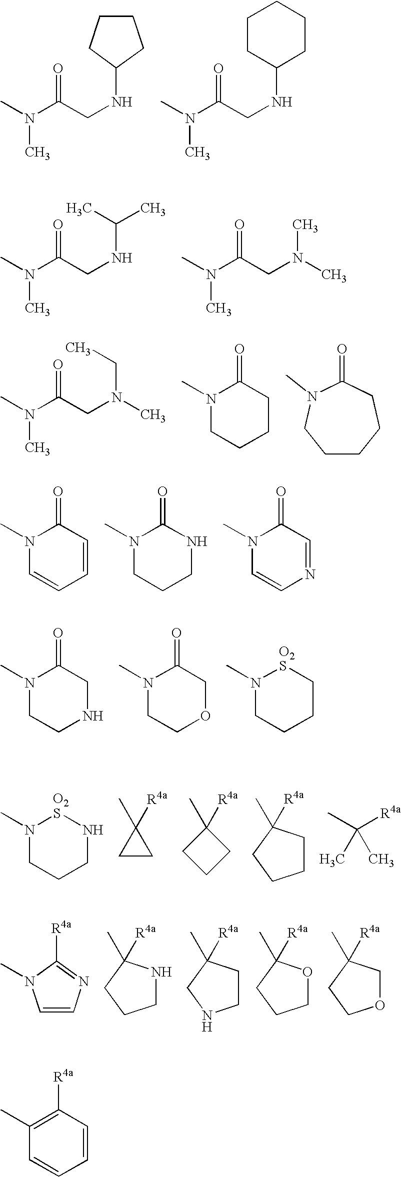
R^{2d}, at each occurrence, is selected from H. C₁₋₄ alkyl substituted with 0-1 R^{4c}, C₃₋₆ cycloalkyl substituted with 0-2 R^{4c}, phenyl substituted with 0-2 R^{4c}, and 5-6 membered aromatic heterocycle consisting of: carbon atoms and 1-4 heteroatoms selected from the group consisting of N, O, and S(O)ₚ, provided that R^{2d} forms other than a N-halo, N—C-halo, S(O)ₚ-halo, O-halo, N—S, S—N, S(O)ₚ—S(O)ₚ, S—O, O—N, O—S, or O—O moiety;
R^{2e}, at each occurrence, is selected from H, C₁₋₄ alkyl substituted with 0-1 R^{4e}, C₃₋₆ cycloalkyl substituted with 0-2 R^{4c}, phenyl, substituted with 0-2 R^{4c}, and 5-6 membered aromatic heterocycle consisting of: carbon atoms and 1-4 heteroatoms selected from the group consisting of N, O, and S(O)ₚ, provided that R^{2e} forms other than a C(O)-halo or C(O)—S(O)ₚ moiety;
R^{4a} is selected from NR^{2d}R^{2d}, CH₂NR^{2d}R^{2d}, CH₂CH₂NR^{2d}R^{2d}, N(→O)R^{2d}R^{2d}, CH₂N(→O)R^{2d}R^{2d}, CH₂OR^{2d}, C(→O)R^{2e}, C(O)NR^{2d}R^{2d}, CH₂C(O)NR^{2d}, NR^{2d}C(O)R^{2e}, CH₂NR^{2d}C(O)R^{2e}, NR^{2d}C(O)NR^{2d}R^{2d}, CH₂NR^{2d}C(O)NR^{2d}R^{2d}, NR^{2d}C(O)OR^{2d}, CH₂NR^{2d}C(O)OR^{2d}, NR^{2d}SO₂R^{2d}, CH₂NR^{2d}SO₂R^{2d}, S(O)ₚR^{2d}, CH₂S(O)ₚR^{2d}, 5-6 membered carbocycle substituted with 0-2 R^{4c}, —(CH₂)-5-6 membered carbocycle substituted with 0-2 R^{4c}, 5-6 membered heterocycle substituted with 0-2 R^{4c} and consisting of: carbon atoms and 1-4 heteroatoms selected from the group consisting of N, O, and S(O)ₚ, and —(CH₂)— 5-6 membered heterocycle substituted with 0-2 R^{4c} and consisting of: carbon atoms and 1-4 heteroatoms selected from the group consisting of N, O, and S(O)ₚ provided that S(O)ₚR^{2d} forms other than S(O)₂H or S(O)H; and
R^{4c} is selected from ═O, OH, OCH₃, OCH₂CH₃, OCH₂CH₂CH₃, OCH(CH₃)₂, CH₃, CH₂CH₃, CH₂CH₂CH₃, CH(CH₃)₂, CH═CH₂, CH≡CH, CH₂OH, CH₂OCH₃, CH₂OCH₂CH₃, CH₂OCH₂CH₂CH₃, CH₂OCH(CH₃)₂, F, Br, Cl, CF₃, NR²R^{2a}, CH₂NR²R^{2a}, C(O)R^{2c}, CH₂C(O)R^{2c}, NR²C(O)R^{2b}, CH₂NR²C(O)R^{2b}, C(O)NR²R^{2a}, CH₂C(O)NR²R^{2a}, SO₂NR²R^{2a}, CH₂SO₂NR²R^{2a}, NR²SO₂R^{5a}, CH₂NR²SO₂R^{5a}, S(O)ₚR^{5a}, and CH₂S(O)ₚR^{5a}.

7. A compound according to claim 6, wherein the compound is selected from:
A-B is selected from: 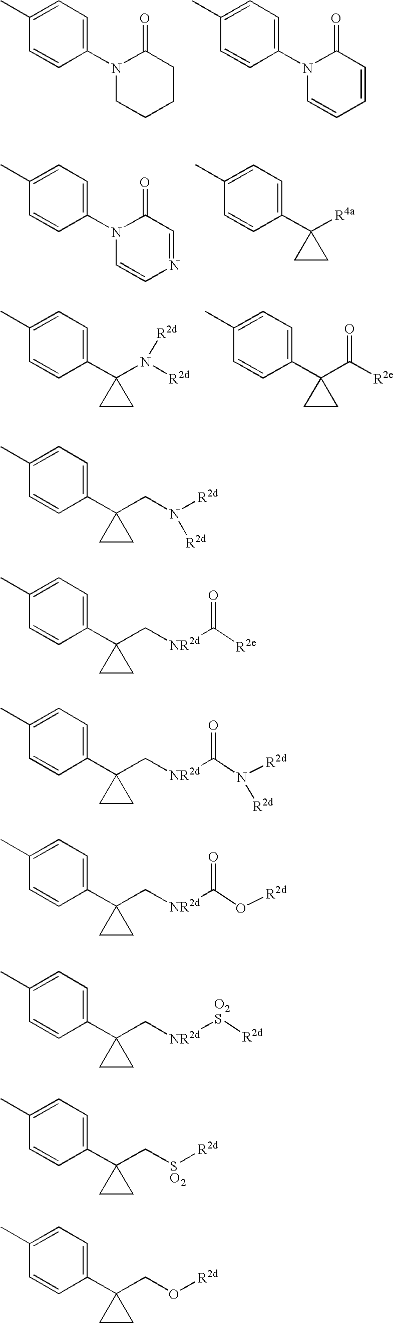 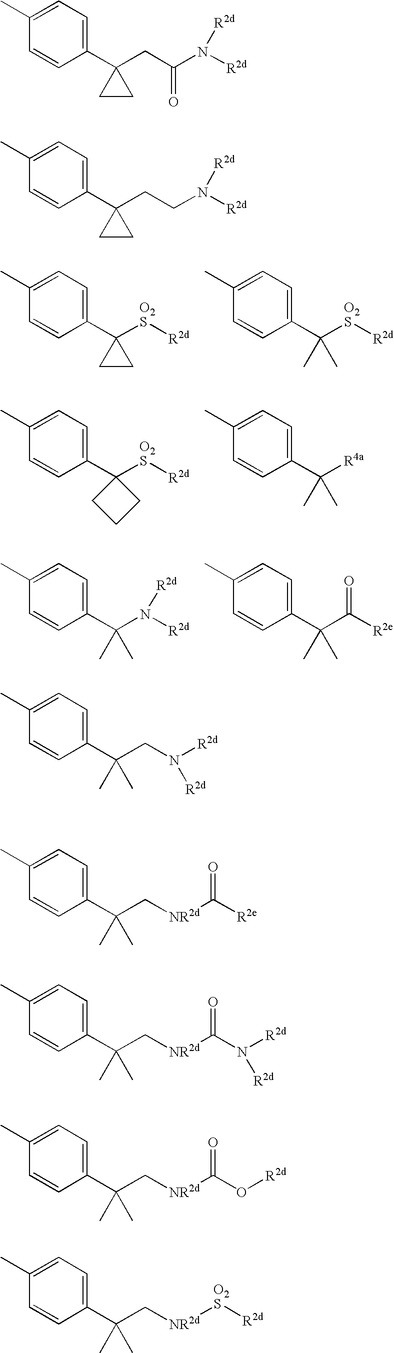 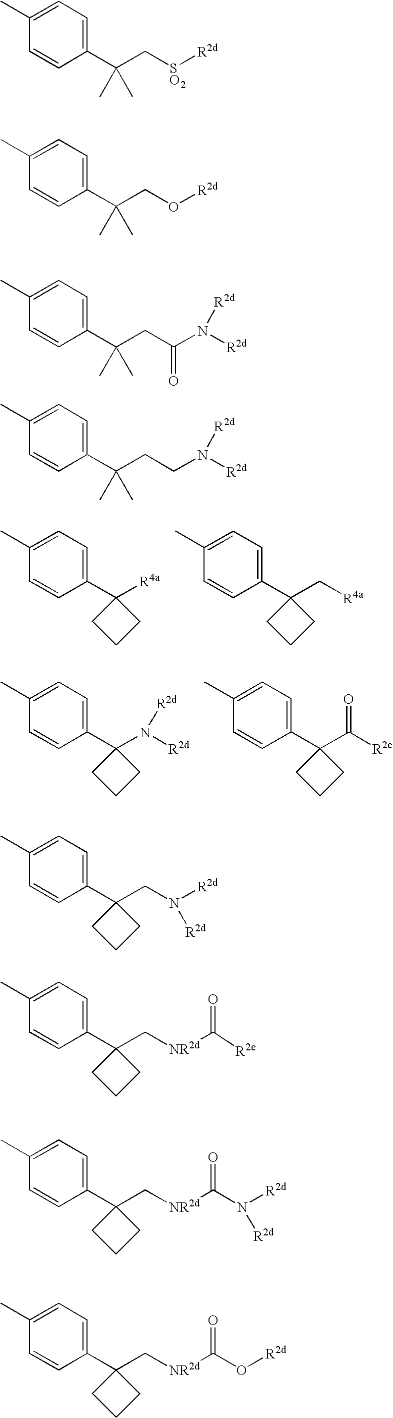 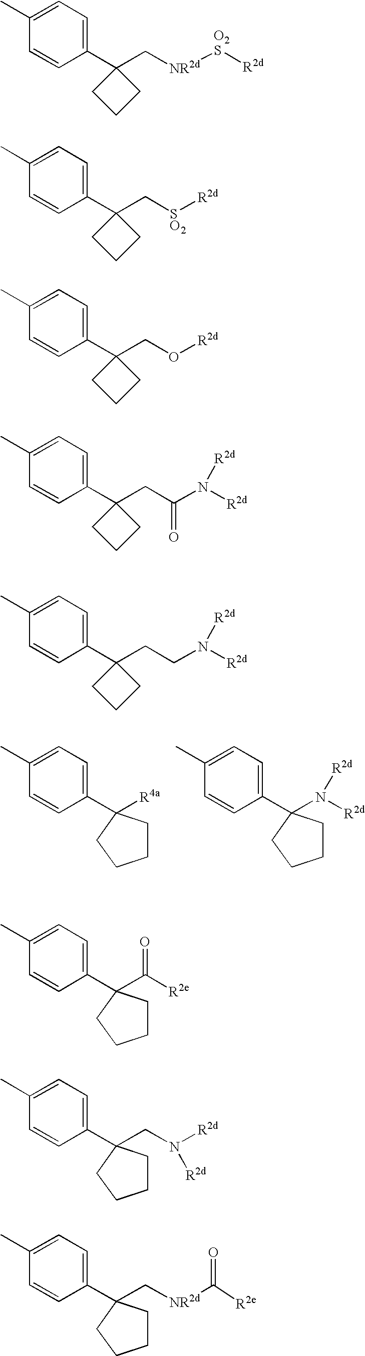 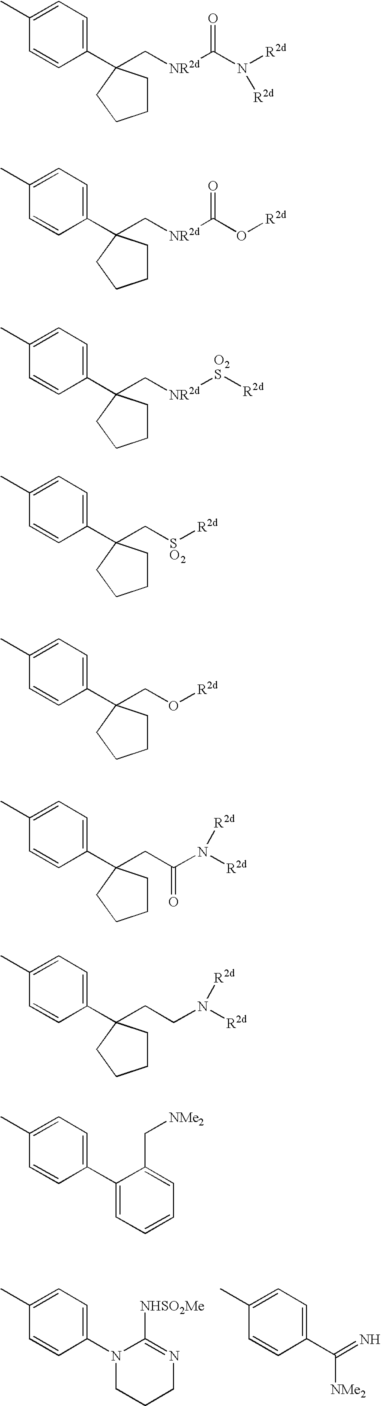 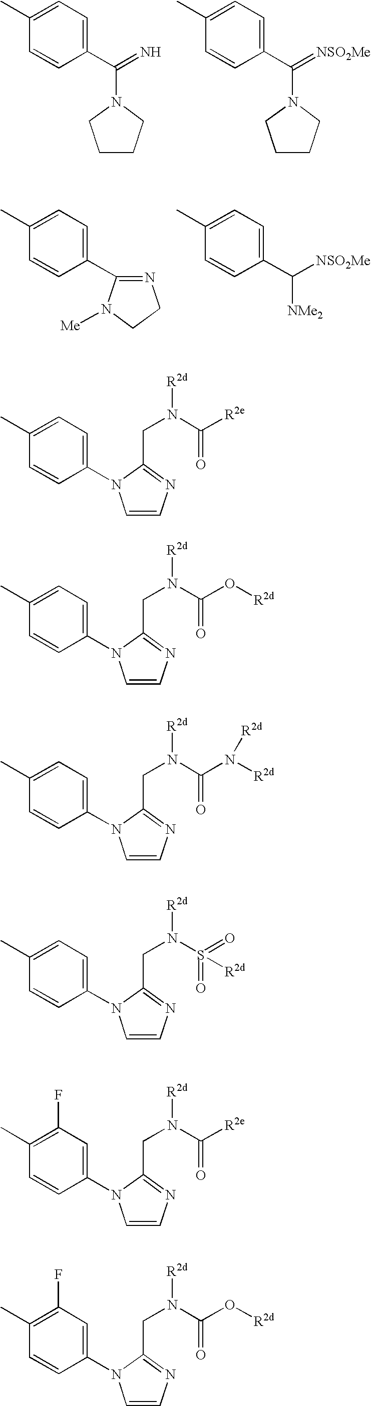 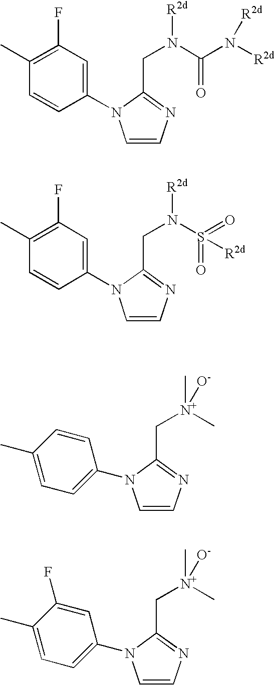
R^{2d}, at each occurrence, is selected from H, CH₃, CH₂CH₃, CH₂CH₂CH₃, CH(CH₃)₂, CH₂CH(CH₃)₂, CH₂CH₂CH(CH₃)₂, CH₂CCH, CH₂CH₂OH, CH₂C(O)NH₂, cyclopropyl, CH₂-cyclopropyl, cyclobutyl, cyclopentyl, and thiazolyl;
R^{2e}, at each occurrence, is selected from CH₃, CH₂CH₃, CH₂CH₂CH₃, CH(CH₃)₂, CH₂CH(CH₃)₂, CH₂CH₂CH(CH₃)₂, CH₂-cyclopropyl, cyclopropyl, and cyclopentyl;
R^{4a} is substituted with 0-2 R^{4c} and selected from morpholine, 1,1-dioxo-thiomorpholine, dihydropyridine, piperidine, piperazine, pyrrolidine, imidazole, imidazoline, imidazolidine, oxazoline, and thiazoline; and
R^{4c} is selected from ═O, OH, OCH₃, and CH₃.

8. A compound according to claim 1, wherein the compound is selected from: trans-N-2-((5-chloropyridin-2-yl)carbamoyl)cyclohexyl)-4-(2-oxopyridin-1(2H)-yl)benzamide, and trans-N-((1S,2S)-2-((4-(2-oxopyridin-1(2H)-yl)phenyl)carbamoyl)cyclohexyl)-4-chlorobenzamide, or a pharmaceutically acceptable salt or solvate form thereof.

9. A pharmaceutical composition, comprising: a pharmaceutically acceptable carrier and a therapeutically effective amount of a compound of claim 1 or a pharmaceutically acceptable salt or solvate form thereof.

10. A method for treating a thromboembolic disorder, comprising: administering to a patient in need thereof a therapeutically effective amount of a compound of claim 1 or a pharmaceutically acceptable salt or solvate form thereof.

11. A method according to claim 10, wherein the thromboembolic disorder is selected from the group consisting of arterial cardiovascular thromboembolic disorders, venous cardiovascular thromboembolic disorders, and thromboembolic disorders in the chambers of the heart.

12. A method according to claim 10, wherein the thromboembolic disorder is selected from unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.
